(19) United States
(12) Patent Application Publication
(10) Pub. No.: US 2016/0118598 A1
(43) Pub. Date: Apr. 28, 2016

(54) ELECTRON TRANSPORT MATERIAL AND ORGANIC ELECTROLUMINESCENT DEVICE USING SAME

(71) Applicant: JNC CORPORATION, Tokyo (JP)

(72) Inventors: Daisuke BABA, Chiba (JP); Yohei ONO, Chiba (JP)

(73) Assignee: JNC CORPORATION, Tokyo (JP)

(21) Appl. No.: 14/781,996

(22) PCT Filed: Apr. 4, 2014
(86) PCT No.: PCT/JP2014/059922

§ 371 Date: Dec. 18, 2015

(30) Foreign Application Priority Data
Apr. 4, 2013 (JP) 2013-078836

(51) Int. Cl. H01L 51/00; C07D 413/14; C07D 417/14; C07D 277/22; C07D 263/32

(52) U.S. Cl. CPC H01L 51/0069; C07D 277/22; H01L 51/0052; H01L 51/0077; C07D 263/32; H01L 51/007; C07D 417/14; H01L 51/0072; H01L 51/0054; H01L 51/0058; H01L 51/0067; C07D 413/14; H01L 51/5072

(57) ABSTRACT

The invention concerns a compound represented by formula (1) below, and an organic EL device using the same. The compound of the invention is useful as an electron transport material that contributes to improvement in service life prolongation, reduction of driving voltage, achievement of high efficiency and so forth, above all, improvement in achieving high efficiency, and can provide an excellent organic EL device:

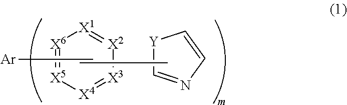

wherein, Ar is an m-valent group derived from aromatic hydrocarbon or an aromatic heterocycle;
X¹ to X⁶ are ═CR¹— or ═N—, at least two of X¹ to X⁶ is ═CR¹—, R¹ in two of ═CR¹— is a bonding hand to be bonded with Ar or an azole ring, and R¹ in ═CR¹— other than the above is hydrogen or alkyl having 1 to 4 carbons;
Y is —O— or —S—; at least one of hydrogen in an azole ring may be replaced by alkyl, phenyl or naphthyl; m is an integer from 2 to 4; and at least one of hydrogen in each ring and alkyl in the formula may be replaced by deuterium.

### TECHNICAL FIELD

The invention relates to a new electron transport material having a thiazolyl or oxazolyl group, an organic electroluminescent device (hereinafter, abbreviated as an organic EL device, or merely as a device in several cases) using the electron transport material, and so forth.

### BACKGROUND ART

An organic EL device has been lately recognized as a next-generation full color flat panel display, and actively researched. In order to promote practical use of the organic EL device, reduction of a power consumption of the device (reduction of voltage and improvement in an external quantum yield) and service life prolongation are essential elements, and a new electron transport material has been developed to achieve the targets. In particular, achievement of low power consumption or service life elongation of blue light-emitting element is a challenge, and various electron transport materials have been investigated so far. As described in Patent literature Nos. 1 to 4 and Non-patent literature No. 1, an art is known in which an organic EL device can be driven at low voltage by using a pyridine derivative or a bipyridine derivative as an electron transport material. A part thereof has been already practically applied, but such an organic EL device has characteristics with which the organic EL device is insufficient to be used for a larger number of displays. Moreover, use of a benzimidazole or benzothiazole derivative for an organic EL device as an electron transport material has also been investigated (see Patent literature Nos. 5 to 7.). In a manner similar to the pyridine derivative or bipyridine derivative, a part thereof has been practically applied, but as the characteristics, a level is insufficient and further improvement has been required.

### CITATION LIST

### Patent Literature

Patent literature No. 1: JP 2003-123983 A
Patent literature No. 2: JP 2002-158093 A
Patent literature No. 3: JP 2009-173642 A
Patent literature No. 4: WO 2007/086552 A
Patent literature No. 5: US 2003/215667 A
Patent literature No. 6: WO 2003/060956 A
Patent literature No. 7: WO 2008/117976 A

### Non-Patent Literature

Non-patent literature No. 1: Proceedings of the 10^{th} International Workshop on Inorganic and Organic Electroluminescence (2000)

### SUMMARY OF THE INVENTION

### Technical Problem

The invention has been made in view of problems of such prior arts. An objective of the invention is to provide an electron transport material that can contribute to improve characteristics required for the organic EL device, such as reduction of driving voltage, achievement of high efficiency, and service life prolongation. Further, an objective of the invention is to provide an organic EL device using the electron transport material.

### Solution to Problem

The present inventors have diligently continued to conduct study, and as a result, have found that use of aromatic hydrocarbon or aromatic heterocycle in which two or more places are replaced by a monovalent group typified by thiazolylphenyl, oxazolylphenyl, thiazolyl pyridyl or oxazolyl pyridyl for an electron transport layer of an organic EL device contributes to improvement of characteristics such as reduction of driving voltage, achievement of high efficiency and service life prolongation, above all, improvement in the high efficiency, and thus have completed the invention based on the finding.

The objective can be attained by each item described below.

Item 1. A compound represented by formula (1) below:

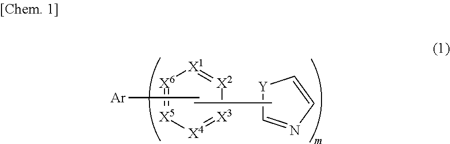

wherein, in formula (1),

Ar is an m-valent group derived from aromatic hydrocarbon having 6 to 40 carbons or an m-valent group derived from an aromatic heterocycle having 2 to 40 carbons, and at least one of hydrogen in the group may be replaced by alkyl having 1 to 12 carbons or cycloalkyl having 3 to 12 carbons;

X¹ to X⁶ are independently ═CR¹— or ═N—, at least two of X¹ to X⁶ is ═CR¹—, R¹ in two of ═CR¹— in X¹ to X⁶ is a bonding hand to be bonded with Ar or an azole ring, and R¹ in ═CR¹— other than the above is hydrogen or alkyl having 1 to 4 carbons;

Y is independently —O— or —S—;

at least one of hydrogen in an azole ring may be replaced by alkyl having 1 to 4 carbons, phenyl or naphthyl;

m is an integer from 2 to 4, and a group formed by an azole ring and a six-membered ring may be identical or different; and

at least one of hydrogen in each ring and alkyl in the formula may be replaced by deuterium.

Item 2. The compound according to item 1, wherein Ar is at least one selected from the group of groups represented by formulas (Ar-1) to (Ar-22) below:

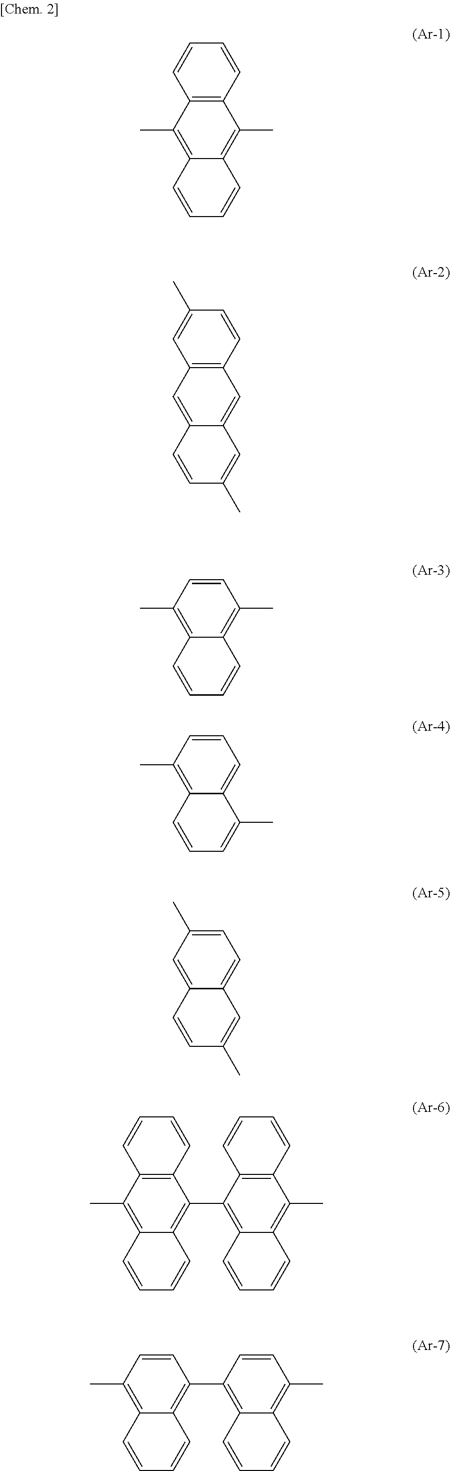

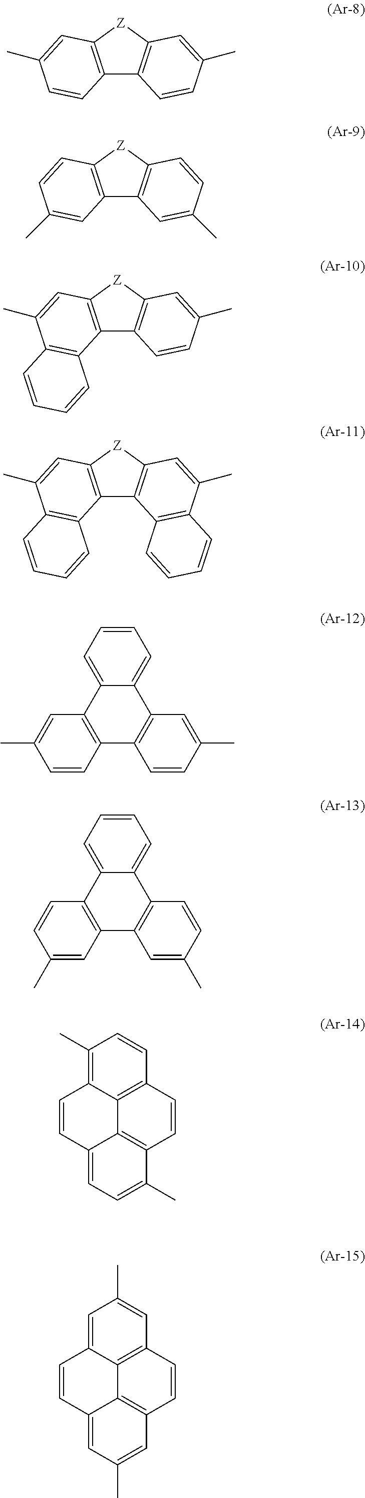

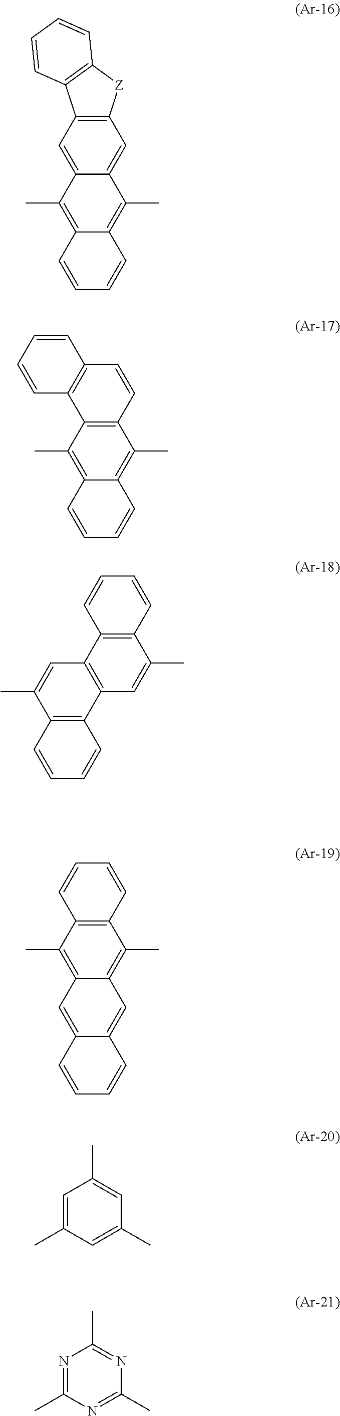

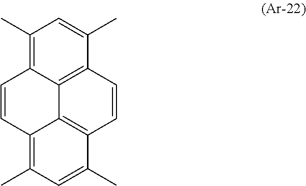

wherein, in formulas (Ar-1) to (Ar-22), Z is independently —O—, —S— or a divalent group represented by formula (2) or (3) below, and at least one of hydrogen in each group may be replaced by alkyl having 1 to 12 carbons, cycloalkyl having 3 to 12 carbons or aryl having 6 to 24 carbons,

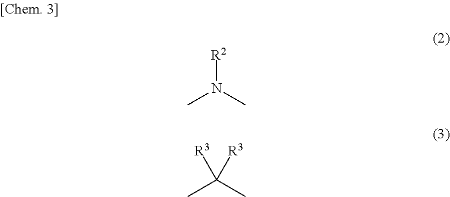

wherein, in formula (2), R² is phenyl, naphthyl, biphenylyl or terphenylyl, and in formula (3), R³ is independently methyl or phenyl, and two of R³ may be linked with each other to form a ring.
Item 3. The compound according to item 1, wherein Ar is one selected from the group of groups represented by formulas (Ar-1) to (Ar-13) below:

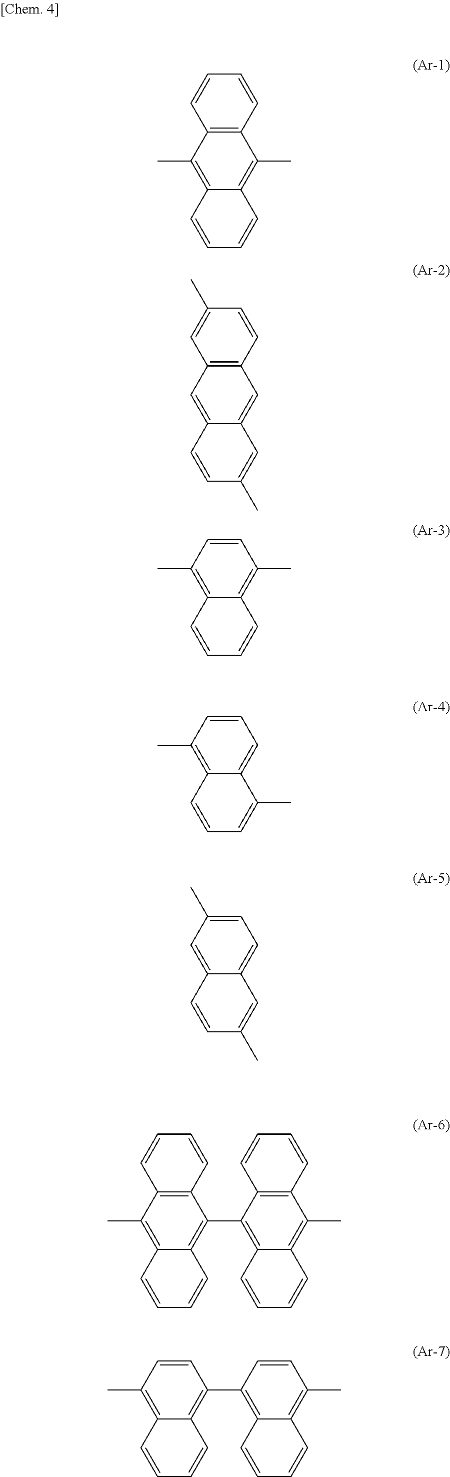

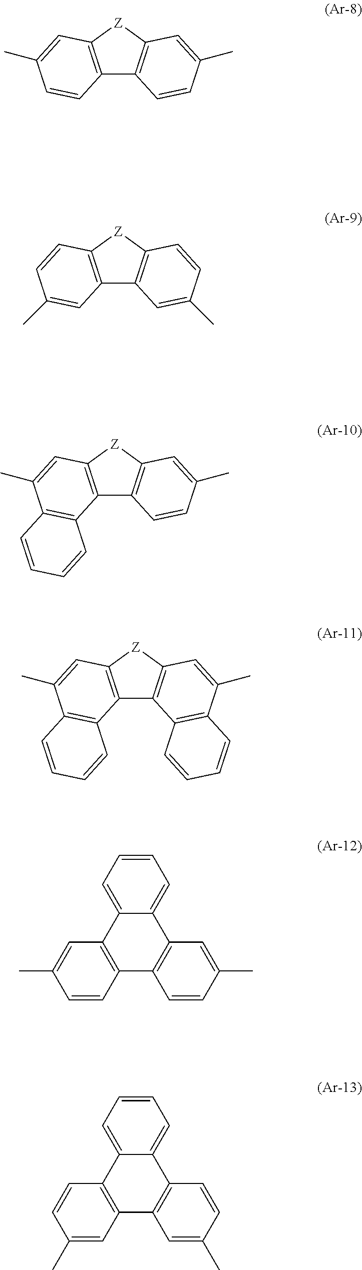

wherein, in formulas (Ar-1) to (Ar-13), Z is independently —O—, —S— or a divalent group represented by formula (2) or (3) below, and at least one of hydrogen in each group may be replaced by alkyl having 1 to 12 carbons, cycloalkyl having 3 to 12 carbons or aryl having 6 to 24 carbons;

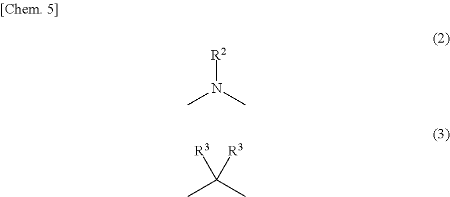

wherein, in formula (2), R² is phenyl, naphthyl, biphenylyl or terphenylyl, and in formula (3), R³ is independently methyl or phenyl, and two of R³ may be linked with each other to form a ring.
Item 4. The compound according to item 1, represented by formula (1-3) below:

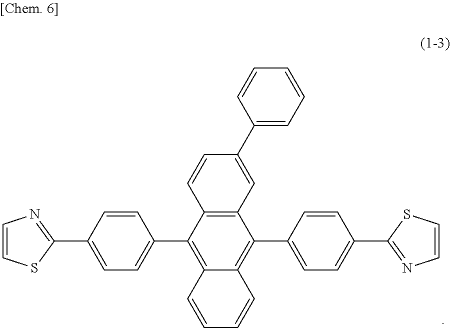 [SMILES: c1ccc(-c2ccc3c(-c4ccc(-c5nccs5)cc4)c4ccccc4c(-c4ccc(-c5nccs5)cc4)c3c2)cc1]

Item 5. The compound according to item 1, represented by one selected from formulas (1-4), (1-21), (1-25), (1-29), (1-37), (1-45), (1-53) and (1-85) below:

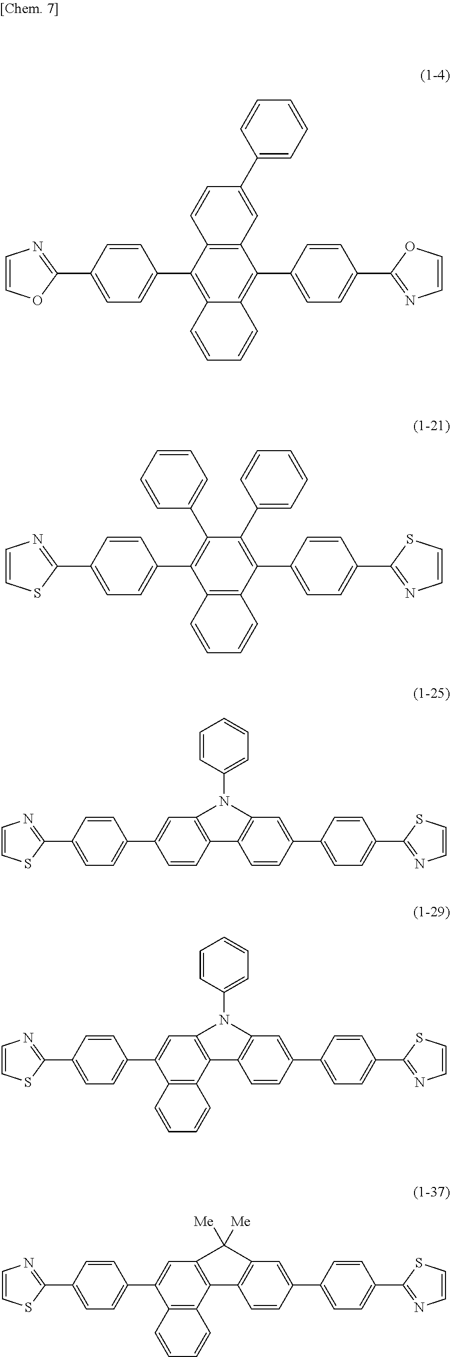 [SMILES: c1ccc(-c2ccc3c(-c4ccc(-c5ncco5)cc4)c4ccccc4c(-c4ccc(-c5ncco5)cc4)c3c2)cc1 ; c1ccc(-c2c(-c3ccccc3)c(-c3ccc(-c4nccs4)cc3)c3ccccc3c2-c2ccc(-c3nccs3)cc2)cc1 ; c1ccc(-n2c3cc(-c4ccc(-c5nccs5)cc4)ccc3c3ccc(-c4ccc(-c5nccs5)cc4)cc32)cc1 ; c1ccc(-n2c3cc(-c4ccc(-c5nccs5)cc4)ccc3c3c4ccccc4c(-c4ccc(-c5nccs5)cc4)cc32)cc1 ; CC1(C)c2cc(-c3ccc(-c4nccs4)cc3)ccc2-c2c1cc(-c1ccc(-c3nccs3)cc1)c1ccccc21]

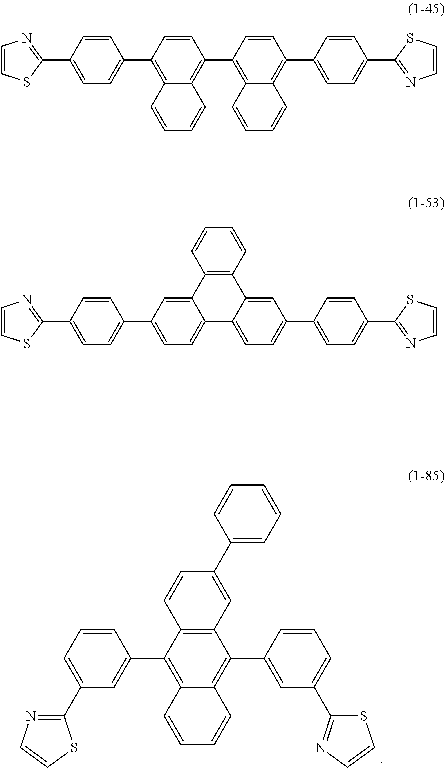 [SMILES: c1ccc2c(-c3ccc(-c4ccc(-c5nccs5)cc4)c4ccccc34)ccc(-c3ccc(-c4nccs4)cc3)c2c1 ; c1ccc2c(c1)c1cc(-c3ccc(-c4nccs4)cc3)ccc1c1ccc(-c3ccc(-c4nccs4)cc3)cc21 ; c1ccc(-c2ccc3c(-c4cccc(-c5nccs5)c4)c4ccccc4c(-c4cccc(-c5nccs5)c4)c3c2)cc1]

Item 6. The compound according to item 1, represented by formula (1-166) or (1-274) below:

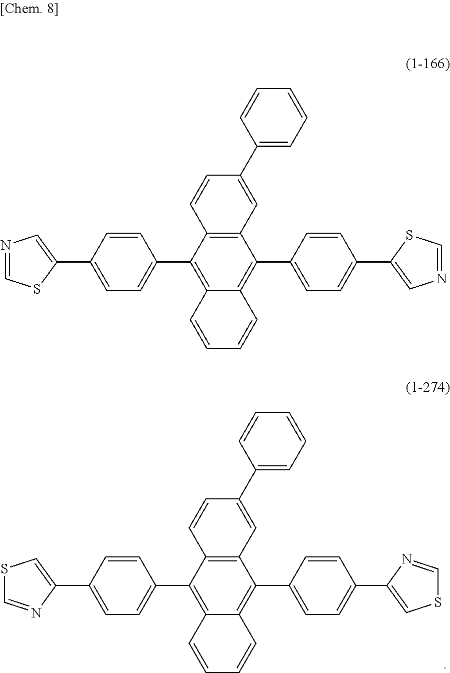 [SMILES: c1ccc(-c2ccc3c(-c4ccc(-c5cncs5)cc4)c4ccccc4c(-c4ccc(-c5cncs5)cc4)c3c2)cc1 ; c1ccc(-c2ccc3c(-c4ccc(-c5cscn5)cc4)c4ccccc4c(-c4ccc(-c5cscn5)cc4)c3c2)cc1]

Item 7. The compound according to item 1, represented by one selected from formulas (1-382), (1-383), (1-404), (1-408), (1-416), (1-424), (1-557), (1-558) and (1-611) below:

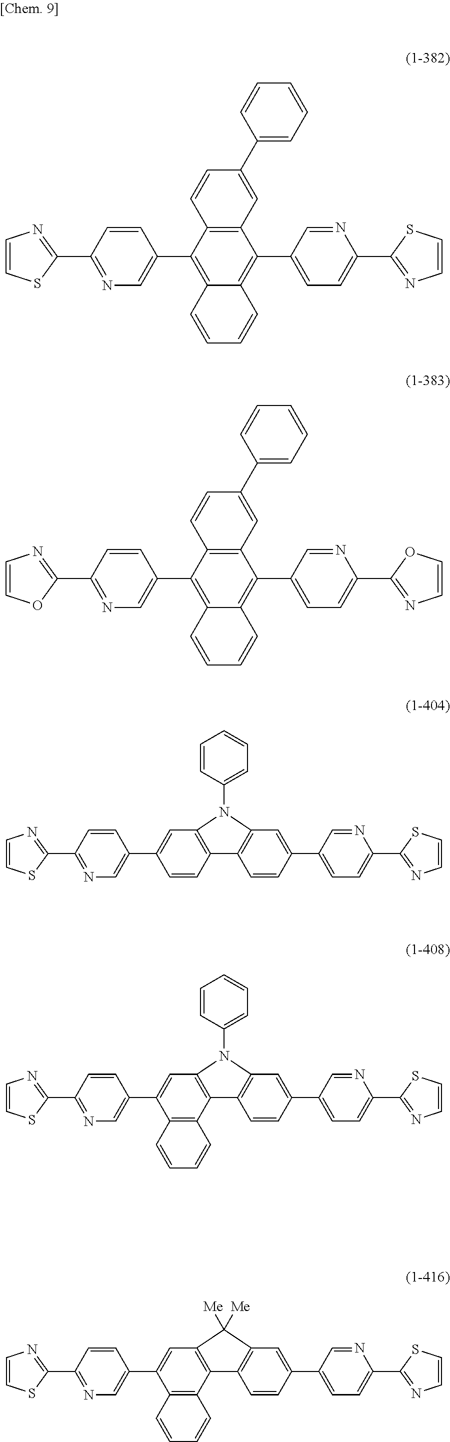 [SMILES: c1ccc(-c2ccc3c(-c4ccc(-c5nccs5)nc4)c4ccccc4c(-c4ccc(-c5nccs5)nc4)c3c2)cc1 ; c1ccc(-c2ccc3c(-c4ccc(-c5ncco5)nc4)c4ccccc4c(-c4ccc(-c5ncco5)nc4)c3c2)cc1 ; c1ccc(-n2c3cc(-c4ccc(-c5nccs5)nc4)ccc3c3ccc(-c4ccc(-c5nccs5)nc4)cc32)cc1 ; c1ccc(-n2c3cc(-c4ccc(-c5nccs5)nc4)ccc3c3c4ccccc4c(-c4ccc(-c5nccs5)nc4)cc32)cc1 ; CC1(C)c2cc(-c3ccc(-c4nccs4)nc3)ccc2-c2c1cc(-c1ccc(-c3nccs3)nc1)c1ccccc21]

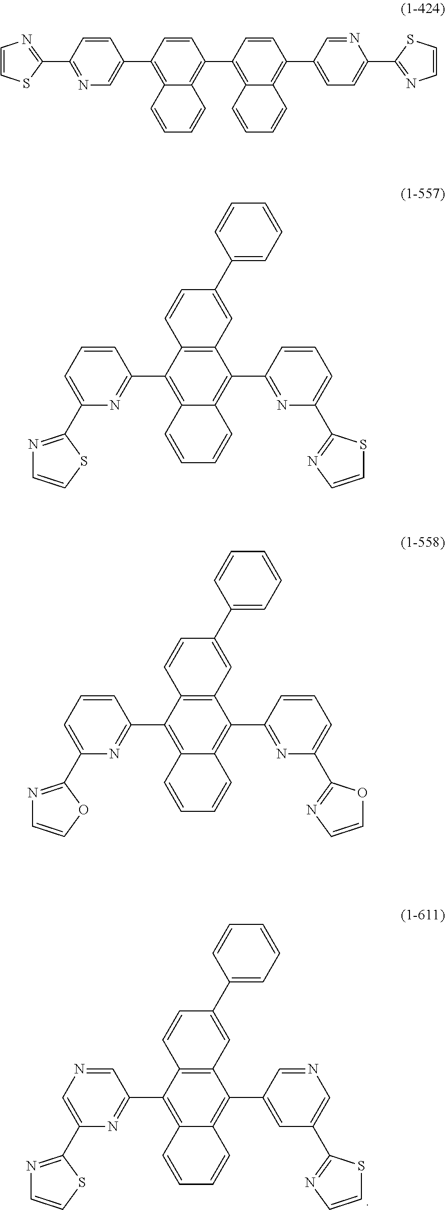 [SMILES: c1ccc2c(-c3ccc(-c4ccc(-c5nccs5)nc4)c4ccccc34)ccc(-c3ccc(-c4nccs4)nc3)c2c1 ; c1ccc(-c2ccc3c(-c4cccc(-c5nccs5)n4)c4ccccc4c(-c4cccc(-c5nccs5)n4)c3c2)cc1 ; c1ccc(-c2ccc3c(-c4cccc(-c5ncco5)n4)c4ccccc4c(-c4cccc(-c5ncco5)n4)c3c2)cc1 ; c1ccc(-c2ccc3c(-c4cncc(-c5nccs5)n4)c4ccccc4c(-c4cncc(-c5nccs5)c4)c3c2)cc1]

Item 8. An electron transport material, containing the compound according to any one of items 1 to 7.
Item 9. An organic electroluminescent device, having a pair of electrodes formed of an anode and a cathode, an emission layer arranged between the pair of electrodes, and an electron transport layer and/or an electron injection layer, containing the electron transport material according to item 8, and arranged between the cathode and the emission layer.
Item 10. The organic electroluminescent device according to item 9, wherein at least one of the electron transport layer and the electron injection layer further contains at least one selected from the group of a quinolinol metal complex, a bipyridine derivative, a phenanthroline derivative and a borane derivative.
Item 11. The organic electroluminescent device according to item 9 or item 10, wherein at least one of the electron transport layer and the electron injection layer further contains at least one selected from the group of alChem.li metal, alChem.line earth metal, rare earth metal, alChem.li metal oxide, alChem.li metal halide, alChem.line earth metal oxide, alChem.line earth metal halide, rare earth metal oxide, rare earth metal halide, an organic complex of alChem.li metal, an organic complex of alChem.line earth metal and an organic complex of rare earth metal.

### Advantageous Effects of Invention

The compound of the invention has features of being stable even if voltage is applied in a thin film state, and high in electron charge transport capacity. The compound of the invention is suitable as an electron charge transport material in an organic EL device. Use of the compound of the invention in an electron transport layer of the organic EL device contributes to improvement of characteristics, such as reduction of driving voltage, achievement of high efficiency, and service life prolongation, and above all, improvement in achieving high efficiency. A high performance display unit for full color display and so forth can be produced by using the organic EL device according to the invention.

### DESCRIPTION OF EMBODIMENTS

The invention will be described in more detail below. In addition, “compound represented by formula (1-1)” herein may be referred to as “compound (1-1),” in several cases, for example. “Compound represented by formula (1-2)” may be referred to as “compound (1-2)” in several cases. A same rule applies to any other formula symbol or formula number.

### Description of Compound

A first aspect of the invention is a compound having thiazolyl or oxazolyl represented by formula (1) below.

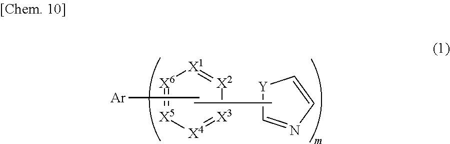

In formula (1), Ar is an m-valent group derived from aromatic hydrocarbon having 6 to 40 carbons or an m-valent group derived from an aromatic heterocycle having 2 to 40 carbons, and at least one of hydrogen in the group may be replaced by alkyl having 1 to 12 carbons or cycloalkyl having 3 to 12 carbons.

In formula (1), at least one of hydrogen in an azole ring may be replaced by alkyl having 1 to 4 carbons, phenyl or naphthyl, and Y is independently —O— or —S—. Moreover, m is an integer from 2 to 4, and preferably 2, a group formed by an azole ring and a six-membered ring may be identical or different, and preferably identical. Furthermore, at least one of hydrogen in each ring and alkyl in the formula may be replaced by deuterium.

In formula (1), X¹ to X⁶ are independently ═CR¹— or ═N—, at least two of X¹ to X⁶ is ═CR¹—, R¹ in two of ═CR¹— in X¹ to X⁶ is a bonding hand to be bonded with Ar or an azole ring, and R¹ in ═CR¹— other than the above is hydrogen or alkyl having 1 to 4 carbons.

Alkyl having 1 to 4 carbons being a substituent of the azole ring is defined in a manner identical with the definition of alkyl having 1 to 4 carbons when R¹ is alkyl, and the alkyl having 1 to 4 carbons may have any of a straight chain or a branched chain. More specifically, the alkyl is a straight-chain alkyl having 1 to 4 carbons or a branched chain alkyl having 3 or 4 carbons. Specific examples preferably include methyl, ethyl, n-propyl, isopropyl, n-butyl, isobutyl, s-butyl or t-butyl, and further preferably methyl, ethyl or t-butyl.

In formula (1), examples of Ar preferably include one selected from the group of groups represented by formulas (Ar-1) to (Ar-22) below, and further preferably one selected from the group of groups represented by formulas (Ar-1) to (Ar-13).

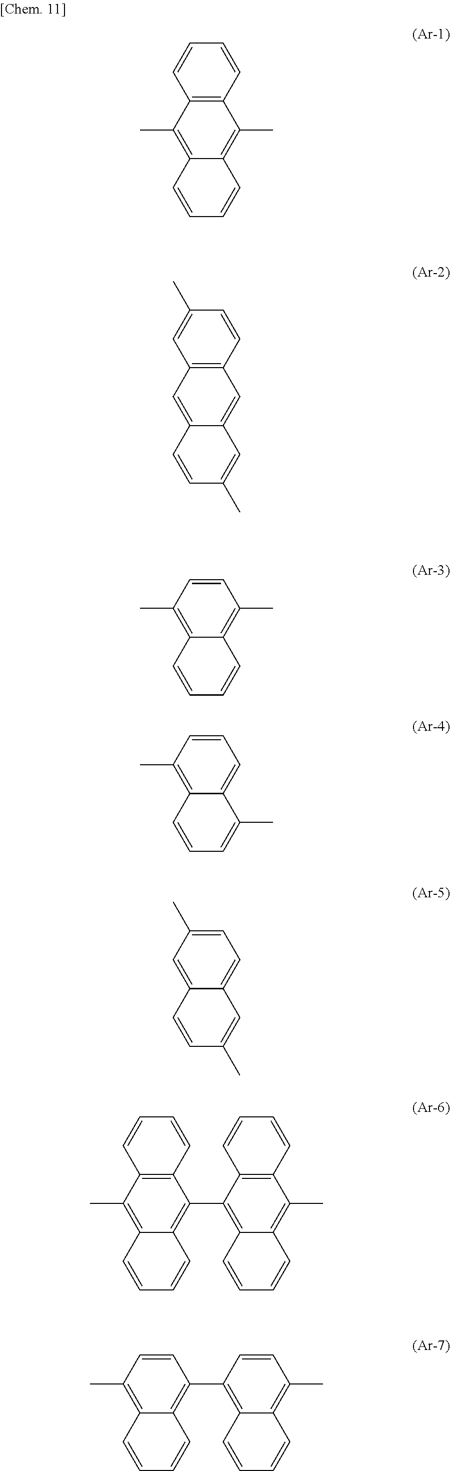

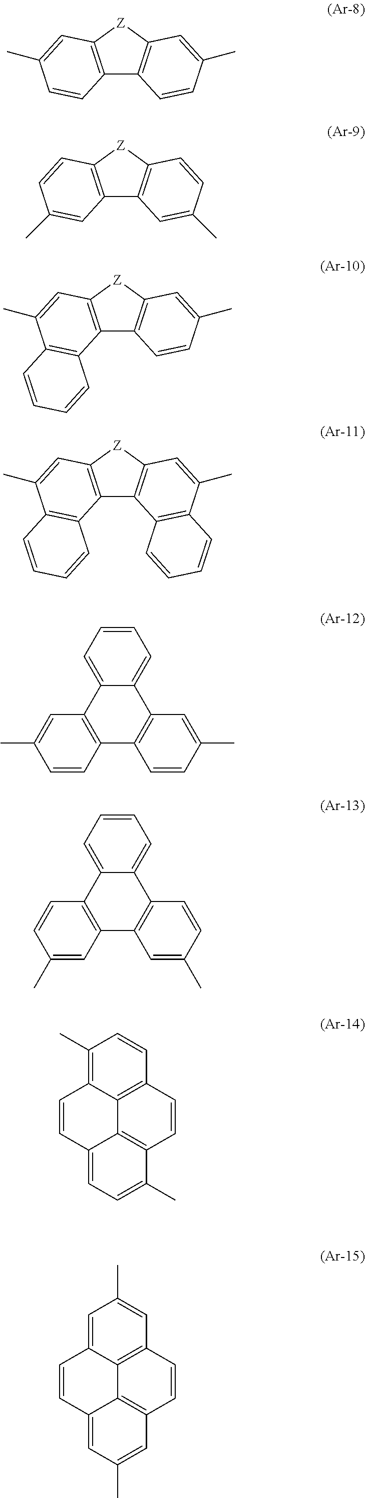

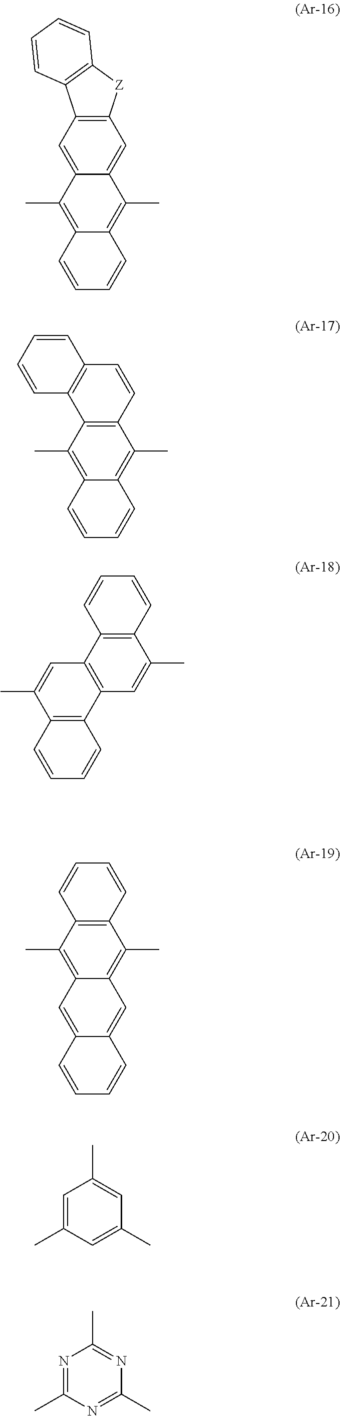

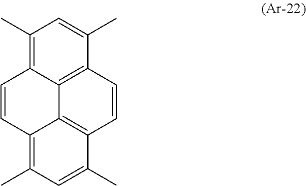

In the formula, Z is independently —O—, —S— or a divalent group represented by formula (2) or (3) below.

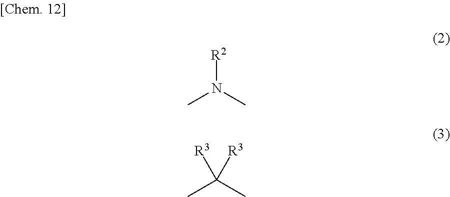

In formula (2), R² is phenyl, naphthyl, biphenylyl or terphenylyl, and in formula (3), R³ is independently methyl or phenyl, two of R³ may be linked with each other to form a ring. Specific examples include structure in which ortho positions of two of phenyl are linked with a single bond to form a spiro ring.

At least one of hydrogen in a group represented by formulas (Ar-1) to (Ar-22) may be replaced by alkyl having 1 to 12 carbons, cycloalkyl having 3 to 12 carbons or aryl having 6 to 24 carbons.

Alkyl having 1 to 12 carbons in which at least one of hydrogen in the group represented by formulas (Ar-1) to (Ar-22) may be replaced is straight-chain alkyl having 1 to 12 carbons or branched-chain alkyl having 3 to 12 carbons. Such alkyl is preferably alkyl having 1 to 6 carbons (branched-chain alkyl having 3 to 6 carbons), and further preferably alkyl having 1 to 4 carbons (branched-chain alkyl having 3 to 4 carbons). Specific examples include methyl, ethyl, n-propyl, isopropyl, n-butyl, isobutyl, s-butyl, t-butyl, n-pentyl, isopentyl, neopentyl, t-pentyl, n-hexyl, 1-methylpentyl, 4-methyl-2-pentyl, 3,3-dimethylbutyl or 2-ethylbutyl, preferably methyl, ethyl, n-propyl, isopropyl, n-butyl, isobutyl, s-butyl or t-butyl, and further preferably methyl, ethyl or t-butyl.

Specific examples of cycloalkyl having 3 to 12 carbons in which at least one of hydrogen in the group represented by formulas (Ar-1) to (Ar-22) may be replaced include cyclopropyl, cyclobutyl, cyclopentyl, cyclohexyl, methylcyclopentyl, cycloheptyl, methylcyclohexyl, cyclooctyl or dimethylcyclohexyl.

Specific examples of aryl having 6 to 24 carbons in which at least one of hydrogen in the group represented by formulas (Ar-1) to (Ar-22) may be replaced include phenyl, (o-, m-, p-) tolyl, (2,3-, 2,4-, 2,5-, 2,6-, 3,4-, 3,5-)xylyl, (2,4,6-trimethylphenyl)mesityl or (o-, m-, p-)cumenyl, each being monocyclic aryl, (2-, 3-, 4-)biphenylyl being bicyclic aryl, (1-, 2-)naphthyl being fused bicyclic aryl, terphenylyl(m-terphenyl-2′-yl, m-terphenyl-4′-yl, m-terphenyl-5′-yl, o-terphenyl-3′-yl, o-terphenyl-4′-yl, p-terphenyl-2′-yl, m-terphenyl-2-yl, m-terphenyl-3-yl, m-terphenyl-4-yl, o-terphenyl-2-yl, o-terphenyl-3-yl, o-terphenyl-4-yl, p-terphenyl-2-yl, p-terphenyl-3-yl or p-terphenyl-4-yl), each being tricyclic aryl, anthracene-(1-, 2-, 9-)yl, acenaphthylene-(1-, 3-, 4-, 5-)yl, fluorene-(1-, 2-, 3-, 4-, 9-)yl, phenalene-(1-, 2-)yl or (1-, 2-, 3-, 4-, 9-)phenanthryl, each being fused tricyclic aryl, triphenylene-(1-, 2-)yl, pyrene-(1-, 2-, 4-)yl or tetracene-(1-, 2-, 5-)yl, each being fused tetracyclic aryl, or perylene-(1-, 2-, 3-)yl being fused pentacyclic aryl.

“Aryl having 6 to 24 carbons” is preferably phenyl, biphenylyl, terphenylyl or naphthyl, and further preferably phenyl, biphenylyl, 1-naphthyl, 2-naphthyl or m-terphenyl-5′-yl.

In formula (1),

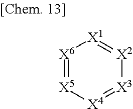

Specific examples of a ring represented by formula 13 preferably include a benzene ring, a pyridine ring, a pyrimidine ring, a pyrazine ring, a pyridazine ring or a triazine ring, and further preferably a benzene ring or a pyridine ring.

In formula (1),

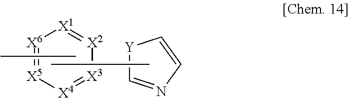

Specific examples of a group represented by formula 14 include: 4-(thiazole-2-yl)phenyl, 4-(thiazole-4-yl)phenyl, 4-(thiazole-5-yl)phenyl, 4-(oxazol-2-yl)phenyl, 4-(oxazol-4-yl)phenyl, 4-(oxazol-5-yl)phenyl, 3-(thiazole-2-yl)phenyl, 3-(thiazole-4-yl)phenyl, 3-(thiazole-5-yl)phenyl, 3-(oxazol-2-yl)phenyl, 3-(oxazol-4-yl)phenyl, 3-(oxazol-5-yl)phenyl, 6-(thiazole-2-yl)pyridine-3-yl, 6-(thiazole-4-yl)pyridine-3-yl, 6-(thiazole-5-yl)pyridine-3-yl, 6-(oxazol-2-yl)pyridine-3-yl, 6-(oxazol-4-yl)pyridine-3-yl, 6-(oxazol-5-yl)pyridine-3-yl, 5-(thiazole-2-yl)pyridine-2-yl, 5-(thiazole-4-yl)pyridine-2-yl, 5-(thiazole-5-yl)pyridine-2-yl, 5-(oxazol-2-yl)pyridine-2-yl, 5-(oxazol-4-yl)pyridine-2-yl, 5-(oxazol-5-yl)pyridine-2-yl, 6-(thiazole-2-yl)pyridine-2-yl, 6-(thiazole-4-yl)pyridine-2-yl, 6-(thiazole-5-yl)pyridine-2-yl, 6-(oxazol-2-yl)pyridine-2-yl, 6-(oxazol-4-yl)pyridine-2yl, 6-(oxazol-5-yl)pyridine-2-yl, 2-(thiazole-2-yl)pyridine-4-yl, 2-(thiazole-4-yl)pyridine-4-yl, 2-(thiazole-5-yl)pyridine-4-yl, 2-(oxazol-2-yl)pyridine-4-yl, 2-(oxazol-4-yl)pyridine-4-yl, 2-(oxazol-5-yl)pyridine-4-yl, 5-(thiazole-2-yl)pyridine-3-yl, 5-(thiazole-4-yl)pyridine-3-yl, 5-(thiazole-5-yl)pyridine-3-yl, 5-(oxazol-2-yl)pyridine-3-yl, 5-(oxazol-4-yl)pyridine-3-yl, 5-(oxazol-5-yl)pyridine-3-yl, 4-(thiazole-2-yl)pyridine-2-yl, 4-(thiazole-4-yl)pyridine-2-yl, 4-(thiazole-5-yl)pyridine-2-yl, 4-(oxazol-2-yl)pyridine-2-yl, 4-(oxazol-4-yl)pyridine-2-yl, 4-(oxazol-5-yl)pyridine-2-yl, 2-(thiazole-2-yl)pyrimidine-5-yl, 2-(thiazole-4-yl)pyrimidine-5-yl, 2-(thiazole-5-yl)pyrimidine-5-yl, 2-(oxazol-2-yl)pyrimidine-5-yl, 2-(oxazol-4-yl)pyrimidine-5-yl, 2-(oxazol-5-yl)pyrimidine-5-yl, 5-(thiazole-2-yl)pyrimidine-2-yl, 5-(thiazole-4-yl)pyrimidine-2-yl, 5-(thiazole-5-yl)pyrimidine-2-yl, 5-(oxazol-2-yl)pyrimidine-2-yl, 5-(oxazol-4-yl)pyrimidine-2-yl, 5-(oxazol-5-yl)pyrimidine-2-yl, 2-(thiazole-2-yl)pyrimidine-4-yl, 2-(thiazole-4-yl)pyrimidine-4-yl, 2-(thiazole-5-yl)pyrimidine-4-yl, 2-(oxazol-2-yl)pyrimidine-4-yl, 2-(oxazol-4-yl)pyrimidine-4-yl, 2-(oxazol-5-yl)pyrimidine-4-yl, 4-(thiazole-2-yl)pyrimidine-2-yl, 4-(thiazole-4-yl)pyrimidine-2-yl, 4-(thiazole-5-yl)pyrimidine-2-yl, 4-(oxazol-2-yl)pyrimidine-2-yl, 4-(oxazol-4-yl)pyrimidine-2-yl, 4-(oxazol-5-yl)pyrimidine-2-yl, 6-(thiazole-2-yl)pyrimidine-4-yl, 6-(thiazole-4-yl)pyrimidine-4-yl, 6-(thiazole-5-yl)pyrimidine-4-yl, 6-(oxazol-2-yl)pyrimidine-4-yl, 6-(oxazol-4-yl)pyrimidine-4-yl, 6-(oxazol-5-yl)pyrimidine-4-yl, 5-(thiazole-2-yl)pyrazine-2-yl, 5-(thiazole-4-yl)pyrazine-2-yl, 5-(thiazole-5-yl)pyrazine-2-yl, 5-(oxazol-2-yl)pyrazine-2-yl, 5-(oxazol-4-yl)pyrazine-2-yl, 5-(oxazol-5-yl)pyrazine-2-yl, 6-(thiazole-2-yl)pyrazine-2-yl, 6-(thiazole-4-yl)pyrazine-2-yl, 6-(thiazole-5-yl)pyrazine-2-yl, 6-(oxazol-2-yl)pyrazine-2-yl, 6-(oxazol-4-yl)pyrazine-2-yl, 6-(oxazol-5-yl)pyrazine-2-yl, 6-(thiazole-2-yl)pyridazine-3-yl, 6-(thiazole-4-yl)pyridazine-3-yl, 6-(thiazole-5-yl)pyridazine-3-yl, 6-(oxazol-2-yl)pyridazine-3-yl, 6-(oxazol-4-yl)pyridazine-3-yl, 6-(oxazol-5-yl)pyridazine-3-yl, 4-(thiazole-2-yl)-1,3,5-triazine-2-yl, 4-(thiazole-4-yl)-1,3,5-triazine-2-yl, 4-(thiazole-5-yl)-1,3,5-triazine-2-yl, 4-(oxazol-2-yl)-1,3,5-triazine-2-yl, 4-(oxazol-4-yl)-1,3,5-triazine-2-yl or 4-(oxazol-5-yl)-1,3,5-triazine-2-yl.

Preferred groups among the groups include 4-(thiazole-2-yl)phenyl, 4-(thiazole-4-yl)phenyl, 4-(thiazole-5-yl)phenyl, 4-(oxazol-2-yl)phenyl, 4-(oxazol-4-yl)phenyl, 4-(oxazol-5-yl)phenyl, 3-(thiazole-2-yl)phenyl, 3-(thiazole-4-yl)phenyl, 3-(thiazole-5-yl)phenyl, 3-(oxazol-2-yl)phenyl, 3-(oxazol-4-yl)phenyl, 3-(oxazol-5-yl)phenyl, 6-(thiazole-2-yl)pyridine-3-yl, 6-(thiazole-4-yl)pyridine-3-yl, 6-(thiazole-5-yl)pyridine-3-yl, 6-(oxazol-2-yl)pyridine-3-yl, 6-(oxazol-4-yl)pyridine-3-yl, 6-(oxazol-5-yl)pyridine-3-yl, 5-(thiazole-2-yl)pyridine-2-yl, 5-(thiazole-4-yl)pyridine-2-yl, 5-(thiazole-5-yl)pyridine-2-yl, 5-(oxazol-2-yl)pyridine-2-yl, 5-(oxazol-4-yl)pyridine-2-yl, 5-(oxazol-5-yl)pyridine-2-yl, 6-(thiazole-2-yl)pyridine-2-yl, 6-(thiazole-4-yl)pyridine-2-yl, 6-(thiazole-5-yl)pyridine-2-yl, 6-(oxazol-2-yl)pyridine-2-yl, 6-(oxazol-4-yl)pyridine-2-yl, 6-(oxazol-5-yl)pyridine-2-yl, 2-(thiazole-2-yl)pyridine-4-yl, 2-(thiazole-4-yl)pyridine-4-yl, 2-(thiazole-5-yl)pyridine-4-yl, 2-(oxazol-2-yl)pyridine-4-yl, 2-(oxazol-4-yl)pyridine-4-yl, 2-(oxazol-5-yl)pyridine-4-yl, 5-(thiazole-2-yl)pyridine-3-yl, 5-(thiazole-4-yl)pyridine-3-yl, 5-(thiazole-5-yl)pyridine-3-yl, 5-(oxazol-2-yl)pyridine-3-yl, 5-(oxazol-4-yl)pyridine-3-yl, 5-(oxazol-5-yl)pyridine-3-yl, 4-(thiazole-2-yl)pyridine-2-yl, 4-(thiazole-4-yl)pyridine-2-yl, 4-(thiazole-5-yl)pyridine-2-yl, 4-(oxazol-2-yl)pyridine-2-yl, 4-(oxazol-4-yl)pyridine-2-yl or 4-(oxazol-5-yl)pyridine-2-yl.

Further preferred groups include 4-(thiazole-2-yl)phenyl, 4-(thiazole-4-yl)phenyl, 4-(thiazole-5-yl)phenyl, 4-(oxazol-2-yl)phenyl, 4-(oxazol-4-yl)phenyl, 4-(oxazol-5-yl)phenyl, 3-(thiazole-2-yl)phenyl, 3-(thiazole-4-yl)phenyl, 3-(thiazole-5-yl)phenyl, 3-(oxazol-2-yl)phenyl, 3-(oxazol-4-yl)phenyl, 3-(oxazol-5-yl)phenyl, 6-(thiazole-2-yl)pyridine-3-yl, 6-(thiazole-4-yl)pyridine-3-yl, 6-(thiazole-5-yl)pyridine-3-yl, 6-(oxazol-2-yl)pyridine-3-yl, 6-(oxazol-4-yl)pyridine-3-yl, 6-(oxazol-5-yl)pyridine-3-yl, 6-(thiazole-2-yl)pyridine-2-yl, 6-(thiazole-4-yl)pyridine-2-yl, 6-(thiazole-5-yl)pyridine-2-yl, 6-(oxazol-2-yl)pyridine-2-yl, 6-(oxazol-4-yl)pyridine-2-yl, 6-(oxazol-5-yl)pyridine-2-yl, 5-(thiazole-2-yl)pyridine-3-yl, 5-(thiazole-4-yl)pyridine-3-yl, 5-(thiazole-5-yl)pyridine-3-yl, 5-(oxazol-2-yl)pyridine-3-yl, 5-(oxazol-4-yl)pyridine-3-yl or 5-(oxazol-5-yl)pyridine-3-yl.

Further preferred groups include 4-(thiazole-2-yl)phenyl, 4-(oxazol-2-yl)phenyl, 3-(thiazole-2-yl)phenyl, 3-(oxazol-2-yl)phenyl, 6-(thiazole-2-yl)pyridine-3-yl, 6-(oxazol-2-yl)pyridine-3-yl, 6-(thiazole-2-yl)pyridine-2-yl, 6-(oxazol-2-yl)pyridine-2-yl, 5-(thiazole-2-yl)pyridine-3-yl or 5-(oxazol-2-yl)pyridine-3-yl, A most preferred group includes 4-(thiazole-2-yl)phenyl.

### Specific Examples of Compound

Specific examples of the compound according to the invention are represented by formulas listed below, but the invention is not limited by disclosure of specific structure.

### Specific Examples of Compound Represented by Formula (1)

Specific examples of a compound represented by formula (1) are shown by formulas (1-1) to (1-500) and (1-511) to (1-934) below.

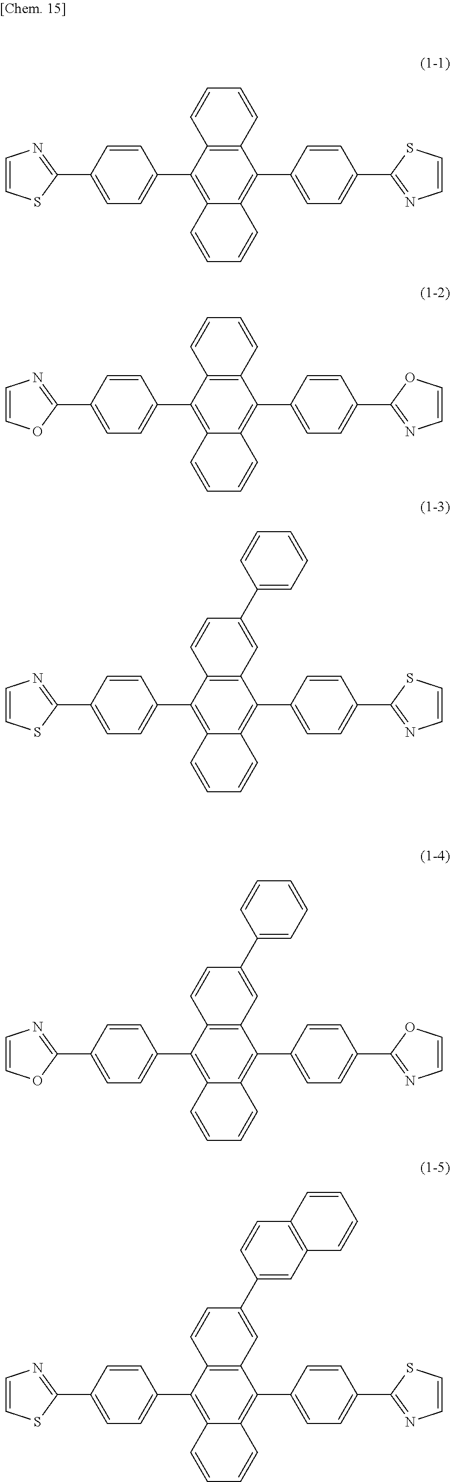

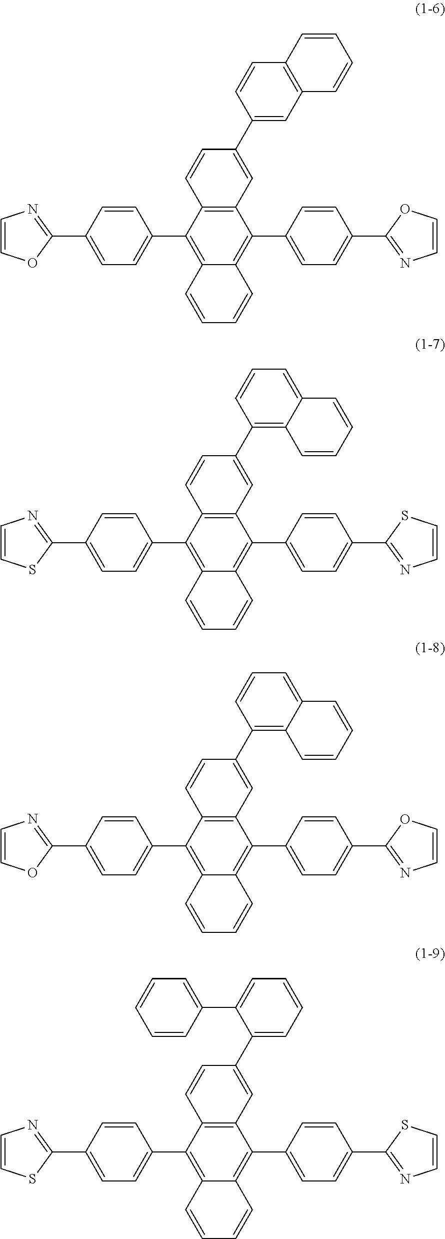

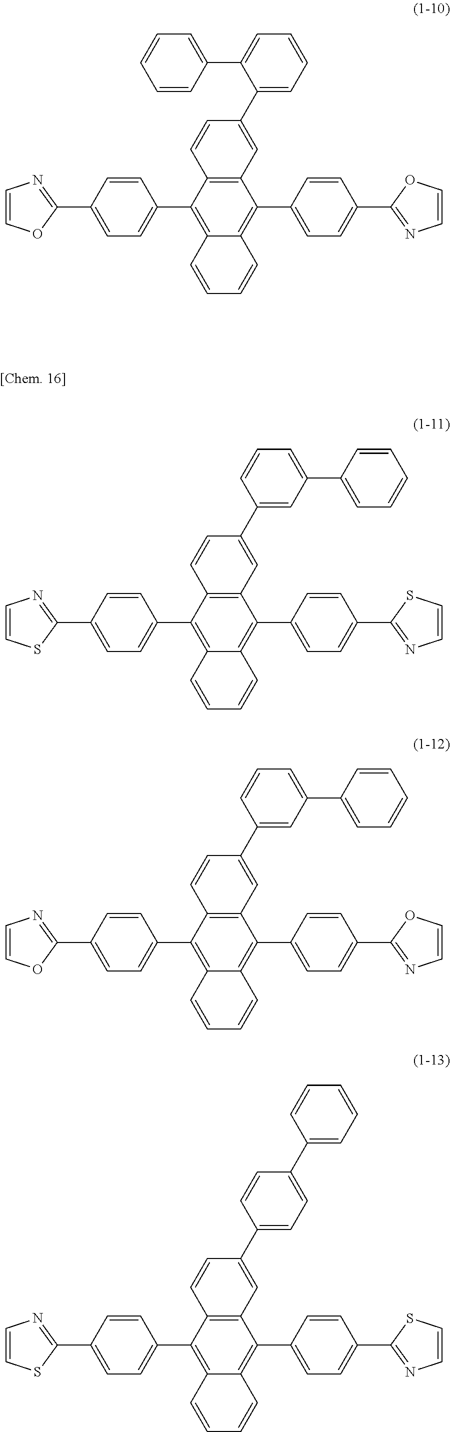 [SMILES: c1ccc(-c2ccccc2-c2ccc3c(-c4ccc(-c5ncco5)cc4)c4ccccc4c(-c4ccc(-c5ncco5)cc4)c3c2)cc1 ; c1ccc(-c2cccc(-c3ccc4c(-c5ccc(-c6nccs6)cc5)c5ccccc5c(-c5ccc(-c6nccs6)cc5)c4c3)c2)cc1 ; c1ccc(-c2cccc(-c3ccc4c(-c5ccc(-c6ncco6)cc5)c5ccccc5c(-c5ccc(-c6ncco6)cc5)c4c3)c2)cc1 ; c1ccc(-c2ccc(-c3ccc4c(-c5ccc(-c6nccs6)cc5)c5ccccc5c(-c5ccc(-c6nccs6)cc5)c4c3)cc2)cc1]

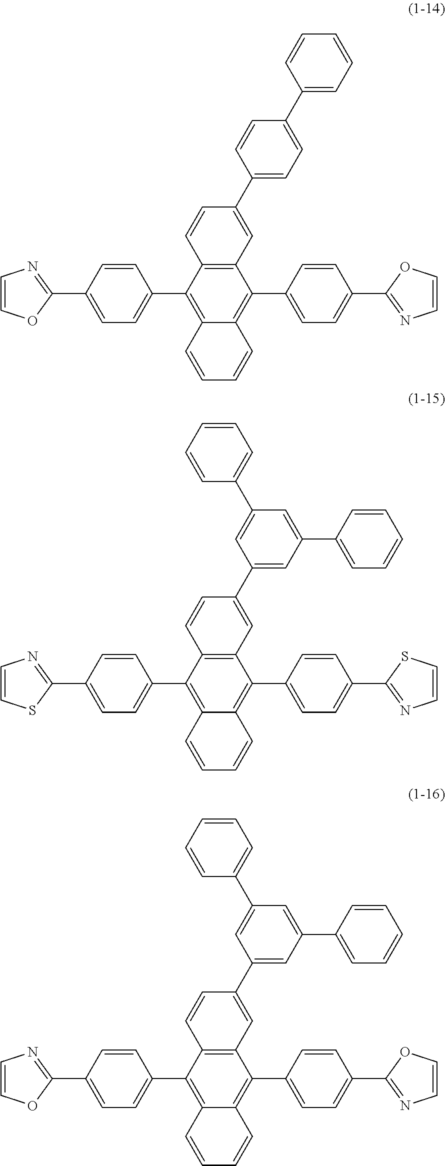

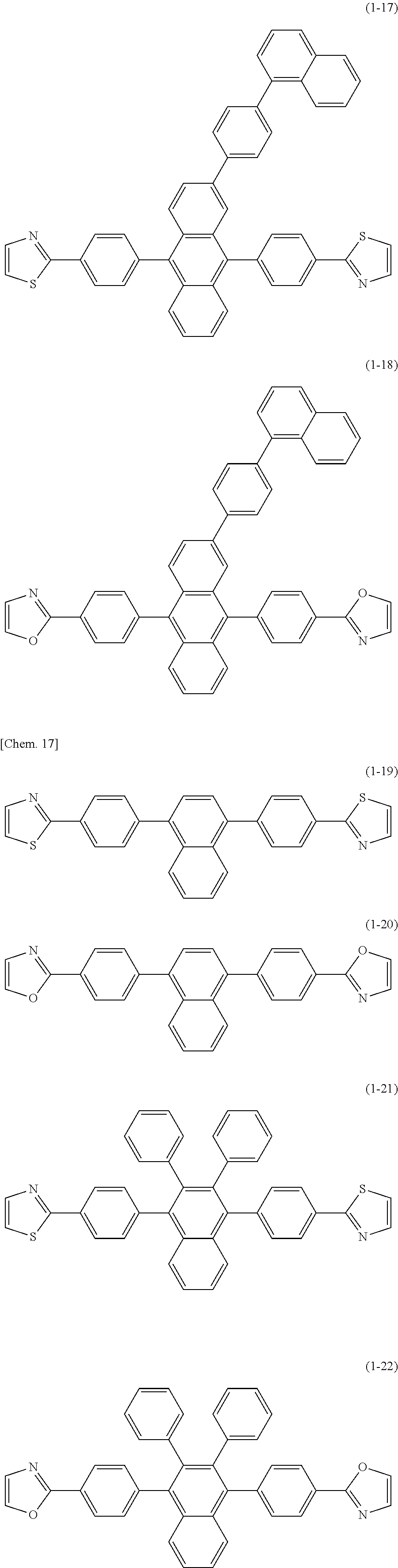 [SMILES: c1ccc2c(-c3ccc(-c4ccc5c(-c6ccc(-c7nccs7)cc6)c6ccccc6c(-c6ccc(-c7nccs7)cc6)c5c4)cc3)cccc2c1 ; c1ccc2c(-c3ccc(-c4ccc5c(-c6ccc(-c7ncco7)cc6)c6ccccc6c(-c6ccc(-c7ncco7)cc6)c5c4)cc3)cccc2c1 ; c1ccc2c(-c3ccc(-c4nccs4)cc3)ccc(-c3ccc(-c4nccs4)cc3)c2c1 ; c1ccc2c(-c3ccc(-c4ncco4)cc3)ccc(-c3ccc(-c4ncco4)cc3)c2c1 ; c1ccc(-c2c(-c3ccccc3)c(-c3ccc(-c4nccs4)cc3)c3ccccc3c2-c2ccc(-c3nccs3)cc2)cc1 ; c1ccc(-c2c(-c3ccccc3)c(-c3ccc(-c4ncco4)cc3)c3ccccc3c2-c2ccc(-c3ncco3)cc2)cc1]

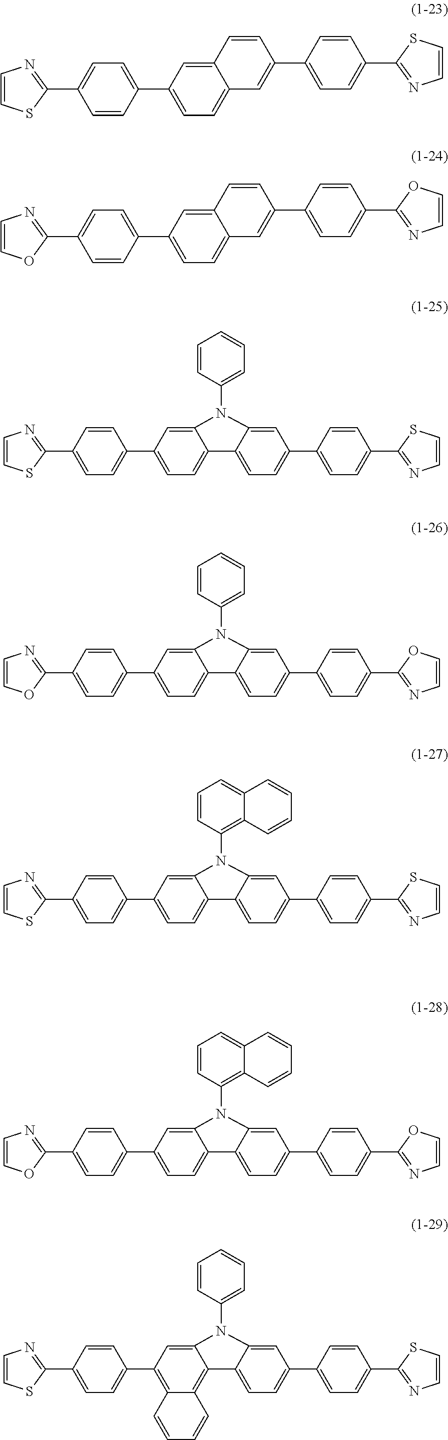

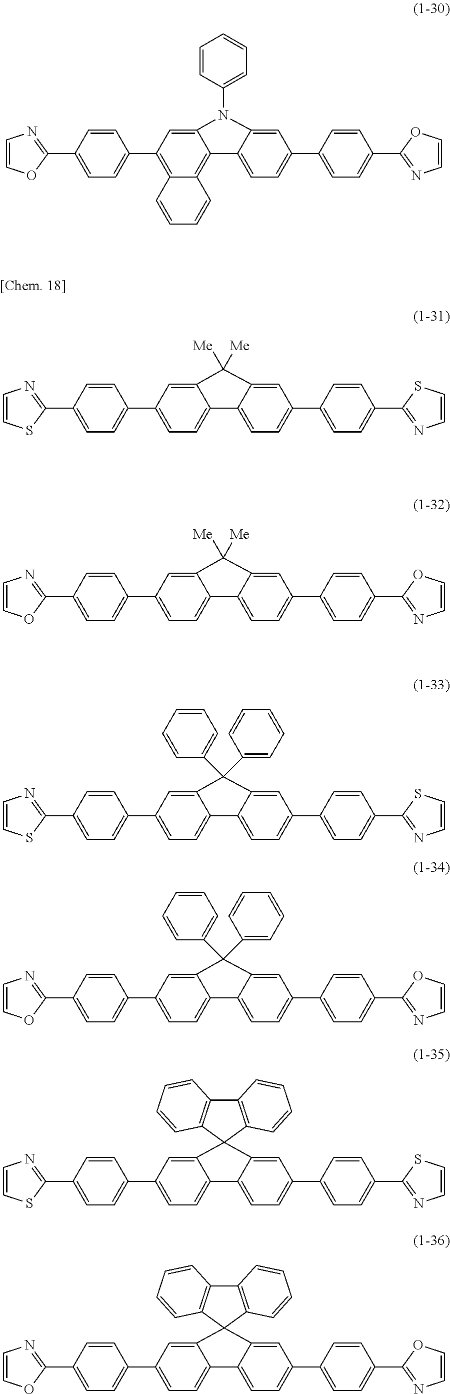

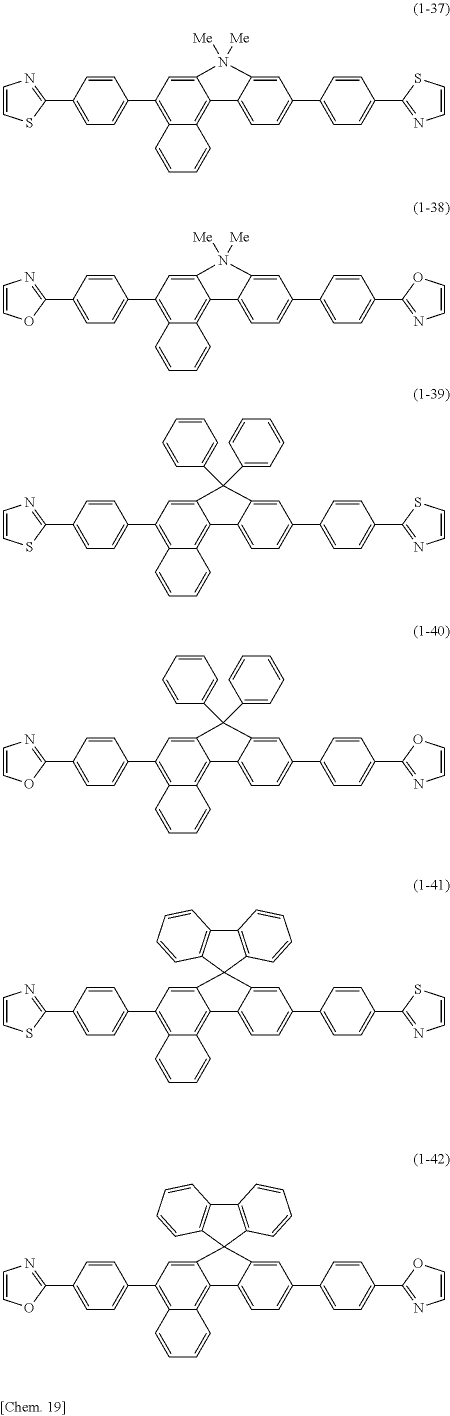

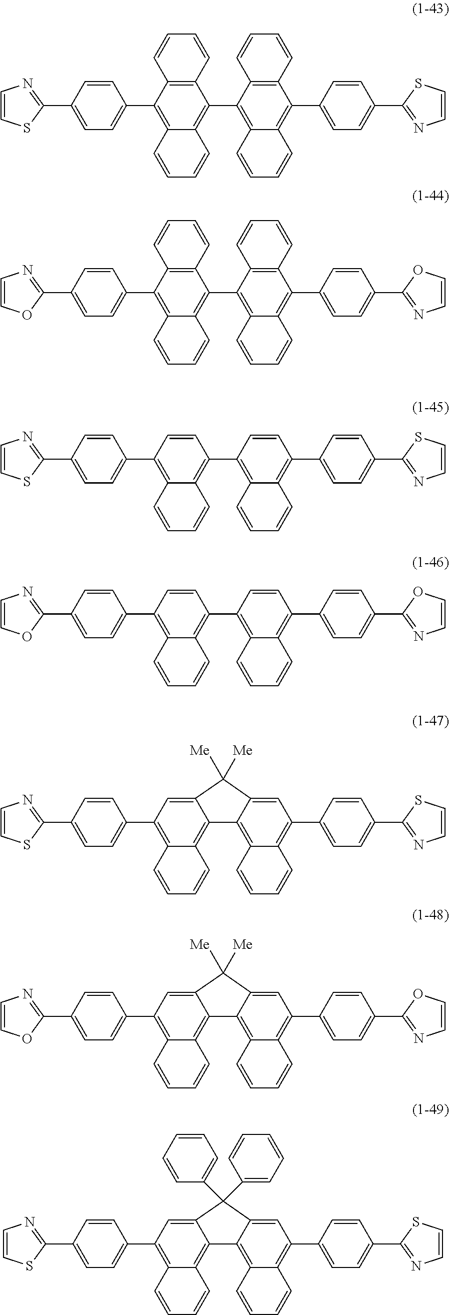

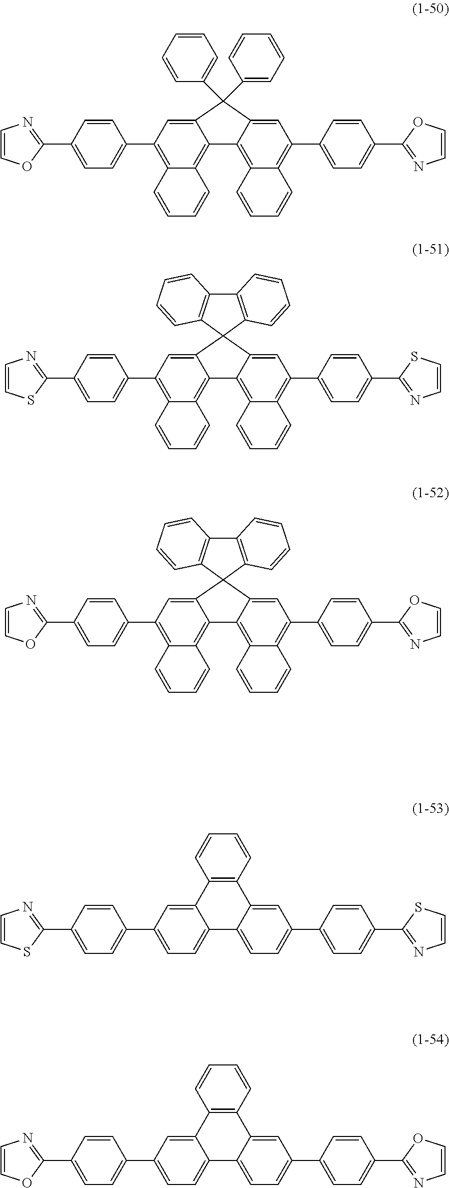 [SMILES: c1ccc(C2(c3ccccc3)c3cc(-c4ccc(-c5ncco5)cc4)c4ccccc4c3-c3c2cc(-c2ccc(-c4ncco4)cc2)c2ccccc32)cc1 ; c1ccc2c(c1)-c1ccccc1C21c2cc(-c3ccc(-c4nccs4)cc3)c3ccccc3c2-c2c1cc(-c1ccc(-c3nccs3)cc1)c1ccccc21 ; c1ccc2c(c1)-c1ccccc1C21c2cc(-c3ccc(-c4ncco4)cc3)c3ccccc3c2-c2c1cc(-c1ccc(-c3ncco3)cc1)c1ccccc21 ; c1ccc2c(c1)c1cc(-c3ccc(-c4nccs4)cc3)ccc1c1ccc(-c3ccc(-c4nccs4)cc3)cc21 ; c1ccc2c(c1)c1cc(-c3ccc(-c4ncco4)cc3)ccc1c1ccc(-c3ccc(-c4ncco4)cc3)cc21]

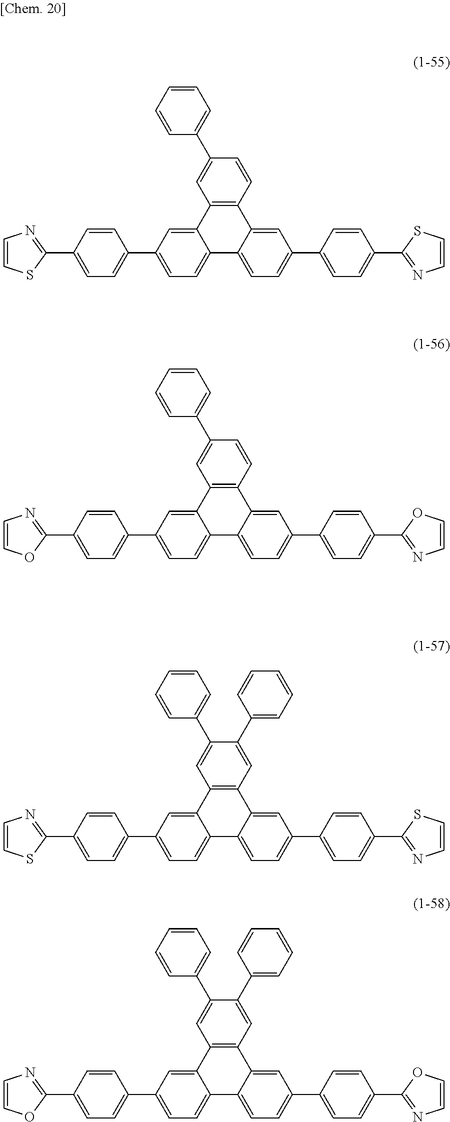 [SMILES: c1ccc(-c2ccc3c4cc(-c5ccc(-c6nccs6)cc5)ccc4c4ccc(-c5ccc(-c6nccs6)cc5)cc4c3c2)cc1 ; c1ccc(-c2ccc3c4cc(-c5ccc(-c6ncco6)cc5)ccc4c4ccc(-c5ccc(-c6ncco6)cc5)cc4c3c2)cc1 ; c1ccc(-c2cc3c4cc(-c5ccc(-c6nccs6)cc5)ccc4c4ccc(-c5ccc(-c6nccs6)cc5)cc4c3cc2-c2ccccc2)cc1 ; c1ccc(-c2cc3c4cc(-c5ccc(-c6ncco6)cc5)ccc4c4ccc(-c5ccc(-c6ncco6)cc5)cc4c3cc2-c2ccccc2)cc1]

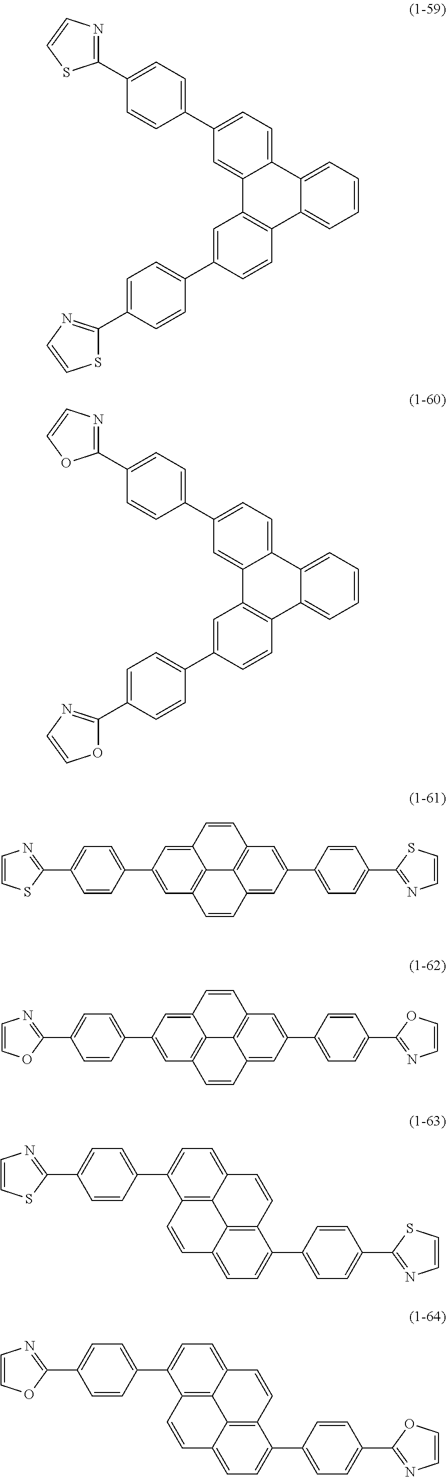

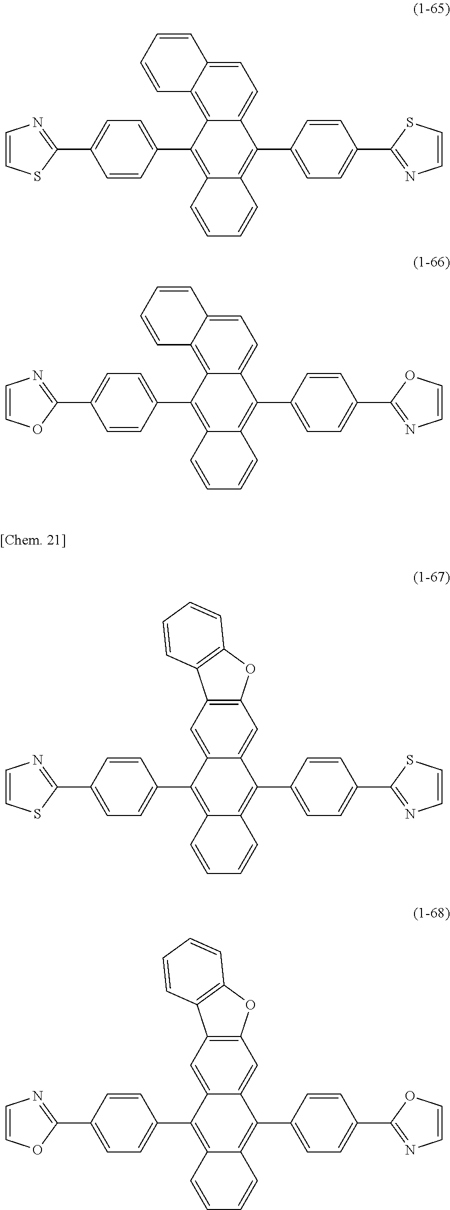 [SMILES: c1ccc2c(c1)ccc1c(-c3ccc(-c4nccs4)cc3)c3ccccc3c(-c3ccc(-c4nccs4)cc3)c12 ; c1ccc2c(c1)ccc1c(-c3ccc(-c4ncco4)cc3)c3ccccc3c(-c3ccc(-c4ncco4)cc3)c12 ; c1ccc2c(c1)oc1cc3c(-c4ccc(-c5nccs5)cc4)c4ccccc4c(-c4ccc(-c5nccs5)cc4)c3cc12 ; c1ccc2c(c1)oc1cc3c(-c4ccc(-c5ncco5)cc4)c4ccccc4c(-c4ccc(-c5ncco5)cc4)c3cc12]

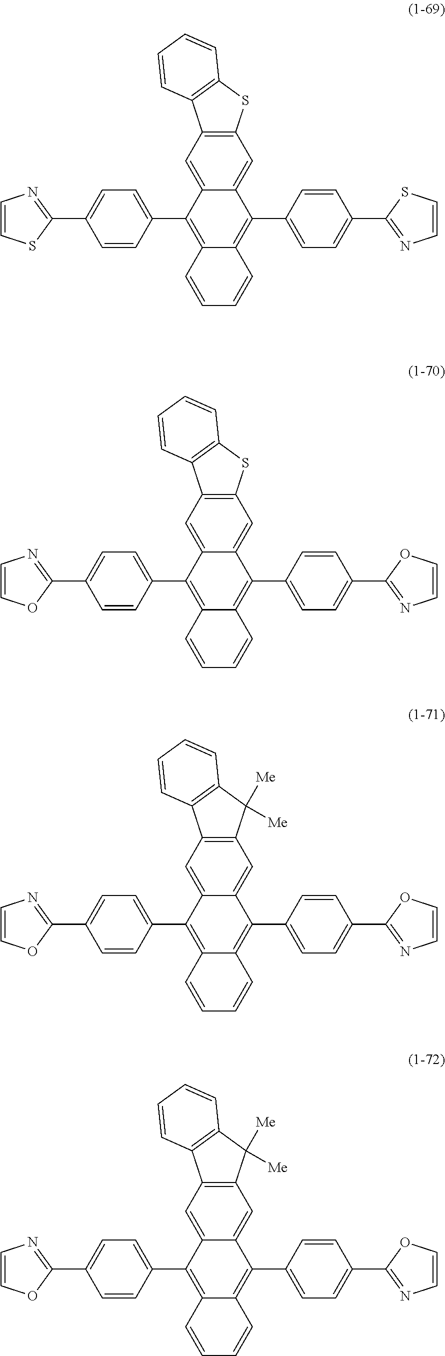 [SMILES: c1ccc2c(c1)sc1cc3c(-c4ccc(-c5nccs5)cc4)c4ccccc4c(-c4ccc(-c5nccs5)cc4)c3cc12 ; c1ccc2c(c1)sc1cc3c(-c4ccc(-c5ncco5)cc4)c4ccccc4c(-c4ccc(-c5ncco5)cc4)c3cc12 ; CC1(C)c2ccccc2-c2cc3c(-c4ccc(-c5ncco5)cc4)c4ccccc4c(-c4ccc(-c5ncco5)cc4)c3cc21 ; CC1(C)c2ccccc2-c2cc3c(-c4ccc(-c5ncco5)cc4)c4ccccc4c(-c4ccc(-c5ncco5)cc4)c3cc21]

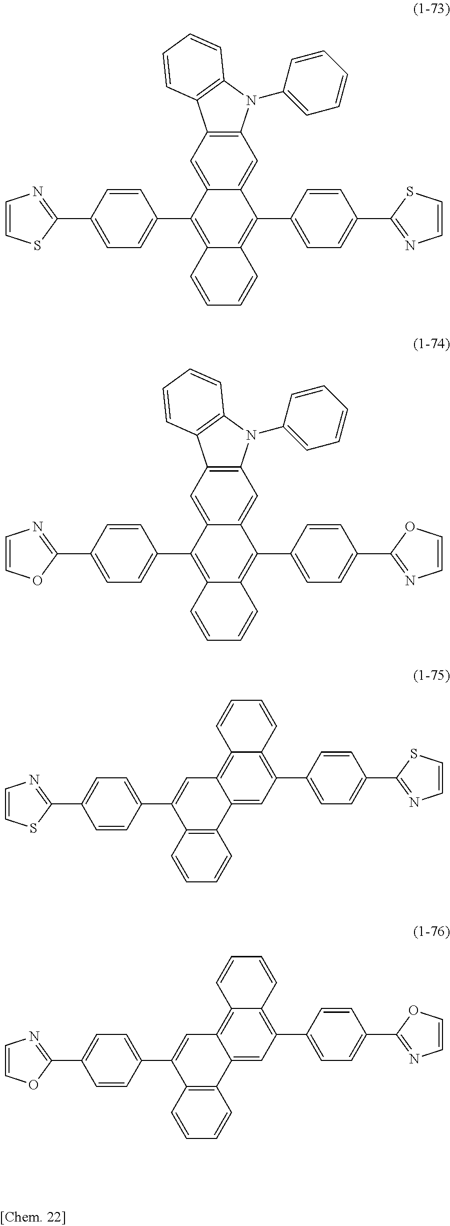 [SMILES: c1ccc(-n2c3ccccc3c3cc4c(-c5ccc(-c6nccs6)cc5)c5ccccc5c(-c5ccc(-c6nccs6)cc5)c4cc32)cc1 ; c1ccc(-n2c3ccccc3c3cc4c(-c5ccc(-c6ncco6)cc5)c5ccccc5c(-c5ccc(-c6ncco6)cc5)c4cc32)cc1 ; c1ccc2c(c1)c(-c1ccc(-c3nccs3)cc1)cc1c3ccccc3c(-c3ccc(-c4nccs4)cc3)cc21 ; c1ccc2c(c1)c(-c1ccc(-c3ncco3)cc1)cc1c3ccccc3c(-c3ccc(-c4ncco4)cc3)cc21]

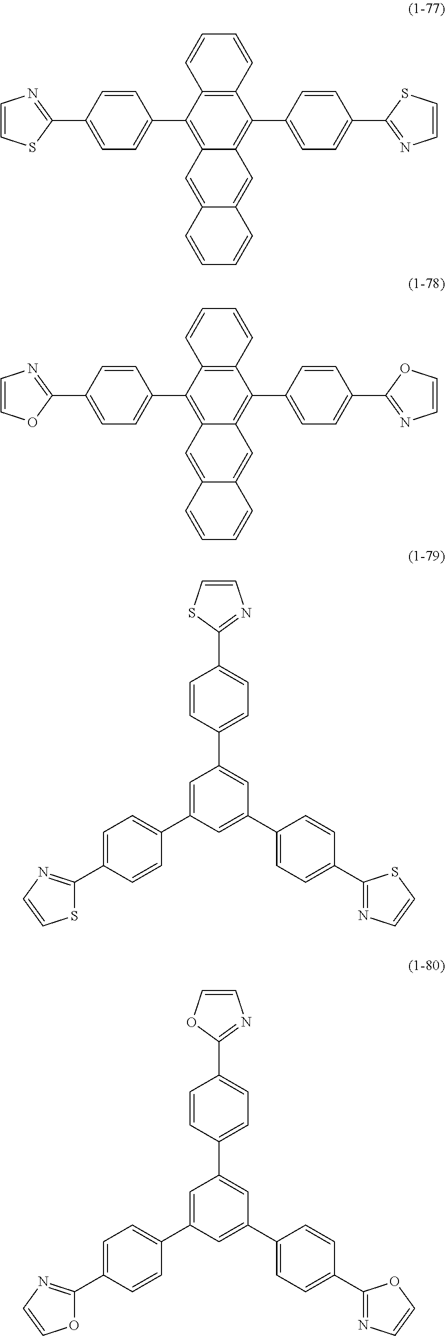

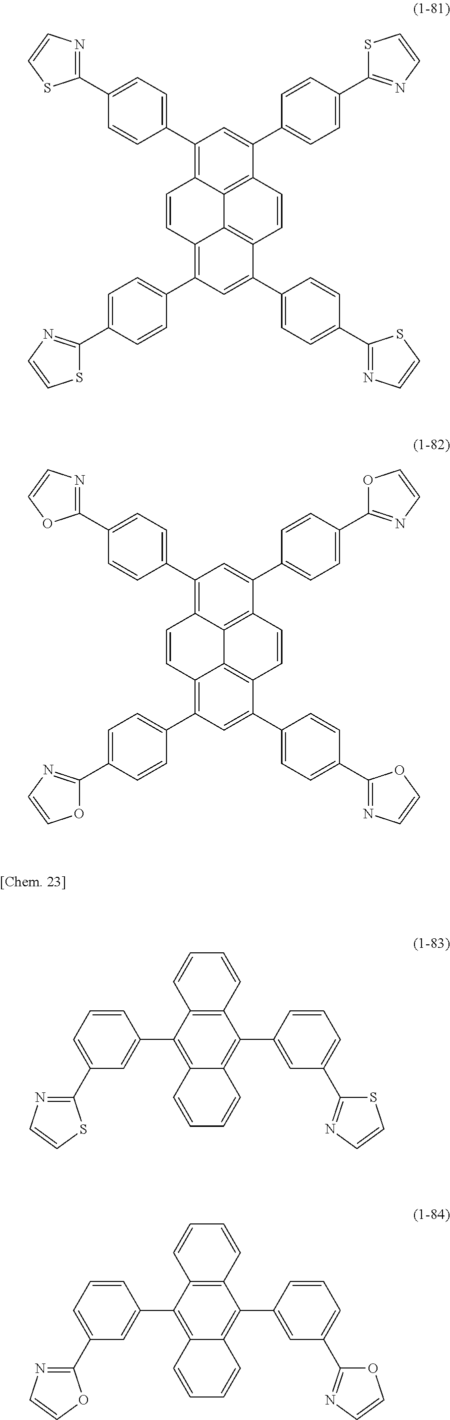 [SMILES: c1csc(-c2ccc(-c3cc(-c4ccc(-c5nccs5)cc4)c4ccc5c(-c6ccc(-c7nccs7)cc6)cc(-c6ccc(-c7nccs7)cc6)c6ccc3c4c65)cc2)n1 ; c1coc(-c2ccc(-c3cc(-c4ccc(-c5ncco5)cc4)c4ccc5c(-c6ccc(-c7ncco7)cc6)cc(-c6ccc(-c7ncco7)cc6)c6ccc3c4c65)cc2)n1 ; c1cc(-c2nccs2)cc(-c2c3ccccc3c(-c3cccc(-c4nccs4)c3)c3ccccc23)c1 ; c1cc(-c2ncco2)cc(-c2c3ccccc3c(-c3cccc(-c4ncco4)c3)c3ccccc23)c1]

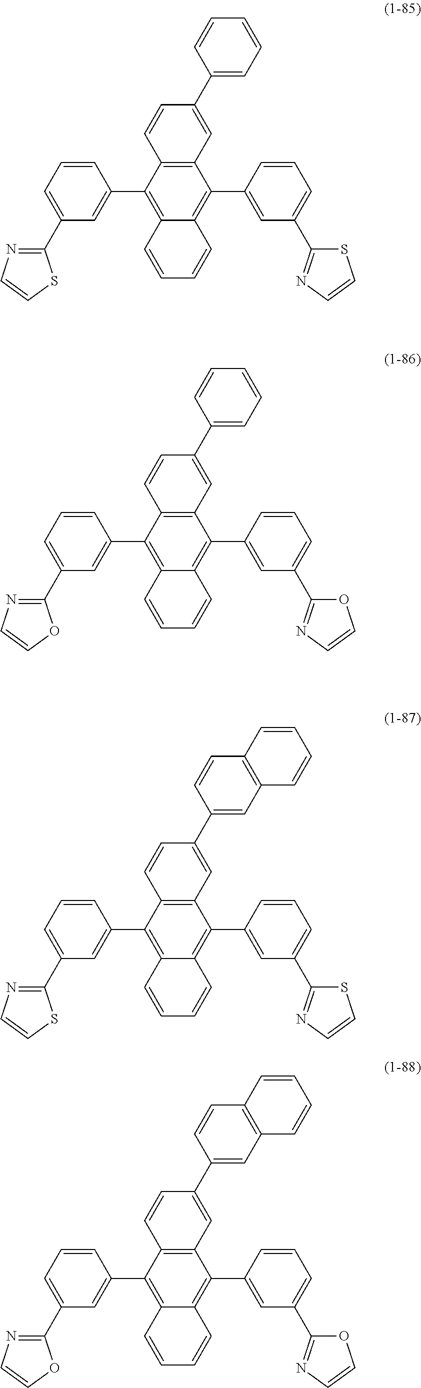 [SMILES: c1ccc(-c2ccc3c(-c4cccc(-c5nccs5)c4)c4ccccc4c(-c4cccc(-c5nccs5)c4)c3c2)cc1 ; c1ccc(-c2ccc3c(-c4cccc(-c5ncco5)c4)c4ccccc4c(-c4cccc(-c5ncco5)c4)c3c2)cc1 ; c1cc(-c2nccs2)cc(-c2c3ccccc3c(-c3cccc(-c4nccs4)c3)c3cc(-c4ccc5ccccc5c4)ccc23)c1 ; c1cc(-c2ncco2)cc(-c2c3ccccc3c(-c3cccc(-c4ncco4)c3)c3cc(-c4ccc5ccccc5c4)ccc23)c1]

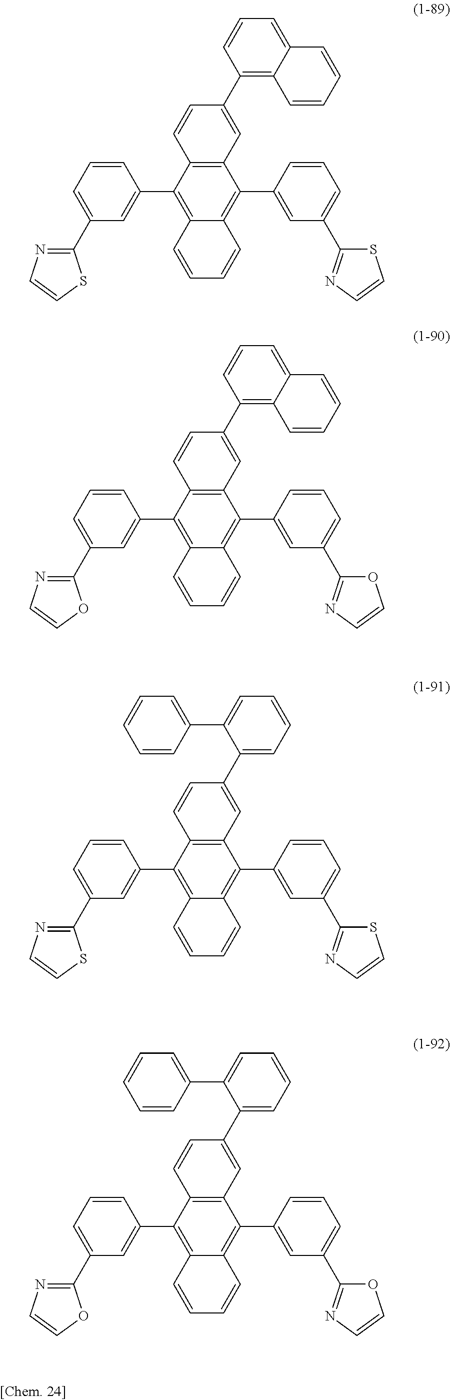 [SMILES: c1cc(-c2nccs2)cc(-c2c3ccccc3c(-c3cccc(-c4nccs4)c3)c3cc(-c4cccc5ccccc45)ccc23)c1 ; c1cc(-c2ncco2)cc(-c2c3ccccc3c(-c3cccc(-c4ncco4)c3)c3cc(-c4cccc5ccccc45)ccc23)c1 ; c1ccc(-c2ccccc2-c2ccc3c(-c4cccc(-c5nccs5)c4)c4ccccc4c(-c4cccc(-c5nccs5)c4)c3c2)cc1 ; c1ccc(-c2ccccc2-c2ccc3c(-c4cccc(-c5ncco5)c4)c4ccccc4c(-c4cccc(-c5ncco5)c4)c3c2)cc1]

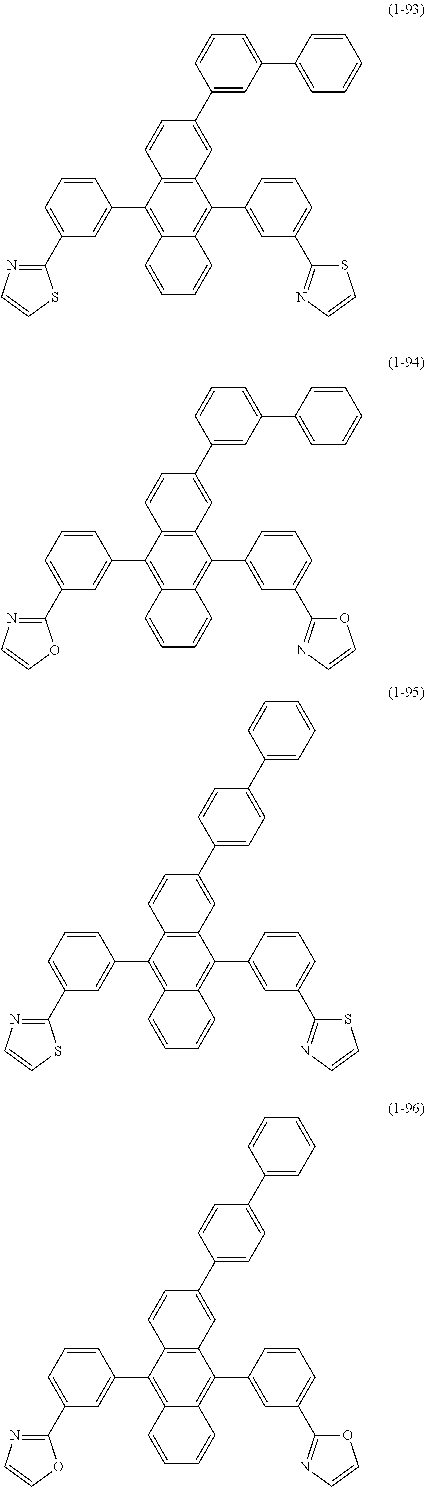

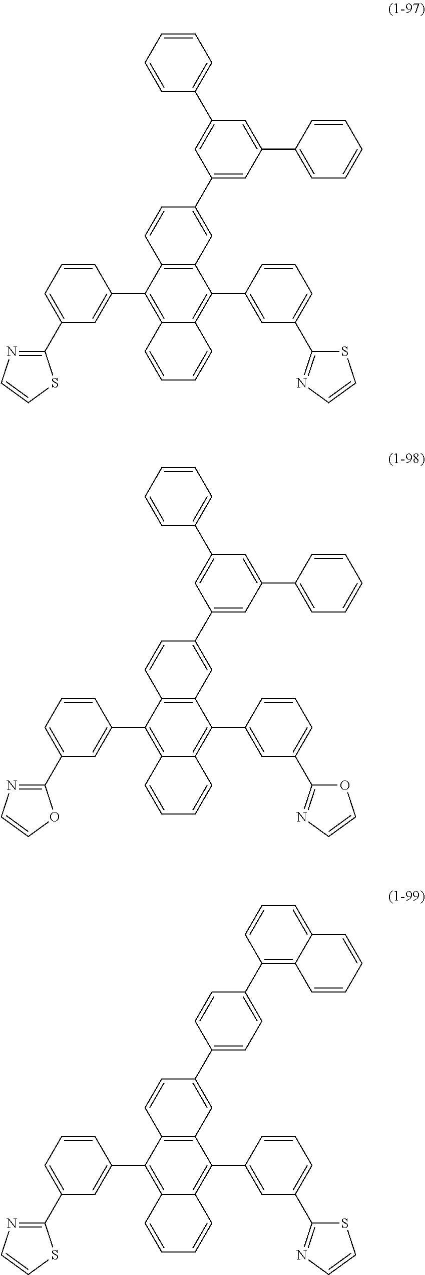 [SMILES: c1ccc(-c2cc(-c3ccccc3)cc(-c3ccc4c(-c5cccc(-c6nccs6)c5)c5ccccc5c(-c5cccc(-c6nccs6)c5)c4c3)c2)cc1 ; c1ccc(-c2cc(-c3ccccc3)cc(-c3ccc4c(-c5cccc(-c6ncco6)c5)c5ccccc5c(-c5cccc(-c6ncco6)c5)c4c3)c2)cc1 ; c1cc(-c2nccs2)cc(-c2c3ccccc3c(-c3cccc(-c4nccs4)c3)c3cc(-c4ccc(-c5cccc6ccccc56)cc4)ccc23)c1]

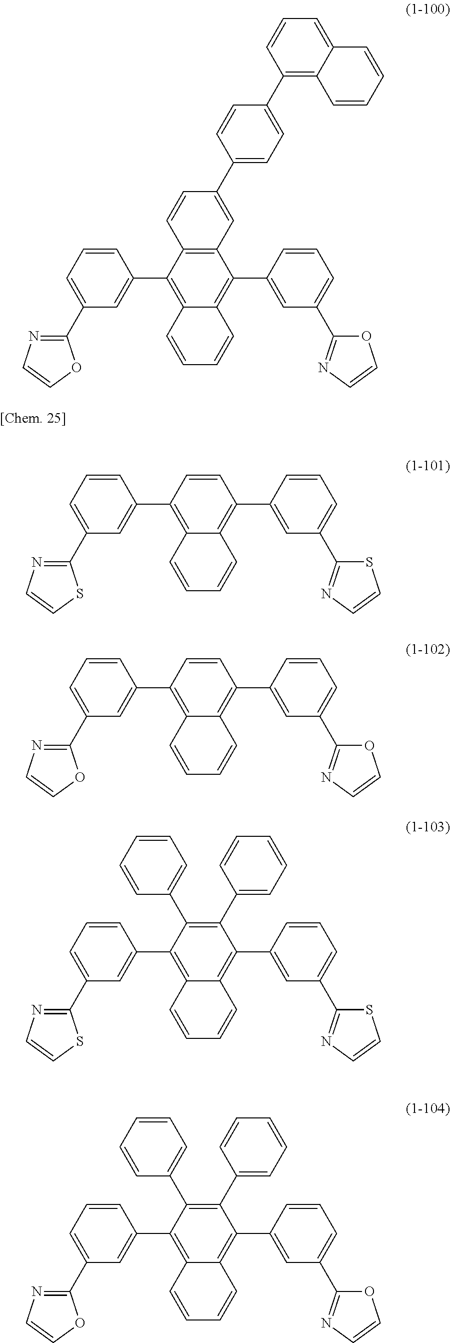 [SMILES: c1cc(-c2ncco2)cc(-c2c3ccccc3c(-c3cccc(-c4ncco4)c3)c3cc(-c4ccc(-c5cccc6ccccc56)cc4)ccc23)c1 ; c1cc(-c2nccs2)cc(-c2ccc(-c3cccc(-c4nccs4)c3)c3ccccc23)c1 ; c1cc(-c2ncco2)cc(-c2ccc(-c3cccc(-c4ncco4)c3)c3ccccc23)c1 ; c1ccc(-c2c(-c3ccccc3)c(-c3cccc(-c4nccs4)c3)c3ccccc3c2-c2cccc(-c3nccs3)c2)cc1 ; c1ccc(-c2c(-c3ccccc3)c(-c3cccc(-c4ncco4)c3)c3ccccc3c2-c2cccc(-c3ncco3)c2)cc1]

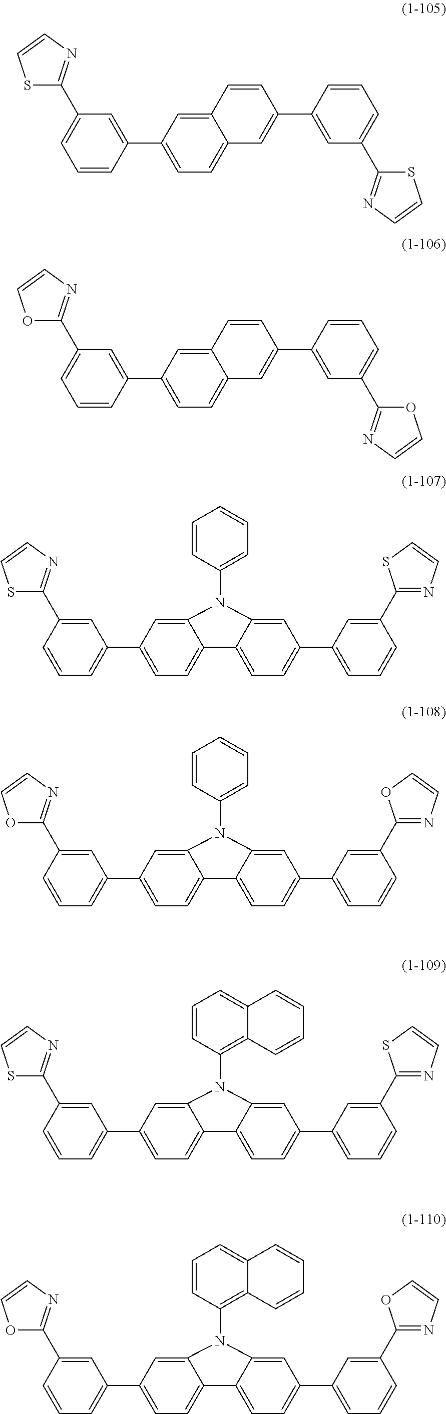 [SMILES: c1cc(-c2ccc3cc(-c4cccc(-c5nccs5)c4)ccc3c2)cc(-c2nccs2)c1 ; c1cc(-c2ccc3cc(-c4cccc(-c5ncco5)c4)ccc3c2)cc(-c2ncco2)c1 ; c1ccc(-n2c3cc(-c4cccc(-c5nccs5)c4)ccc3c3ccc(-c4cccc(-c5nccs5)c4)cc32)cc1 ; c1ccc(-n2c3cc(-c4cccc(-c5ncco5)c4)ccc3c3ccc(-c4cccc(-c5ncco5)c4)cc32)cc1 ; c1cc(-c2ccc3c4ccc(-c5cccc(-c6nccs6)c5)cc4n(-c4cccc5ccccc45)c3c2)cc(-c2nccs2)c1 ; c1cc(-c2ccc3c4ccc(-c5cccc(-c6ncco6)c5)cc4n(-c4cccc5ccccc45)c3c2)cc(-c2ncco2)c1]

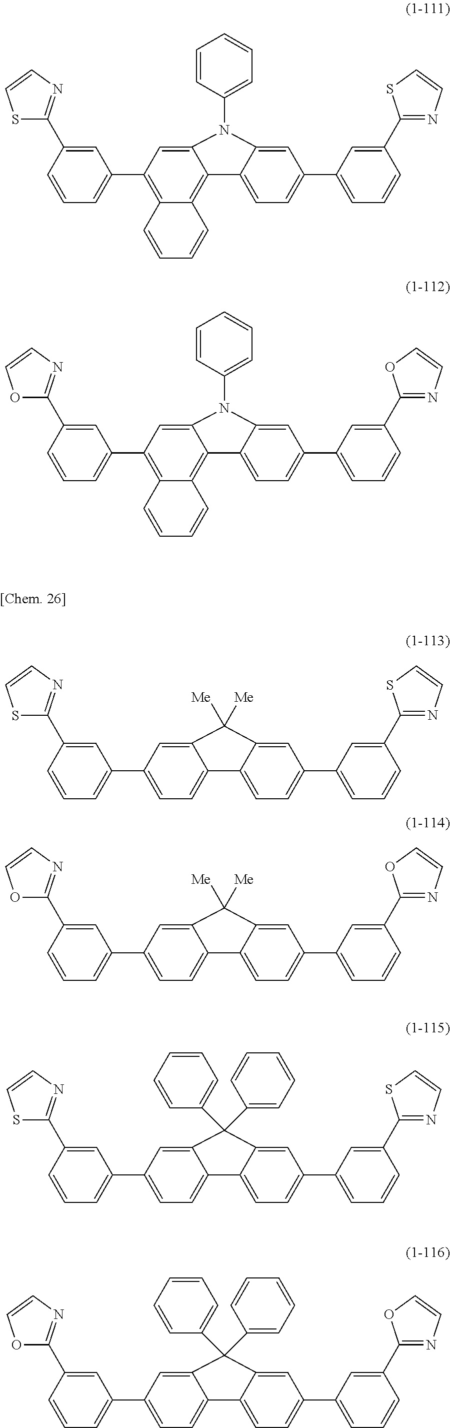 [SMILES: c1ccc(-n2c3cc(-c4cccc(-c5nccs5)c4)ccc3c3c4ccccc4c(-c4cccc(-c5nccs5)c4)cc32)cc1 ; c1ccc(-n2c3cc(-c4cccc(-c5ncco5)c4)ccc3c3c4ccccc4c(-c4cccc(-c5ncco5)c4)cc32)cc1 ; CC1(C)c2cc(-c3cccc(-c4nccs4)c3)ccc2-c2ccc(-c3cccc(-c4nccs4)c3)cc21 ; CC1(C)c2cc(-c3cccc(-c4ncco4)c3)ccc2-c2ccc(-c3cccc(-c4ncco4)c3)cc21 ; c1ccc(C2(c3ccccc3)c3cc(-c4cccc(-c5nccs5)c4)ccc3-c3ccc(-c4cccc(-c5nccs5)c4)cc32)cc1 ; c1ccc(C2(c3ccccc3)c3cc(-c4cccc(-c5ncco5)c4)ccc3-c3ccc(-c4cccc(-c5ncco5)c4)cc32)cc1]

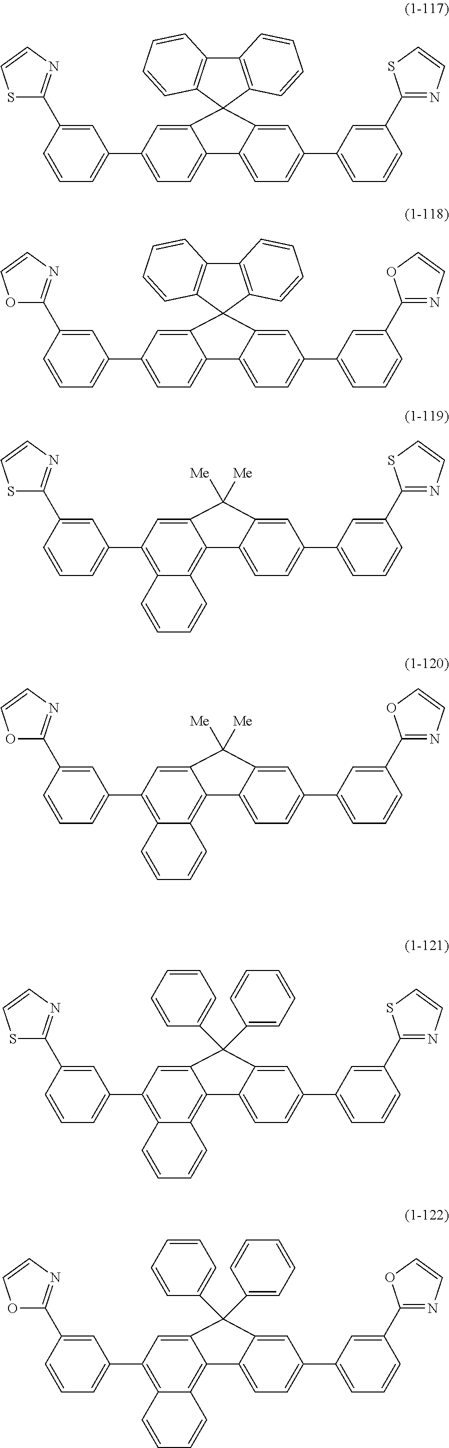

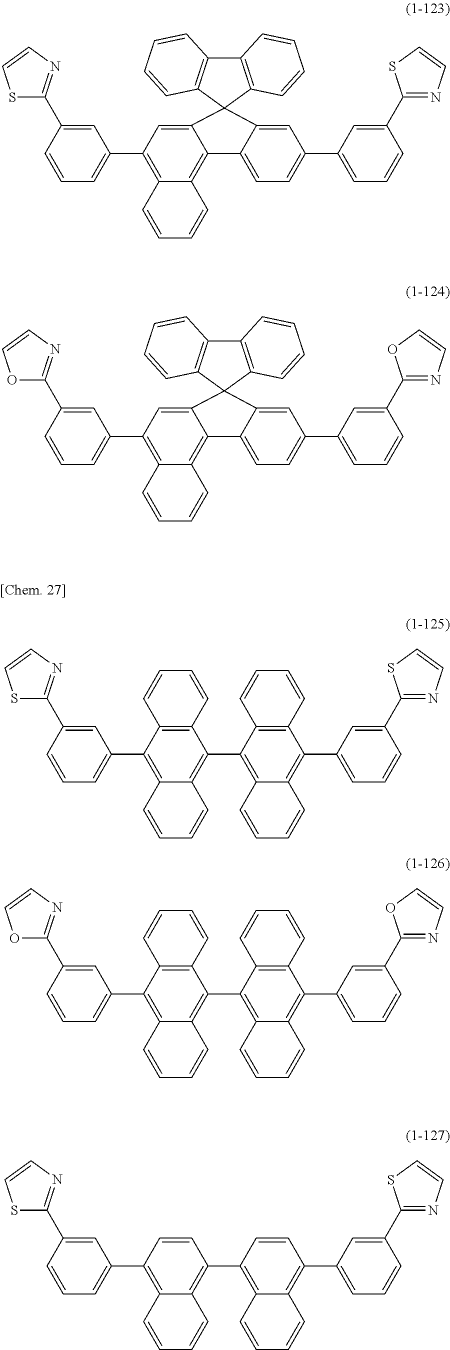 [SMILES: c1cc(-c2ccc3c(c2)C2(c4ccccc4-c4ccccc42)c2cc(-c4cccc(-c5nccs5)c4)c4ccccc4c2-3)cc(-c2nccs2)c1 ; c1cc(-c2ccc3c(c2)C2(c4ccccc4-c4ccccc42)c2cc(-c4cccc(-c5ncco5)c4)c4ccccc4c2-3)cc(-c2ncco2)c1 ; c1cc(-c2nccs2)cc(-c2c3ccccc3c(-c3c4ccccc4c(-c4cccc(-c5nccs5)c4)c4ccccc34)c3ccccc23)c1 ; c1cc(-c2ncco2)cc(-c2c3ccccc3c(-c3c4ccccc4c(-c4cccc(-c5ncco5)c4)c4ccccc34)c3ccccc23)c1 ; c1cc(-c2nccs2)cc(-c2ccc(-c3ccc(-c4cccc(-c5nccs5)c4)c4ccccc34)c3ccccc23)c1]

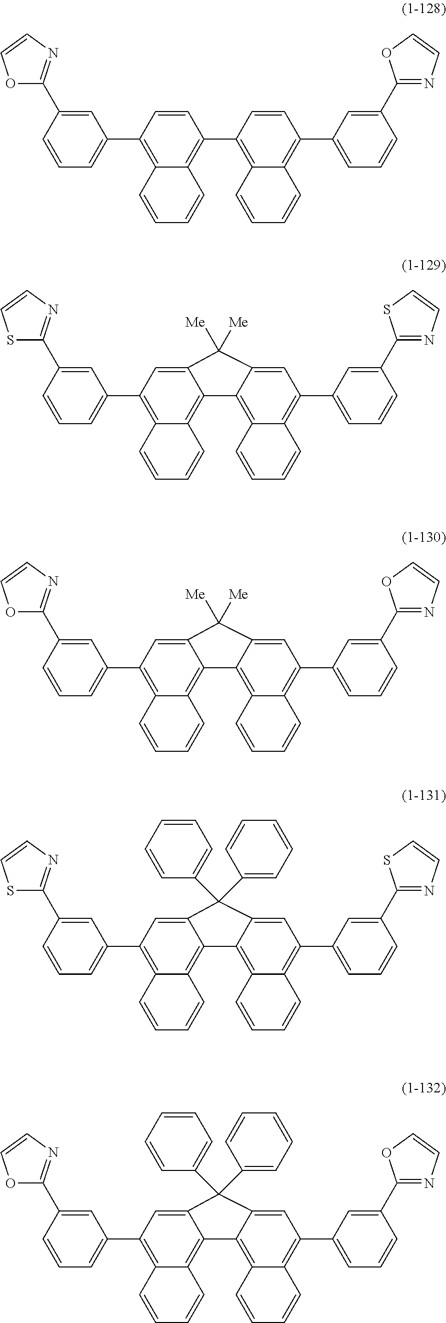 [SMILES: c1cc(-c2ncco2)cc(-c2ccc(-c3ccc(-c4cccc(-c5ncco5)c4)c4ccccc34)c3ccccc23)c1 ; CC1(C)c2cc(-c3cccc(-c4nccs4)c3)c3ccccc3c2-c2c1cc(-c1cccc(-c3nccs3)c1)c1ccccc21 ; CC1(C)c2cc(-c3cccc(-c4ncco4)c3)c3ccccc3c2-c2c1cc(-c1cccc(-c3ncco3)c1)c1ccccc21 ; c1ccc(C2(c3ccccc3)c3cc(-c4cccc(-c5nccs5)c4)c4ccccc4c3-c3c2cc(-c2cccc(-c4nccs4)c2)c2ccccc32)cc1 ; c1ccc(C2(c3ccccc3)c3cc(-c4cccc(-c5ncco5)c4)c4ccccc4c3-c3c2cc(-c2cccc(-c4ncco4)c2)c2ccccc32)cc1]

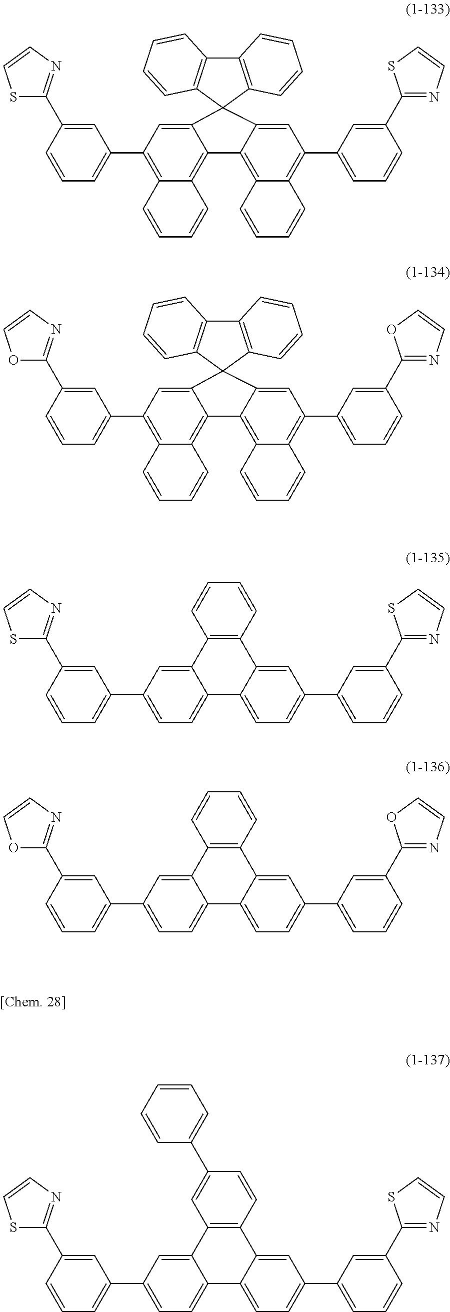 [SMILES: c1cc(-c2nccs2)cc(-c2cc3c(c4ccccc24)-c2c(cc(-c4cccc(-c5nccs5)c4)c4ccccc24)C32c3ccccc3-c3ccccc32)c1 ; c1cc(-c2ncco2)cc(-c2cc3c(c4ccccc24)-c2c(cc(-c4cccc(-c5ncco5)c4)c4ccccc24)C32c3ccccc3-c3ccccc32)c1 ; c1cc(-c2ccc3c4ccc(-c5cccc(-c6nccs6)c5)cc4c4ccccc4c3c2)cc(-c2nccs2)c1 ; c1cc(-c2ccc3c4ccc(-c5cccc(-c6ncco6)c5)cc4c4ccccc4c3c2)cc(-c2ncco2)c1 ; c1ccc(-c2ccc3c4cc(-c5cccc(-c6nccs6)c5)ccc4c4ccc(-c5cccc(-c6nccs6)c5)cc4c3c2)cc1]

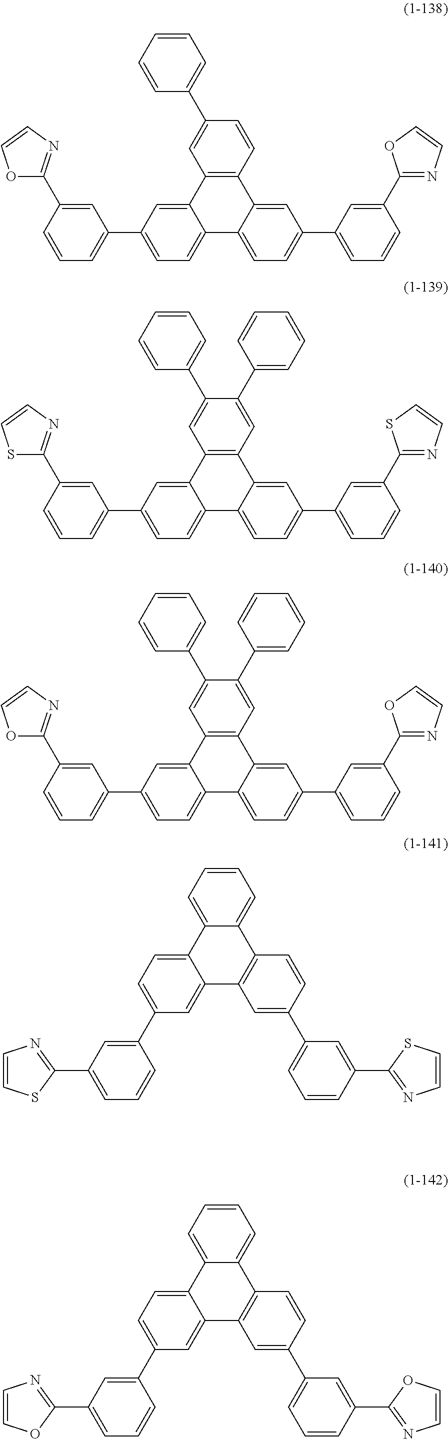 [SMILES: c1ccc(-c2ccc3c4cc(-c5cccc(-c6ncco6)c5)ccc4c4ccc(-c5cccc(-c6ncco6)c5)cc4c3c2)cc1 ; c1ccc(-c2cc3c4cc(-c5cccc(-c6nccs6)c5)ccc4c4ccc(-c5cccc(-c6nccs6)c5)cc4c3cc2-c2ccccc2)cc1 ; c1ccc(-c2cc3c4cc(-c5cccc(-c6ncco6)c5)ccc4c4ccc(-c5cccc(-c6ncco6)c5)cc4c3cc2-c2ccccc2)cc1 ; c1cc(-c2ccc3c4ccccc4c4ccc(-c5cccc(-c6nccs6)c5)cc4c3c2)cc(-c2nccs2)c1 ; c1cc(-c2ccc3c4ccccc4c4ccc(-c5cccc(-c6ncco6)c5)cc4c3c2)cc(-c2ncco2)c1]

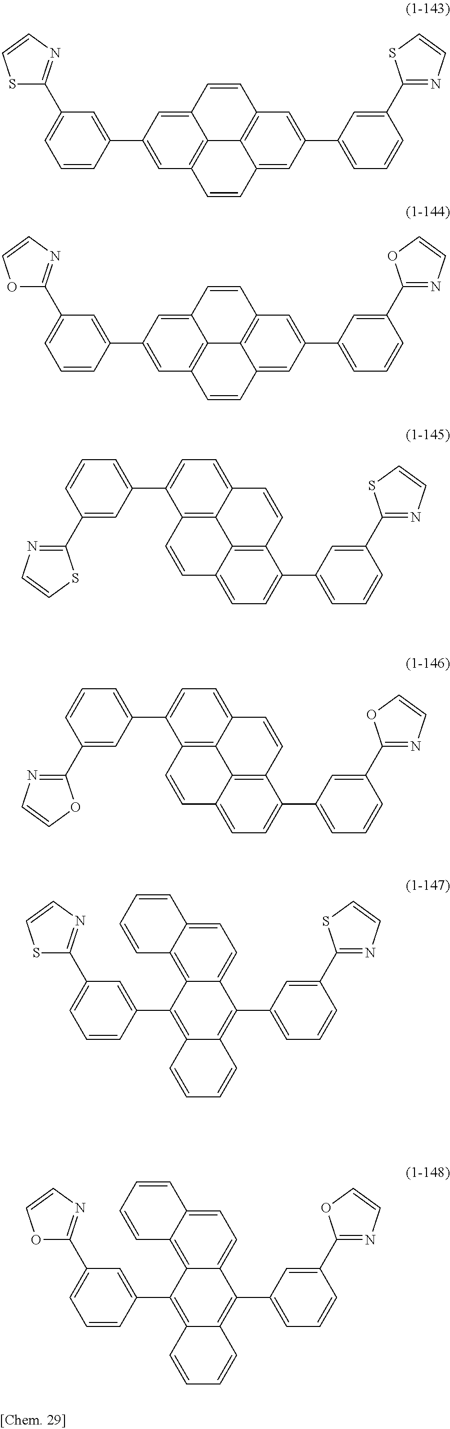 [SMILES: c1cc(-c2cc3ccc4cc(-c5cccc(-c6nccs6)c5)cc5ccc(c2)c3c45)cc(-c2nccs2)c1 ; c1cc(-c2cc3ccc4cc(-c5cccc(-c6ncco6)c5)cc5ccc(c2)c3c45)cc(-c2ncco2)c1 ; c1cc(-c2nccs2)cc(-c2ccc3ccc4c(-c5cccc(-c6nccs6)c5)ccc5ccc2c3c54)c1 ; c1cc(-c2ncco2)cc(-c2ccc3ccc4c(-c5cccc(-c6ncco6)c5)ccc5ccc2c3c54)c1 ; c1cc(-c2nccs2)cc(-c2c3ccccc3c(-c3cccc(-c4nccs4)c3)c3c2ccc2ccccc23)c1 ; c1cc(-c2ncco2)cc(-c2c3ccccc3c(-c3cccc(-c4ncco4)c3)c3c2ccc2ccccc23)c1]

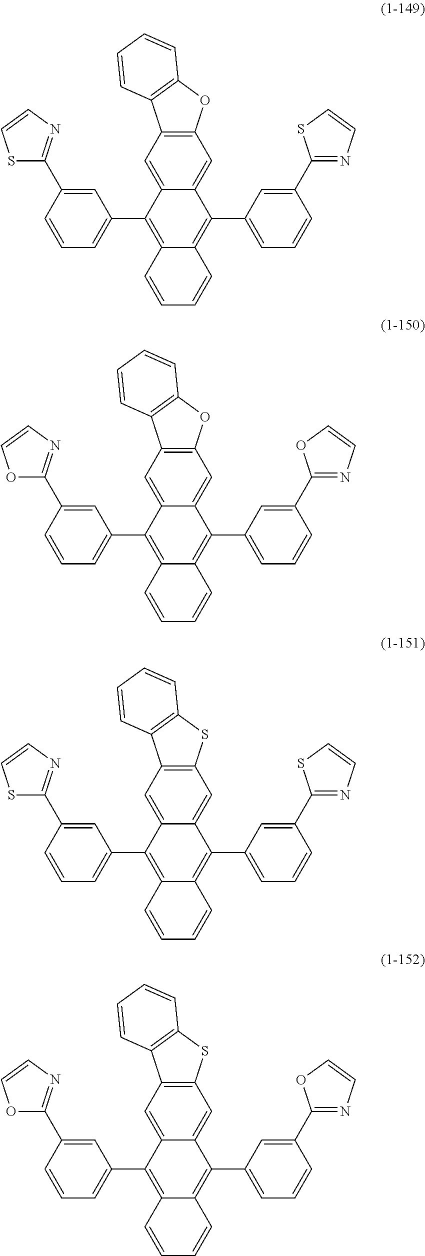 [SMILES: c1cc(-c2nccs2)cc(-c2c3ccccc3c(-c3cccc(-c4nccs4)c3)c3cc4c(cc23)oc2ccccc24)c1 ; c1cc(-c2ncco2)cc(-c2c3ccccc3c(-c3cccc(-c4ncco4)c3)c3cc4c(cc23)oc2ccccc24)c1 ; c1cc(-c2nccs2)cc(-c2c3ccccc3c(-c3cccc(-c4nccs4)c3)c3cc4c(cc23)sc2ccccc24)c1 ; c1cc(-c2ncco2)cc(-c2c3ccccc3c(-c3cccc(-c4ncco4)c3)c3cc4c(cc23)sc2ccccc24)c1]

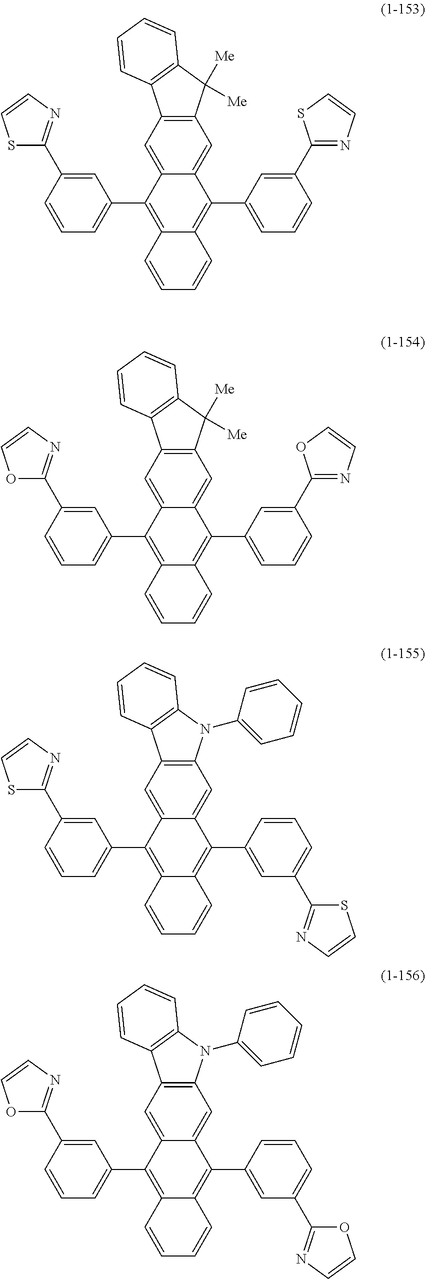

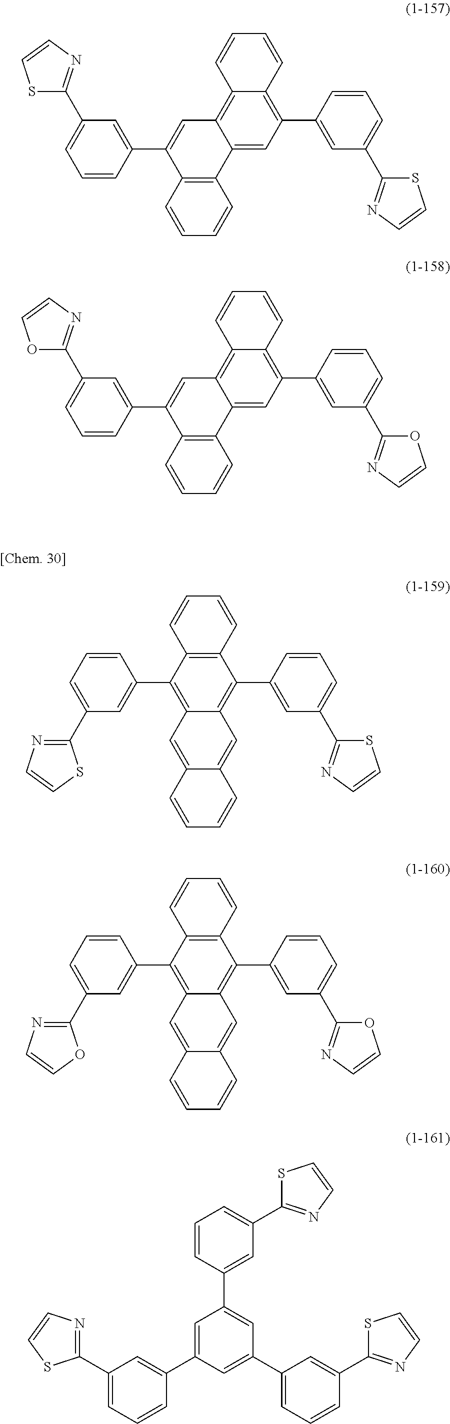 [SMILES: c1cc(-c2nccs2)cc(-c2cc3c4ccccc4c(-c4cccc(-c5nccs5)c4)cc3c3ccccc23)c1 ; c1cc(-c2ncco2)cc(-c2cc3c4ccccc4c(-c4cccc(-c5ncco5)c4)cc3c3ccccc23)c1 ; c1cc(-c2nccs2)cc(-c2c3ccccc3c(-c3cccc(-c4nccs4)c3)c3cc4ccccc4cc23)c1 ; c1cc(-c2ncco2)cc(-c2c3ccccc3c(-c3cccc(-c4ncco4)c3)c3cc4ccccc4cc23)c1 ; c1cc(-c2cc(-c3cccc(-c4nccs4)c3)cc(-c3cccc(-c4nccs4)c3)c2)cc(-c2nccs2)c1]

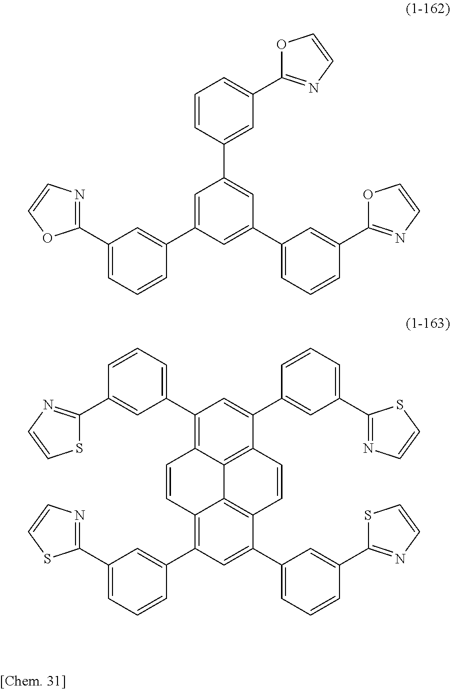 [SMILES: c1cc(-c2cc(-c3cccc(-c4ncco4)c3)cc(-c3cccc(-c4ncco4)c3)c2)cc(-c2ncco2)c1 ; c1cc(-c2nccs2)cc(-c2cc(-c3cccc(-c4nccs4)c3)c3ccc4c(-c5cccc(-c6nccs6)c5)cc(-c5cccc(-c6nccs6)c5)c5ccc2c3c54)c1]

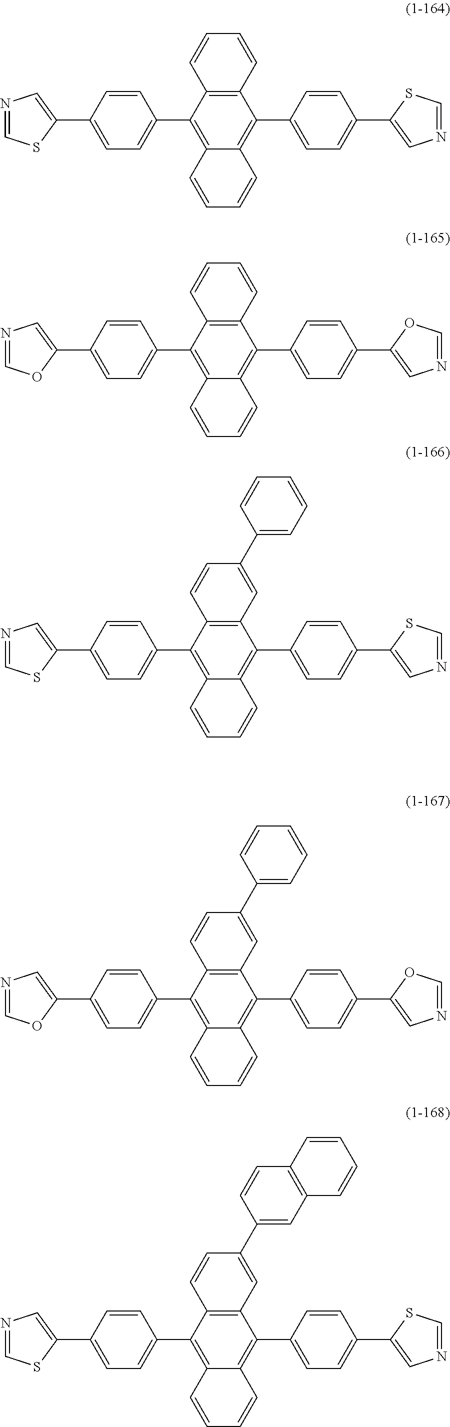

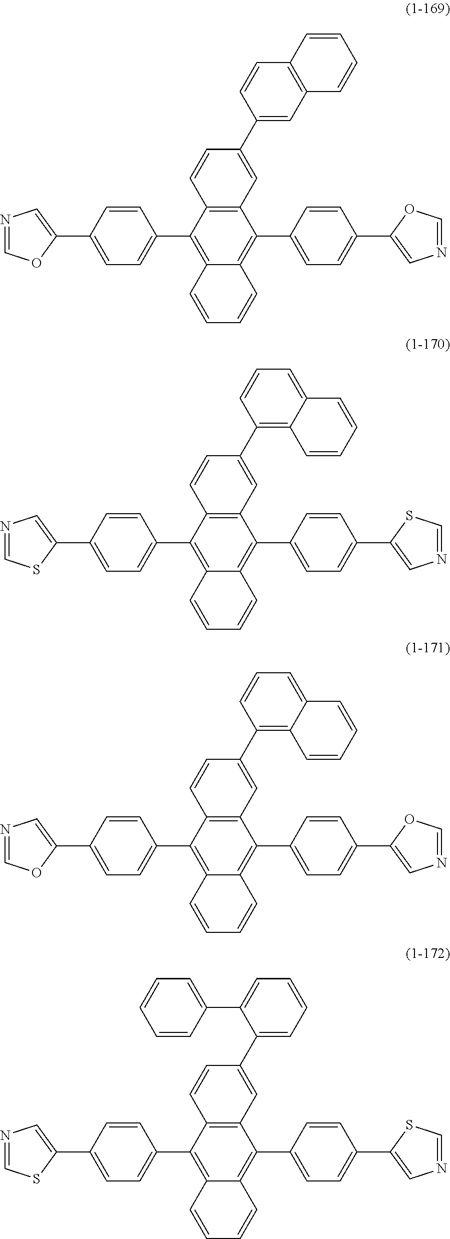

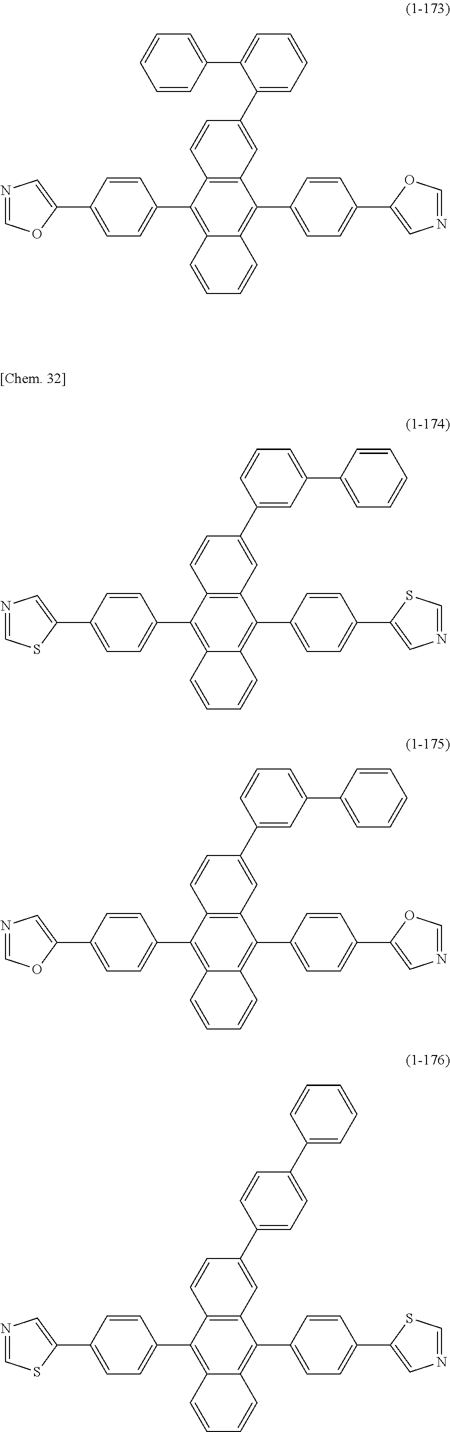 [SMILES: c1ccc(-c2ccccc2-c2ccc3c(-c4ccc(-c5cnco5)cc4)c4ccccc4c(-c4ccc(-c5cnco5)cc4)c3c2)cc1 ; c1ccc(-c2cccc(-c3ccc4c(-c5ccc(-c6cncs6)cc5)c5ccccc5c(-c5ccc(-c6cncs6)cc5)c4c3)c2)cc1 ; c1ccc(-c2cccc(-c3ccc4c(-c5ccc(-c6cnco6)cc5)c5ccccc5c(-c5ccc(-c6cnco6)cc5)c4c3)c2)cc1 ; c1ccc(-c2ccc(-c3ccc4c(-c5ccc(-c6cncs6)cc5)c5ccccc5c(-c5ccc(-c6cncs6)cc5)c4c3)cc2)cc1]

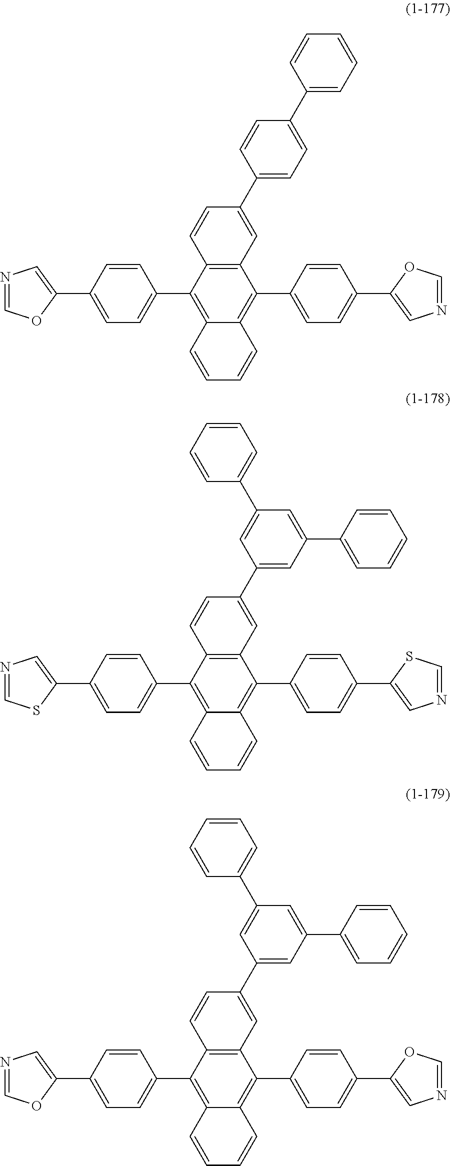

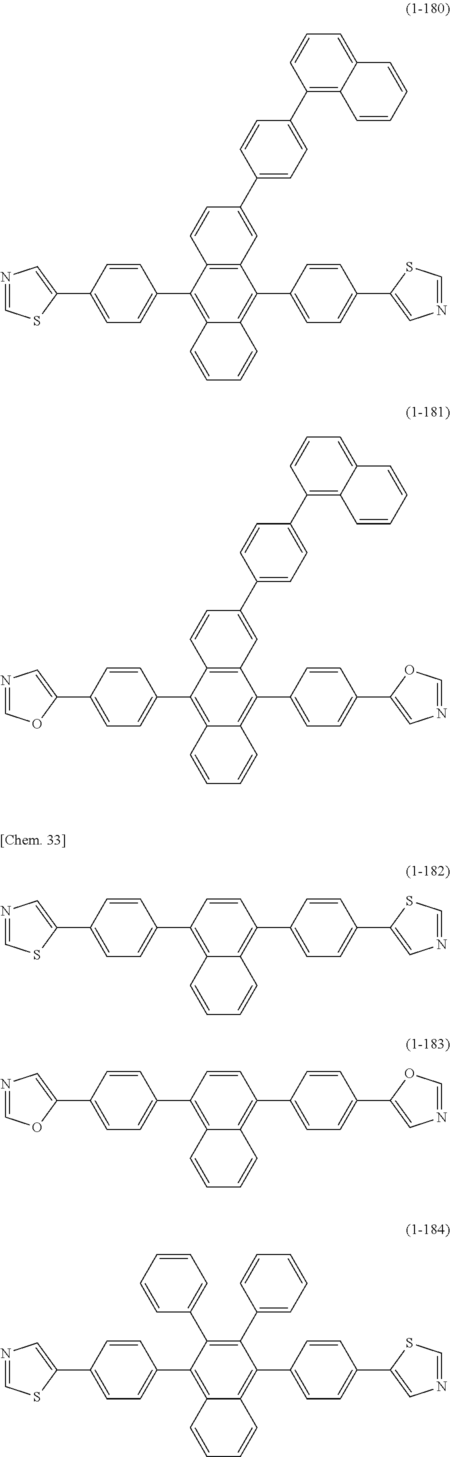 [SMILES: c1ccc2c(-c3ccc(-c4ccc5c(-c6ccc(-c7cncs7)cc6)c6ccccc6c(-c6ccc(-c7cncs7)cc6)c5c4)cc3)cccc2c1 ; c1ccc2c(-c3ccc(-c4ccc5c(-c6ccc(-c7cnco7)cc6)c6ccccc6c(-c6ccc(-c7cnco7)cc6)c5c4)cc3)cccc2c1 ; c1ccc2c(-c3ccc(-c4cncs4)cc3)ccc(-c3ccc(-c4cncs4)cc3)c2c1 ; c1ccc2c(-c3ccc(-c4cnco4)cc3)ccc(-c3ccc(-c4cnco4)cc3)c2c1 ; c1ccc(-c2c(-c3ccccc3)c(-c3ccc(-c4cncs4)cc3)c3ccccc3c2-c2ccc(-c3cncs3)cc2)cc1]

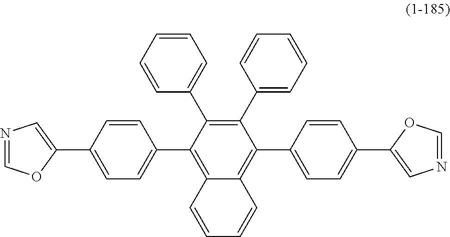 [SMILES: c1ccc(-c2c(-c3ccccc3)c(-c3ccc(-c4cnco4)cc3)c3ccccc3c2-c2ccc(-c3cnco3)cc2)cc1]

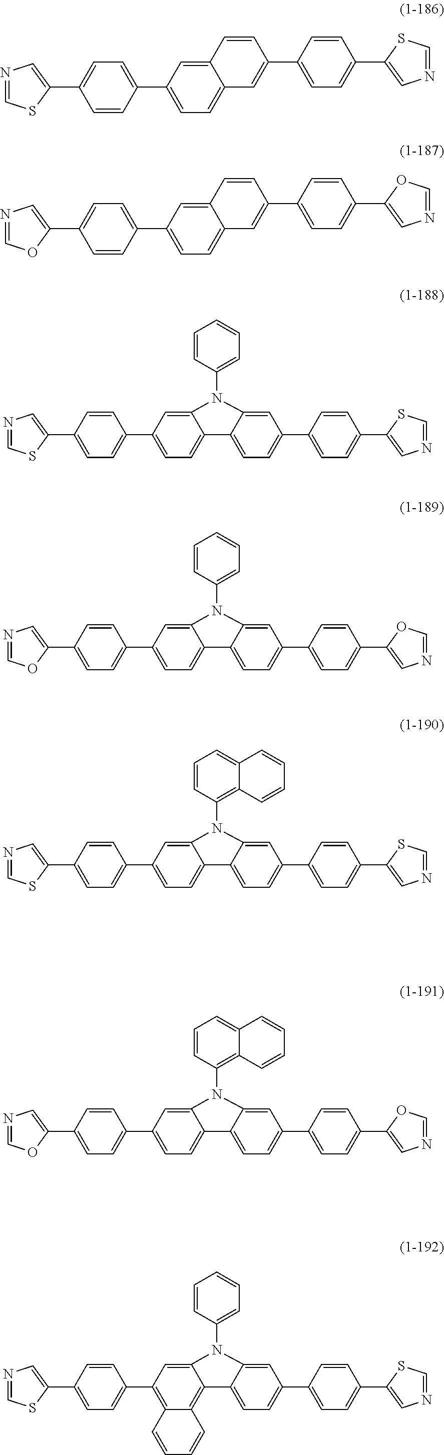

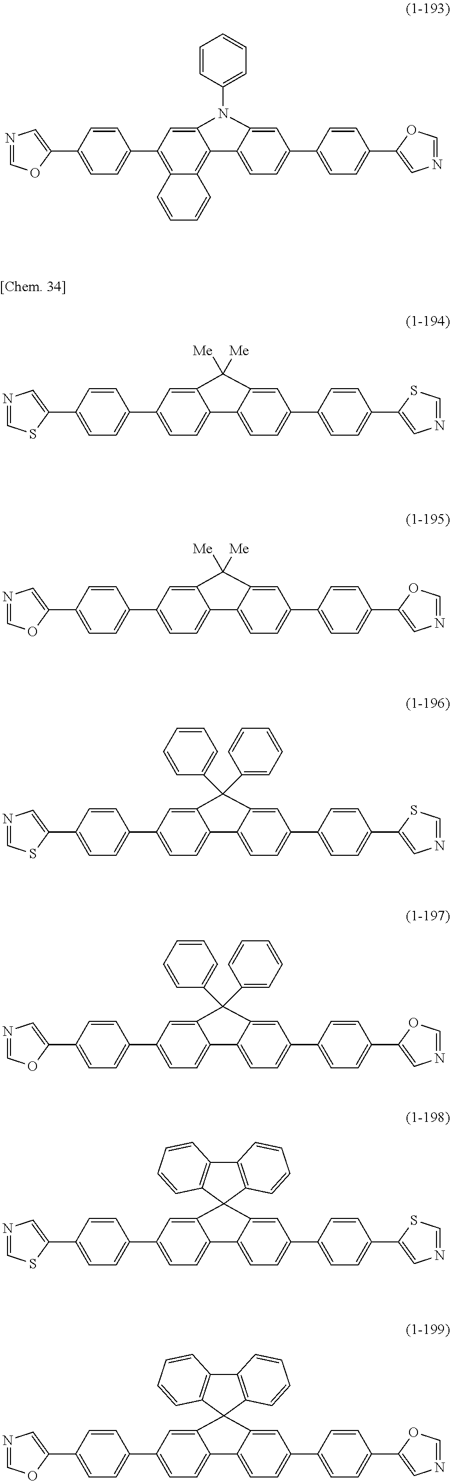

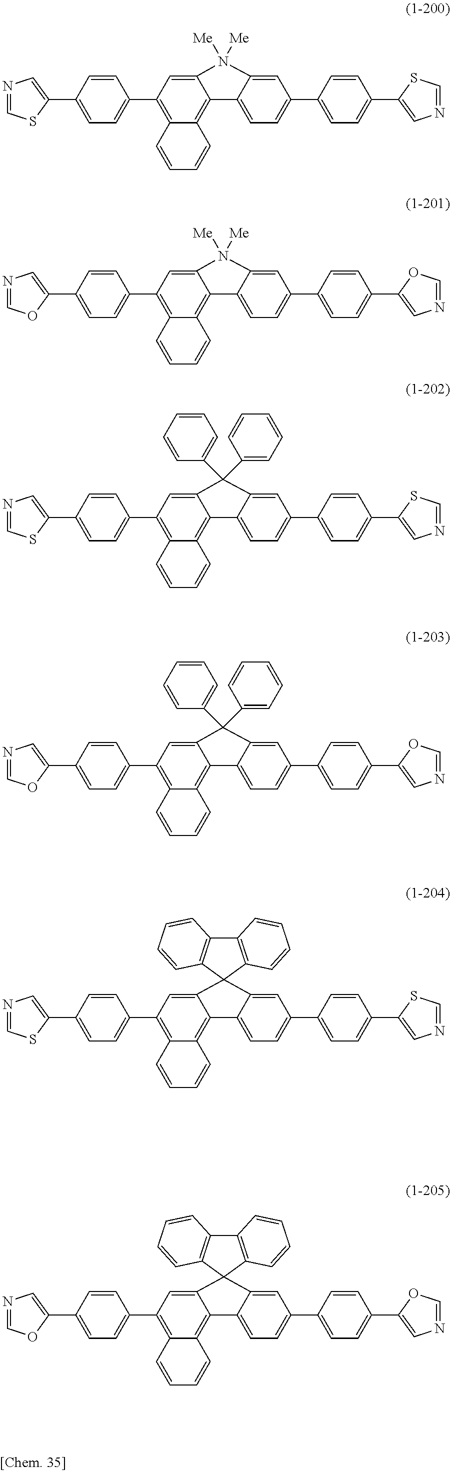

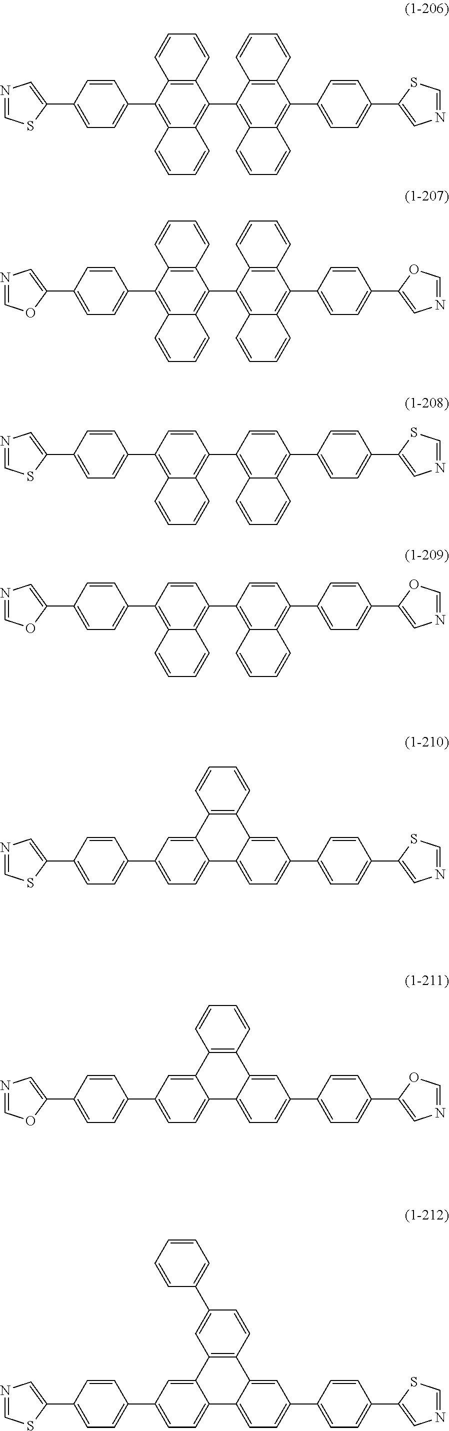

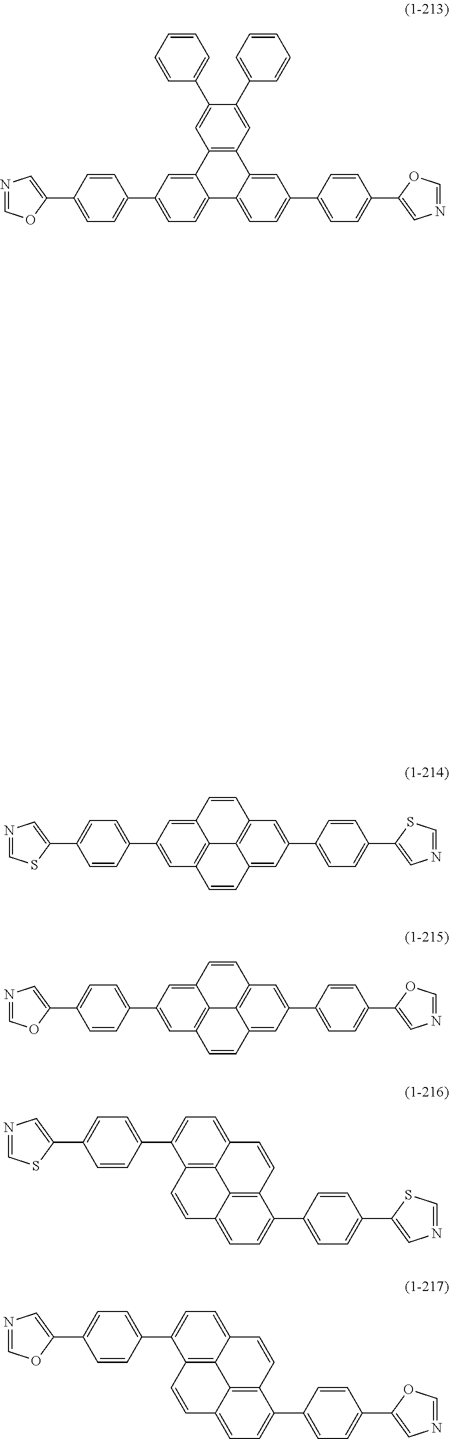 [SMILES: c1ccc(-c2cc3c4cc(-c5ccc(-c6cnco6)cc5)ccc4c4ccc(-c5ccc(-c6cnco6)cc5)cc4c3cc2-c2ccccc2)cc1 ; c1ncc(-c2ccc(-c3cc4ccc5cc(-c6ccc(-c7cncs7)cc6)cc6ccc(c3)c4c56)cc2)s1 ; c1ncc(-c2ccc(-c3cc4ccc5cc(-c6ccc(-c7cnco7)cc6)cc6ccc(c3)c4c56)cc2)o1 ; c1ncc(-c2ccc(-c3ccc4ccc5c(-c6ccc(-c7cncs7)cc6)ccc6ccc3c4c65)cc2)s1 ; c1ncc(-c2ccc(-c3ccc4ccc5c(-c6ccc(-c7cnco7)cc6)ccc6ccc3c4c65)cc2)o1]

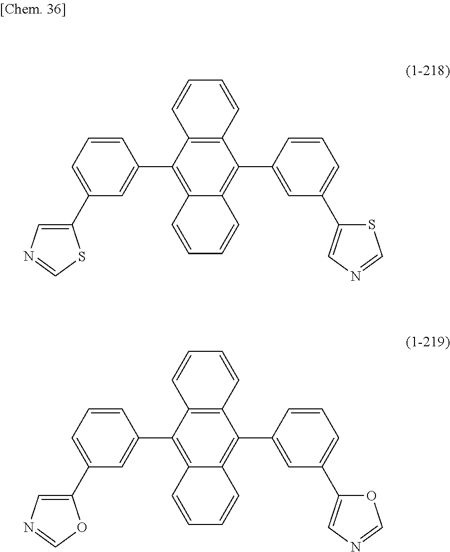 [SMILES: c1cc(-c2cncs2)cc(-c2c3ccccc3c(-c3cccc(-c4cncs4)c3)c3ccccc23)c1 ; c1cc(-c2cnco2)cc(-c2c3ccccc3c(-c3cccc(-c4cnco4)c3)c3ccccc23)c1]

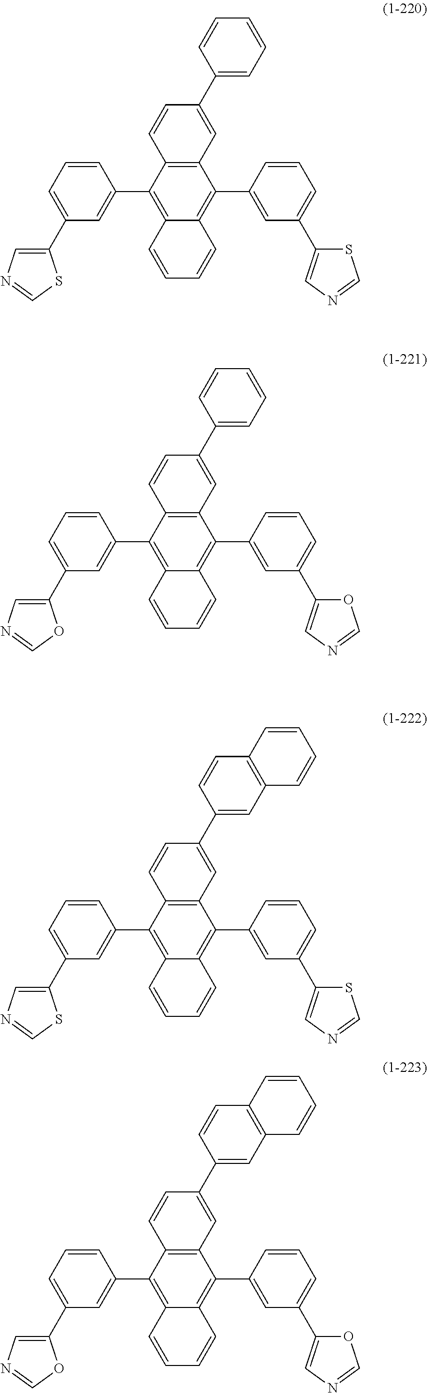 [SMILES: c1ccc(-c2ccc3c(-c4cccc(-c5cncs5)c4)c4ccccc4c(-c4cccc(-c5cncs5)c4)c3c2)cc1 ; c1ccc(-c2ccc3c(-c4cccc(-c5cnco5)c4)c4ccccc4c(-c4cccc(-c5cnco5)c4)c3c2)cc1 ; c1cc(-c2cncs2)cc(-c2c3ccccc3c(-c3cccc(-c4cncs4)c3)c3cc(-c4ccc5ccccc5c4)ccc23)c1 ; c1cc(-c2cnco2)cc(-c2c3ccccc3c(-c3cccc(-c4cnco4)c3)c3cc(-c4ccc5ccccc5c4)ccc23)c1]

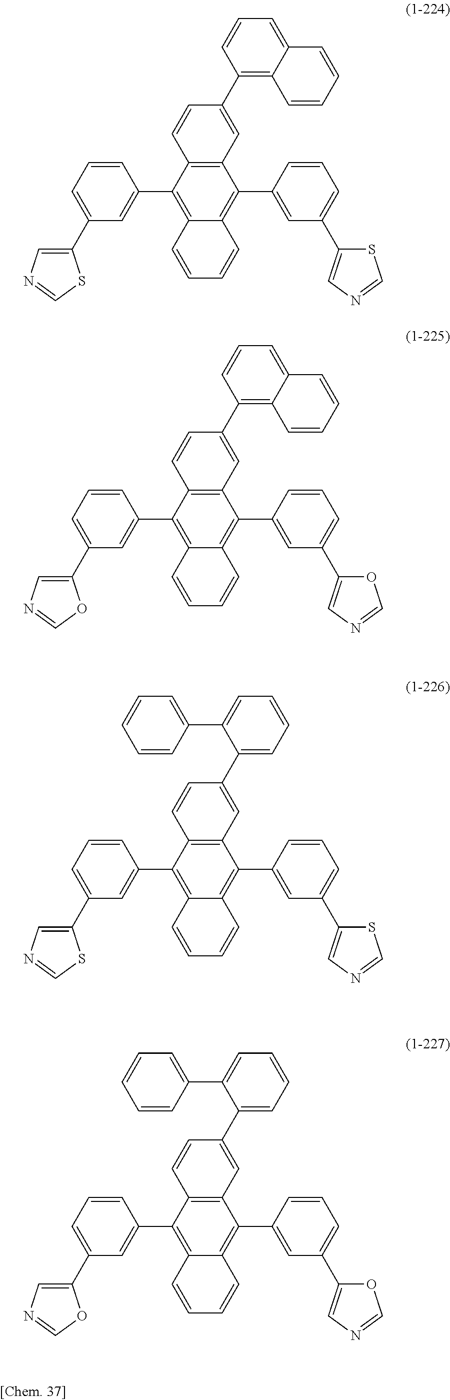 [SMILES: c1cc(-c2cncs2)cc(-c2c3ccccc3c(-c3cccc(-c4cncs4)c3)c3cc(-c4cccc5ccccc45)ccc23)c1 ; c1cc(-c2cnco2)cc(-c2c3ccccc3c(-c3cccc(-c4cnco4)c3)c3cc(-c4cccc5ccccc45)ccc23)c1 ; c1ccc(-c2ccccc2-c2ccc3c(-c4cccc(-c5cncs5)c4)c4ccccc4c(-c4cccc(-c5cncs5)c4)c3c2)cc1 ; c1ccc(-c2ccccc2-c2ccc3c(-c4cccc(-c5cnco5)c4)c4ccccc4c(-c4cccc(-c5cnco5)c4)c3c2)cc1]

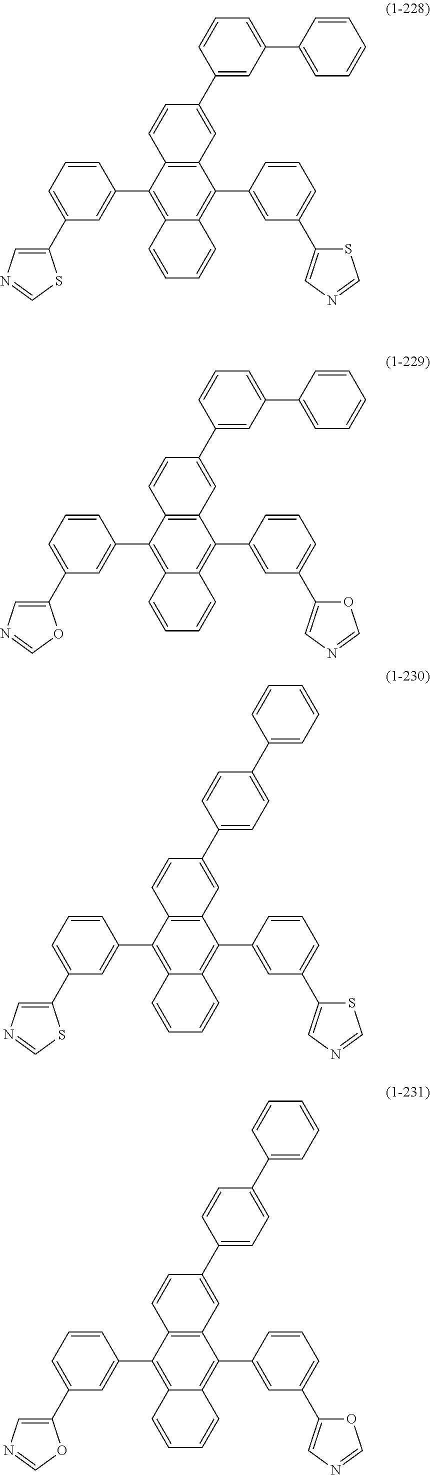

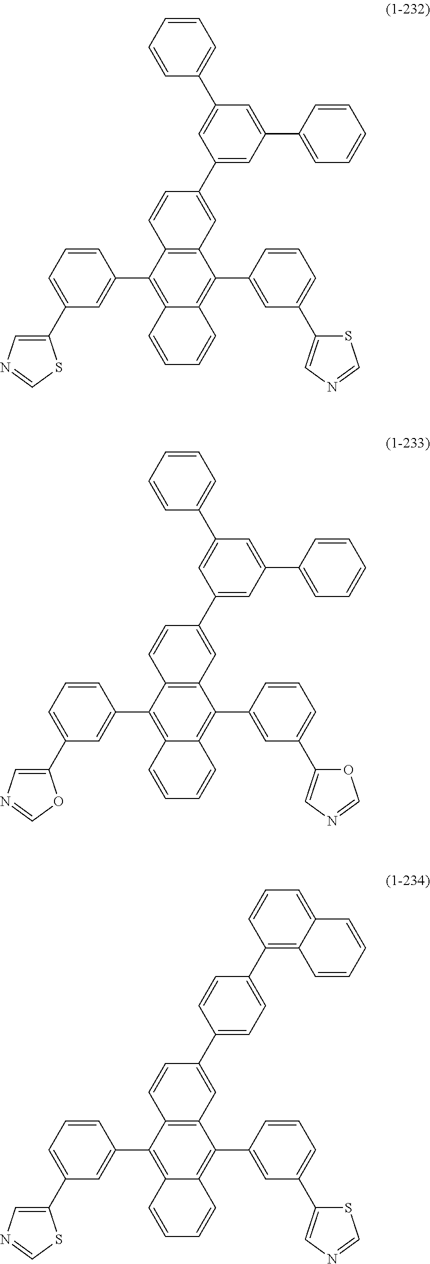 [SMILES: c1ccc(-c2cc(-c3ccccc3)cc(-c3ccc4c(-c5cccc(-c6cncs6)c5)c5ccccc5c(-c5cccc(-c6cncs6)c5)c4c3)c2)cc1 ; c1ccc(-c2cc(-c3ccccc3)cc(-c3ccc4c(-c5cccc(-c6cnco6)c5)c5ccccc5c(-c5cccc(-c6cnco6)c5)c4c3)c2)cc1 ; c1cc(-c2cncs2)cc(-c2c3ccccc3c(-c3cccc(-c4cncs4)c3)c3cc(-c4ccc(-c5cccc6ccccc56)cc4)ccc23)c1]

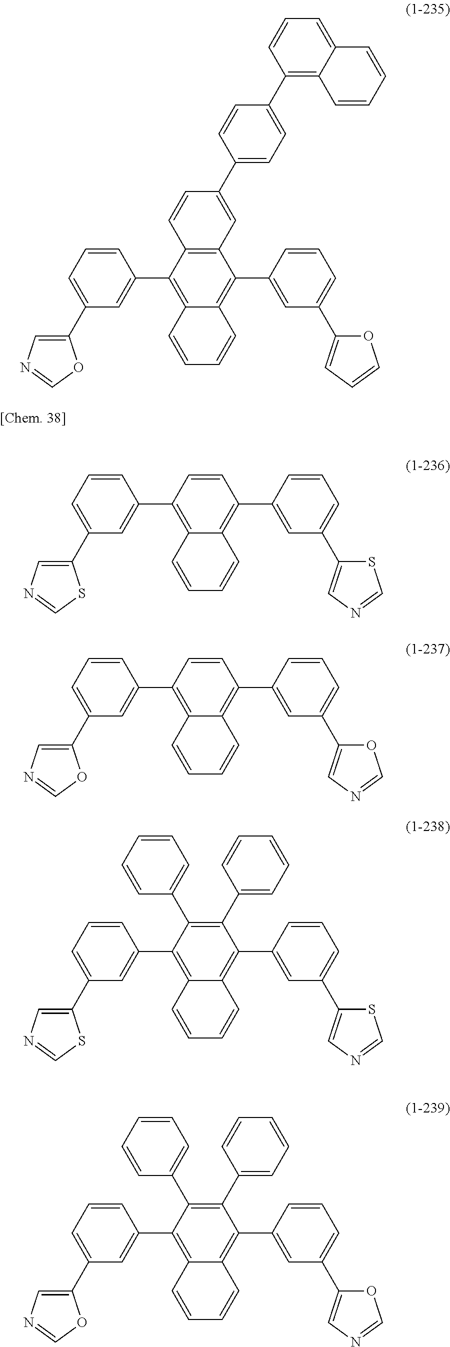 [SMILES: c1cc(-c2cnco2)cc(-c2c3ccccc3c(-c3cccc(-c4ccco4)c3)c3cc(-c4ccc(-c5cccc6ccccc56)cc4)ccc23)c1 ; c1cc(-c2cncs2)cc(-c2ccc(-c3cccc(-c4cncs4)c3)c3ccccc23)c1 ; c1cc(-c2cnco2)cc(-c2ccc(-c3cccc(-c4cnco4)c3)c3ccccc23)c1 ; c1ccc(-c2c(-c3ccccc3)c(-c3cccc(-c4cncs4)c3)c3ccccc3c2-c2cccc(-c3cncs3)c2)cc1 ; c1ccc(-c2c(-c3ccccc3)c(-c3cccc(-c4cnco4)c3)c3ccccc3c2-c2cccc(-c3cnco3)c2)cc1]

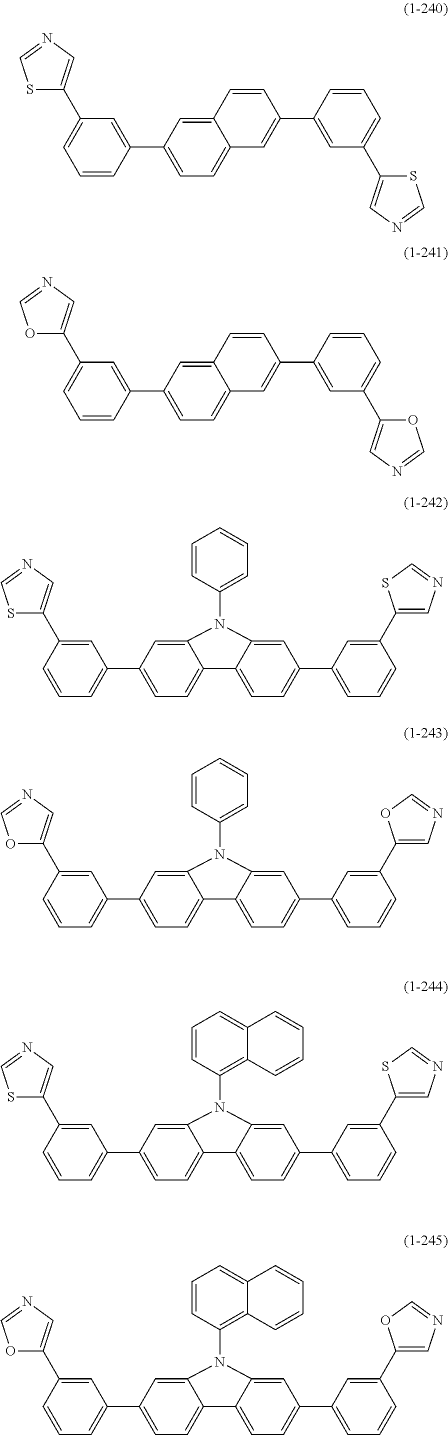 [SMILES: c1cc(-c2ccc3cc(-c4cccc(-c5cncs5)c4)ccc3c2)cc(-c2cncs2)c1 ; c1cc(-c2ccc3cc(-c4cccc(-c5cnco5)c4)ccc3c2)cc(-c2cnco2)c1 ; c1ccc(-n2c3cc(-c4cccc(-c5cncs5)c4)ccc3c3ccc(-c4cccc(-c5cncs5)c4)cc32)cc1 ; c1ccc(-n2c3cc(-c4cccc(-c5cnco5)c4)ccc3c3ccc(-c4cccc(-c5cnco5)c4)cc32)cc1 ; c1cc(-c2ccc3c4ccc(-c5cccc(-c6cncs6)c5)cc4n(-c4cccc5ccccc45)c3c2)cc(-c2cncs2)c1 ; c1cc(-c2ccc3c4ccc(-c5cccc(-c6cnco6)c5)cc4n(-c4cccc5ccccc45)c3c2)cc(-c2cnco2)c1]

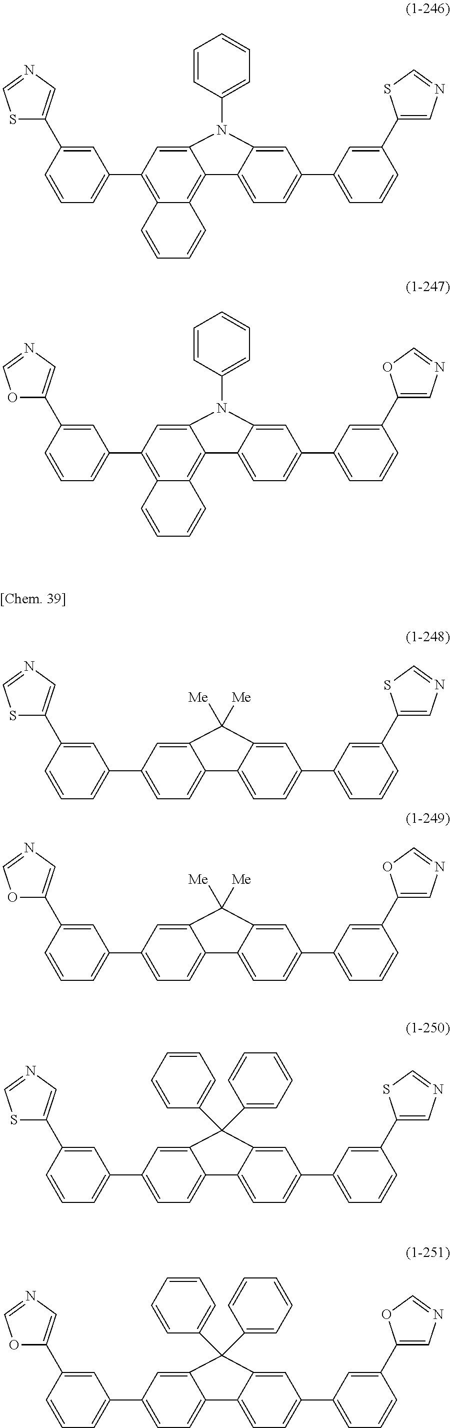 [SMILES: c1ccc(-n2c3cc(-c4cccc(-c5cncs5)c4)ccc3c3c4ccccc4c(-c4cccc(-c5cncs5)c4)cc32)cc1 ; c1ccc(-n2c3cc(-c4cccc(-c5cnco5)c4)ccc3c3c4ccccc4c(-c4cccc(-c5cnco5)c4)cc32)cc1 ; CC1(C)c2cc(-c3cccc(-c4cncs4)c3)ccc2-c2ccc(-c3cccc(-c4cncs4)c3)cc21 ; CC1(C)c2cc(-c3cccc(-c4cnco4)c3)ccc2-c2ccc(-c3cccc(-c4cnco4)c3)cc21 ; c1ccc(C2(c3ccccc3)c3cc(-c4cccc(-c5cncs5)c4)ccc3-c3ccc(-c4cccc(-c5cncs5)c4)cc32)cc1 ; c1ccc(C2(c3ccccc3)c3cc(-c4cccc(-c5cnco5)c4)ccc3-c3ccc(-c4cccc(-c5cnco5)c4)cc32)cc1]

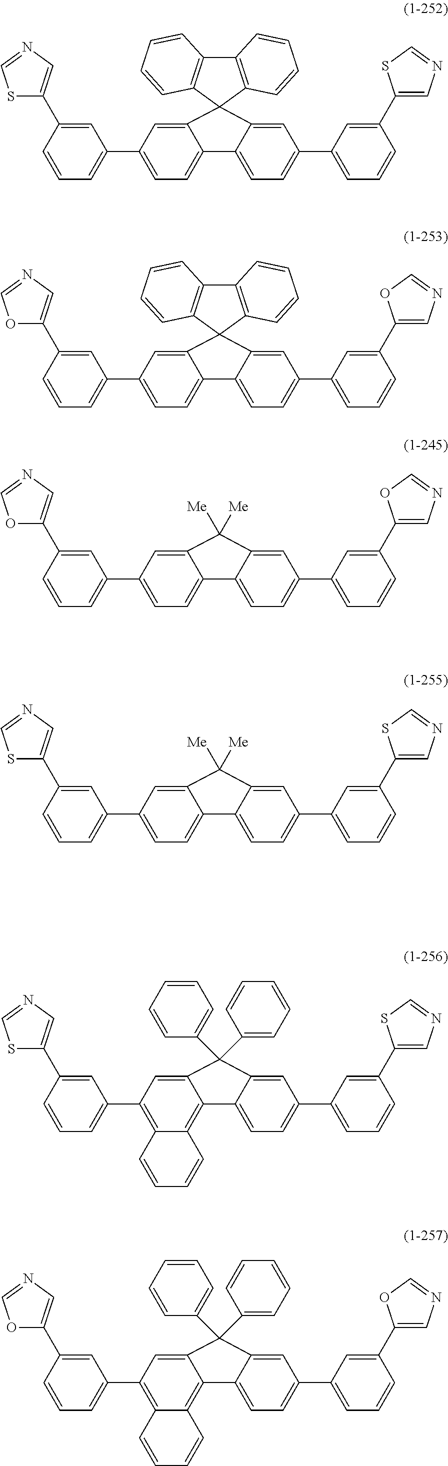 [SMILES: c1cc(-c2ccc3c(c2)C2(c4ccccc4-c4ccccc42)c2cc(-c4cccc(-c5cncs5)c4)ccc2-3)cc(-c2cncs2)c1 ; c1cc(-c2ccc3c(c2)C2(c4ccccc4-c4ccccc42)c2cc(-c4cccc(-c5cnco5)c4)ccc2-3)cc(-c2cnco2)c1 ; CC1(C)c2cc(-c3cccc(-c4cnco4)c3)ccc2-c2ccc(-c3cccc(-c4cnco4)c3)cc21 ; CC1(C)c2cc(-c3cccc(-c4cncs4)c3)ccc2-c2ccc(-c3cccc(-c4cncs4)c3)cc21 ; c1ccc(C2(c3ccccc3)c3cc(-c4cccc(-c5cncs5)c4)ccc3-c3c2cc(-c2cccc(-c4cncs4)c2)c2ccccc32)cc1 ; c1ccc(C2(c3ccccc3)c3cc(-c4cccc(-c5cnco5)c4)ccc3-c3c2cc(-c2cccc(-c4cnco4)c2)c2ccccc32)cc1]

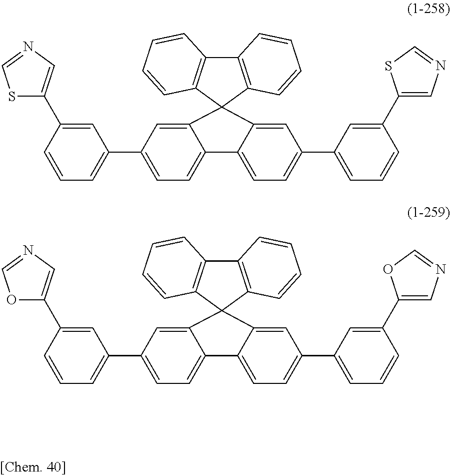 [SMILES: c1cc(-c2ccc3c(c2)C2(c4ccccc4-c4ccccc42)c2cc(-c4cccc(-c5cncs5)c4)ccc2-3)cc(-c2cncs2)c1 ; c1cc(-c2ccc3c(c2)C2(c4ccccc4-c4ccccc42)c2cc(-c4cccc(-c5cnco5)c4)ccc2-3)cc(-c2cnco2)c1]

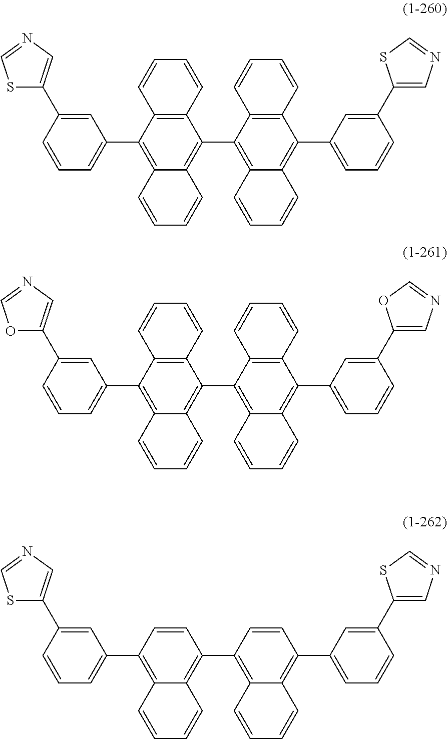 [SMILES: c1cc(-c2cncs2)cc(-c2c3ccccc3c(-c3c4ccccc4c(-c4cccc(-c5cncs5)c4)c4ccccc34)c3ccccc23)c1 ; c1cc(-c2cnco2)cc(-c2c3ccccc3c(-c3c4ccccc4c(-c4cccc(-c5cnco5)c4)c4ccccc34)c3ccccc23)c1 ; c1cc(-c2cncs2)cc(-c2ccc(-c3ccc(-c4cccc(-c5cncs5)c4)c4ccccc34)c3ccccc23)c1]

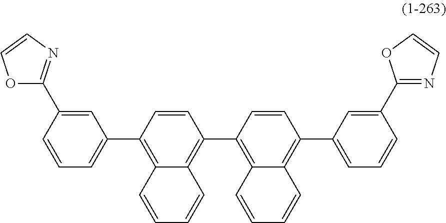 [SMILES: c1cc(-c2ncco2)cc(-c2ccc(-c3ccc(-c4cccc(-c5ncco5)c4)c4ccccc34)c3ccccc23)c1]

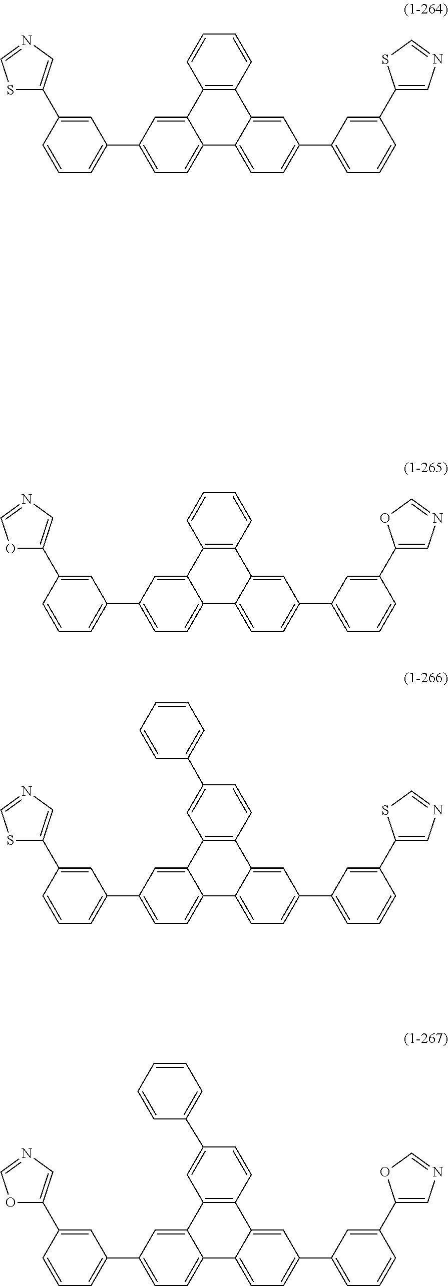 [SMILES: c1cc(-c2ccc3c4ccc(-c5cccc(-c6cncs6)c5)cc4c4ccccc4c3c2)cc(-c2cncs2)c1 ; c1cc(-c2ccc3c4ccc(-c5cccc(-c6cnco6)c5)cc4c4ccccc4c3c2)cc(-c2cnco2)c1 ; c1ccc(-c2ccc3c4cc(-c5cccc(-c6cncs6)c5)ccc4c4ccc(-c5cccc(-c6cncs6)c5)cc4c3c2)cc1 ; c1ccc(-c2ccc3c4cc(-c5cccc(-c6cnco6)c5)ccc4c4ccc(-c5cccc(-c6cnco6)c5)cc4c3c2)cc1]

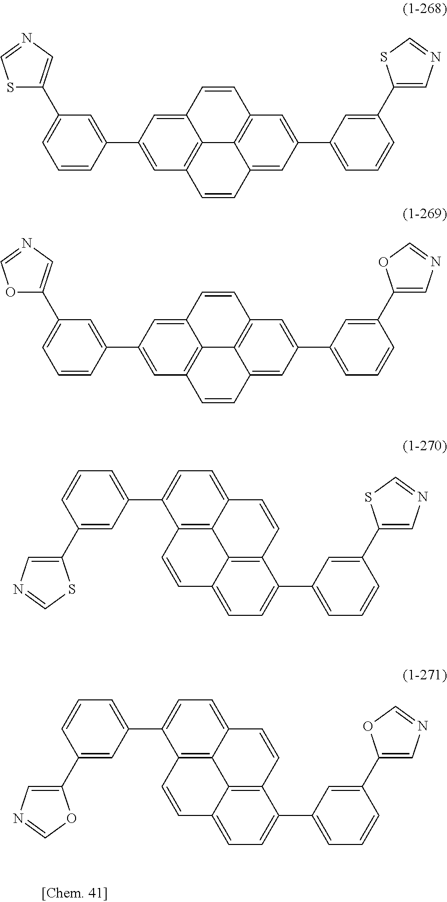 [SMILES: c1cc(-c2cc3ccc4cc(-c5cccc(-c6cncs6)c5)cc5ccc(c2)c3c45)cc(-c2cncs2)c1 ; c1cc(-c2cc3ccc4cc(-c5cccc(-c6cnco6)c5)cc5ccc(c2)c3c45)cc(-c2cnco2)c1 ; c1cc(-c2cncs2)cc(-c2ccc3ccc4c(-c5cccc(-c6cncs6)c5)ccc5ccc2c3c54)c1 ; c1cc(-c2cnco2)cc(-c2ccc3ccc4c(-c5cccc(-c6cnco6)c5)ccc5ccc2c3c54)c1]

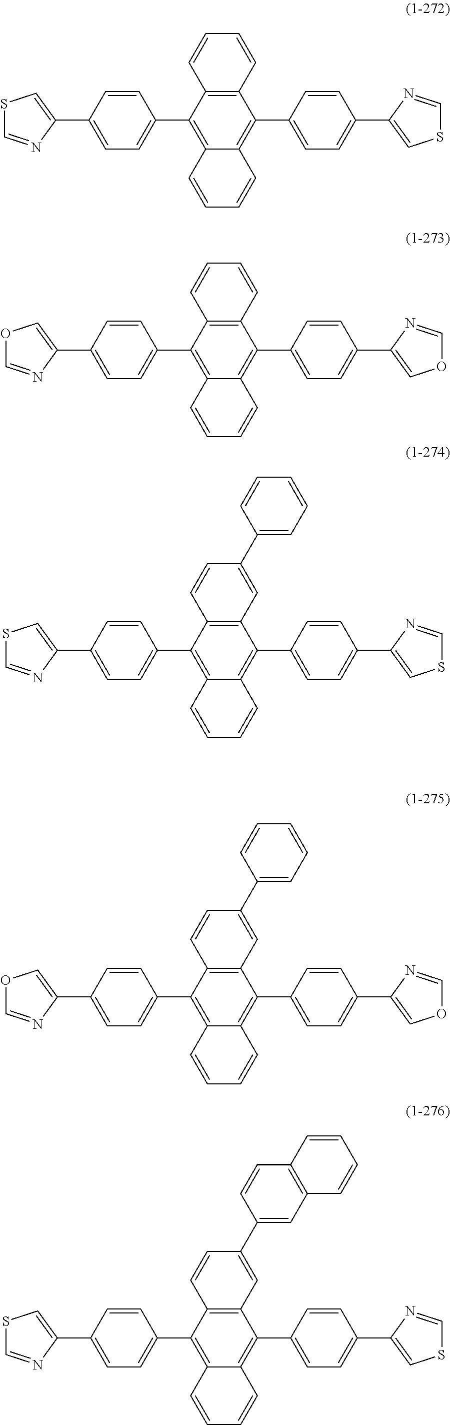

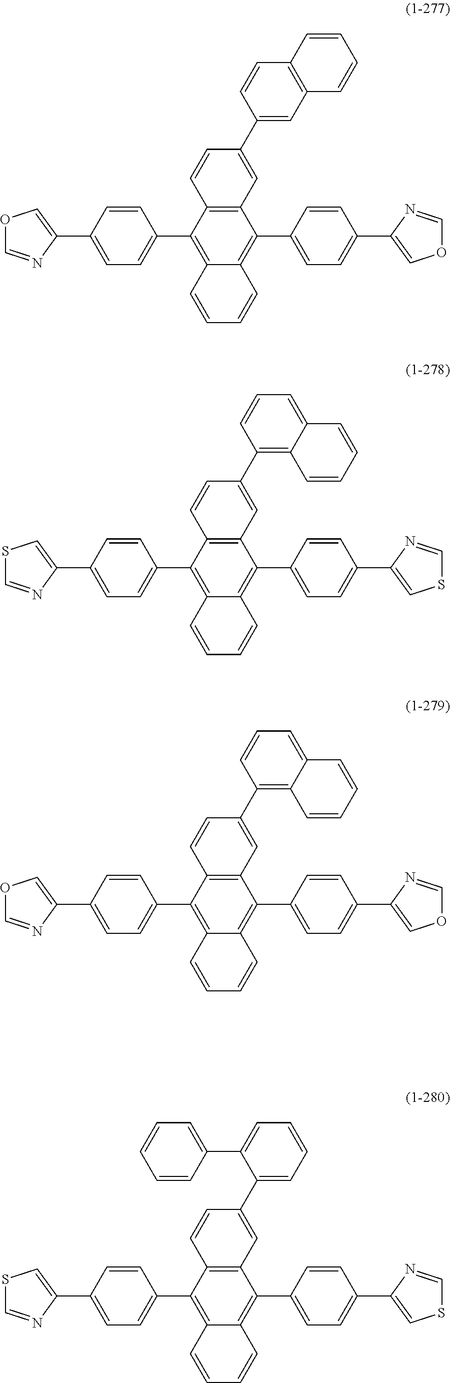 [SMILES: c1ccc2cc(-c3ccc4c(-c5ccc(-c6cocn6)cc5)c5ccccc5c(-c5ccc(-c6cocn6)cc5)c4c3)ccc2c1 ; c1ccc2c(-c3ccc4c(-c5ccc(-c6cscn6)cc5)c5ccccc5c(-c5ccc(-c6cscn6)cc5)c4c3)cccc2c1 ; c1ccc2c(-c3ccc4c(-c5ccc(-c6cocn6)cc5)c5ccccc5c(-c5ccc(-c6cocn6)cc5)c4c3)cccc2c1 ; c1ccc(-c2ccccc2-c2ccc3c(-c4ccc(-c5cscn5)cc4)c4ccccc4c(-c4ccc(-c5cscn5)cc4)c3c2)cc1]

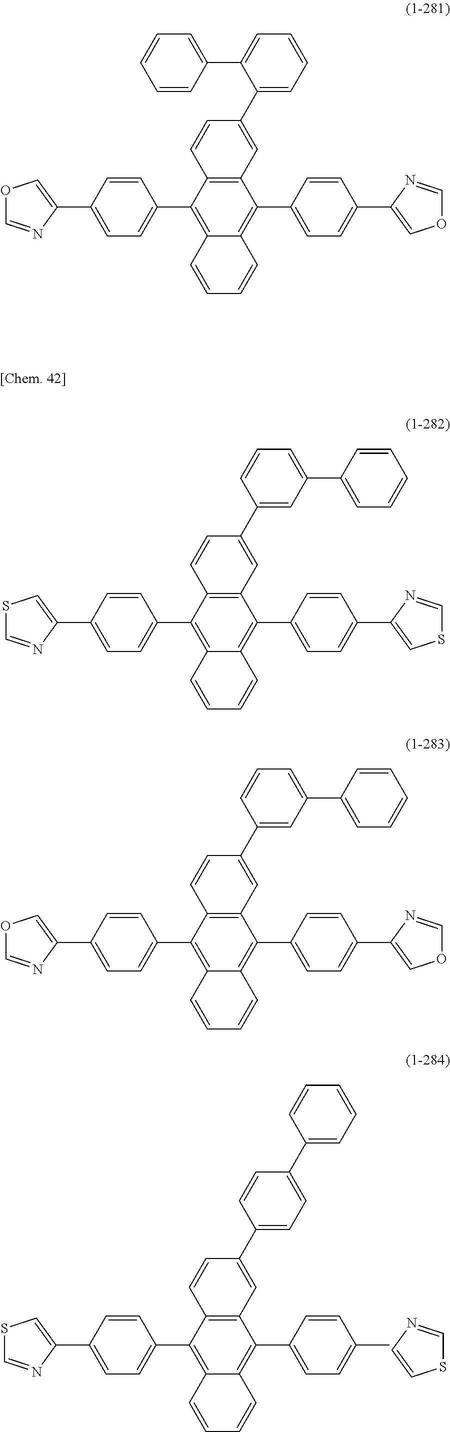

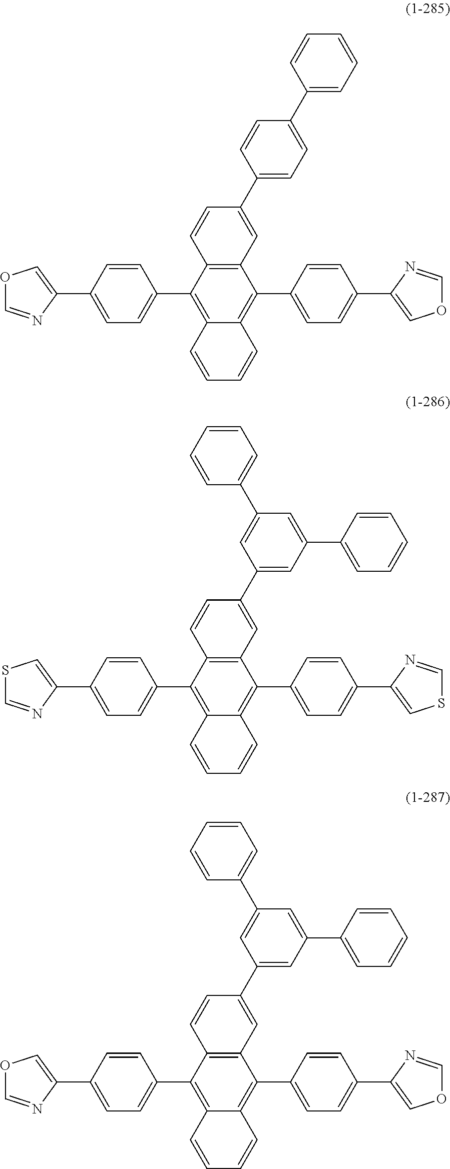

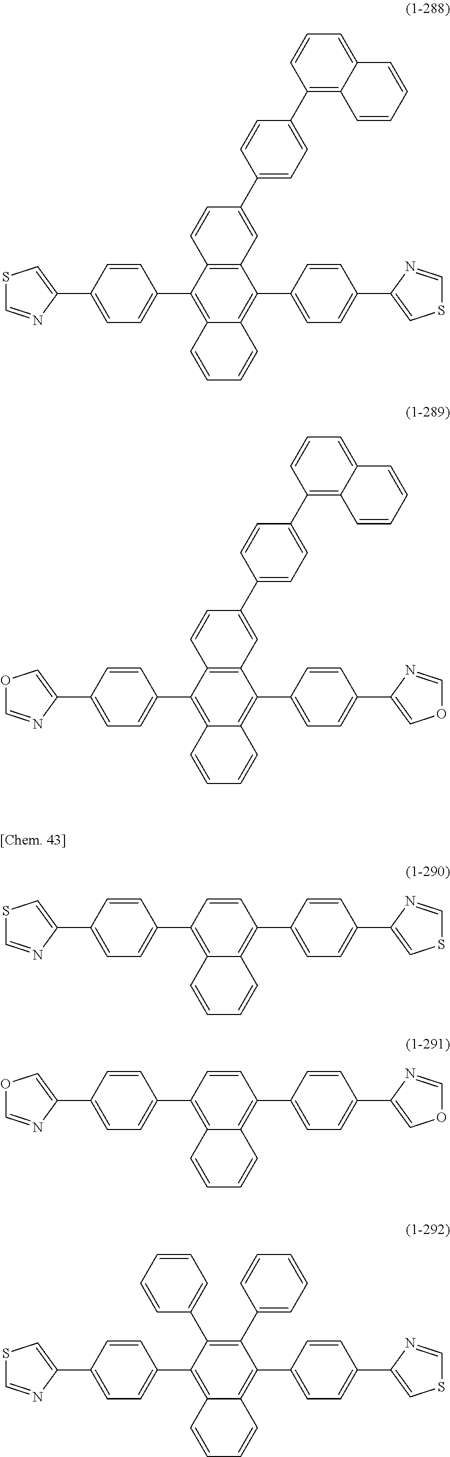 [SMILES: c1ccc2c(-c3ccc(-c4ccc5c(-c6ccc(-c7cscn7)cc6)c6ccccc6c(-c6ccc(-c7cscn7)cc6)c5c4)cc3)cccc2c1 ; c1ccc2c(-c3ccc(-c4ccc5c(-c6ccc(-c7cocn7)cc6)c6ccccc6c(-c6ccc(-c7cocn7)cc6)c5c4)cc3)cccc2c1 ; c1ccc2c(-c3ccc(-c4cscn4)cc3)ccc(-c3ccc(-c4cscn4)cc3)c2c1 ; c1ccc2c(-c3ccc(-c4cocn4)cc3)ccc(-c3ccc(-c4cocn4)cc3)c2c1 ; c1ccc(-c2c(-c3ccccc3)c(-c3ccc(-c4cscn4)cc3)c3ccccc3c2-c2ccc(-c3cscn3)cc2)cc1]

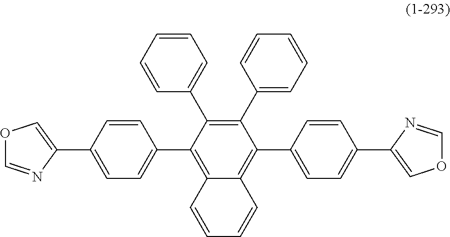 [SMILES: c1ccc(-c2c(-c3ccccc3)c(-c3ccc(-c4cocn4)cc3)c3ccccc3c2-c2ccc(-c3cocn3)cc2)cc1]

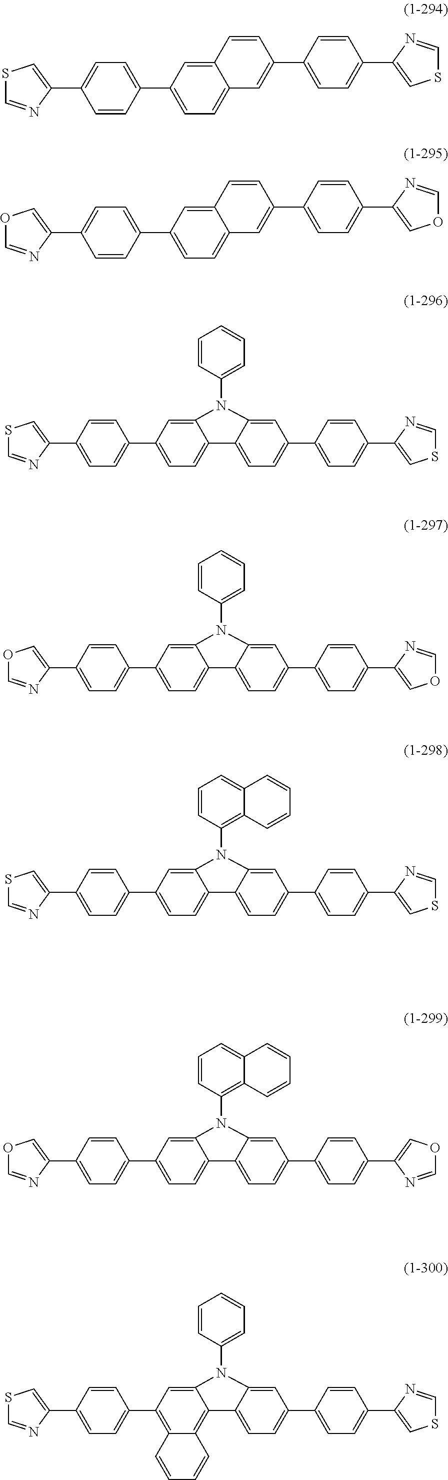

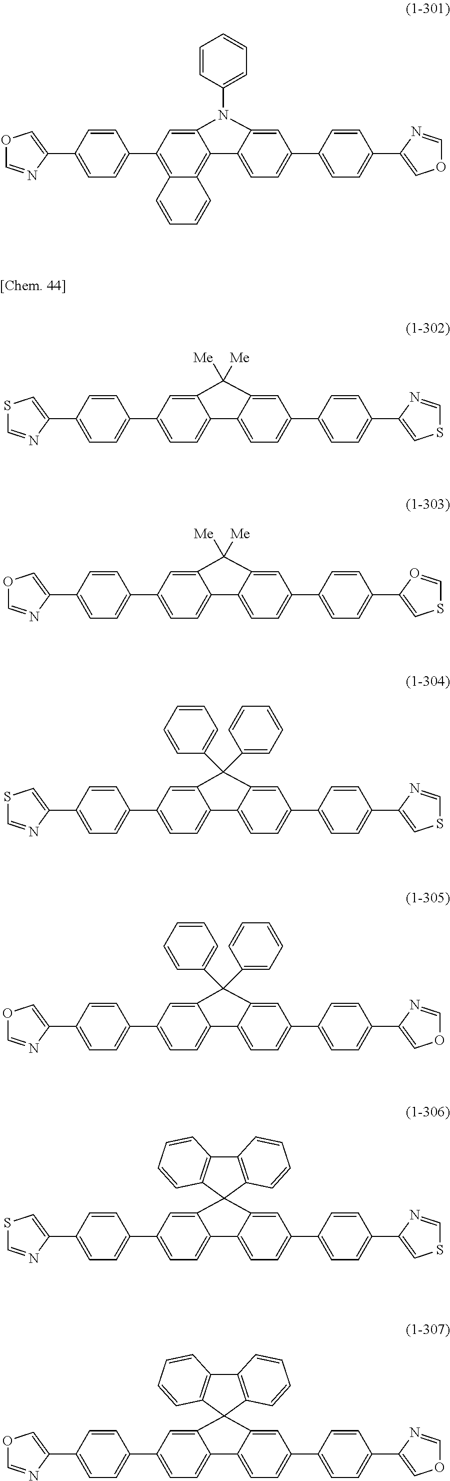

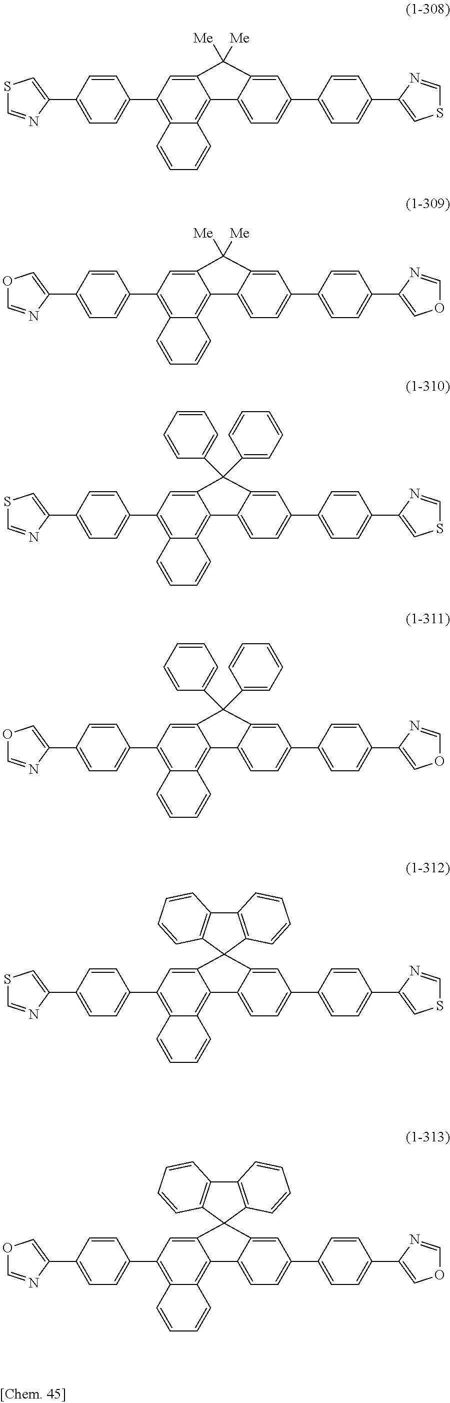

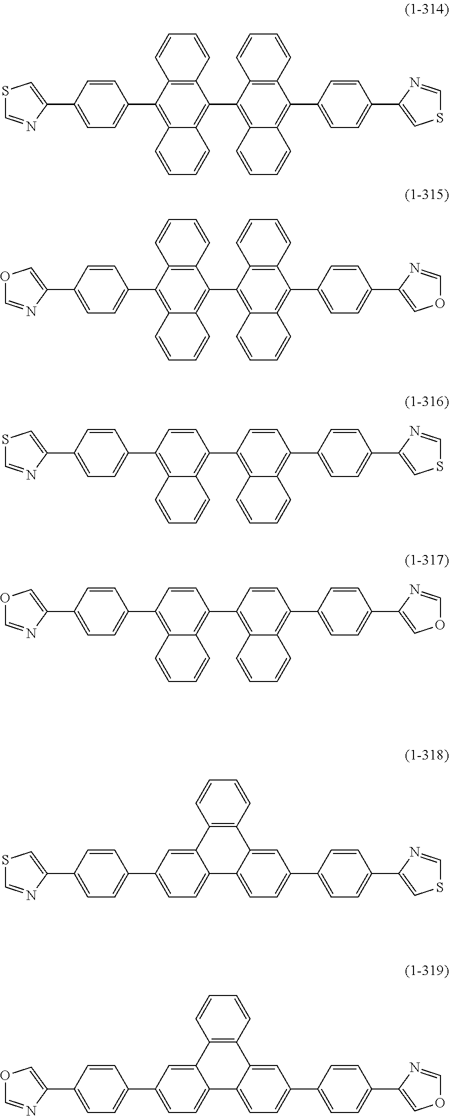 [SMILES: c1ccc2c(-c3c4ccccc4c(-c4ccc(-c5cscn5)cc4)c4ccccc34)c3ccccc3c(-c3ccc(-c4cscn4)cc3)c2c1 ; c1ccc2c(-c3c4ccccc4c(-c4ccc(-c5cocn5)cc4)c4ccccc34)c3ccccc3c(-c3ccc(-c4cocn4)cc3)c2c1 ; c1ccc2c(-c3ccc(-c4ccc(-c5cscn5)cc4)c4ccccc34)ccc(-c3ccc(-c4cscn4)cc3)c2c1 ; c1ccc2c(-c3ccc(-c4ccc(-c5cocn5)cc4)c4ccccc34)ccc(-c3ccc(-c4cocn4)cc3)c2c1 ; c1ccc2c(c1)c1cc(-c3ccc(-c4cscn4)cc3)ccc1c1ccc(-c3ccc(-c4cscn4)cc3)cc21 ; c1ccc2c(c1)c1cc(-c3ccc(-c4cocn4)cc3)ccc1c1ccc(-c3ccc(-c4cocn4)cc3)cc21]

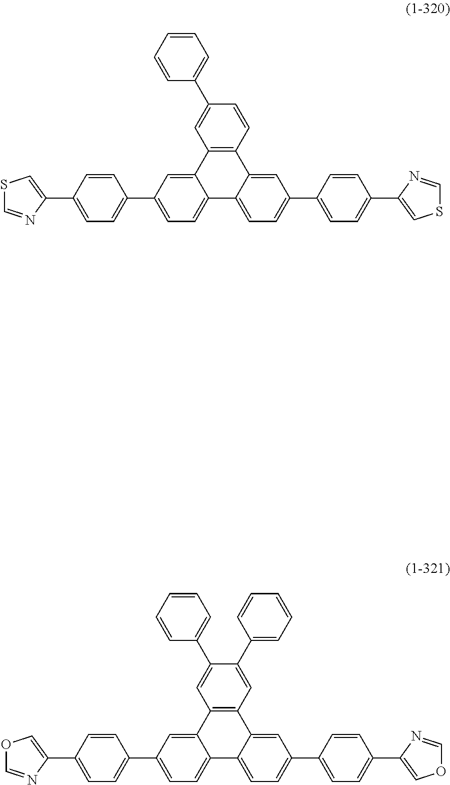 [SMILES: c1ccc(-c2ccc3c4cc(-c5ccc(-c6cscn6)cc5)ccc4c4ccc(-c5ccc(-c6cscn6)cc5)cc4c3c2)cc1 ; c1ccc(-c2cc3c4cc(-c5ccc(-c6cocn6)cc5)ccc4c4ccc(-c5ccc(-c6cocn6)cc5)cc4c3cc2-c2ccccc2)cc1]

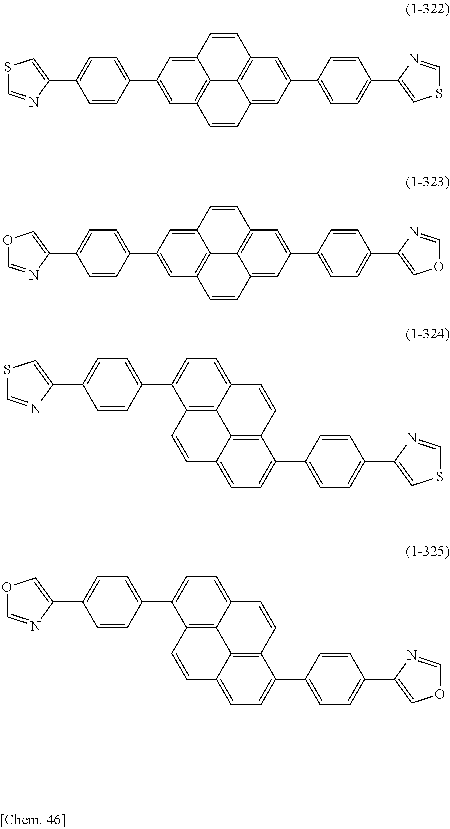 [SMILES: c1nc(-c2ccc(-c3cc4ccc5cc(-c6ccc(-c7cscn7)cc6)cc6ccc(c3)c4c56)cc2)cs1 ; c1nc(-c2ccc(-c3cc4ccc5cc(-c6ccc(-c7cocn7)cc6)cc6ccc(c3)c4c56)cc2)co1 ; c1nc(-c2ccc(-c3ccc4ccc5c(-c6ccc(-c7cscn7)cc6)ccc6ccc3c4c65)cc2)cs1 ; c1nc(-c2ccc(-c3ccc4ccc5c(-c6ccc(-c7cocn7)cc6)ccc6ccc3c4c65)cc2)co1]

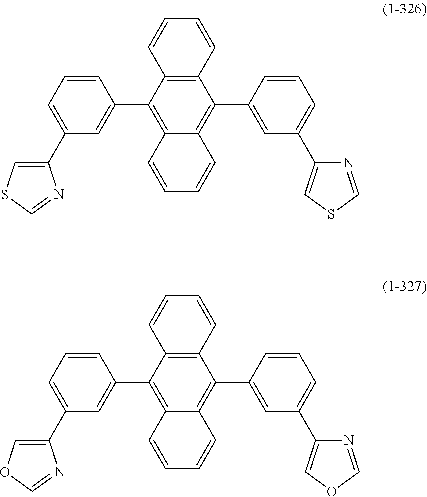 [SMILES: c1cc(-c2cscn2)cc(-c2c3ccccc3c(-c3cccc(-c4cscn4)c3)c3ccccc23)c1 ; c1cc(-c2cocn2)cc(-c2c3ccccc3c(-c3cccc(-c4cocn4)c3)c3ccccc23)c1]

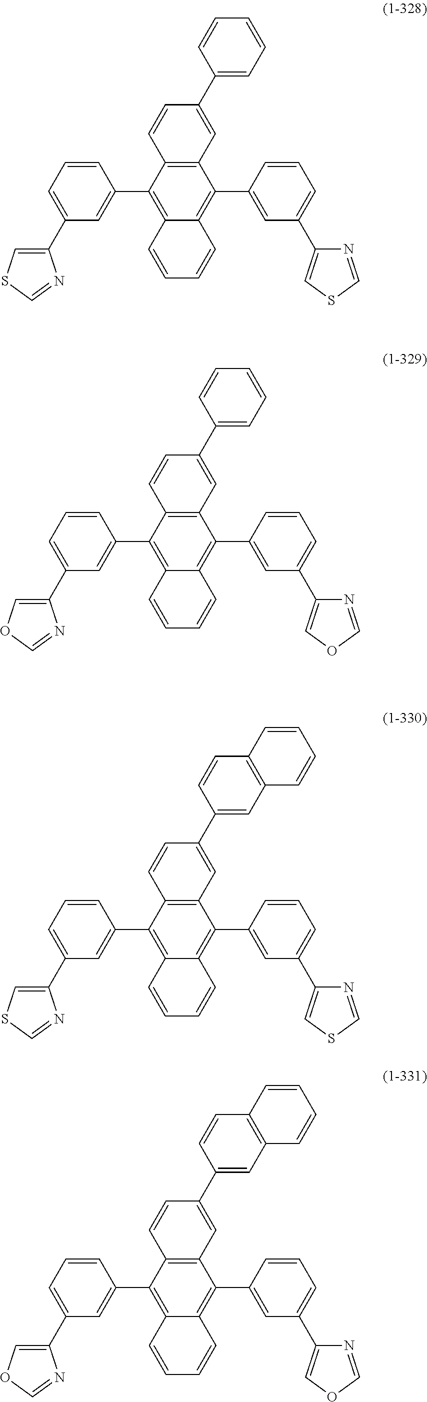 [SMILES: c1ccc(-c2ccc3c(-c4cccc(-c5cscn5)c4)c4ccccc4c(-c4cccc(-c5cscn5)c4)c3c2)cc1 ; c1ccc(-c2ccc3c(-c4cccc(-c5cocn5)c4)c4ccccc4c(-c4cccc(-c5cocn5)c4)c3c2)cc1 ; c1cc(-c2cscn2)cc(-c2c3ccccc3c(-c3cccc(-c4cscn4)c3)c3cc(-c4ccc5ccccc5c4)ccc23)c1 ; c1cc(-c2cocn2)cc(-c2c3ccccc3c(-c3cccc(-c4cocn4)c3)c3cc(-c4ccc5ccccc5c4)ccc23)c1]

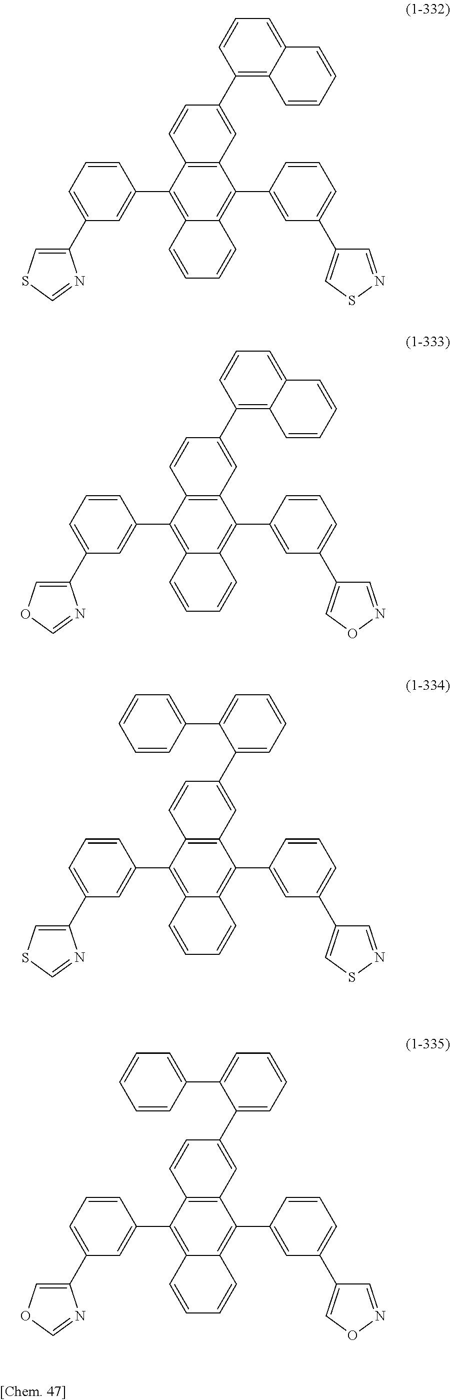 [SMILES: c1cc(-c2cnsc2)cc(-c2c3ccccc3c(-c3cccc(-c4cscn4)c3)c3ccc(-c4cccc5ccccc45)cc23)c1 ; c1cc(-c2cnoc2)cc(-c2c3ccccc3c(-c3cccc(-c4cocn4)c3)c3ccc(-c4cccc5ccccc45)cc23)c1 ; c1ccc(-c2ccccc2-c2ccc3c(-c4cccc(-c5cscn5)c4)c4ccccc4c(-c4cccc(-c5cnsc5)c4)c3c2)cc1 ; c1ccc(-c2ccccc2-c2ccc3c(-c4cccc(-c5cocn5)c4)c4ccccc4c(-c4cccc(-c5cnoc5)c4)c3c2)cc1]

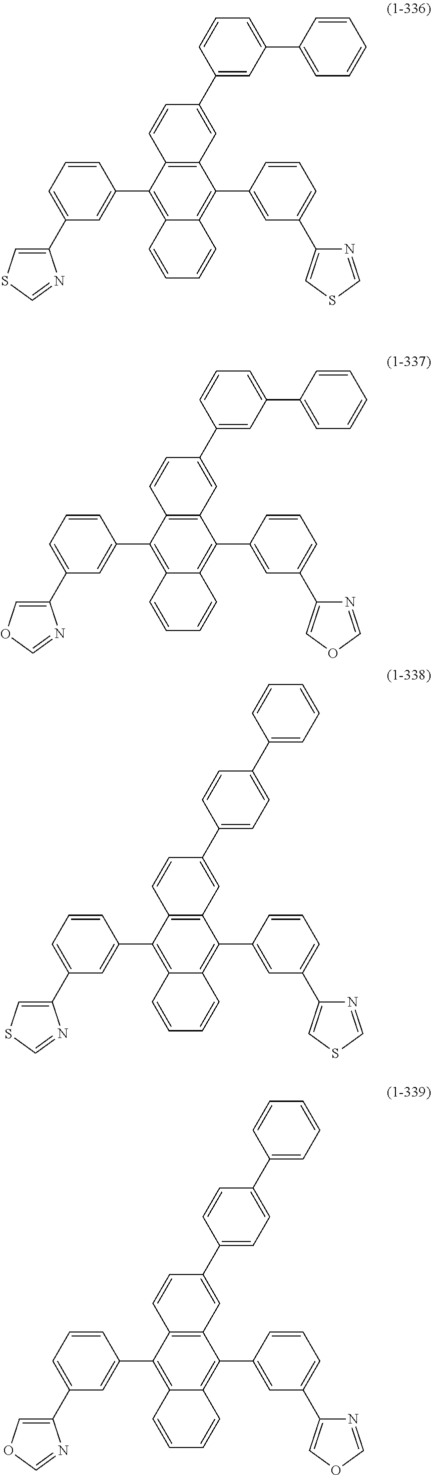

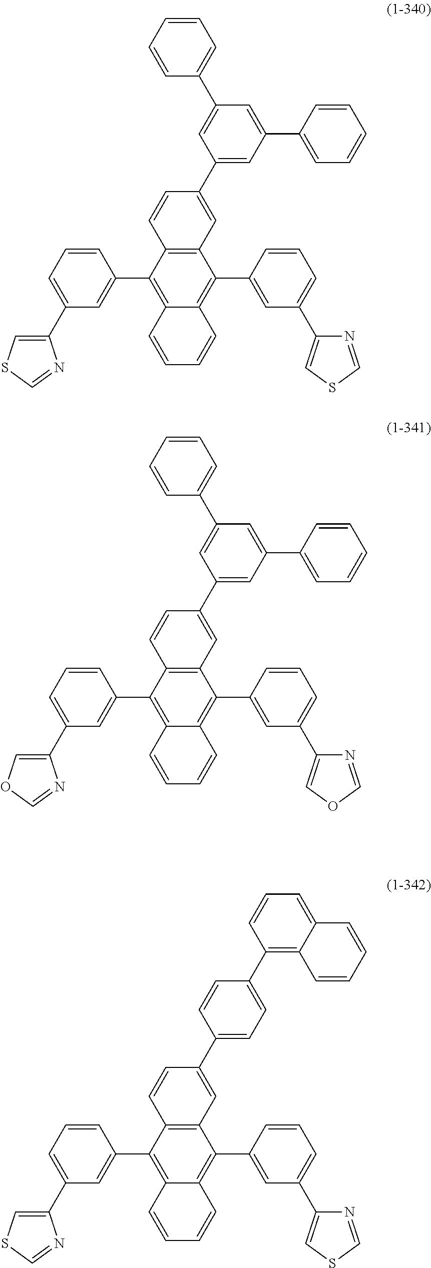 [SMILES: c1ccc(-c2cc(-c3ccccc3)cc(-c3ccc4c(-c5cccc(-c6cscn6)c5)c5ccccc5c(-c5cccc(-c6cscn6)c5)c4c3)c2)cc1 ; c1ccc(-c2cc(-c3ccccc3)cc(-c3ccc4c(-c5cccc(-c6cocn6)c5)c5ccccc5c(-c5cccc(-c6cocn6)c5)c4c3)c2)cc1 ; c1cc(-c2cscn2)cc(-c2c3ccccc3c(-c3cccc(-c4cscn4)c3)c3cc(-c4ccc(-c5cccc6ccccc56)cc4)ccc23)c1]

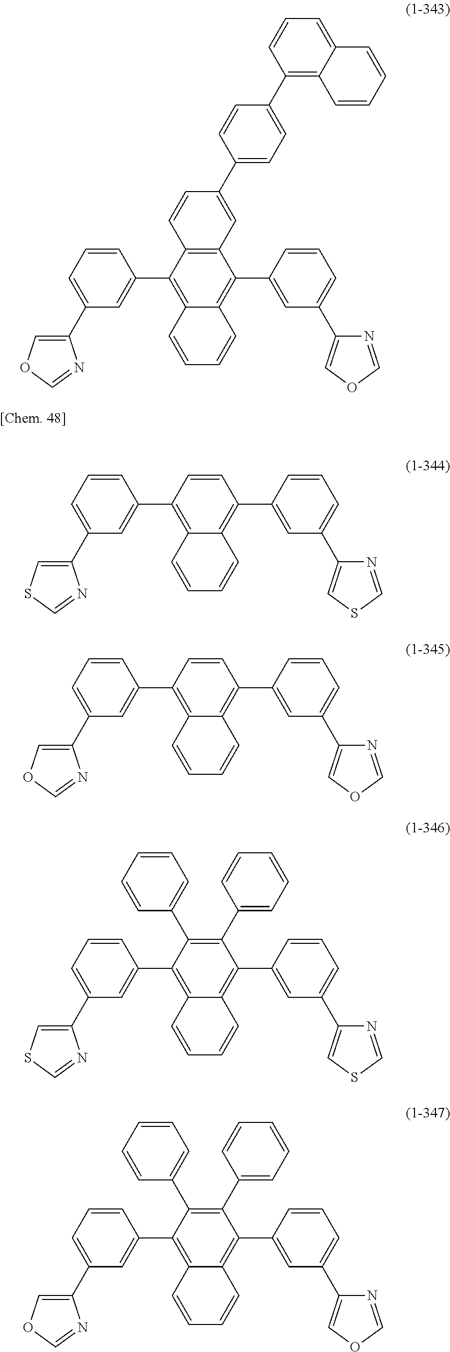 [SMILES: c1cc(-c2cocn2)cc(-c2c3ccccc3c(-c3cccc(-c4cocn4)c3)c3cc(-c4ccc(-c5cccc6ccccc56)cc4)ccc23)c1 ; c1cc(-c2cscn2)cc(-c2ccc(-c3cccc(-c4cscn4)c3)c3ccccc23)c1 ; c1cc(-c2cocn2)cc(-c2ccc(-c3cccc(-c4cocn4)c3)c3ccccc23)c1 ; c1ccc(-c2c(-c3ccccc3)c(-c3cccc(-c4cscn4)c3)c3ccccc3c2-c2cccc(-c3cscn3)c2)cc1 ; c1ccc(-c2c(-c3ccccc3)c(-c3cccc(-c4cocn4)c3)c3ccccc3c2-c2cccc(-c3cocn3)c2)cc1]

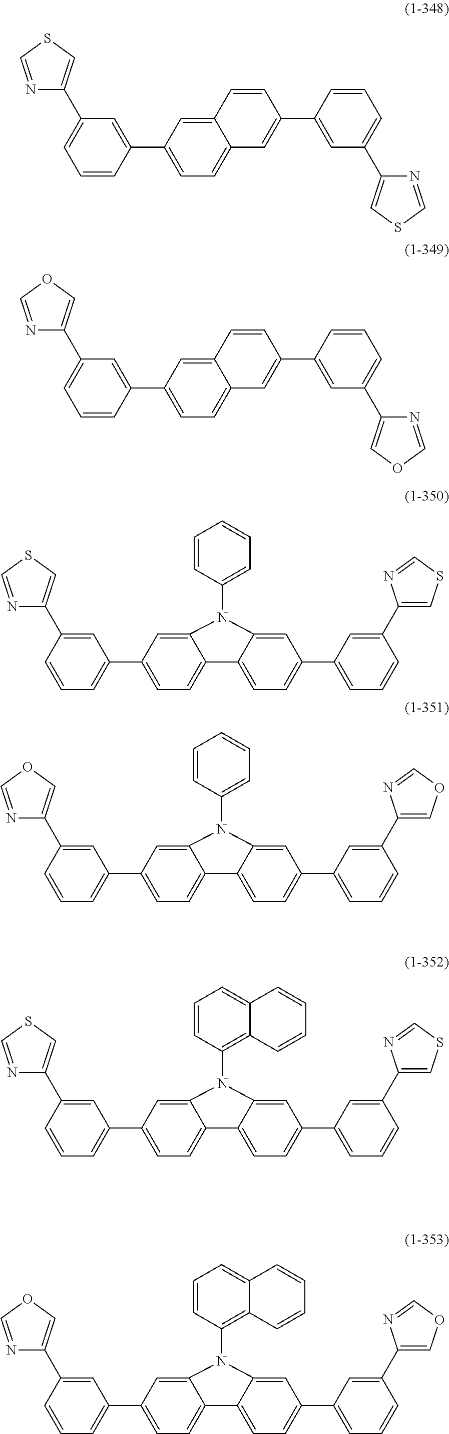 [SMILES: c1cc(-c2ccc3cc(-c4cccc(-c5cscn5)c4)ccc3c2)cc(-c2cscn2)c1 ; c1cc(-c2ccc3cc(-c4cccc(-c5cocn5)c4)ccc3c2)cc(-c2cocn2)c1 ; c1ccc(-n2c3cc(-c4cccc(-c5cscn5)c4)ccc3c3ccc(-c4cccc(-c5cscn5)c4)cc32)cc1 ; c1ccc(-n2c3cc(-c4cccc(-c5cocn5)c4)ccc3c3ccc(-c4cccc(-c5cocn5)c4)cc32)cc1 ; c1cc(-c2ccc3c4ccc(-c5cccc(-c6cscn6)c5)cc4n(-c4cccc5ccccc45)c3c2)cc(-c2cscn2)c1 ; c1cc(-c2ccc3c4ccc(-c5cccc(-c6cocn6)c5)cc4n(-c4cccc5ccccc45)c3c2)cc(-c2cocn2)c1]

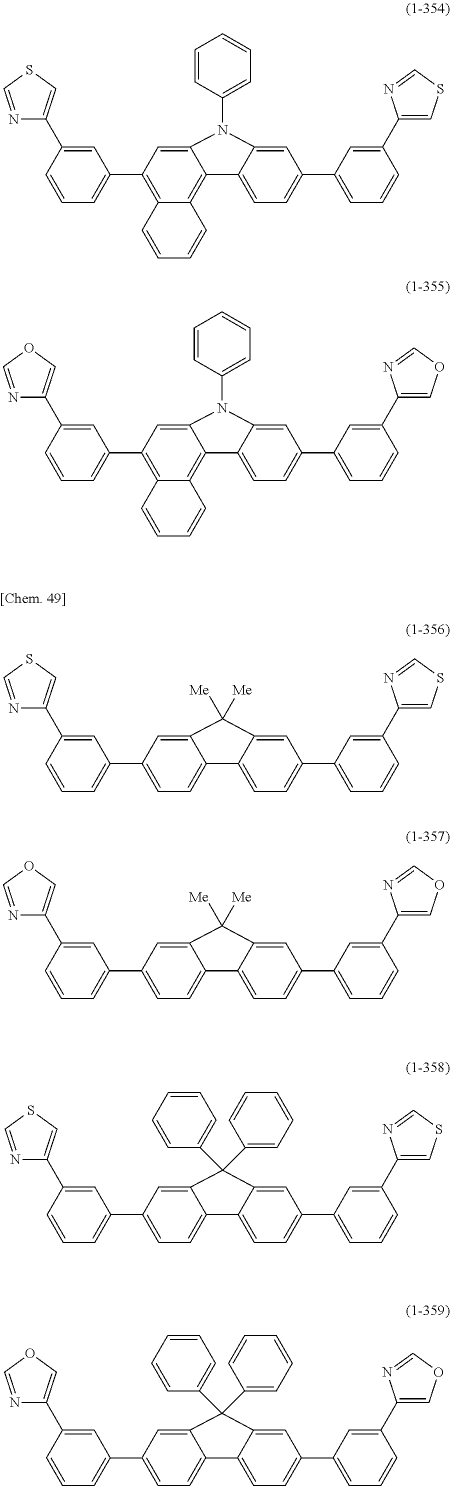 [SMILES: c1ccc(-n2c3cc(-c4cccc(-c5cscn5)c4)ccc3c3c4ccccc4c(-c4cccc(-c5cscn5)c4)cc32)cc1 ; c1ccc(-n2c3cc(-c4cccc(-c5cocn5)c4)ccc3c3c4ccccc4c(-c4cccc(-c5cocn5)c4)cc32)cc1 ; CC1(C)c2cc(-c3cccc(-c4cscn4)c3)ccc2-c2ccc(-c3cccc(-c4cscn4)c3)cc21 ; CC1(C)c2cc(-c3cccc(-c4cocn4)c3)ccc2-c2ccc(-c3cccc(-c4cocn4)c3)cc21 ; c1ccc(C2(c3ccccc3)c3cc(-c4cccc(-c5cscn5)c4)ccc3-c3ccc(-c4cccc(-c5cscn5)c4)cc32)cc1 ; c1ccc(C2(c3ccccc3)c3cc(-c4cccc(-c5cocn5)c4)ccc3-c3ccc(-c4cccc(-c5cocn5)c4)cc32)cc1]

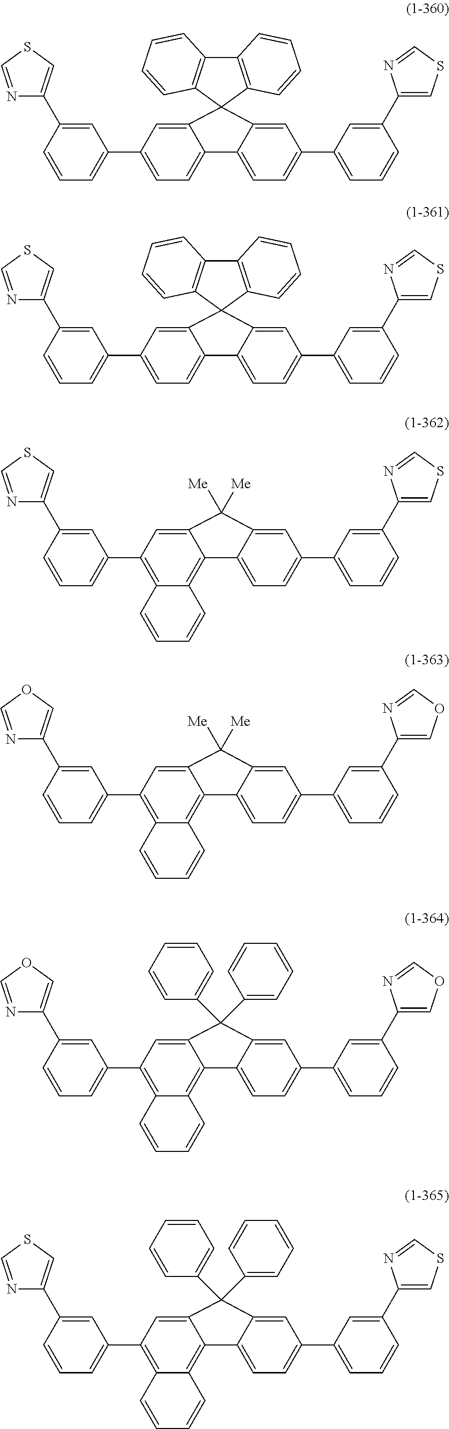

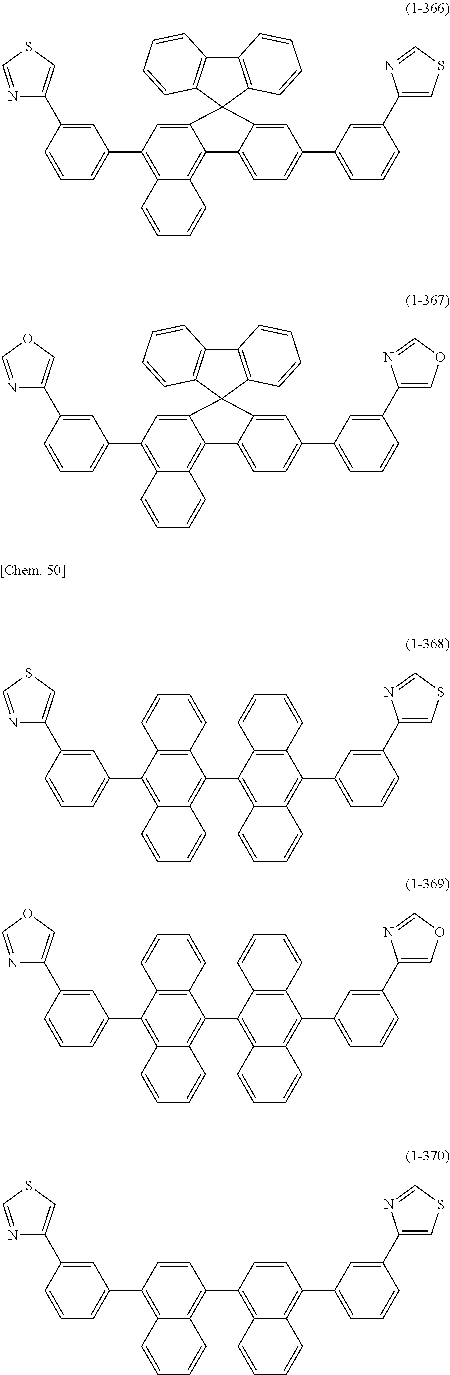 [SMILES: c1cc(-c2ccc3c(c2)C2(c4ccccc4-c4ccccc42)c2cc(-c4cccc(-c5cscn5)c4)c4ccccc4c2-3)cc(-c2cscn2)c1 ; c1cc(-c2ccc3c(c2)C2(c4ccccc4-c4ccccc42)c2cc(-c4cccc(-c5cocn5)c4)c4ccccc4c2-3)cc(-c2cocn2)c1 ; c1cc(-c2cscn2)cc(-c2c3ccccc3c(-c3c4ccccc4c(-c4cccc(-c5cscn5)c4)c4ccccc34)c3ccccc23)c1 ; c1cc(-c2cocn2)cc(-c2c3ccccc3c(-c3c4ccccc4c(-c4cccc(-c5cocn5)c4)c4ccccc34)c3ccccc23)c1 ; c1cc(-c2cscn2)cc(-c2ccc(-c3ccc(-c4cccc(-c5cscn5)c4)c4ccccc34)c3ccccc23)c1]

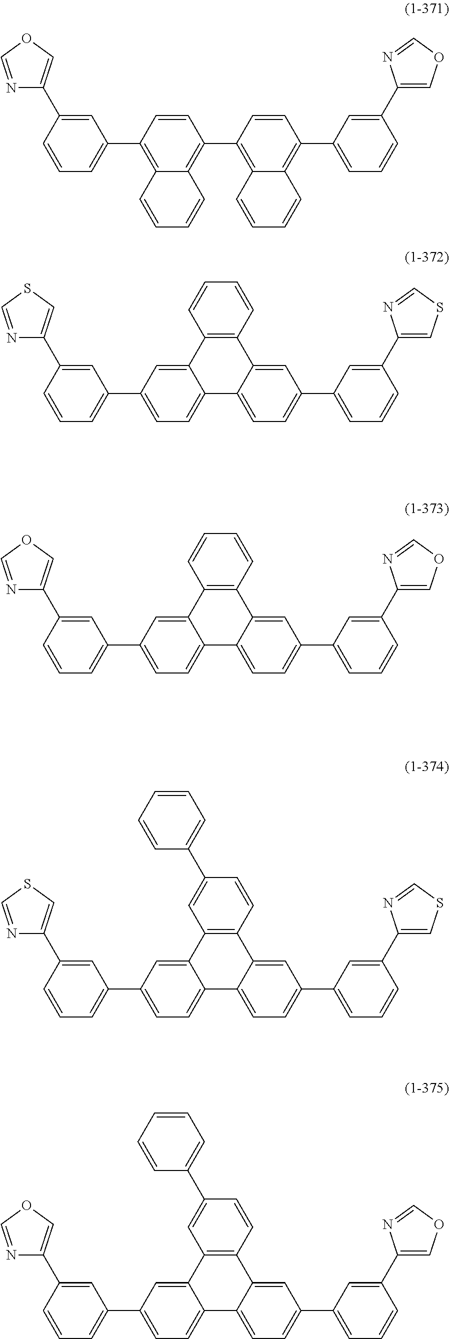 [SMILES: c1cc(-c2cocn2)cc(-c2ccc(-c3ccc(-c4cccc(-c5cocn5)c4)c4ccccc34)c3ccccc23)c1 ; c1cc(-c2ccc3c4ccc(-c5cccc(-c6cscn6)c5)cc4c4ccccc4c3c2)cc(-c2cscn2)c1 ; c1cc(-c2ccc3c4ccc(-c5cccc(-c6cocn6)c5)cc4c4ccccc4c3c2)cc(-c2cocn2)c1 ; c1ccc(-c2ccc3c4cc(-c5cccc(-c6cscn6)c5)ccc4c4ccc(-c5cccc(-c6cscn6)c5)cc4c3c2)cc1 ; c1ccc(-c2ccc3c4cc(-c5cccc(-c6cocn6)c5)ccc4c4ccc(-c5cccc(-c6cocn6)c5)cc4c3c2)cc1]

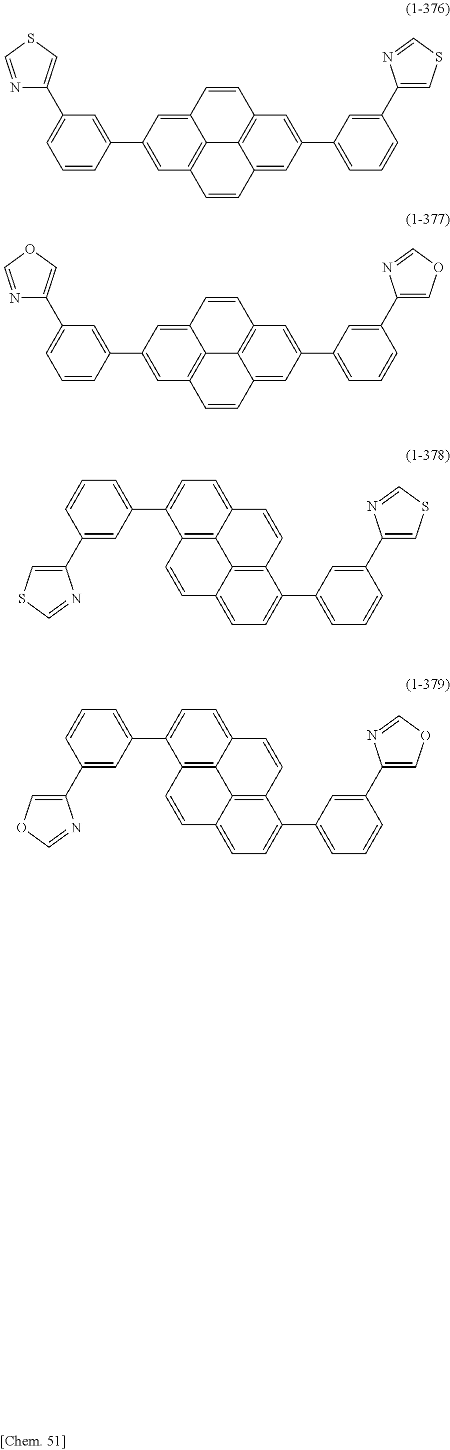 [SMILES: c1cc(-c2cc3ccc4cc(-c5cccc(-c6cscn6)c5)cc5ccc(c2)c3c45)cc(-c2cscn2)c1 ; c1cc(-c2cc3ccc4cc(-c5cccc(-c6cocn6)c5)cc5ccc(c2)c3c45)cc(-c2cocn2)c1 ; c1cc(-c2cscn2)cc(-c2ccc3ccc4c(-c5cccc(-c6cscn6)c5)ccc5ccc2c3c54)c1 ; c1cc(-c2cocn2)cc(-c2ccc3ccc4c(-c5cccc(-c6cocn6)c5)ccc5ccc2c3c54)c1]

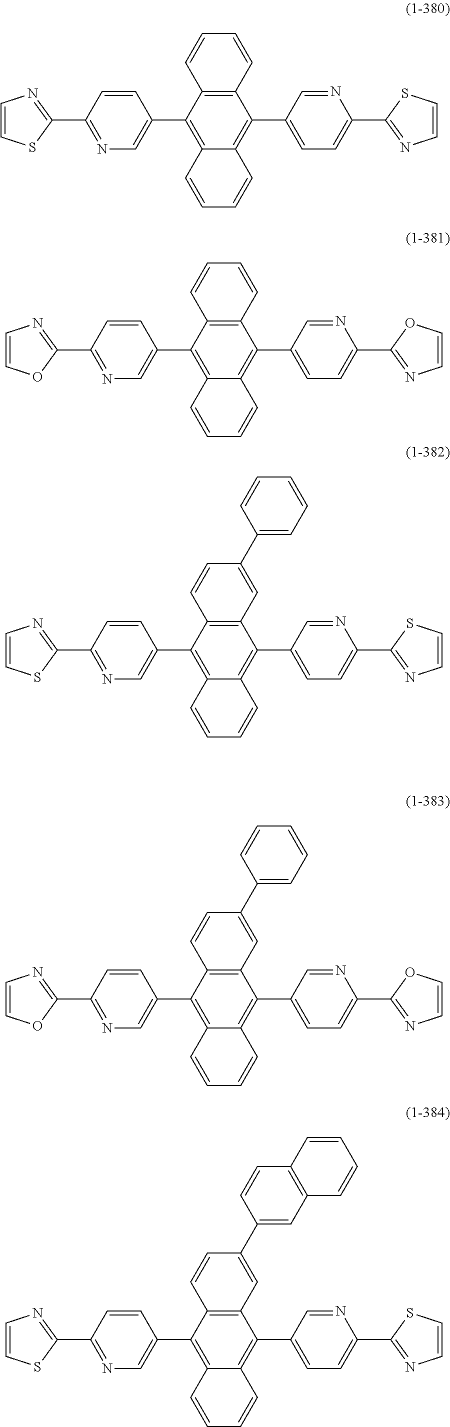

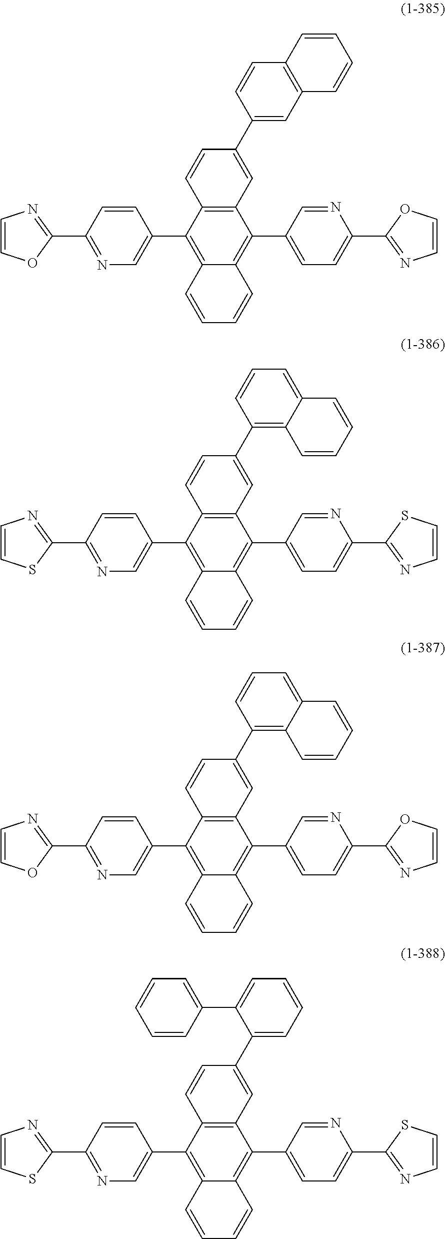

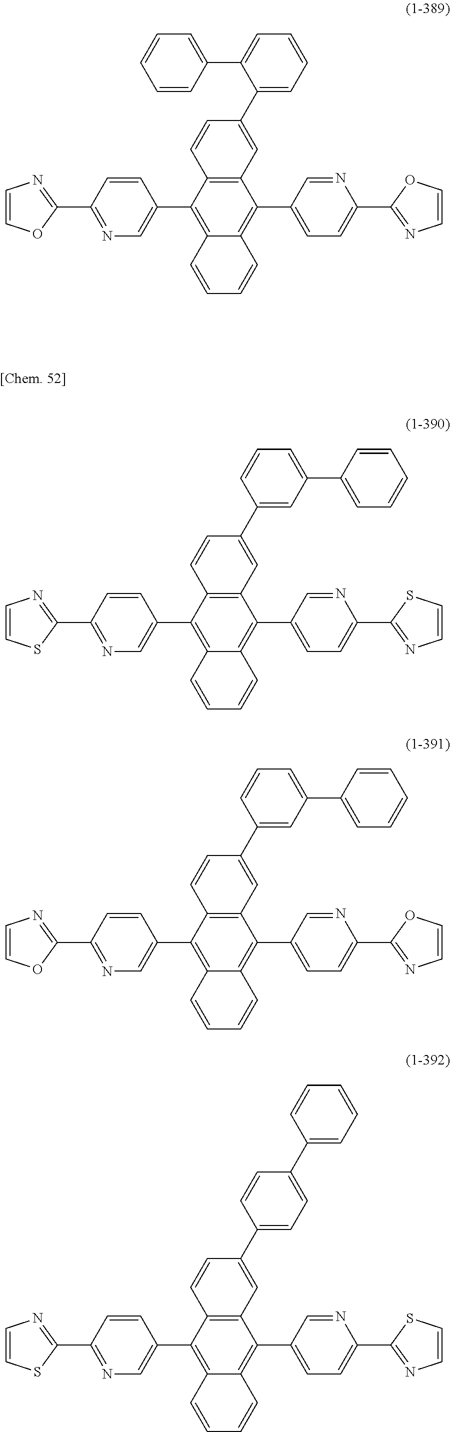 [SMILES: c1ccc(-c2ccccc2-c2ccc3c(-c4ccc(-c5ncco5)nc4)c4ccccc4c(-c4ccc(-c5ncco5)nc4)c3c2)cc1 ; c1ccc(-c2cccc(-c3ccc4c(-c5ccc(-c6nccs6)nc5)c5ccccc5c(-c5ccc(-c6nccs6)nc5)c4c3)c2)cc1 ; c1ccc(-c2cccc(-c3ccc4c(-c5ccc(-c6ncco6)nc5)c5ccccc5c(-c5ccc(-c6ncco6)nc5)c4c3)c2)cc1 ; c1ccc(-c2ccc(-c3ccc4c(-c5ccc(-c6nccs6)nc5)c5ccccc5c(-c5ccc(-c6nccs6)nc5)c4c3)cc2)cc1]

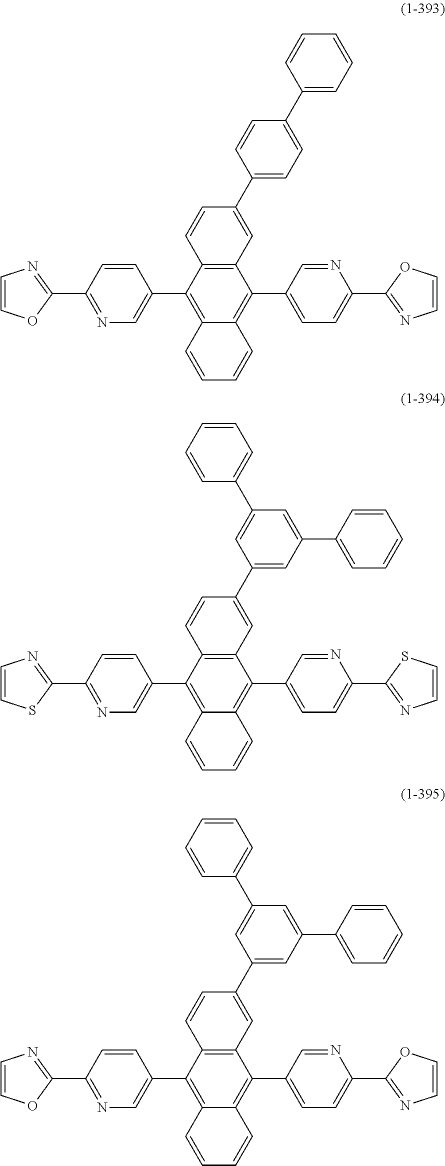

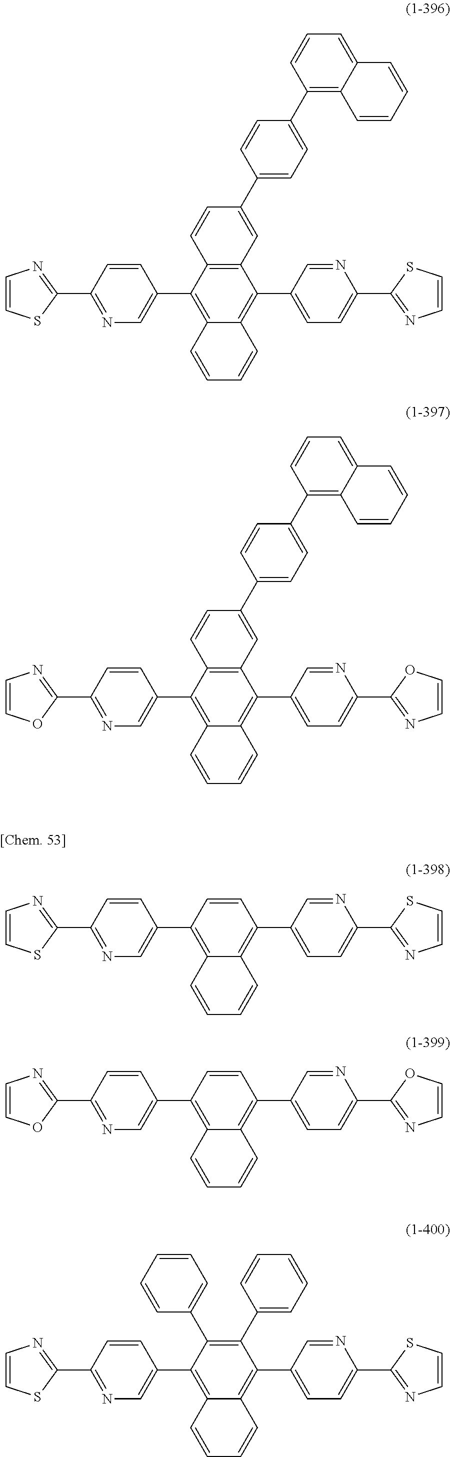 [SMILES: c1ccc2c(-c3ccc(-c4ccc5c(-c6ccc(-c7nccs7)nc6)c6ccccc6c(-c6ccc(-c7nccs7)nc6)c5c4)cc3)cccc2c1 ; c1ccc2c(-c3ccc(-c4ccc5c(-c6ccc(-c7ncco7)nc6)c6ccccc6c(-c6ccc(-c7ncco7)nc6)c5c4)cc3)cccc2c1 ; c1ccc2c(-c3ccc(-c4nccs4)nc3)ccc(-c3ccc(-c4nccs4)nc3)c2c1 ; c1ccc2c(-c3ccc(-c4ncco4)nc3)ccc(-c3ccc(-c4ncco4)nc3)c2c1 ; c1ccc(-c2c(-c3ccccc3)c(-c3ccc(-c4nccs4)nc3)c3ccccc3c2-c2ccc(-c3nccs3)nc2)cc1]

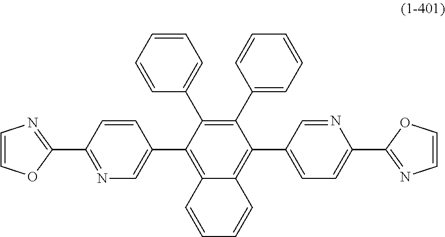 [SMILES: c1ccc(-c2c(-c3ccccc3)c(-c3ccc(-c4ncco4)nc3)c3ccccc3c2-c2ccc(-c3ncco3)nc2)cc1]

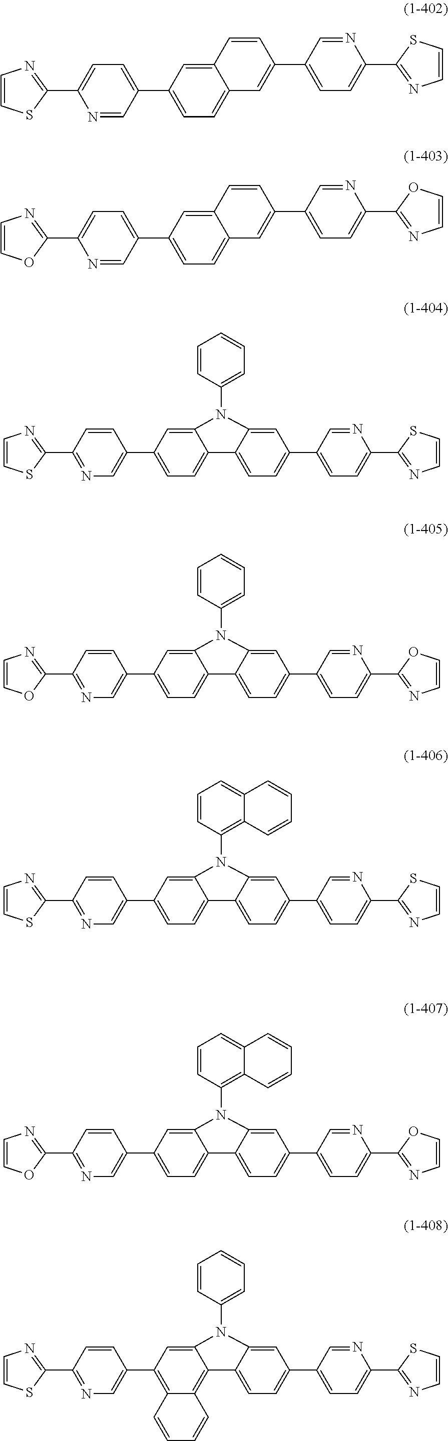

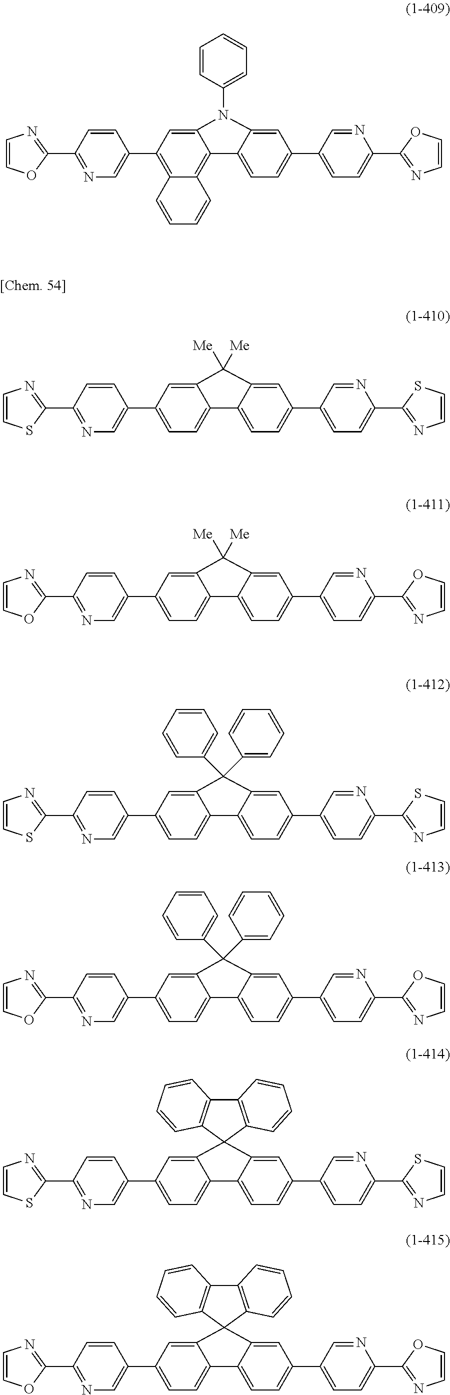

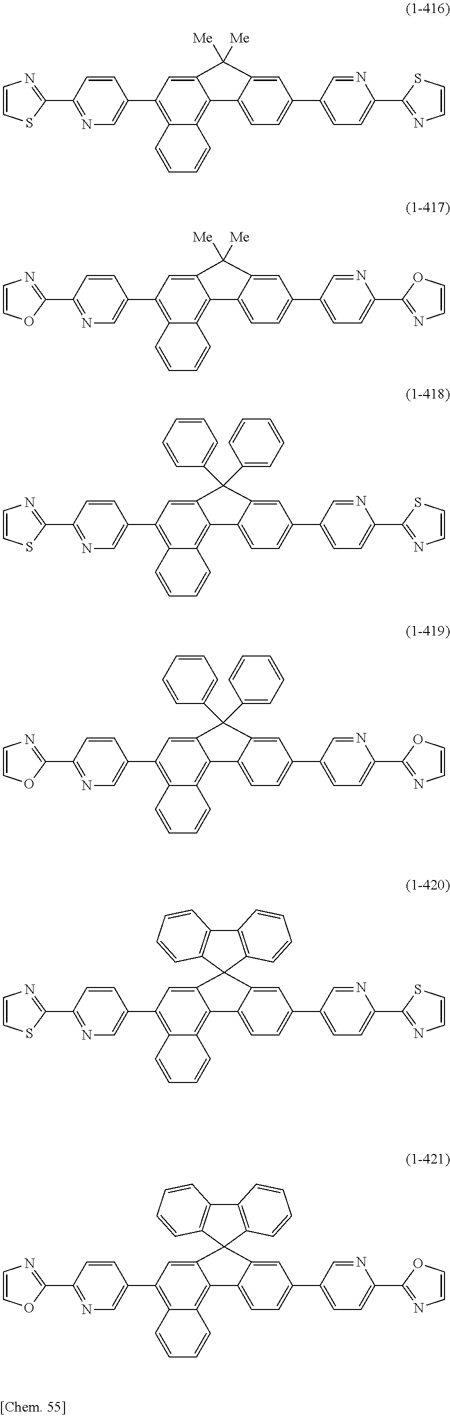

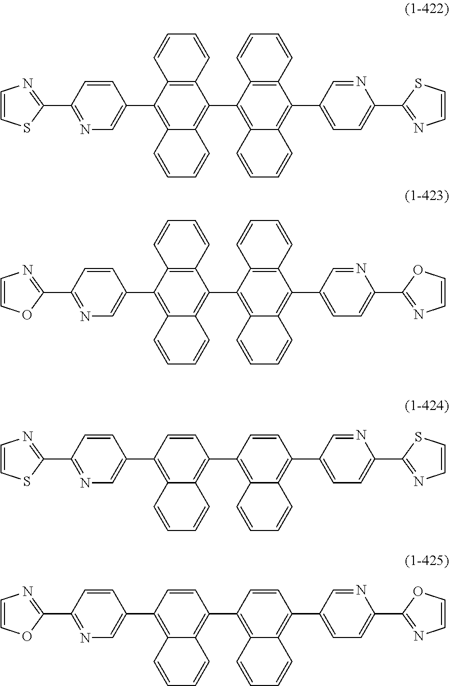 [SMILES: c1ccc2c(-c3c4ccccc4c(-c4ccc(-c5nccs5)nc4)c4ccccc34)c3ccccc3c(-c3ccc(-c4nccs4)nc3)c2c1 ; c1ccc2c(-c3c4ccccc4c(-c4ccc(-c5ncco5)nc4)c4ccccc34)c3ccccc3c(-c3ccc(-c4ncco4)nc3)c2c1 ; c1ccc2c(-c3ccc(-c4ccc(-c5nccs5)nc4)c4ccccc34)ccc(-c3ccc(-c4nccs4)nc3)c2c1 ; c1ccc2c(-c3ccc(-c4ccc(-c5ncco5)nc4)c4ccccc34)ccc(-c3ccc(-c4ncco4)nc3)c2c1]

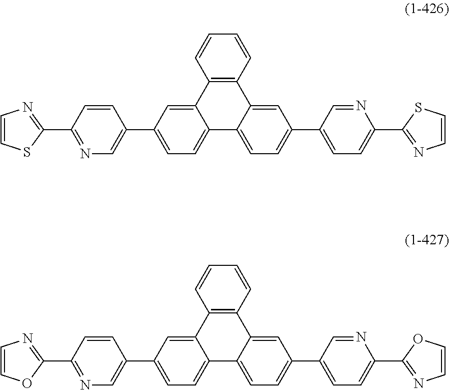 [SMILES: c1ccc2c(c1)c1cc(-c3ccc(-c4nccs4)nc3)ccc1c1ccc(-c3ccc(-c4nccs4)nc3)cc21 ; c1ccc2c(c1)c1cc(-c3ccc(-c4ncco4)nc3)ccc1c1ccc(-c3ccc(-c4ncco4)nc3)cc21]

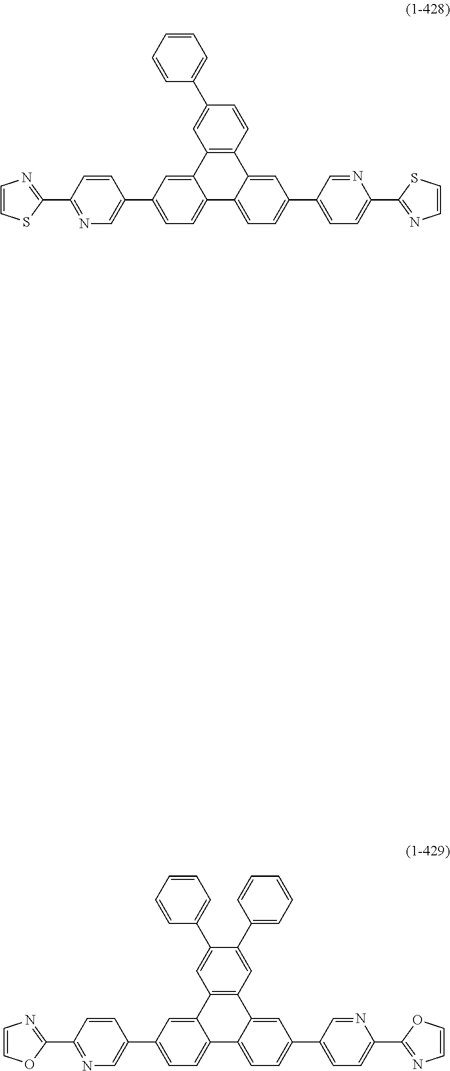 [SMILES: c1ccc(-c2ccc3c4cc(-c5ccc(-c6nccs6)nc5)ccc4c4ccc(-c5ccc(-c6nccs6)nc5)cc4c3c2)cc1 ; c1ccc(-c2cc3c4cc(-c5ccc(-c6ncco6)nc5)ccc4c4ccc(-c5ccc(-c6ncco6)nc5)cc4c3cc2-c2ccccc2)cc1]

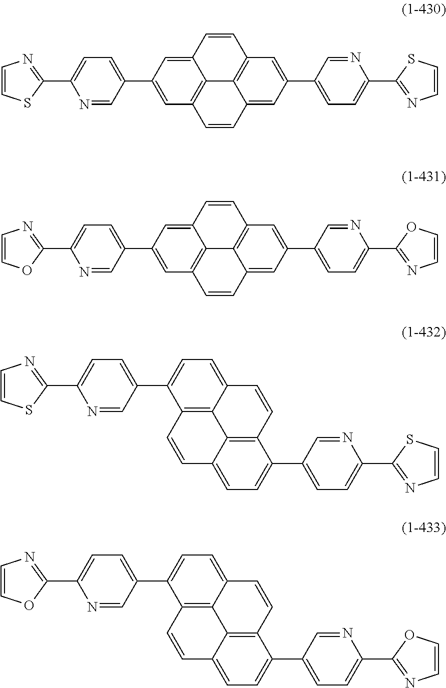 [SMILES: c1csc(-c2ccc(-c3cc4ccc5cc(-c6ccc(-c7nccs7)nc6)cc6ccc(c3)c4c56)cn2)n1 ; c1coc(-c2ccc(-c3cc4ccc5cc(-c6ccc(-c7ncco7)nc6)cc6ccc(c3)c4c56)cn2)n1 ; c1csc(-c2ccc(-c3ccc4ccc5c(-c6ccc(-c7nccs7)nc6)ccc6ccc3c4c65)cn2)n1 ; c1coc(-c2ccc(-c3ccc4ccc5c(-c6ccc(-c7ncco7)nc6)ccc6ccc3c4c65)cn2)n1]

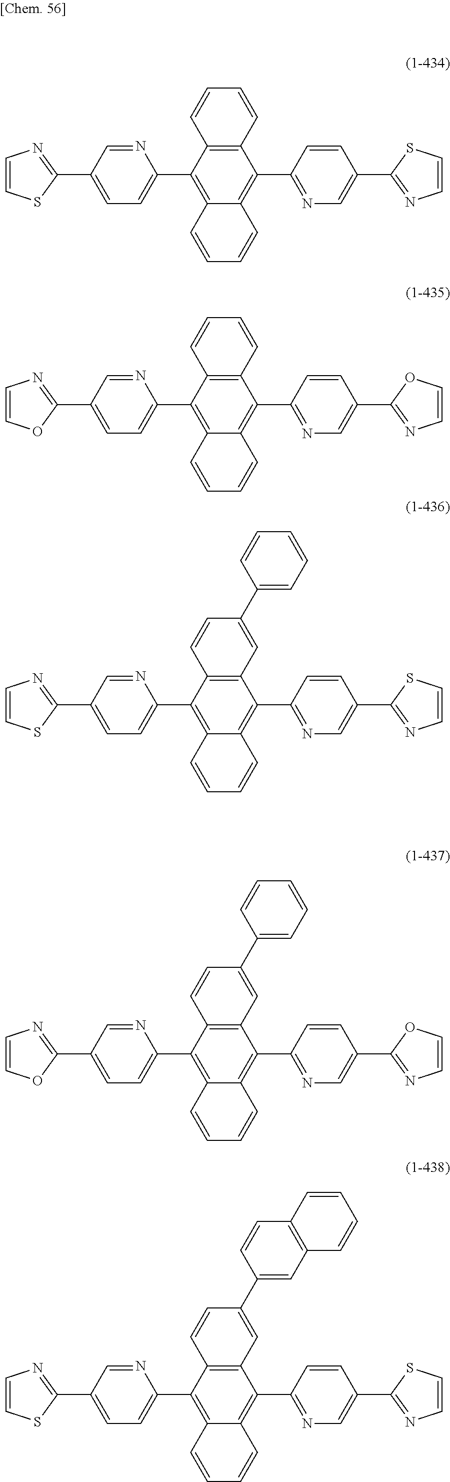

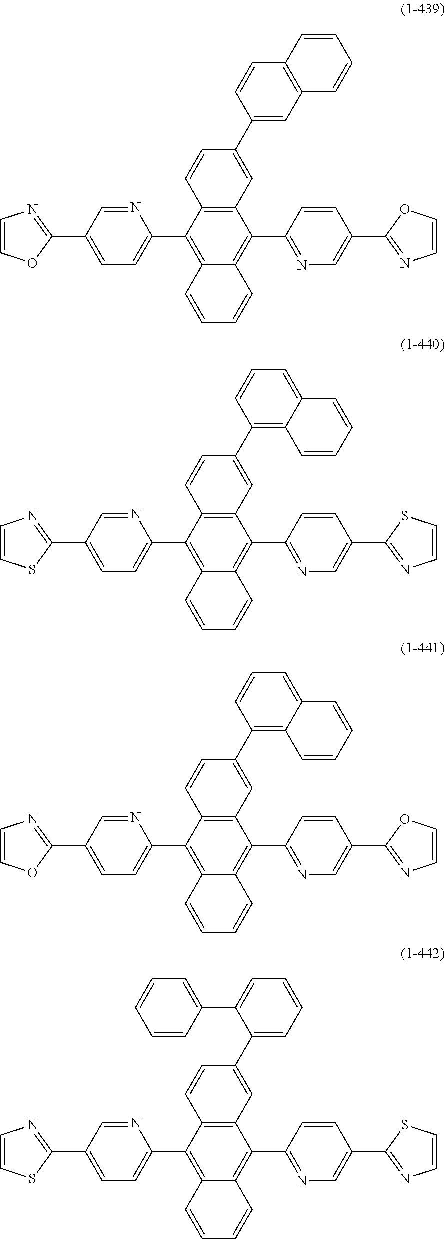

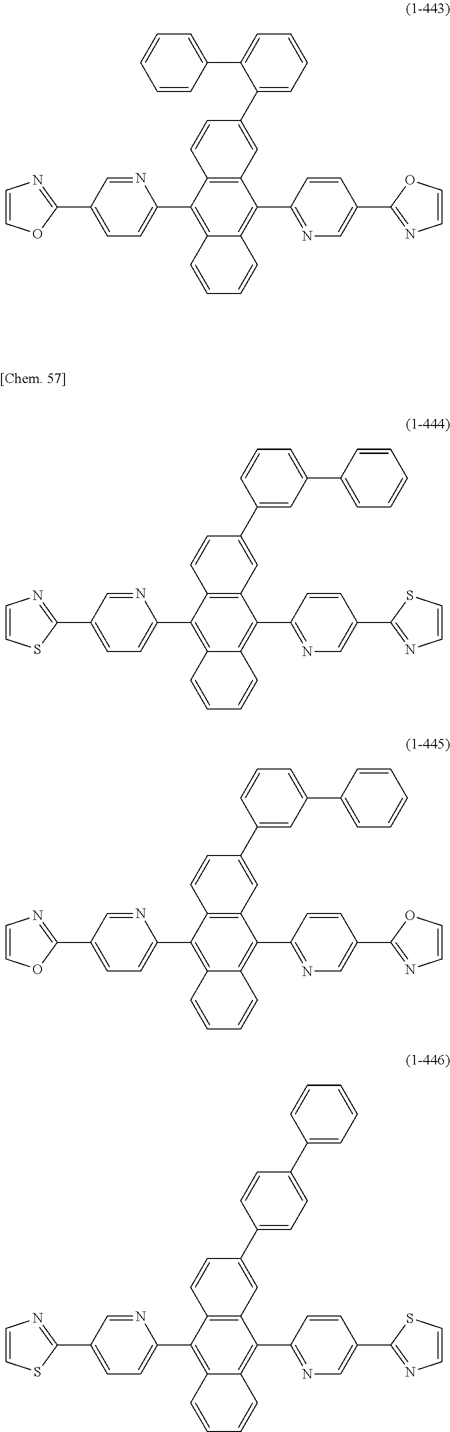 [SMILES: c1ccc(-c2ccccc2-c2ccc3c(-c4ccc(-c5ncco5)cn4)c4ccccc4c(-c4ccc(-c5ncco5)cn4)c3c2)cc1 ; c1ccc(-c2cccc(-c3ccc4c(-c5ccc(-c6nccs6)cn5)c5ccccc5c(-c5ccc(-c6nccs6)cn5)c4c3)c2)cc1 ; c1ccc(-c2cccc(-c3ccc4c(-c5ccc(-c6ncco6)cn5)c5ccccc5c(-c5ccc(-c6ncco6)cn5)c4c3)c2)cc1 ; c1ccc(-c2ccc(-c3ccc4c(-c5ccc(-c6nccs6)cn5)c5ccccc5c(-c5ccc(-c6nccs6)cn5)c4c3)cc2)cc1]

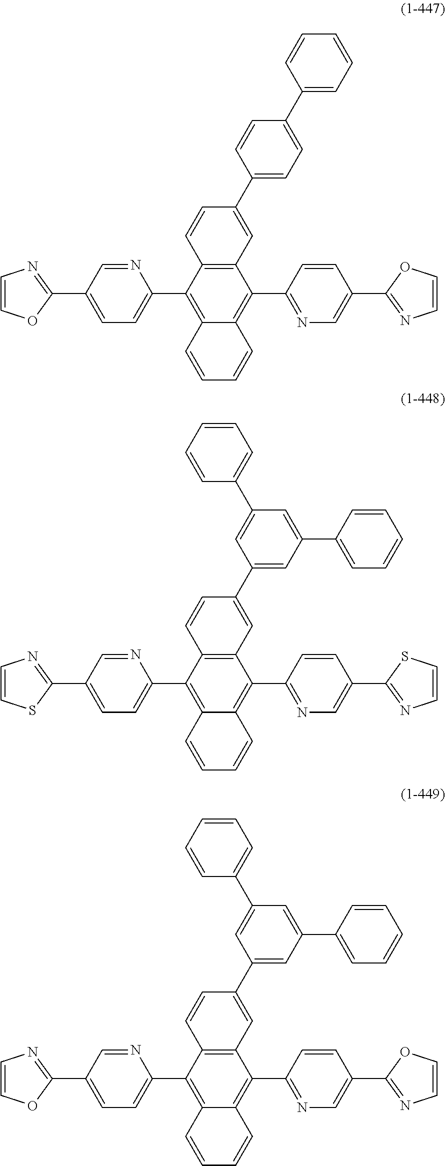

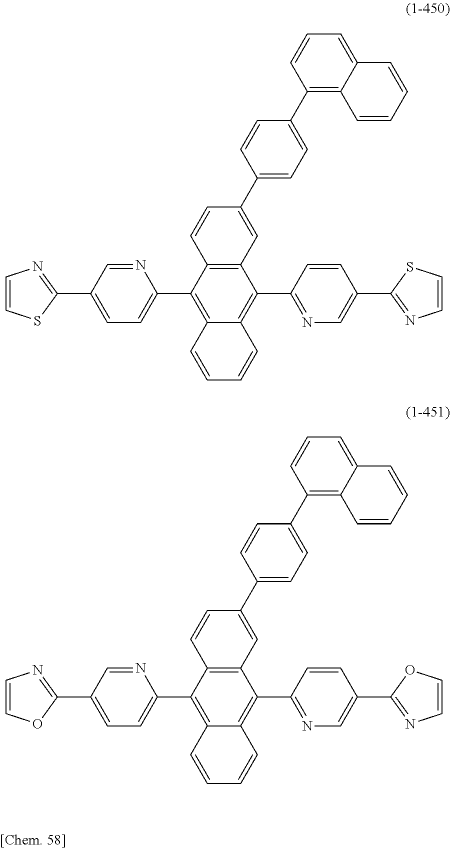 [SMILES: c1ccc2c(-c3ccc(-c4ccc5c(-c6ccc(-c7nccs7)cn6)c6ccccc6c(-c6ccc(-c7nccs7)cn6)c5c4)cc3)cccc2c1 ; c1ccc2c(-c3ccc(-c4ccc5c(-c6ccc(-c7ncco7)cn6)c6ccccc6c(-c6ccc(-c7ncco7)cn6)c5c4)cc3)cccc2c1]

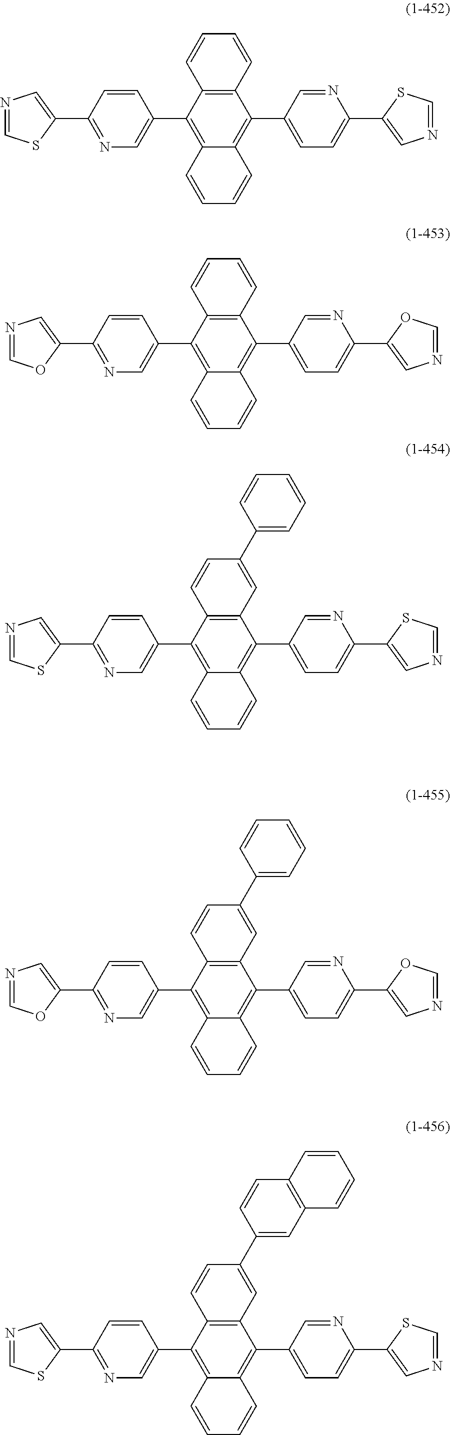 [SMILES: c1ccc2c(-c3ccc(-c4cncs4)nc3)c3ccccc3c(-c3ccc(-c4cncs4)nc3)c2c1 ; c1ccc2c(-c3ccc(-c4cnco4)nc3)c3ccccc3c(-c3ccc(-c4cnco4)nc3)c2c1 ; c1ccc(-c2ccc3c(-c4ccc(-c5cncs5)nc4)c4ccccc4c(-c4ccc(-c5cncs5)nc4)c3c2)cc1 ; c1ccc(-c2ccc3c(-c4ccc(-c5cnco5)nc4)c4ccccc4c(-c4ccc(-c5cnco5)nc4)c3c2)cc1 ; c1ccc2cc(-c3ccc4c(-c5ccc(-c6cncs6)nc5)c5ccccc5c(-c5ccc(-c6cncs6)nc5)c4c3)ccc2c1]

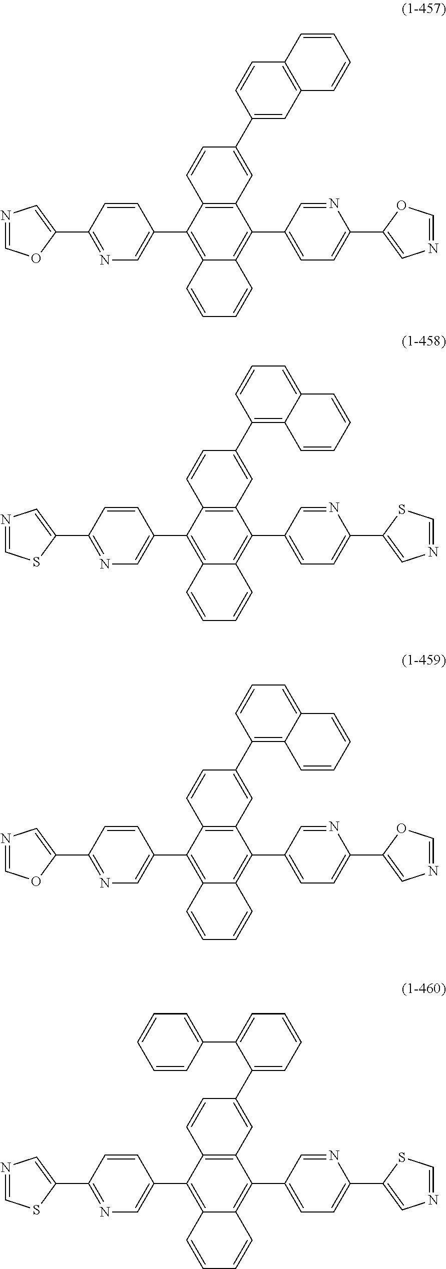 [SMILES: c1ccc2cc(-c3ccc4c(-c5ccc(-c6cnco6)nc5)c5ccccc5c(-c5ccc(-c6cnco6)nc5)c4c3)ccc2c1 ; c1ccc2c(-c3ccc4c(-c5ccc(-c6cncs6)nc5)c5ccccc5c(-c5ccc(-c6cncs6)nc5)c4c3)cccc2c1 ; c1ccc2c(-c3ccc4c(-c5ccc(-c6cnco6)nc5)c5ccccc5c(-c5ccc(-c6cnco6)nc5)c4c3)cccc2c1 ; c1ccc(-c2ccccc2-c2ccc3c(-c4ccc(-c5cncs5)nc4)c4ccccc4c(-c4ccc(-c5cncs5)nc4)c3c2)cc1]

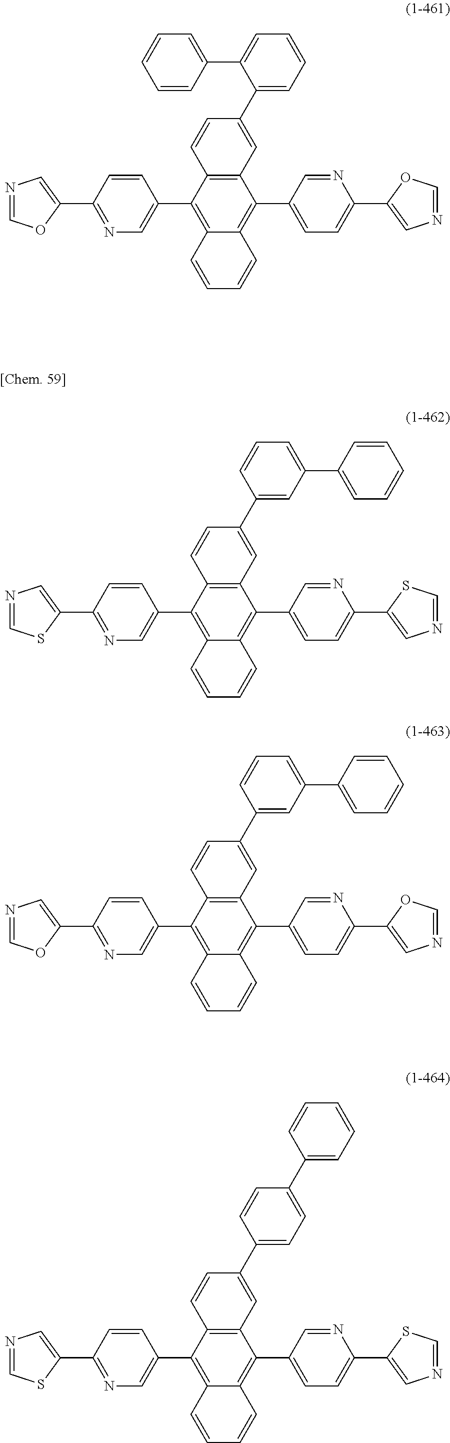 [SMILES: c1ccc(-c2ccccc2-c2ccc3c(-c4ccc(-c5cnco5)nc4)c4ccccc4c(-c4ccc(-c5cnco5)nc4)c3c2)cc1 ; c1ccc(-c2cccc(-c3ccc4c(-c5ccc(-c6cncs6)nc5)c5ccccc5c(-c5ccc(-c6cncs6)nc5)c4c3)c2)cc1 ; c1ccc(-c2cccc(-c3ccc4c(-c5ccc(-c6cnco6)nc5)c5ccccc5c(-c5ccc(-c6cnco6)nc5)c4c3)c2)cc1 ; c1ccc(-c2ccc(-c3ccc4c(-c5ccc(-c6cncs6)nc5)c5ccccc5c(-c5ccc(-c6cncs6)nc5)c4c3)cc2)cc1]

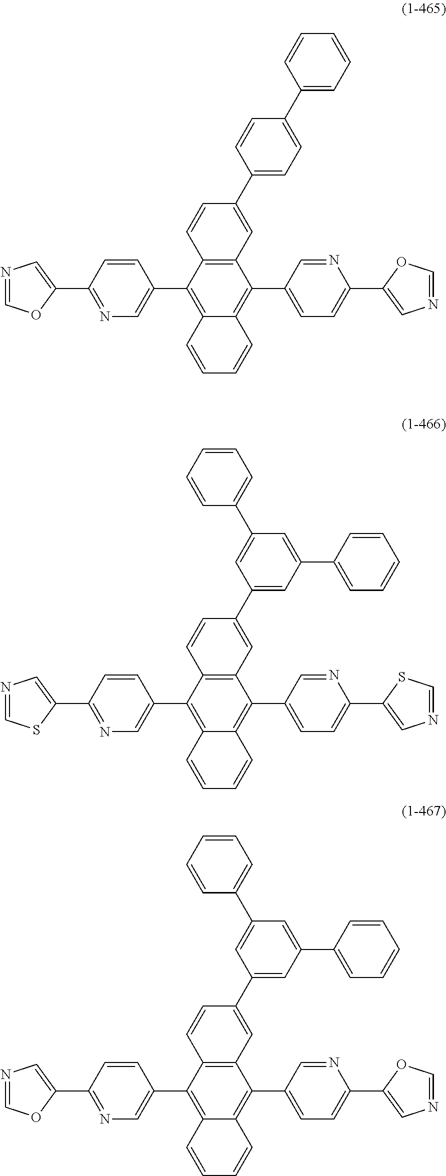 [SMILES: c1ccc(-c2ccc(-c3ccc4c(-c5ccc(-c6cnco6)nc5)c5ccccc5c(-c5ccc(-c6cnco6)nc5)c4c3)cc2)cc1 ; c1ccc(-c2cc(-c3ccccc3)cc(-c3ccc4c(-c5ccc(-c6cncs6)nc5)c5ccccc5c(-c5ccc(-c6cncs6)nc5)c4c3)c2)cc1 ; c1ccc(-c2cc(-c3ccccc3)cc(-c3ccc4c(-c5ccc(-c6cnco6)nc5)c5ccccc5c(-c5ccc(-c6cnco6)nc5)c4c3)c2)cc1]

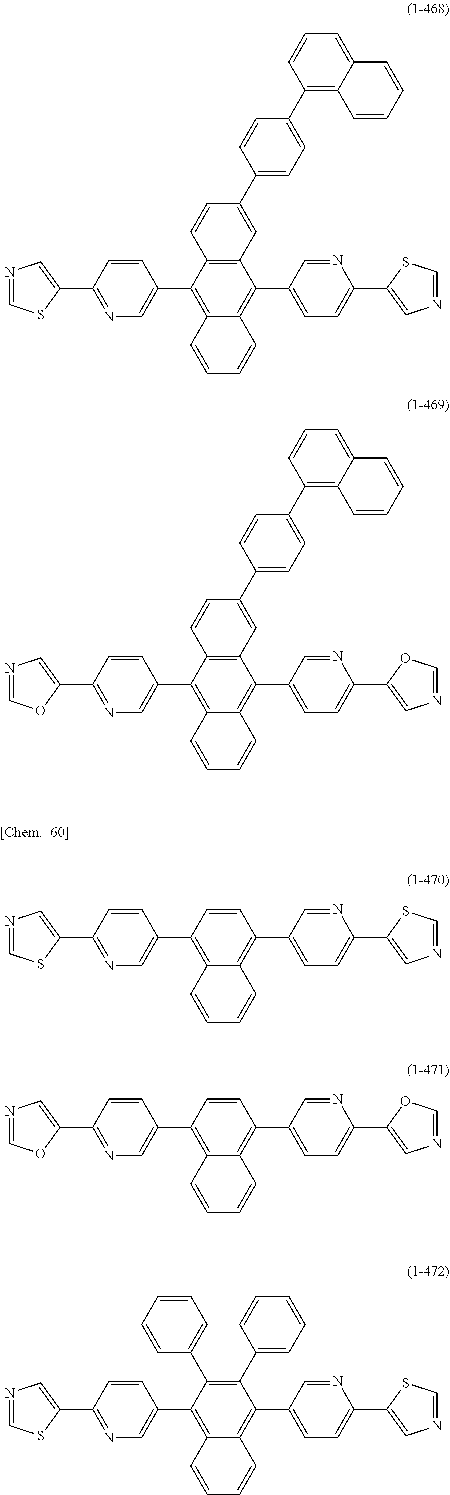 [SMILES: c1ccc2c(-c3ccc(-c4ccc5c(-c6ccc(-c7cncs7)nc6)c6ccccc6c(-c6ccc(-c7cncs7)nc6)c5c4)cc3)cccc2c1 ; c1ccc2c(-c3ccc(-c4ccc5c(-c6ccc(-c7cnco7)nc6)c6ccccc6c(-c6ccc(-c7cnco7)nc6)c5c4)cc3)cccc2c1 ; c1ccc2c(-c3ccc(-c4cncs4)nc3)ccc(-c3ccc(-c4cncs4)nc3)c2c1 ; c1ccc2c(-c3ccc(-c4cnco4)nc3)ccc(-c3ccc(-c4cnco4)nc3)c2c1 ; c1ccc(-c2c(-c3ccccc3)c(-c3ccc(-c4cncs4)nc3)c3ccccc3c2-c2ccc(-c3cncs3)nc2)cc1]

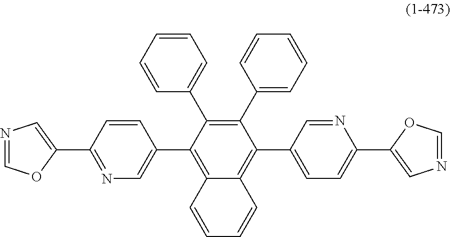 [SMILES: c1ccc(-c2c(-c3ccccc3)c(-c3ccc(-c4cnco4)nc3)c3ccccc3c2-c2ccc(-c3cnco3)nc2)cc1]

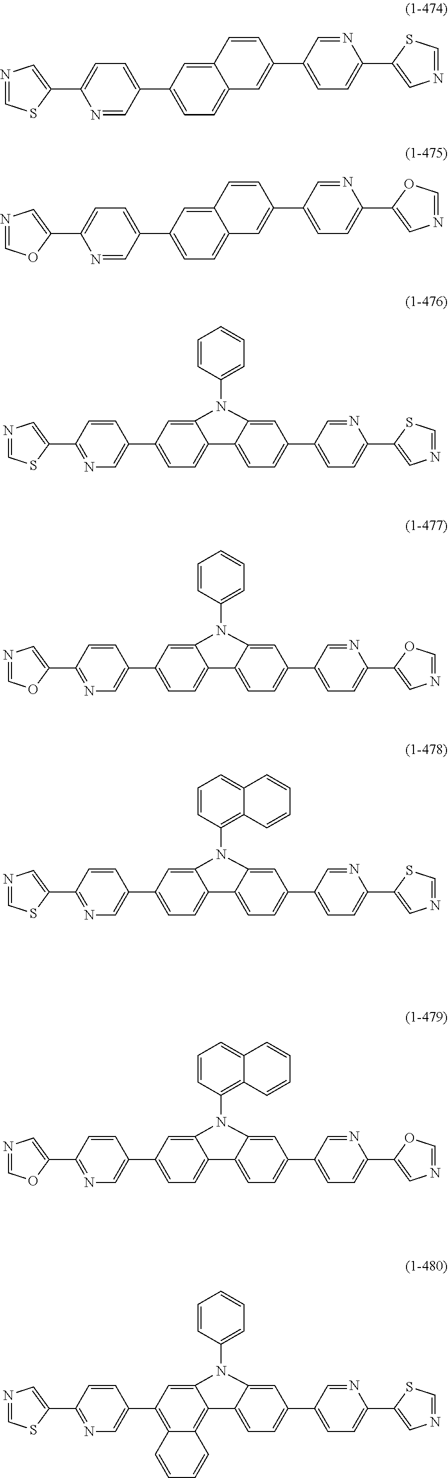

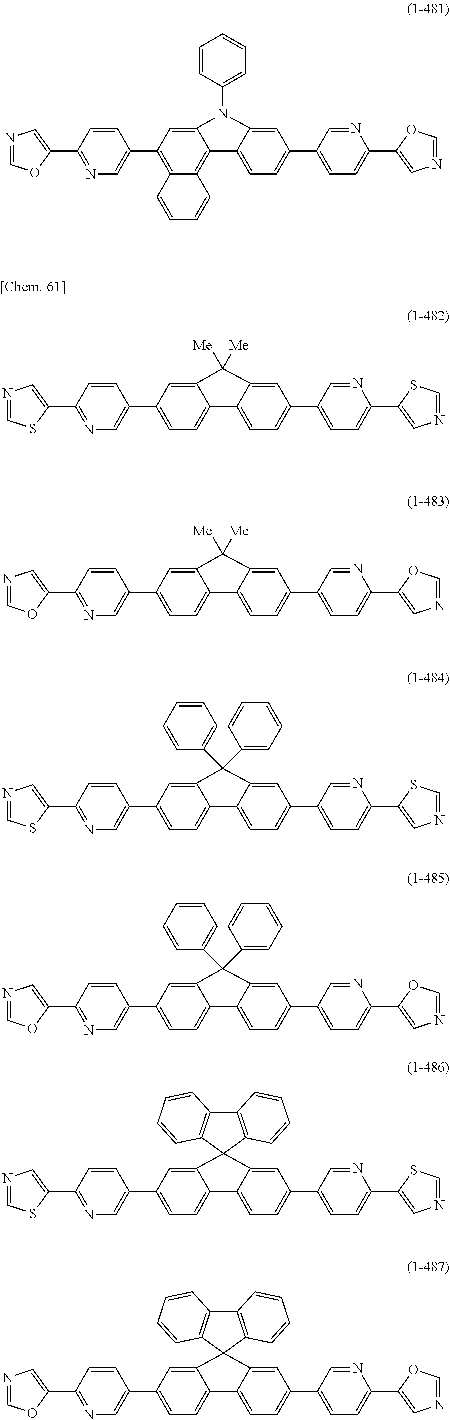

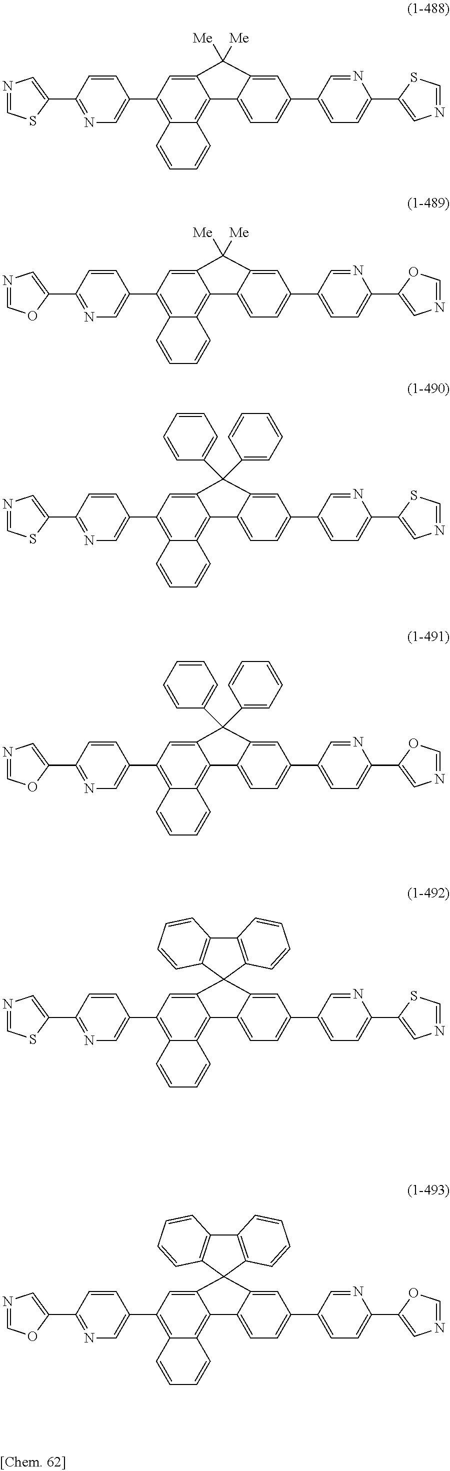

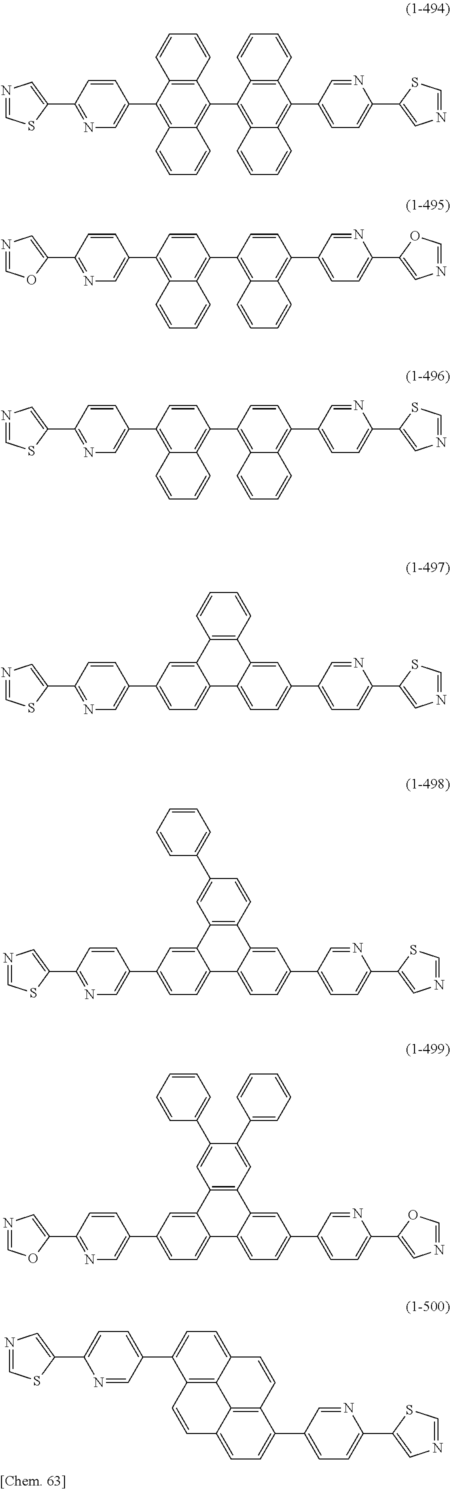

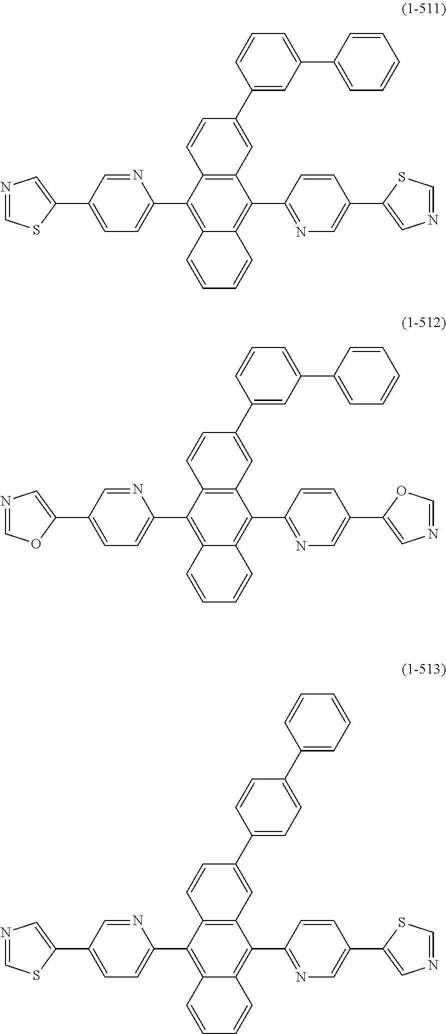 [SMILES: c1ccc(-c2cccc(-c3ccc4c(-c5ccc(-c6cncs6)cn5)c5ccccc5c(-c5ccc(-c6cncs6)cn5)c4c3)c2)cc1 ; c1ccc(-c2cccc(-c3ccc4c(-c5ccc(-c6cnco6)cn5)c5ccccc5c(-c5ccc(-c6cnco6)cn5)c4c3)c2)cc1 ; c1ccc(-c2ccc(-c3ccc4c(-c5ccc(-c6cncs6)cn5)c5ccccc5c(-c5ccc(-c6cncs6)cn5)c4c3)cc2)cc1]

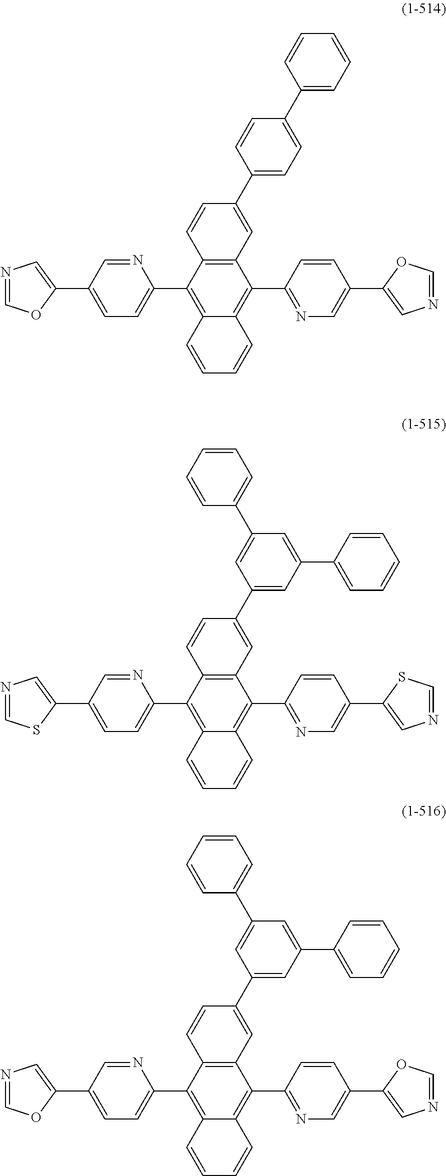 [SMILES: c1ccc(-c2ccc(-c3ccc4c(-c5ccc(-c6cnco6)cn5)c5ccccc5c(-c5ccc(-c6cnco6)cn5)c4c3)cc2)cc1 ; c1ccc(-c2cc(-c3ccccc3)cc(-c3ccc4c(-c5ccc(-c6cncs6)cn5)c5ccccc5c(-c5ccc(-c6cncs6)cn5)c4c3)c2)cc1 ; c1ccc(-c2cc(-c3ccccc3)cc(-c3ccc4c(-c5ccc(-c6cnco6)cn5)c5ccccc5c(-c5ccc(-c6cnco6)cn5)c4c3)c2)cc1]

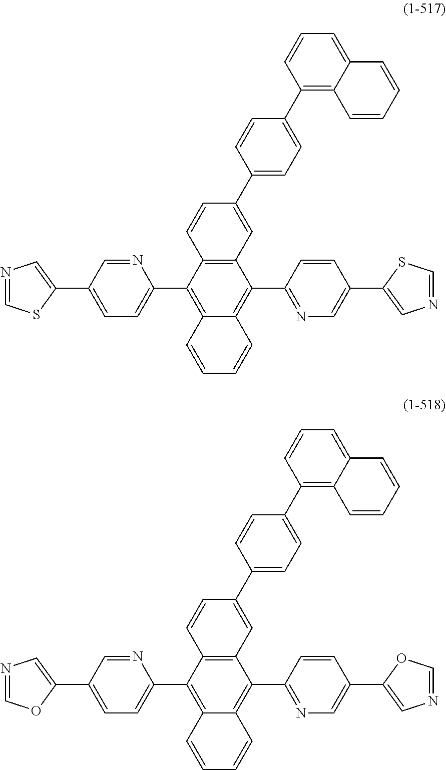 [SMILES: c1ccc2c(-c3ccc(-c4ccc5c(-c6ccc(-c7cncs7)cn6)c6ccccc6c(-c6ccc(-c7cncs7)cn6)c5c4)cc3)cccc2c1 ; c1ccc2c(-c3ccc(-c4ccc5c(-c6ccc(-c7cnco7)cn6)c6ccccc6c(-c6ccc(-c7cnco7)cn6)c5c4)cc3)cccc2c1]

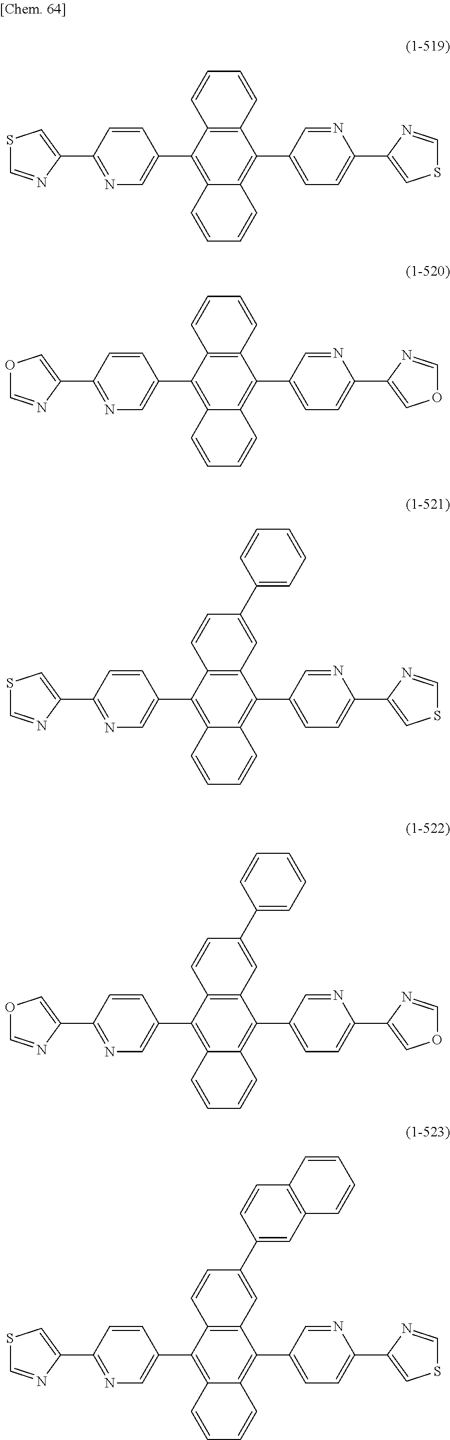 [SMILES: c1ccc2c(-c3ccc(-c4cscn4)nc3)c3ccccc3c(-c3ccc(-c4cscn4)nc3)c2c1 ; c1ccc2c(-c3ccc(-c4cocn4)nc3)c3ccccc3c(-c3ccc(-c4cocn4)nc3)c2c1 ; c1ccc(-c2ccc3c(-c4ccc(-c5cscn5)nc4)c4ccccc4c(-c4ccc(-c5cscn5)nc4)c3c2)cc1 ; c1ccc(-c2ccc3c(-c4ccc(-c5cocn5)nc4)c4ccccc4c(-c4ccc(-c5cocn5)nc4)c3c2)cc1 ; c1ccc2cc(-c3ccc4c(-c5ccc(-c6cscn6)nc5)c5ccccc5c(-c5ccc(-c6cscn6)nc5)c4c3)ccc2c1]

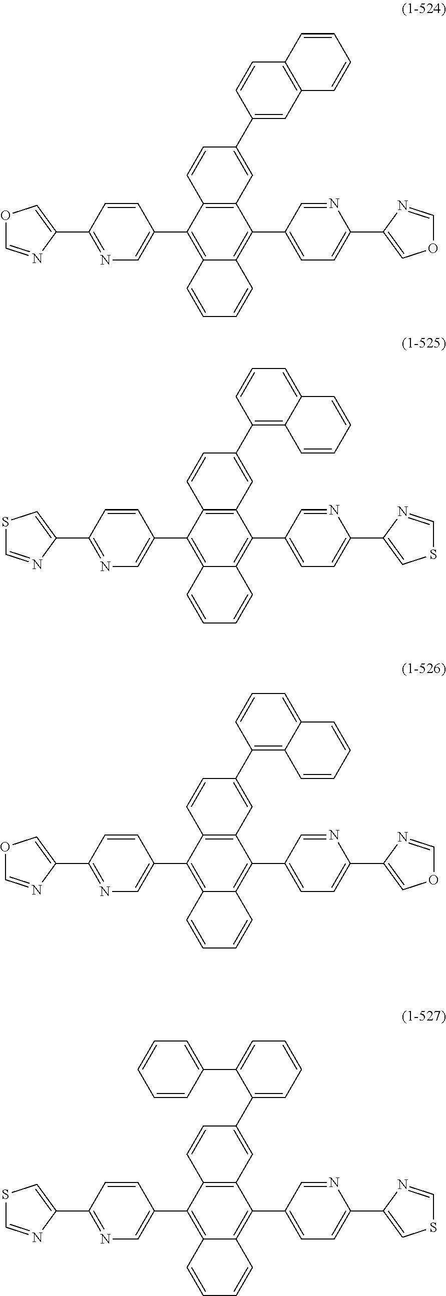 [SMILES: c1ccc2cc(-c3ccc4c(-c5ccc(-c6cocn6)nc5)c5ccccc5c(-c5ccc(-c6cocn6)nc5)c4c3)ccc2c1 ; c1ccc2c(-c3ccc4c(-c5ccc(-c6cscn6)nc5)c5ccccc5c(-c5ccc(-c6cscn6)nc5)c4c3)cccc2c1 ; c1ccc2c(-c3ccc4c(-c5ccc(-c6cocn6)nc5)c5ccccc5c(-c5ccc(-c6cocn6)nc5)c4c3)cccc2c1 ; c1ccc(-c2ccccc2-c2ccc3c(-c4ccc(-c5cscn5)nc4)c4ccccc4c(-c4ccc(-c5cscn5)nc4)c3c2)cc1]

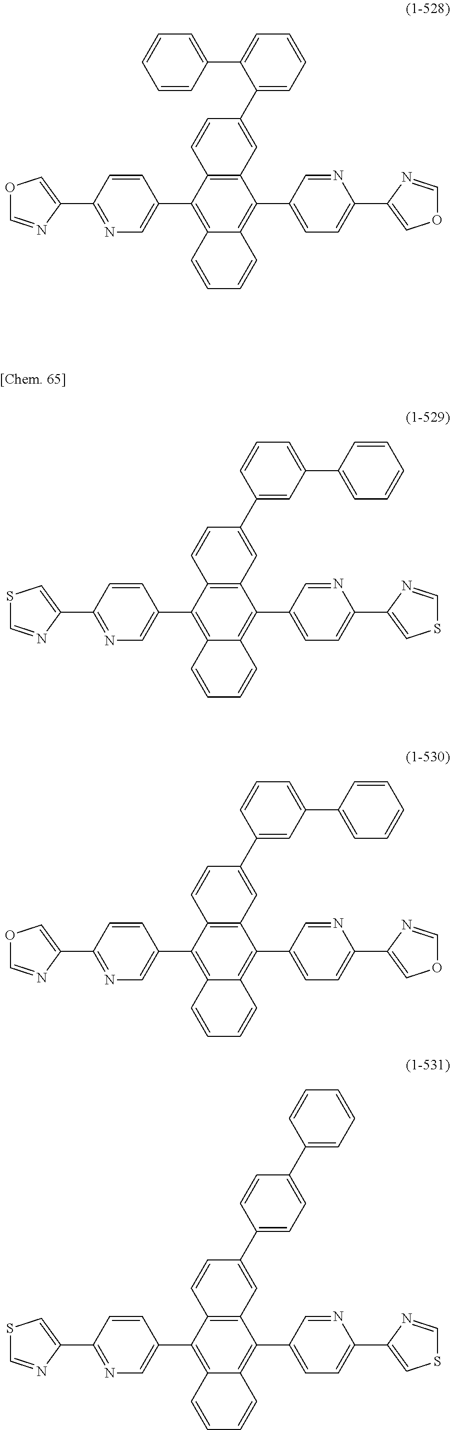 [SMILES: c1ccc(-c2ccccc2-c2ccc3c(-c4ccc(-c5cocn5)nc4)c4ccccc4c(-c4ccc(-c5cocn5)nc4)c3c2)cc1 ; c1ccc(-c2cccc(-c3ccc4c(-c5ccc(-c6cscn6)nc5)c5ccccc5c(-c5ccc(-c6cscn6)nc5)c4c3)c2)cc1 ; c1ccc(-c2cccc(-c3ccc4c(-c5ccc(-c6cocn6)nc5)c5ccccc5c(-c5ccc(-c6cocn6)nc5)c4c3)c2)cc1 ; c1ccc(-c2ccc(-c3ccc4c(-c5ccc(-c6cscn6)nc5)c5ccccc5c(-c5ccc(-c6cscn6)nc5)c4c3)cc2)cc1]

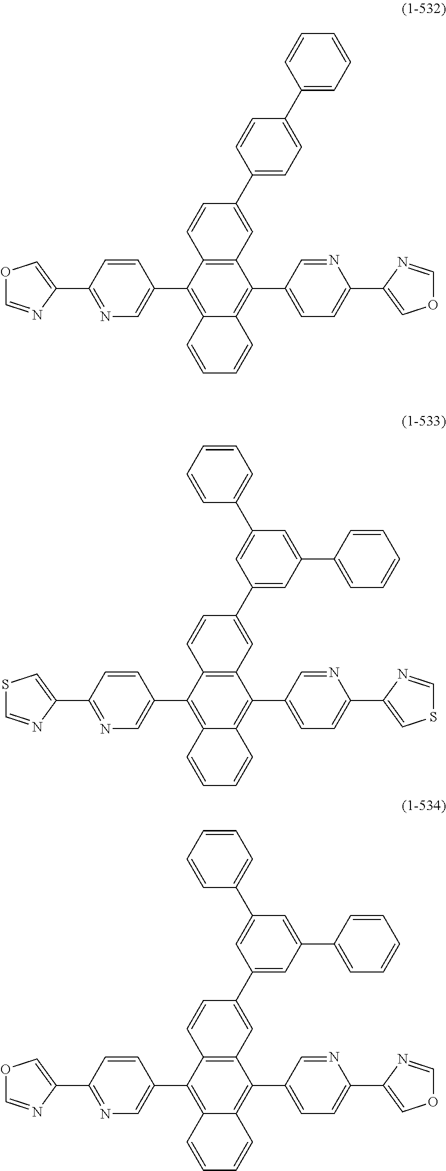

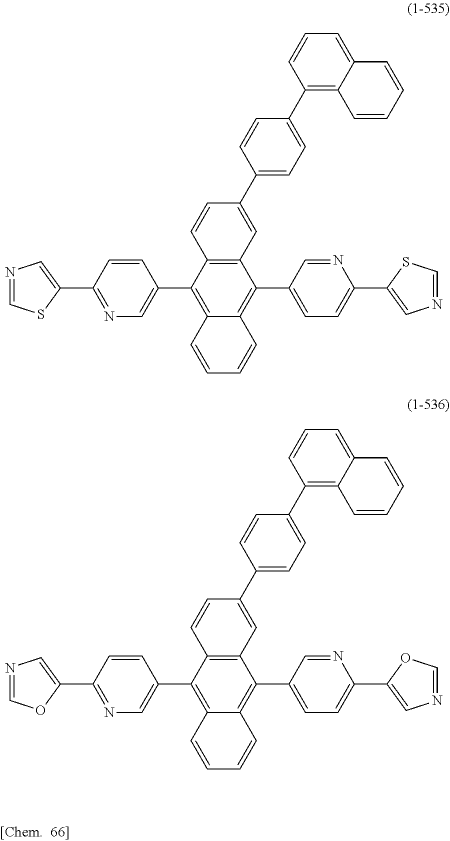 [SMILES: c1ccc2c(-c3ccc(-c4ccc5c(-c6ccc(-c7cncs7)nc6)c6ccccc6c(-c6ccc(-c7cncs7)nc6)c5c4)cc3)cccc2c1 ; c1ccc2c(-c3ccc(-c4ccc5c(-c6ccc(-c7cnco7)nc6)c6ccccc6c(-c6ccc(-c7cnco7)nc6)c5c4)cc3)cccc2c1]

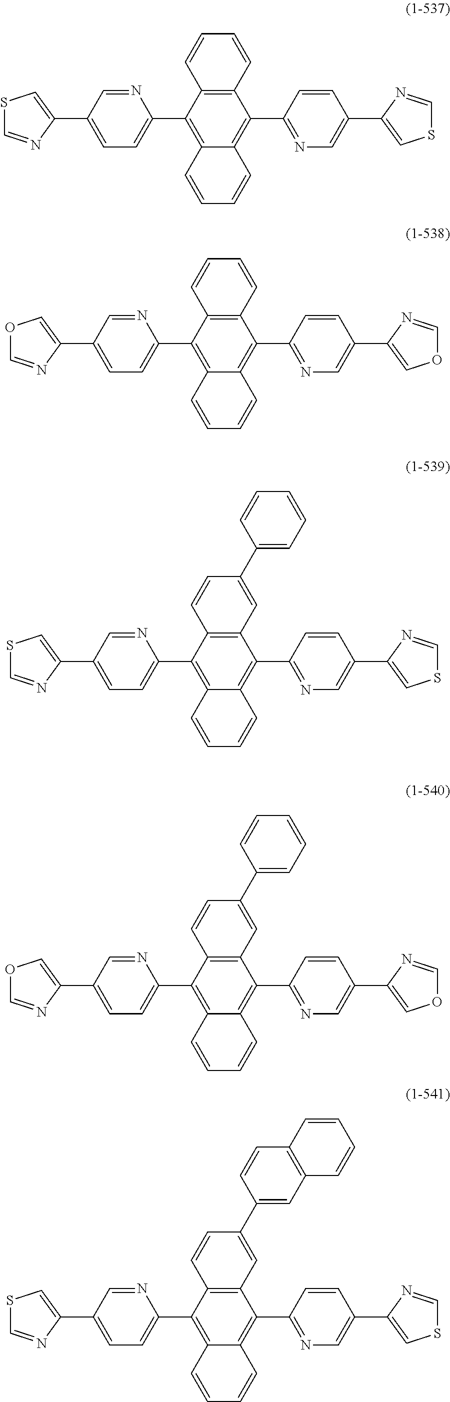 [SMILES: c1ccc2c(-c3ccc(-c4cscn4)cn3)c3ccccc3c(-c3ccc(-c4cscn4)cn3)c2c1 ; c1ccc2c(-c3ccc(-c4cocn4)cn3)c3ccccc3c(-c3ccc(-c4cocn4)cn3)c2c1 ; c1ccc(-c2ccc3c(-c4ccc(-c5cscn5)cn4)c4ccccc4c(-c4ccc(-c5cscn5)cn4)c3c2)cc1 ; c1ccc(-c2ccc3c(-c4ccc(-c5cocn5)cn4)c4ccccc4c(-c4ccc(-c5cocn5)cn4)c3c2)cc1 ; c1ccc2cc(-c3ccc4c(-c5ccc(-c6cscn6)cn5)c5ccccc5c(-c5ccc(-c6cscn6)cn5)c4c3)ccc2c1]

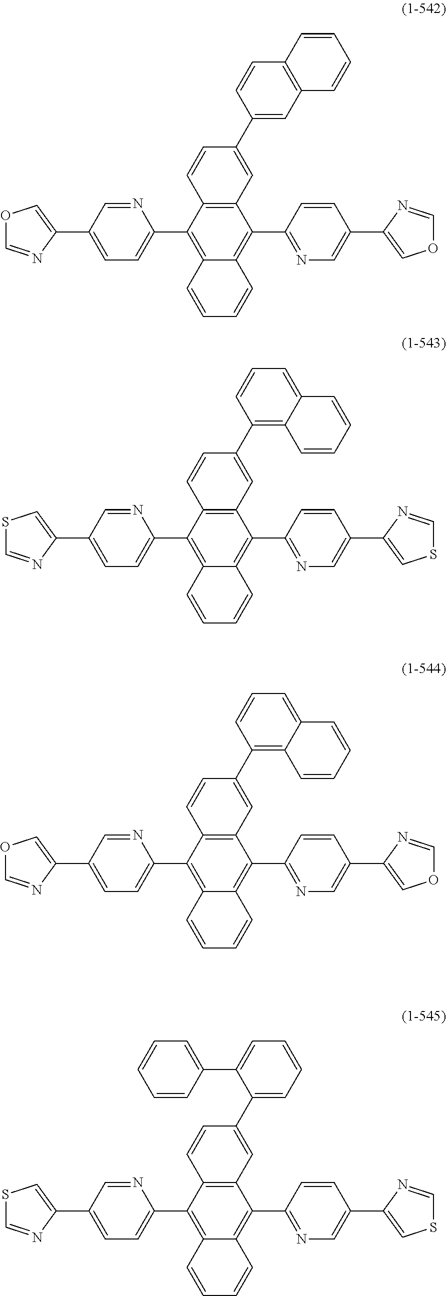 [SMILES: c1ccc2cc(-c3ccc4c(-c5ccc(-c6cocn6)cn5)c5ccccc5c(-c5ccc(-c6cocn6)cn5)c4c3)ccc2c1 ; c1ccc2c(-c3ccc4c(-c5ccc(-c6cscn6)cn5)c5ccccc5c(-c5ccc(-c6cscn6)cn5)c4c3)cccc2c1 ; c1ccc2c(-c3ccc4c(-c5ccc(-c6cocn6)cn5)c5ccccc5c(-c5ccc(-c6cocn6)cn5)c4c3)cccc2c1 ; c1ccc(-c2ccccc2-c2ccc3c(-c4ccc(-c5cscn5)cn4)c4ccccc4c(-c4ccc(-c5cscn5)cn4)c3c2)cc1]

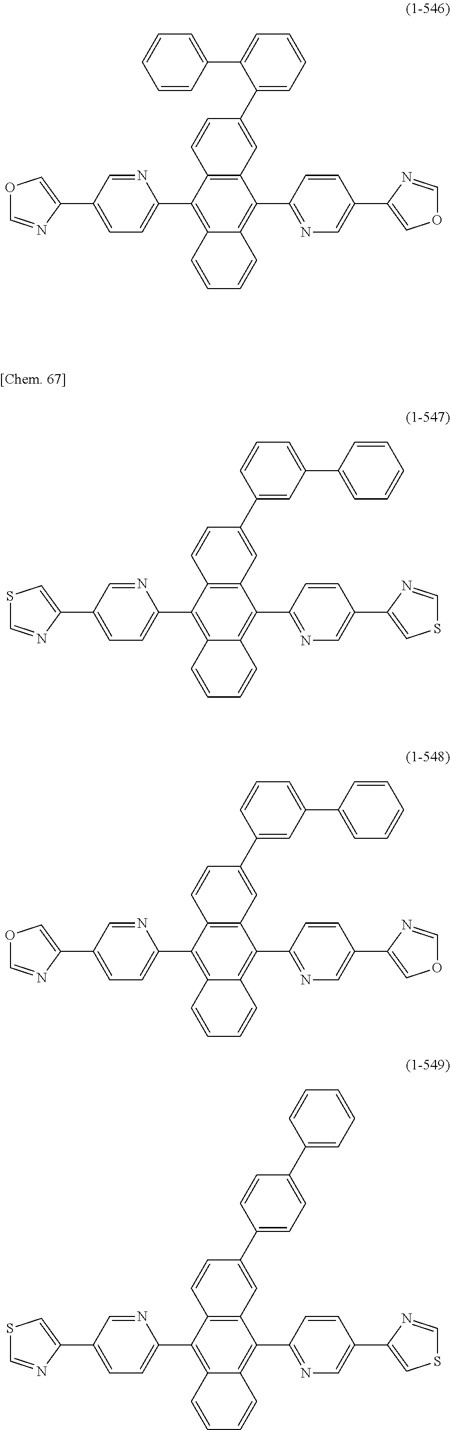 [SMILES: c1ccc(-c2ccccc2-c2ccc3c(-c4ccc(-c5cocn5)cn4)c4ccccc4c(-c4ccc(-c5cocn5)cn4)c3c2)cc1 ; c1ccc(-c2cccc(-c3ccc4c(-c5ccc(-c6cscn6)cn5)c5ccccc5c(-c5ccc(-c6cscn6)cn5)c4c3)c2)cc1 ; c1ccc(-c2cccc(-c3ccc4c(-c5ccc(-c6cocn6)cn5)c5ccccc5c(-c5ccc(-c6cocn6)cn5)c4c3)c2)cc1 ; c1ccc(-c2ccc(-c3ccc4c(-c5ccc(-c6cscn6)cn5)c5ccccc5c(-c5ccc(-c6cscn6)cn5)c4c3)cc2)cc1]

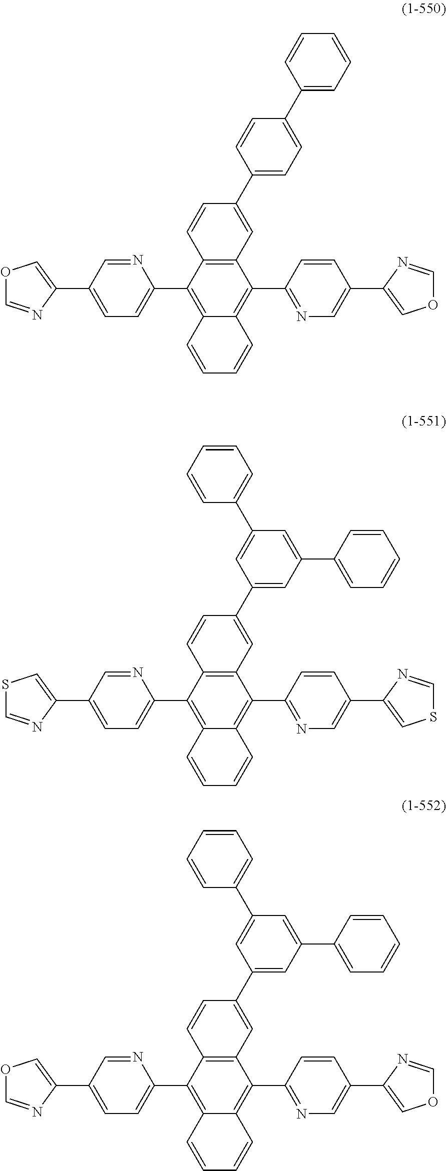

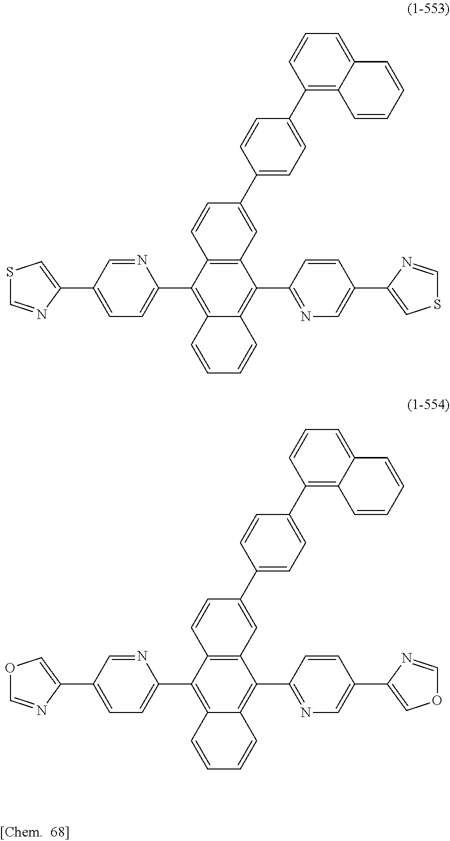 [SMILES: c1ccc2c(-c3ccc(-c4ccc5c(-c6ccc(-c7cscn7)cn6)c6ccccc6c(-c6ccc(-c7cscn7)cn6)c5c4)cc3)cccc2c1 ; c1ccc2c(-c3ccc(-c4ccc5c(-c6ccc(-c7cocn7)cn6)c6ccccc6c(-c6ccc(-c7cocn7)cn6)c5c4)cc3)cccc2c1]

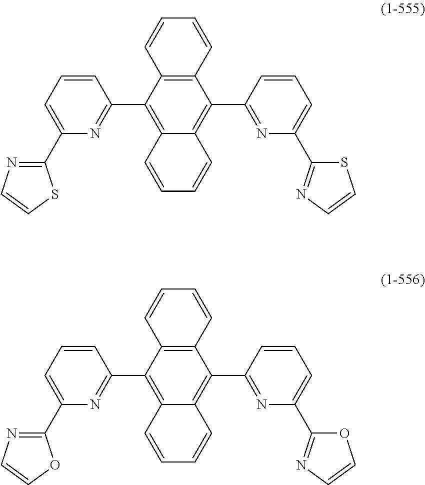 [SMILES: c1cc(-c2nccs2)nc(-c2c3ccccc3c(-c3cccc(-c4nccs4)n3)c3ccccc23)c1 ; c1cc(-c2ncco2)nc(-c2c3ccccc3c(-c3cccc(-c4ncco4)n3)c3ccccc23)c1]

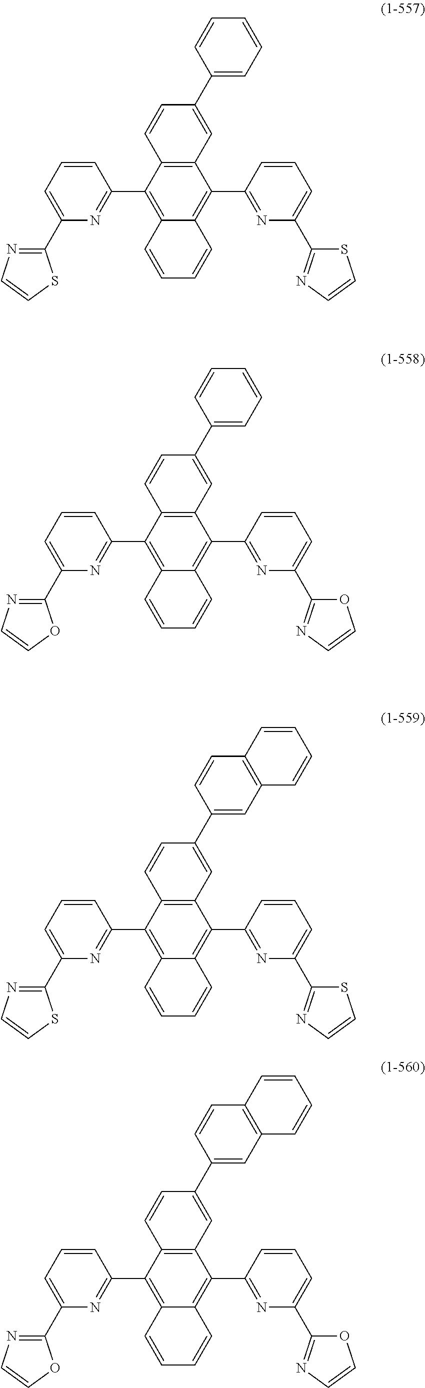 [SMILES: c1ccc(-c2ccc3c(-c4cccc(-c5nccs5)n4)c4ccccc4c(-c4cccc(-c5nccs5)n4)c3c2)cc1 ; c1ccc(-c2ccc3c(-c4cccc(-c5ncco5)n4)c4ccccc4c(-c4cccc(-c5ncco5)n4)c3c2)cc1 ; c1cc(-c2nccs2)nc(-c2c3ccccc3c(-c3cccc(-c4nccs4)n3)c3cc(-c4ccc5ccccc5c4)ccc23)c1 ; c1cc(-c2ncco2)nc(-c2c3ccccc3c(-c3cccc(-c4ncco4)n3)c3cc(-c4ccc5ccccc5c4)ccc23)c1]

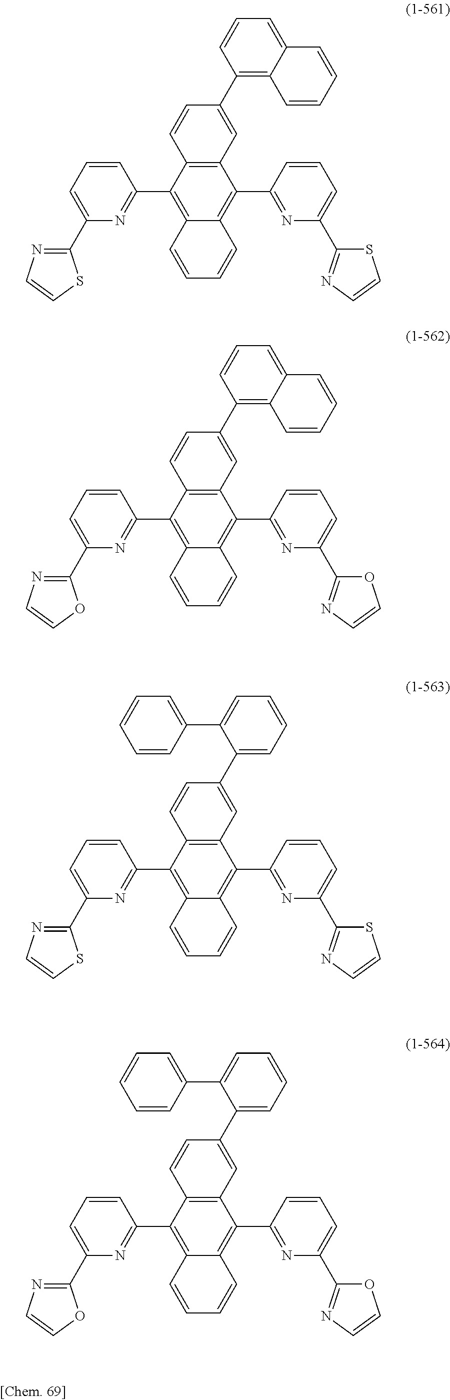 [SMILES: c1cc(-c2nccs2)nc(-c2c3ccccc3c(-c3cccc(-c4nccs4)n3)c3cc(-c4cccc5ccccc45)ccc23)c1 ; c1cc(-c2ncco2)nc(-c2c3ccccc3c(-c3cccc(-c4ncco4)n3)c3cc(-c4cccc5ccccc45)ccc23)c1 ; c1ccc(-c2ccccc2-c2ccc3c(-c4cccc(-c5nccs5)n4)c4ccccc4c(-c4cccc(-c5nccs5)n4)c3c2)cc1 ; c1ccc(-c2ccccc2-c2ccc3c(-c4cccc(-c5ncco5)n4)c4ccccc4c(-c4cccc(-c5ncco5)n4)c3c2)cc1]

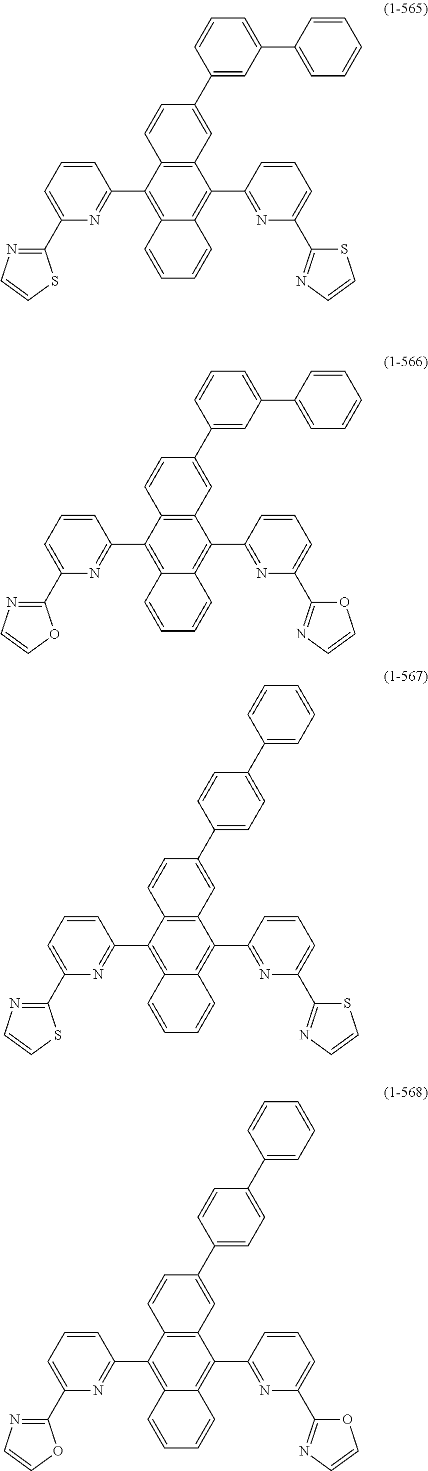

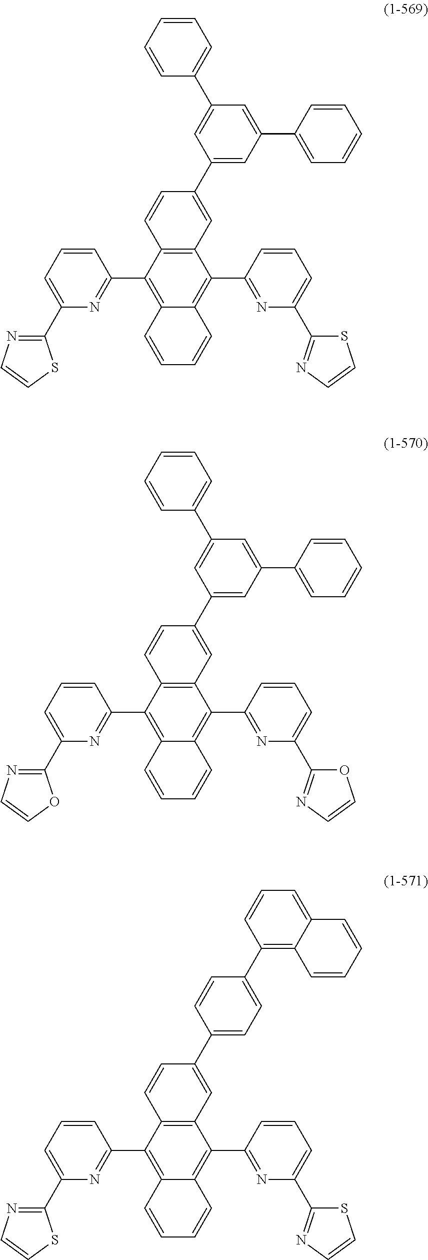 [SMILES: c1ccc(-c2cc(-c3ccccc3)cc(-c3ccc4c(-c5cccc(-c6nccs6)n5)c5ccccc5c(-c5cccc(-c6nccs6)n5)c4c3)c2)cc1 ; c1ccc(-c2cc(-c3ccccc3)cc(-c3ccc4c(-c5cccc(-c6ncco6)n5)c5ccccc5c(-c5cccc(-c6ncco6)n5)c4c3)c2)cc1 ; c1cc(-c2nccs2)nc(-c2c3ccccc3c(-c3cccc(-c4nccs4)n3)c3cc(-c4ccc(-c5cccc6ccccc56)cc4)ccc23)c1]

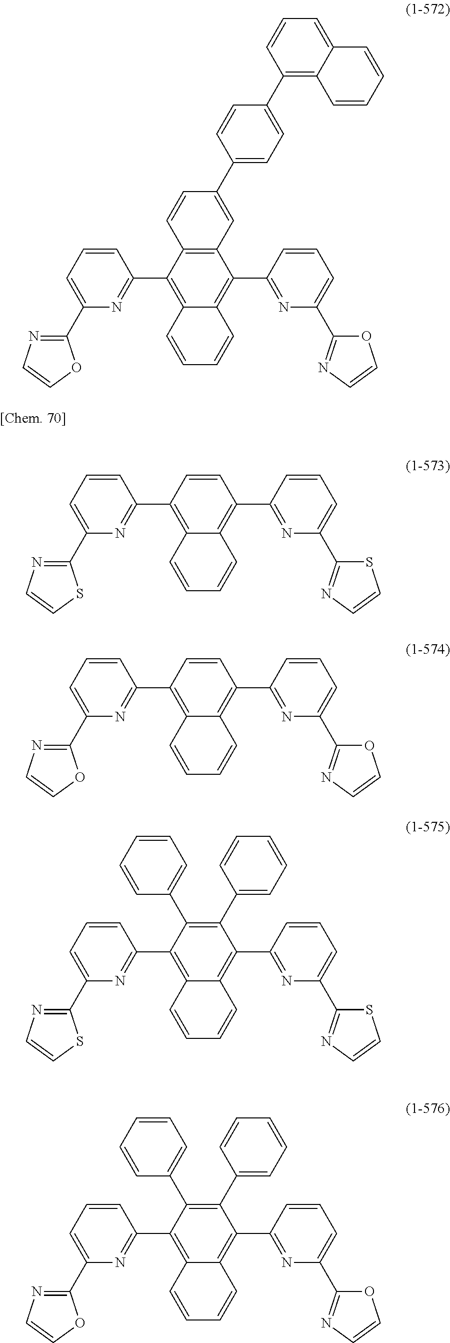 [SMILES: c1cc(-c2ncco2)nc(-c2c3ccccc3c(-c3cccc(-c4ncco4)n3)c3cc(-c4ccc(-c5cccc6ccccc56)cc4)ccc23)c1 ; c1cc(-c2nccs2)nc(-c2ccc(-c3cccc(-c4nccs4)n3)c3ccccc23)c1 ; c1cc(-c2ncco2)nc(-c2ccc(-c3cccc(-c4ncco4)n3)c3ccccc23)c1 ; c1ccc(-c2c(-c3ccccc3)c(-c3cccc(-c4nccs4)n3)c3ccccc3c2-c2cccc(-c3nccs3)n2)cc1 ; c1ccc(-c2c(-c3ccccc3)c(-c3cccc(-c4ncco4)n3)c3ccccc3c2-c2cccc(-c3ncco3)n2)cc1]

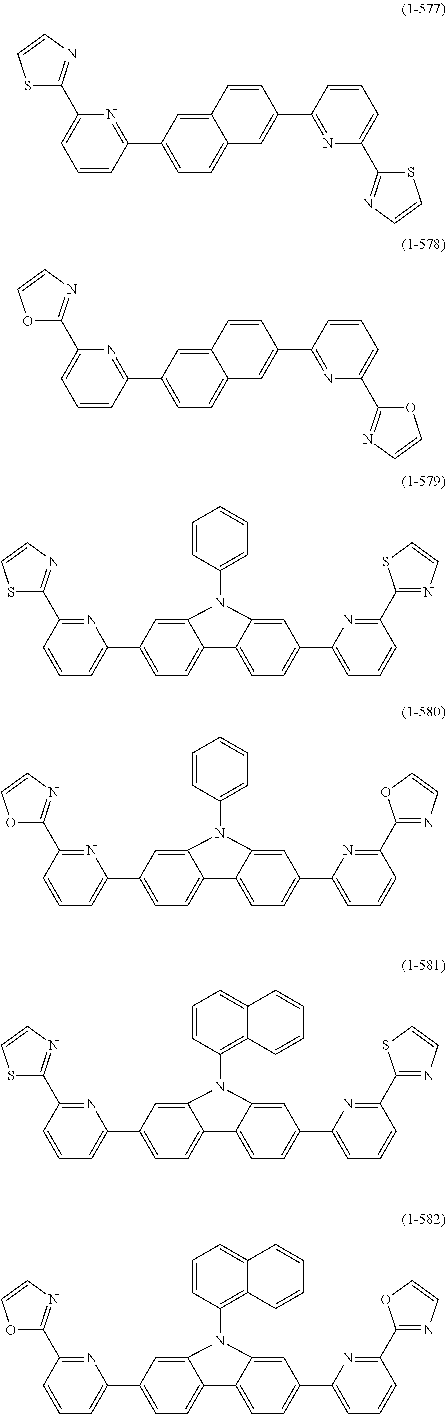 [SMILES: c1cc(-c2ccc3cc(-c4cccc(-c5nccs5)n4)ccc3c2)nc(-c2nccs2)c1 ; c1cc(-c2ccc3cc(-c4cccc(-c5ncco5)n4)ccc3c2)nc(-c2ncco2)c1 ; c1ccc(-n2c3cc(-c4cccc(-c5nccs5)n4)ccc3c3ccc(-c4cccc(-c5nccs5)n4)cc32)cc1 ; c1ccc(-n2c3cc(-c4cccc(-c5ncco5)n4)ccc3c3ccc(-c4cccc(-c5ncco5)n4)cc32)cc1 ; c1cc(-c2ccc3c4ccc(-c5cccc(-c6nccs6)n5)cc4n(-c4cccc5ccccc45)c3c2)nc(-c2nccs2)c1 ; c1cc(-c2ccc3c4ccc(-c5cccc(-c6ncco6)n5)cc4n(-c4cccc5ccccc45)c3c2)nc(-c2ncco2)c1]

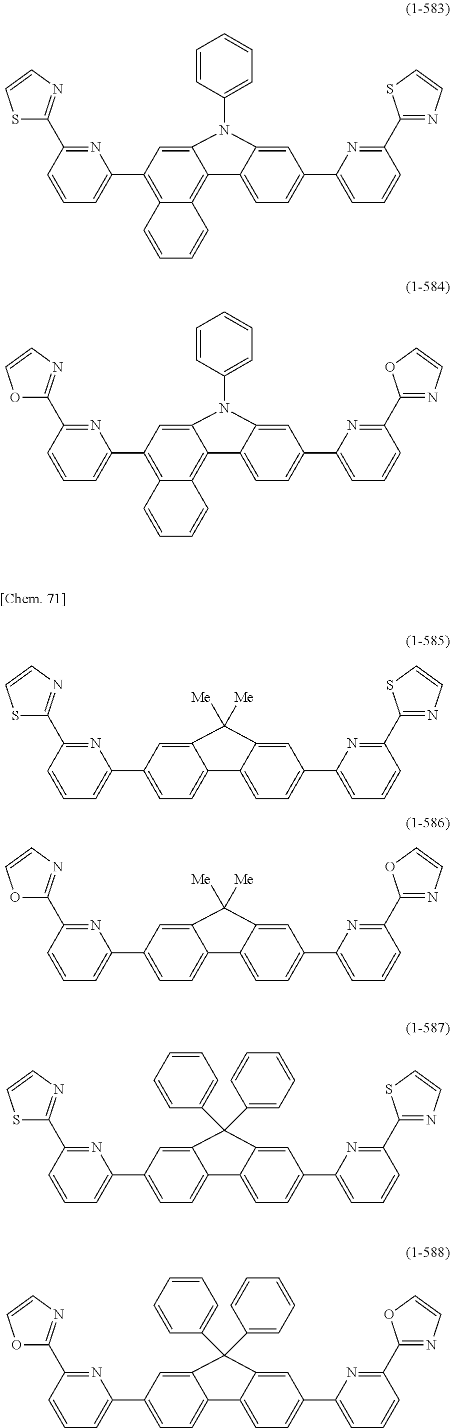 [SMILES: c1ccc(-n2c3cc(-c4cccc(-c5nccs5)n4)ccc3c3c4ccccc4c(-c4cccc(-c5nccs5)n4)cc32)cc1 ; c1ccc(-n2c3cc(-c4cccc(-c5ncco5)n4)ccc3c3c4ccccc4c(-c4cccc(-c5ncco5)n4)cc32)cc1 ; CC1(C)c2cc(-c3cccc(-c4nccs4)n3)ccc2-c2ccc(-c3cccc(-c4nccs4)n3)cc21 ; CC1(C)c2cc(-c3cccc(-c4ncco4)n3)ccc2-c2ccc(-c3cccc(-c4ncco4)n3)cc21 ; c1ccc(C2(c3ccccc3)c3cc(-c4cccc(-c5nccs5)n4)ccc3-c3ccc(-c4cccc(-c5nccs5)n4)cc32)cc1 ; c1ccc(C2(c3ccccc3)c3cc(-c4cccc(-c5ncco5)n4)ccc3-c3ccc(-c4cccc(-c5ncco5)n4)cc32)cc1]

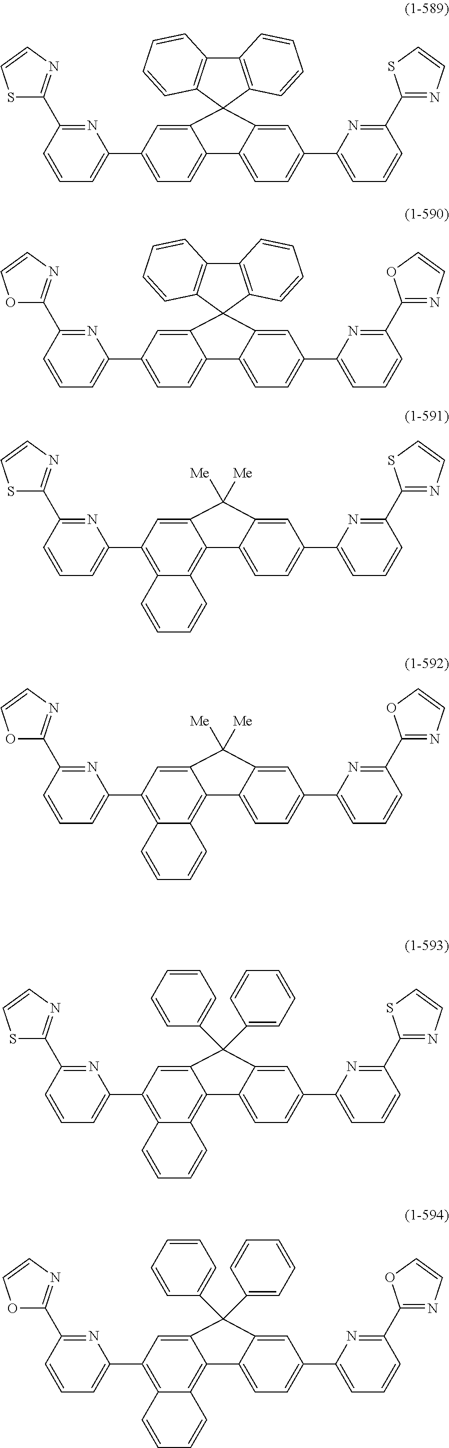

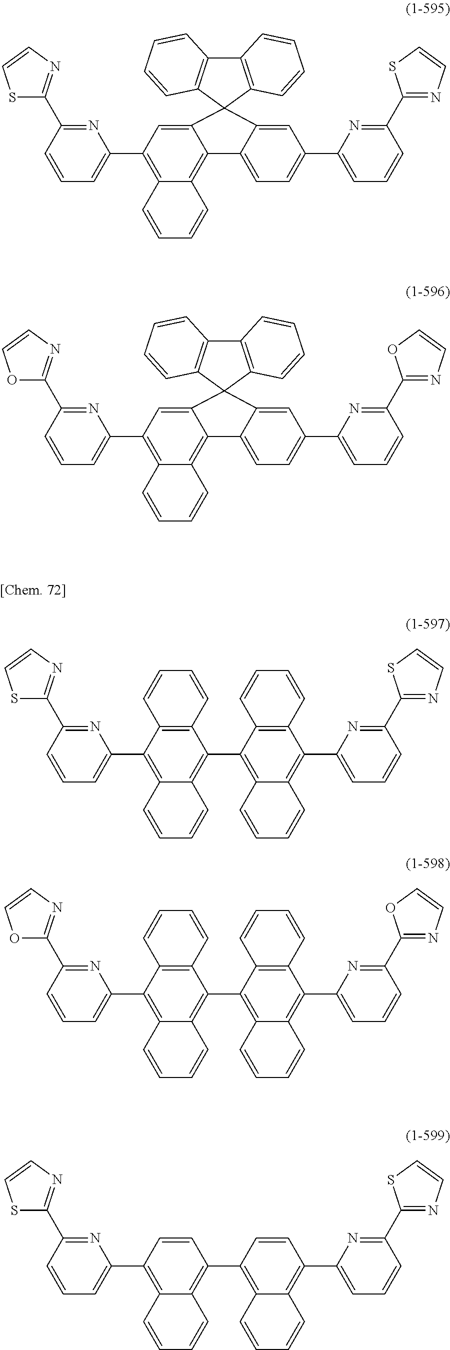 [SMILES: c1cc(-c2ccc3c(c2)C2(c4ccccc4-c4ccccc42)c2cc(-c4cccc(-c5nccs5)n4)c4ccccc4c2-3)nc(-c2nccs2)c1 ; c1cc(-c2ccc3c(c2)C2(c4ccccc4-c4ccccc42)c2cc(-c4cccc(-c5ncco5)n4)c4ccccc4c2-3)nc(-c2ncco2)c1 ; c1cc(-c2nccs2)nc(-c2c3ccccc3c(-c3c4ccccc4c(-c4cccc(-c5nccs5)n4)c4ccccc34)c3ccccc23)c1 ; c1cc(-c2ncco2)nc(-c2c3ccccc3c(-c3c4ccccc4c(-c4cccc(-c5ncco5)n4)c4ccccc34)c3ccccc23)c1 ; c1cc(-c2nccs2)nc(-c2ccc(-c3ccc(-c4cccc(-c5nccs5)n4)c4ccccc34)c3ccccc23)c1]

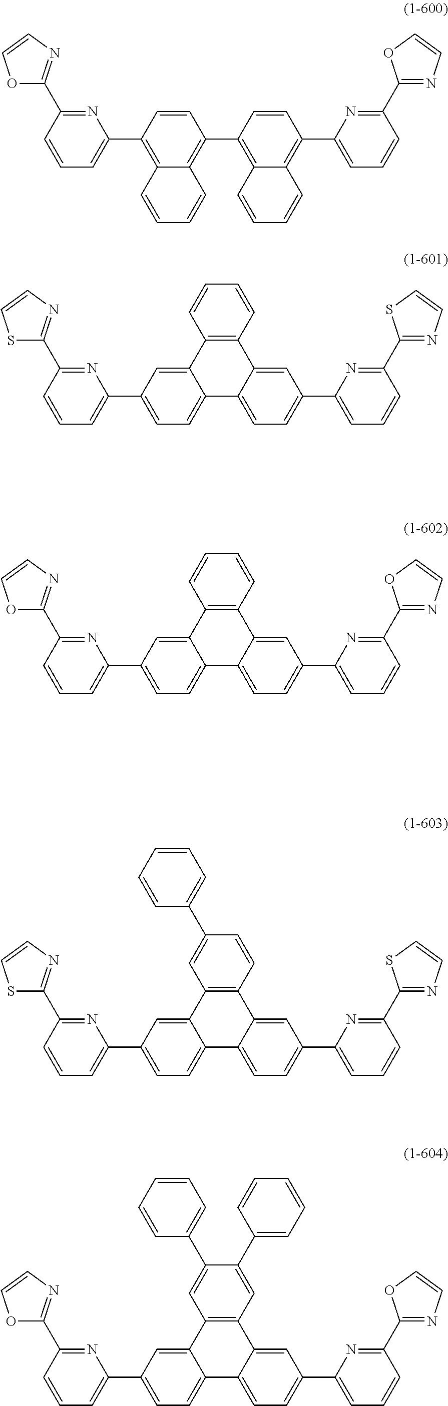 [SMILES: c1cc(-c2ncco2)nc(-c2ccc(-c3ccc(-c4cccc(-c5ncco5)n4)c4ccccc34)c3ccccc23)c1 ; c1cc(-c2ccc3c4ccc(-c5cccc(-c6nccs6)n5)cc4c4ccccc4c3c2)nc(-c2nccs2)c1 ; c1cc(-c2ccc3c4ccc(-c5cccc(-c6ncco6)n5)cc4c4ccccc4c3c2)nc(-c2ncco2)c1 ; c1ccc(-c2ccc3c(c2)c2cc(-c4cccc(-c5nccs5)n4)ccc2c2ccc(-c4cccc(-c5nccs5)n4)cc23)cc1 ; c1ccc(-c2cc3c4cc(-c5cccc(-c6ncco6)n5)ccc4c4ccc(-c5cccc(-c6ncco6)n5)cc4c3cc2-c2ccccc2)cc1]

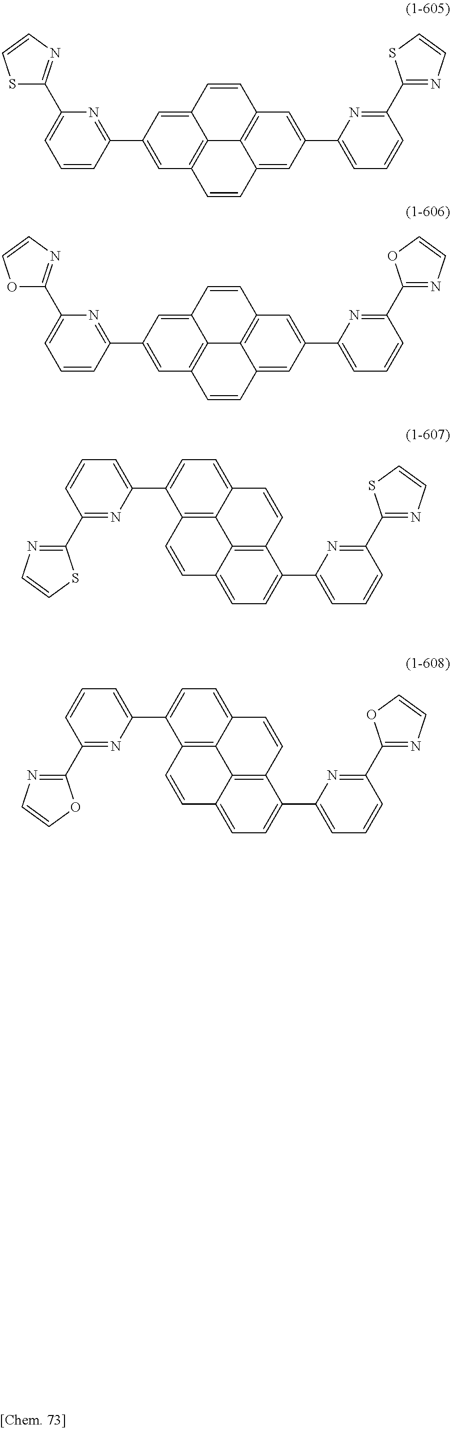 [SMILES: c1cc(-c2cc3ccc4cc(-c5cccc(-c6nccs6)n5)cc5ccc(c2)c3c45)nc(-c2nccs2)c1 ; c1cc(-c2cc3ccc4cc(-c5cccc(-c6ncco6)n5)cc5ccc(c2)c3c45)nc(-c2ncco2)c1 ; c1cc(-c2nccs2)nc(-c2ccc3ccc4c(-c5cccc(-c6nccs6)n5)ccc5ccc2c3c54)c1 ; c1cc(-c2ncco2)nc(-c2ccc3ccc4c(-c5cccc(-c6ncco6)n5)ccc5ccc2c3c54)c1]

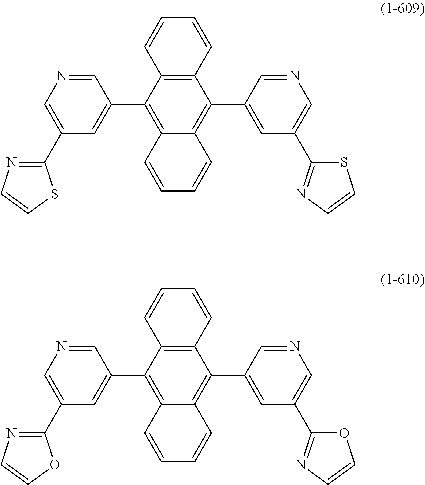 [SMILES: c1ccc2c(-c3cncc(-c4nccs4)c3)c3ccccc3c(-c3cncc(-c4nccs4)c3)c2c1 ; c1ccc2c(-c3cncc(-c4ncco4)c3)c3ccccc3c(-c3cncc(-c4ncco4)c3)c2c1]

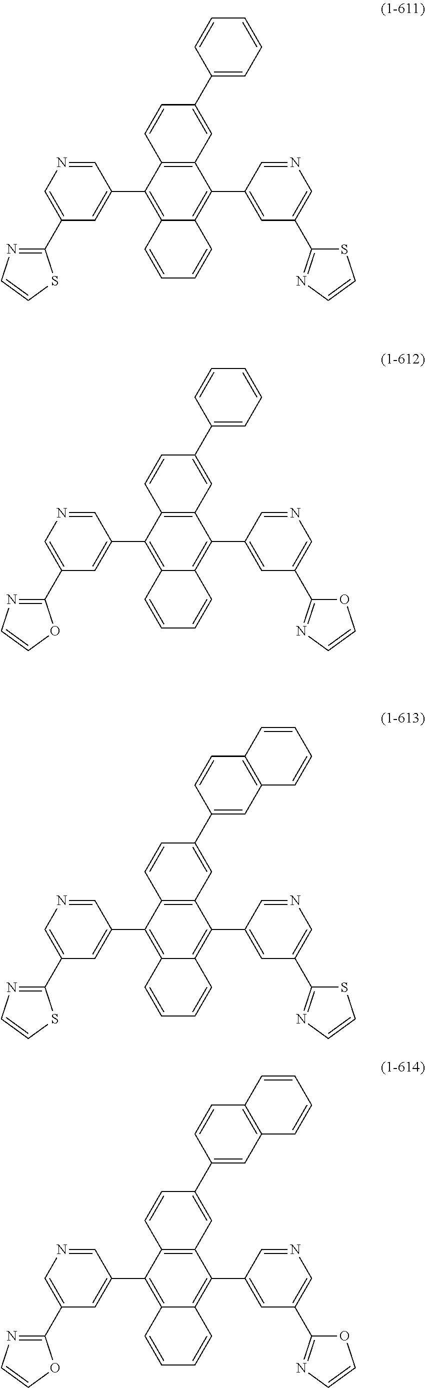 [SMILES: c1ccc(-c2ccc3c(-c4cncc(-c5nccs5)c4)c4ccccc4c(-c4cncc(-c5nccs5)c4)c3c2)cc1 ; c1ccc(-c2ccc3c(-c4cncc(-c5ncco5)c4)c4ccccc4c(-c4cncc(-c5ncco5)c4)c3c2)cc1 ; c1ccc2cc(-c3ccc4c(-c5cncc(-c6nccs6)c5)c5ccccc5c(-c5cncc(-c6nccs6)c5)c4c3)ccc2c1 ; c1ccc2cc(-c3ccc4c(-c5cncc(-c6ncco6)c5)c5ccccc5c(-c5cncc(-c6ncco6)c5)c4c3)ccc2c1]

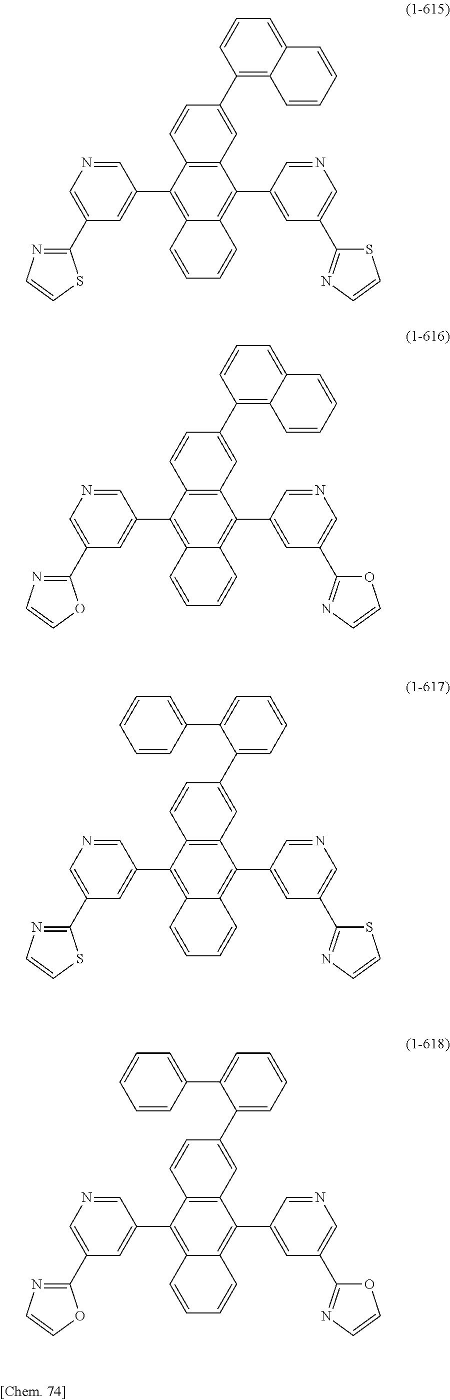 [SMILES: c1ccc2c(-c3ccc4c(-c5cncc(-c6nccs6)c5)c5ccccc5c(-c5cncc(-c6nccs6)c5)c4c3)cccc2c1 ; c1ccc2c(-c3ccc4c(-c5cncc(-c6ncco6)c5)c5ccccc5c(-c5cncc(-c6ncco6)c5)c4c3)cccc2c1 ; c1ccc(-c2ccccc2-c2ccc3c(-c4cncc(-c5nccs5)c4)c4ccccc4c(-c4cncc(-c5nccs5)c4)c3c2)cc1 ; c1ccc(-c2ccccc2-c2ccc3c(-c4cncc(-c5ncco5)c4)c4ccccc4c(-c4cncc(-c5ncco5)c4)c3c2)cc1]

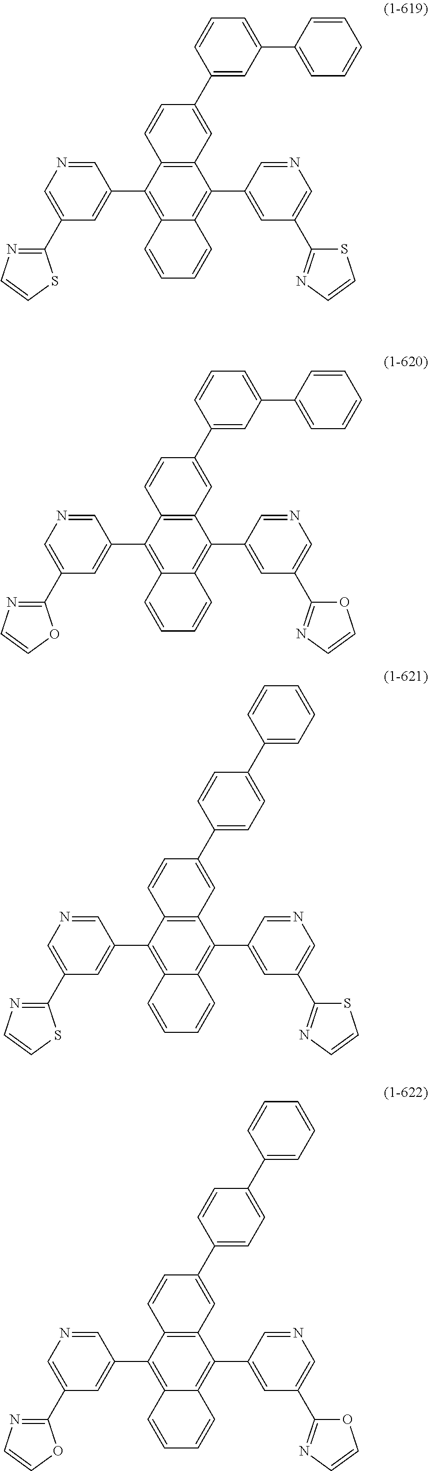

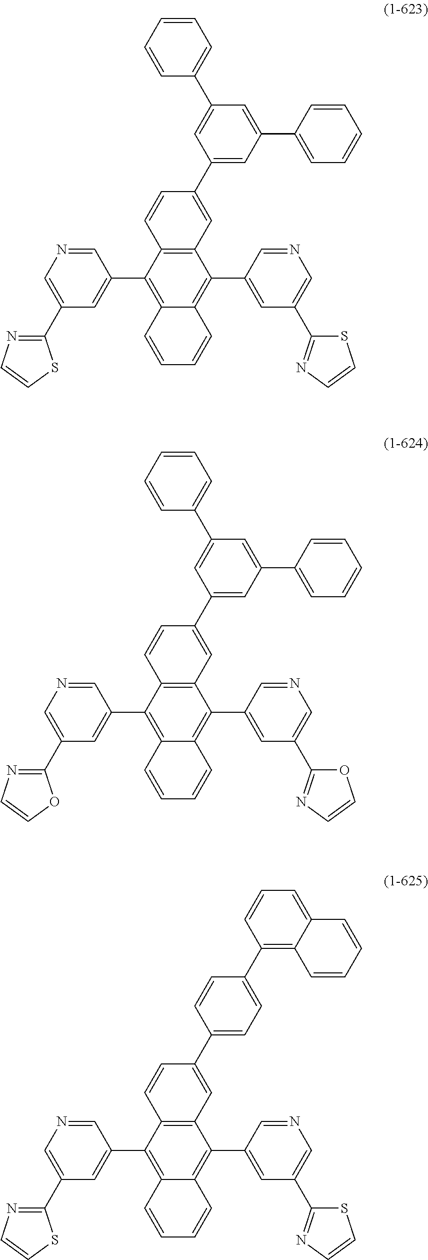 [SMILES: c1ccc(-c2cc(-c3ccccc3)cc(-c3ccc4c(-c5cncc(-c6nccs6)c5)c5ccccc5c(-c5cncc(-c6nccs6)c5)c4c3)c2)cc1 ; c1ccc(-c2cc(-c3ccccc3)cc(-c3ccc4c(-c5cncc(-c6ncco6)c5)c5ccccc5c(-c5cncc(-c6ncco6)c5)c4c3)c2)cc1 ; c1ccc2c(-c3ccc(-c4ccc5c(-c6cncc(-c7nccs7)c6)c6ccccc6c(-c6cncc(-c7nccs7)c6)c5c4)cc3)cccc2c1]

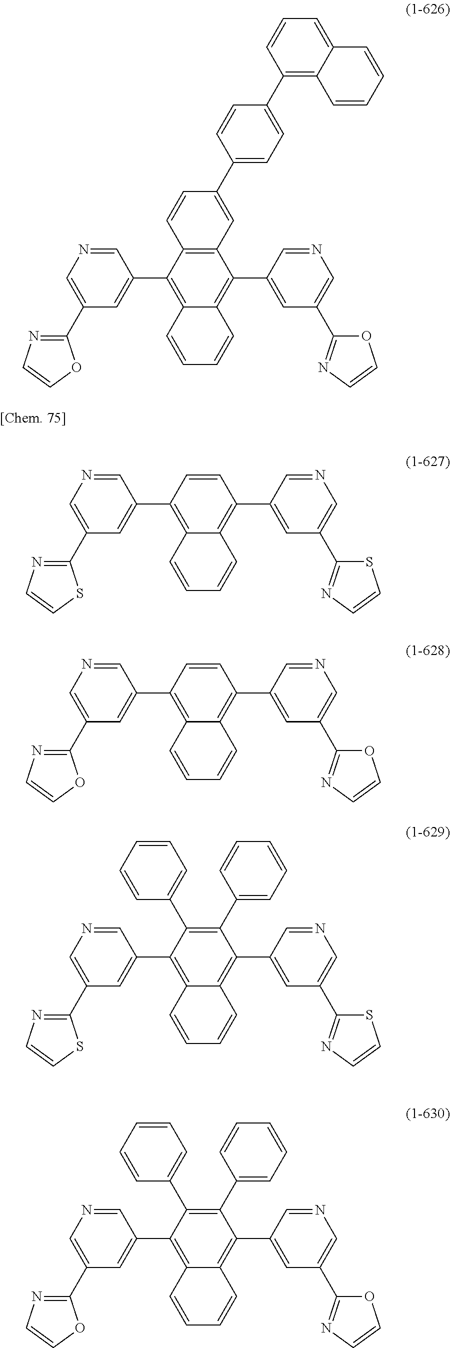 [SMILES: c1ccc2c(-c3ccc(-c4ccc5c(-c6cncc(-c7ncco7)c6)c6ccccc6c(-c6cncc(-c7ncco7)c6)c5c4)cc3)cccc2c1 ; c1ccc2c(-c3cncc(-c4nccs4)c3)ccc(-c3cncc(-c4nccs4)c3)c2c1 ; c1ccc2c(-c3cncc(-c4ncco4)c3)ccc(-c3cncc(-c4ncco4)c3)c2c1 ; c1ccc(-c2c(-c3ccccc3)c(-c3cncc(-c4nccs4)c3)c3ccccc3c2-c2cncc(-c3nccs3)c2)cc1 ; c1ccc(-c2c(-c3ccccc3)c(-c3cncc(-c4ncco4)c3)c3ccccc3c2-c2cncc(-c3ncco3)c2)cc1]

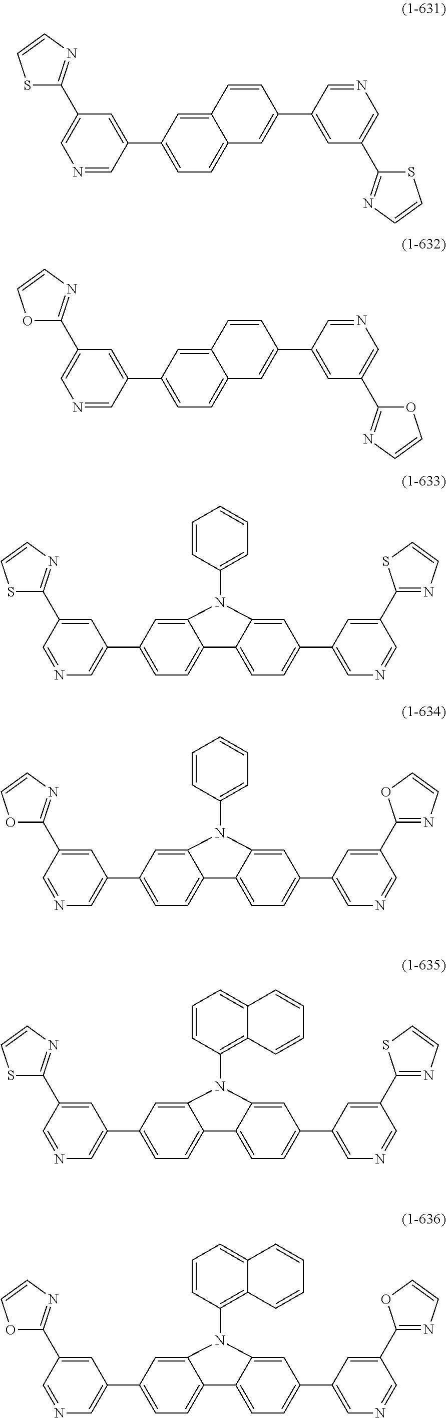 [SMILES: c1csc(-c2cncc(-c3ccc4cc(-c5cncc(-c6nccs6)c5)ccc4c3)c2)n1 ; c1coc(-c2cncc(-c3ccc4cc(-c5cncc(-c6ncco6)c5)ccc4c3)c2)n1 ; c1ccc(-n2c3cc(-c4cncc(-c5nccs5)c4)ccc3c3ccc(-c4cncc(-c5nccs5)c4)cc32)cc1 ; c1ccc(-n2c3cc(-c4cncc(-c5ncco5)c4)ccc3c3ccc(-c4cncc(-c5ncco5)c4)cc32)cc1 ; c1ccc2c(-n3c4cc(-c5cncc(-c6nccs6)c5)ccc4c4ccc(-c5cncc(-c6nccs6)c5)cc43)cccc2c1 ; c1ccc2c(-n3c4cc(-c5cncc(-c6ncco6)c5)ccc4c4ccc(-c5cncc(-c6ncco6)c5)cc43)cccc2c1]

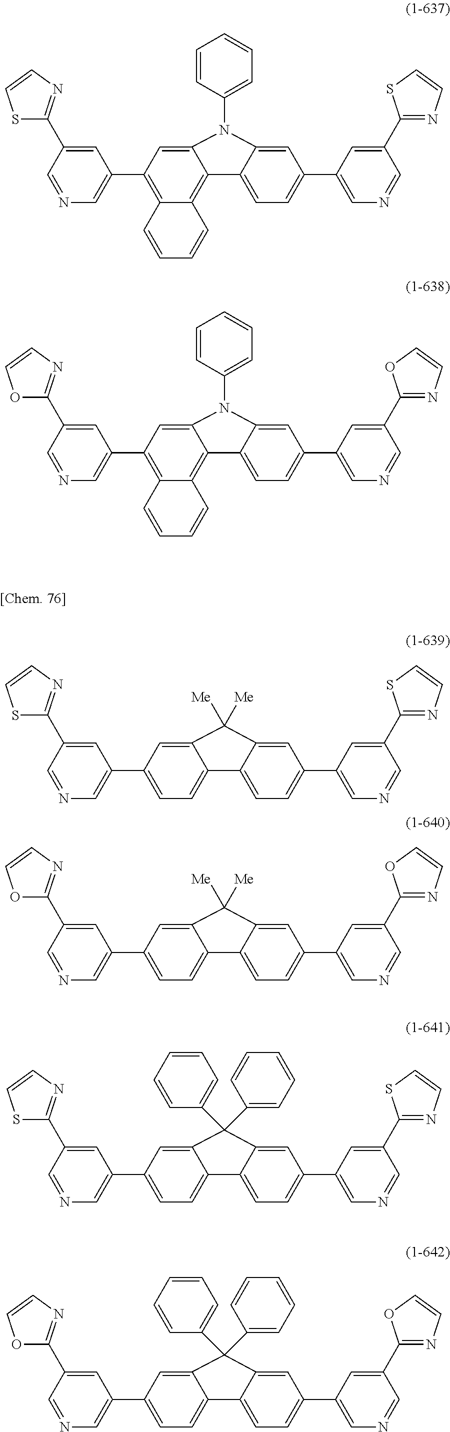 [SMILES: c1ccc(-n2c3cc(-c4cncc(-c5nccs5)c4)ccc3c3c4ccccc4c(-c4cncc(-c5nccs5)c4)cc32)cc1 ; c1ccc(-n2c3cc(-c4cncc(-c5ncco5)c4)ccc3c3c4ccccc4c(-c4cncc(-c5ncco5)c4)cc32)cc1 ; CC1(C)c2cc(-c3cncc(-c4nccs4)c3)ccc2-c2ccc(-c3cncc(-c4nccs4)c3)cc21 ; CC1(C)c2cc(-c3cncc(-c4ncco4)c3)ccc2-c2ccc(-c3cncc(-c4ncco4)c3)cc21 ; c1ccc(C2(c3ccccc3)c3cc(-c4cncc(-c5nccs5)c4)ccc3-c3ccc(-c4cncc(-c5nccs5)c4)cc32)cc1 ; c1ccc(C2(c3ccccc3)c3cc(-c4cncc(-c5ncco5)c4)ccc3-c3ccc(-c4cncc(-c5ncco5)c4)cc32)cc1]

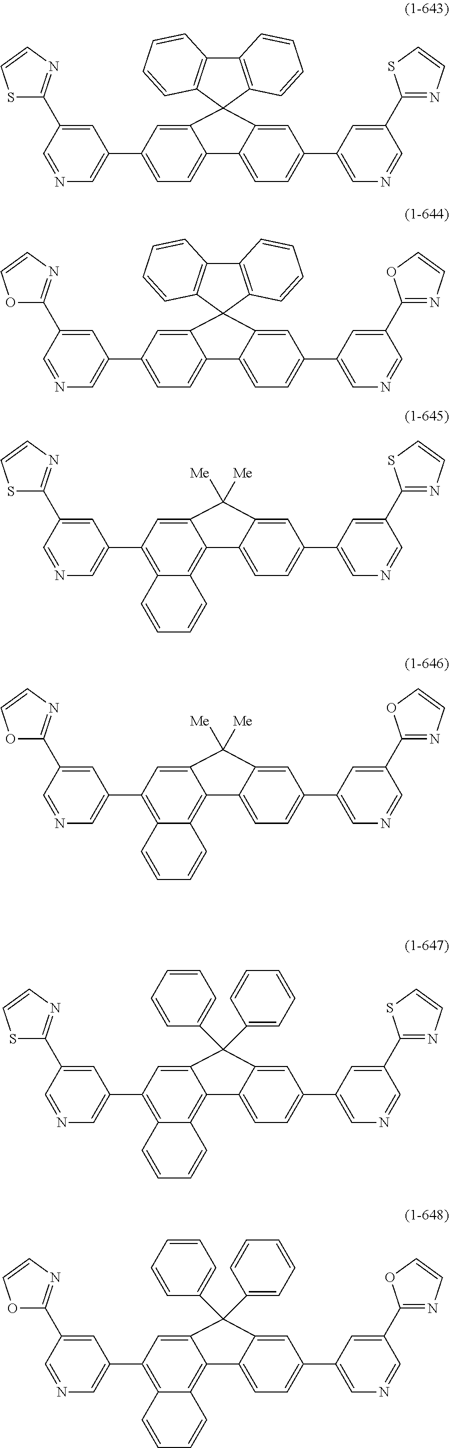

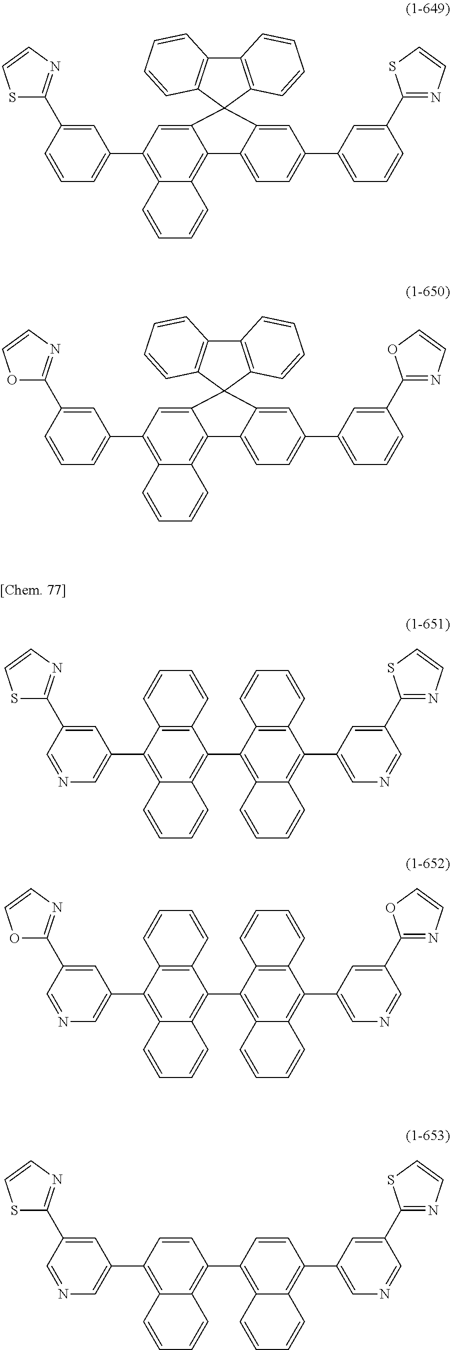 [SMILES: c1cc(-c2ccc3c(c2)C2(c4ccccc4-c4ccccc42)c2cc(-c4cccc(-c5nccs5)c4)c4ccccc4c2-3)cc(-c2nccs2)c1 ; c1cc(-c2ccc3c(c2)C2(c4ccccc4-c4ccccc42)c2cc(-c4cccc(-c5ncco5)c4)c4ccccc4c2-3)cc(-c2ncco2)c1 ; c1ccc2c(-c3c4ccccc4c(-c4cncc(-c5nccs5)c4)c4ccccc34)c3ccccc3c(-c3cncc(-c4nccs4)c3)c2c1 ; c1ccc2c(-c3c4ccccc4c(-c4cncc(-c5ncco5)c4)c4ccccc34)c3ccccc3c(-c3cncc(-c4ncco4)c3)c2c1 ; c1ccc2c(-c3ccc(-c4cncc(-c5nccs5)c4)c4ccccc34)ccc(-c3cncc(-c4nccs4)c3)c2c1]

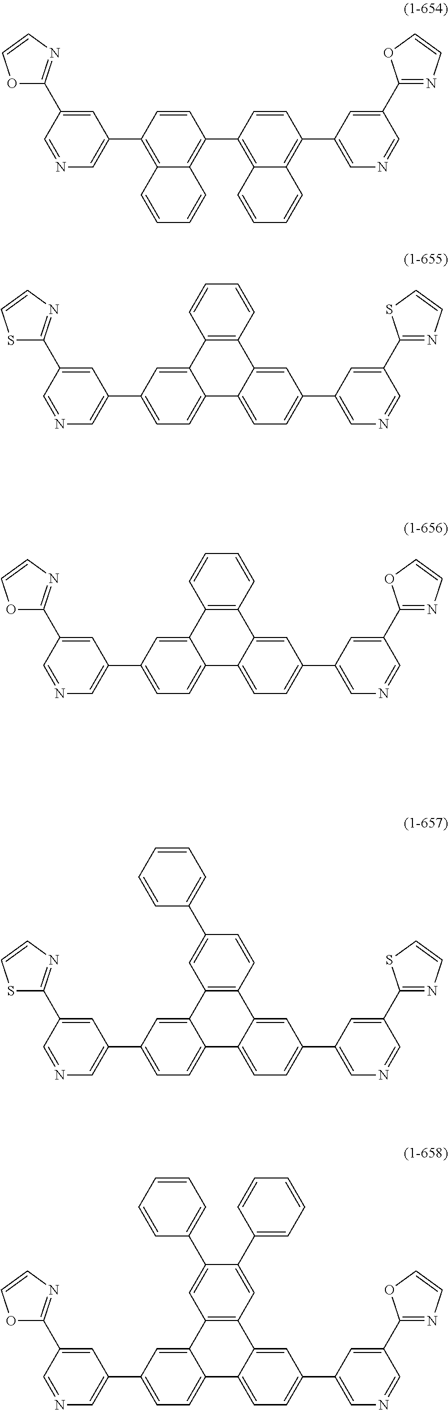 [SMILES: c1ccc2c(-c3ccc(-c4cncc(-c5ncco5)c4)c4ccccc34)ccc(-c3cncc(-c4ncco4)c3)c2c1 ; c1ccc2c(c1)c1cc(-c3cncc(-c4nccs4)c3)ccc1c1ccc(-c3cncc(-c4nccs4)c3)cc21 ; c1ccc2c(c1)c1cc(-c3cncc(-c4ncco4)c3)ccc1c1ccc(-c3cncc(-c4ncco4)c3)cc21 ; c1ccc(-c2ccc3c4cc(-c5cncc(-c6nccs6)c5)ccc4c4ccc(-c5cncc(-c6nccs6)c5)cc4c3c2)cc1 ; c1ccc(-c2cc3c4cc(-c5cncc(-c6ncco6)c5)ccc4c4ccc(-c5cncc(-c6ncco6)c5)cc4c3cc2-c2ccccc2)cc1]

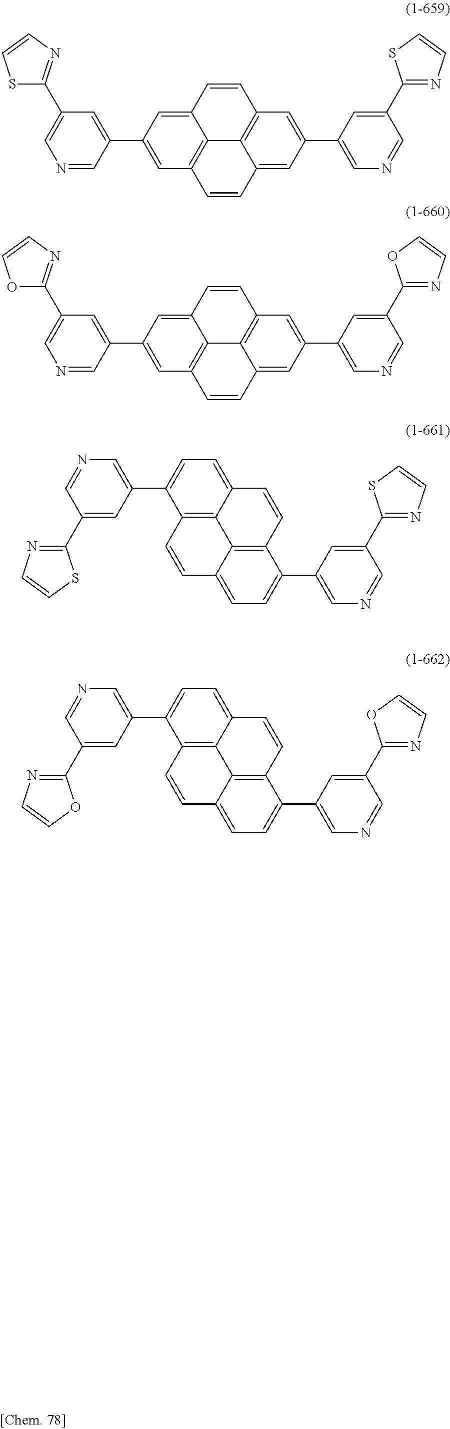 [SMILES: c1csc(-c2cncc(-c3cc4ccc5cc(-c6cncc(-c7nccs7)c6)cc6ccc(c3)c4c56)c2)n1 ; c1coc(-c2cncc(-c3cc4ccc5cc(-c6cncc(-c7ncco7)c6)cc6ccc(c3)c4c56)c2)n1 ; c1csc(-c2cncc(-c3ccc4ccc5c(-c6cncc(-c7nccs7)c6)ccc6ccc3c4c65)c2)n1 ; c1coc(-c2cncc(-c3ccc4ccc5c(-c6cncc(-c7ncco7)c6)ccc6ccc3c4c65)c2)n1]

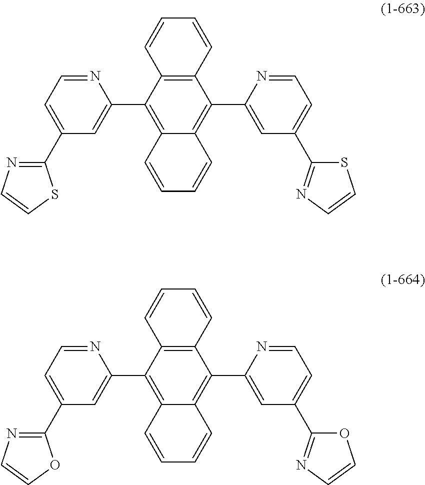 [SMILES: c1ccc2c(-c3cc(-c4nccs4)ccn3)c3ccccc3c(-c3cc(-c4nccs4)ccn3)c2c1 ; c1ccc2c(-c3cc(-c4ncco4)ccn3)c3ccccc3c(-c3cc(-c4ncco4)ccn3)c2c1]

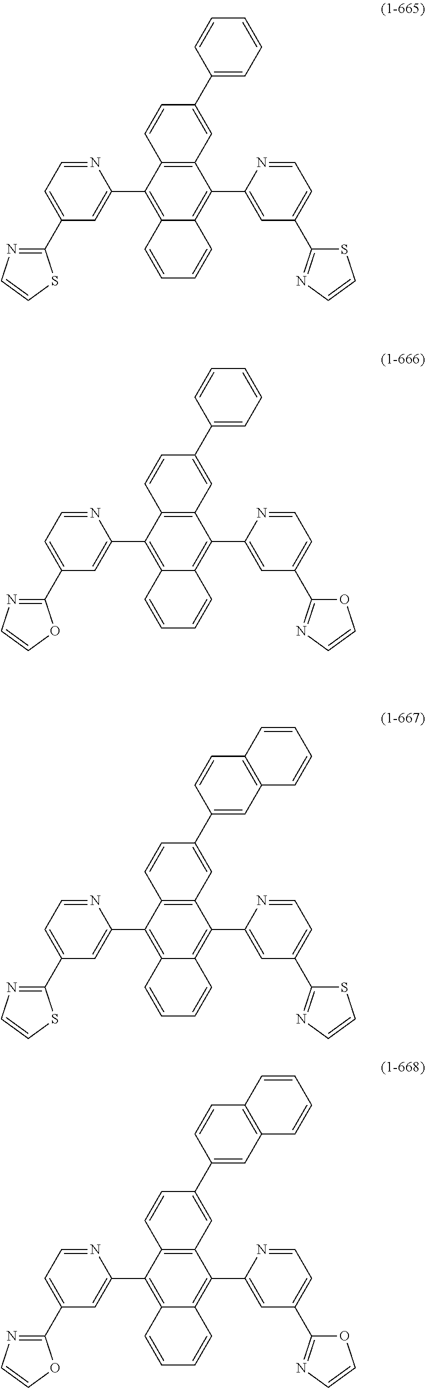 [SMILES: c1ccc(-c2ccc3c(-c4cc(-c5nccs5)ccn4)c4ccccc4c(-c4cc(-c5nccs5)ccn4)c3c2)cc1 ; c1ccc(-c2ccc3c(-c4cc(-c5ncco5)ccn4)c4ccccc4c(-c4cc(-c5ncco5)ccn4)c3c2)cc1 ; c1ccc2cc(-c3ccc4c(-c5cc(-c6nccs6)ccn5)c5ccccc5c(-c5cc(-c6nccs6)ccn5)c4c3)ccc2c1 ; c1ccc2cc(-c3ccc4c(-c5cc(-c6ncco6)ccn5)c5ccccc5c(-c5cc(-c6ncco6)ccn5)c4c3)ccc2c1]

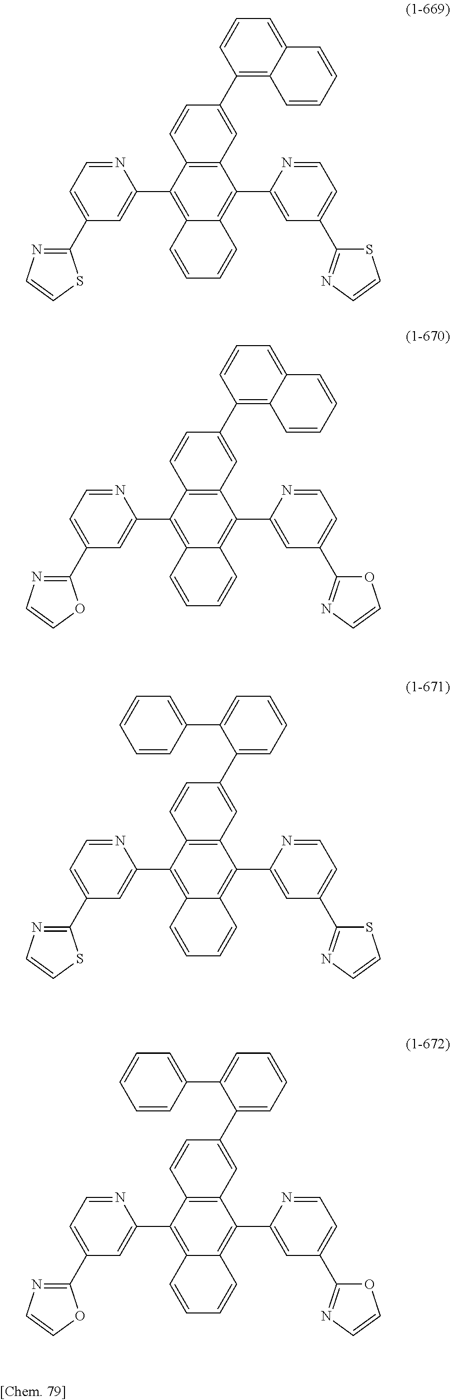 [SMILES: c1ccc2c(-c3ccc4c(-c5cc(-c6nccs6)ccn5)c5ccccc5c(-c5cc(-c6nccs6)ccn5)c4c3)cccc2c1 ; c1ccc2c(-c3ccc4c(-c5cc(-c6ncco6)ccn5)c5ccccc5c(-c5cc(-c6ncco6)ccn5)c4c3)cccc2c1 ; c1ccc(-c2ccccc2-c2ccc3c(-c4cc(-c5nccs5)ccn4)c4ccccc4c(-c4cc(-c5nccs5)ccn4)c3c2)cc1 ; c1ccc(-c2ccccc2-c2ccc3c(-c4cc(-c5ncco5)ccn4)c4ccccc4c(-c4cc(-c5ncco5)ccn4)c3c2)cc1]

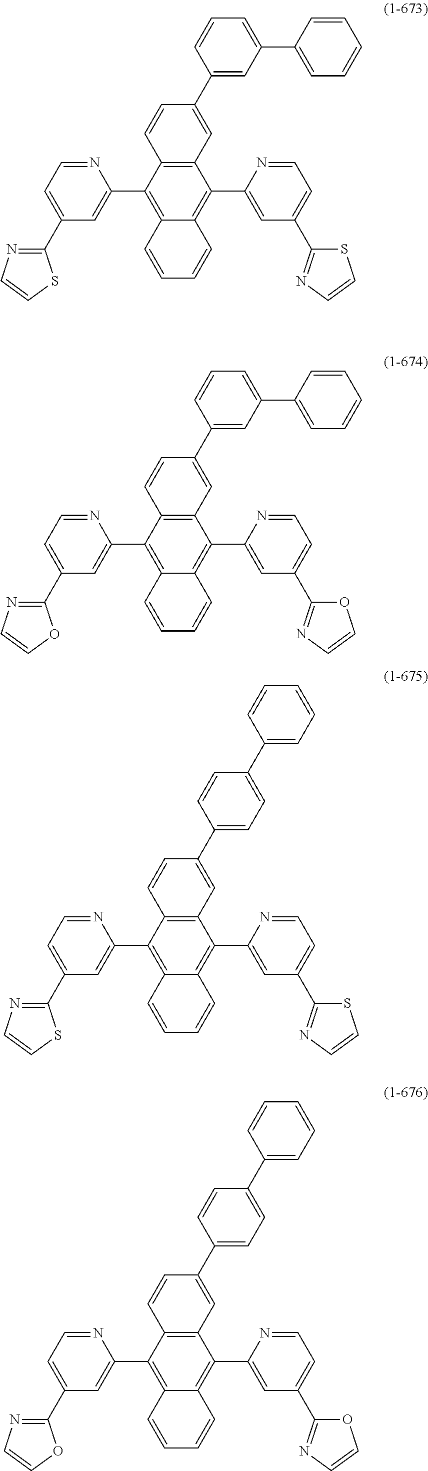

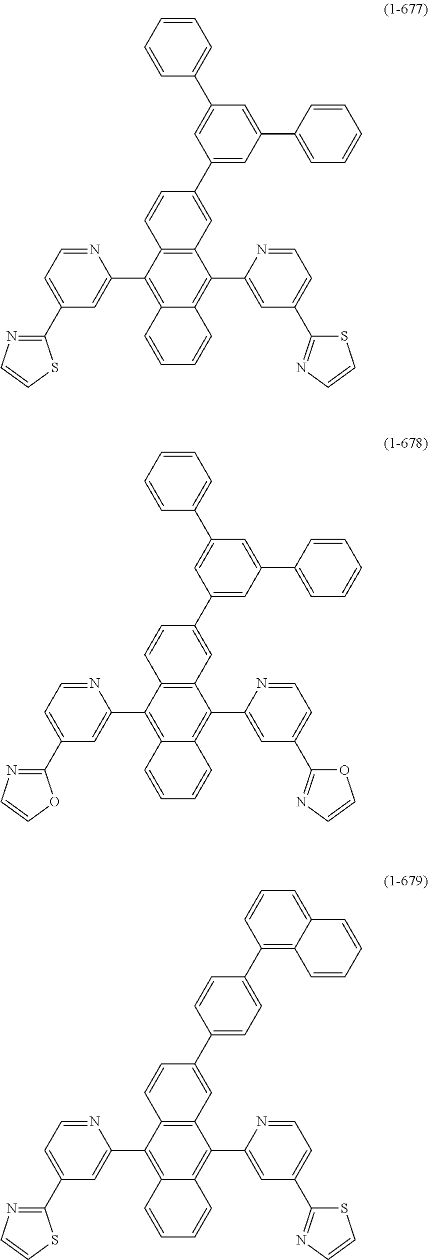 [SMILES: c1ccc(-c2cc(-c3ccccc3)cc(-c3ccc4c(-c5cc(-c6nccs6)ccn5)c5ccccc5c(-c5cc(-c6nccs6)ccn5)c4c3)c2)cc1 ; c1ccc(-c2cc(-c3ccccc3)cc(-c3ccc4c(-c5cc(-c6ncco6)ccn5)c5ccccc5c(-c5cc(-c6ncco6)ccn5)c4c3)c2)cc1 ; c1ccc2c(-c3ccc(-c4ccc5c(-c6cc(-c7nccs7)ccn6)c6ccccc6c(-c6cc(-c7nccs7)ccn6)c5c4)cc3)cccc2c1]

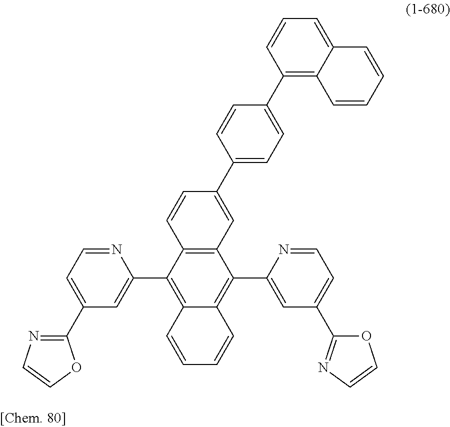 [SMILES: c1ccc2c(-c3ccc(-c4ccc5c(-c6cc(-c7ncco7)ccn6)c6ccccc6c(-c6cc(-c7ncco7)ccn6)c5c4)cc3)cccc2c1]

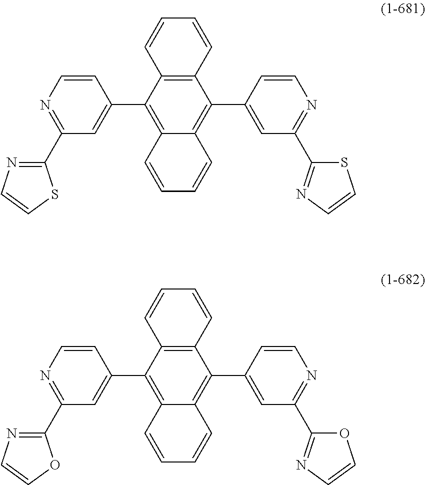 [SMILES: c1ccc2c(-c3ccnc(-c4nccs4)c3)c3ccccc3c(-c3ccnc(-c4nccs4)c3)c2c1 ; c1ccc2c(-c3ccnc(-c4ncco4)c3)c3ccccc3c(-c3ccnc(-c4ncco4)c3)c2c1]

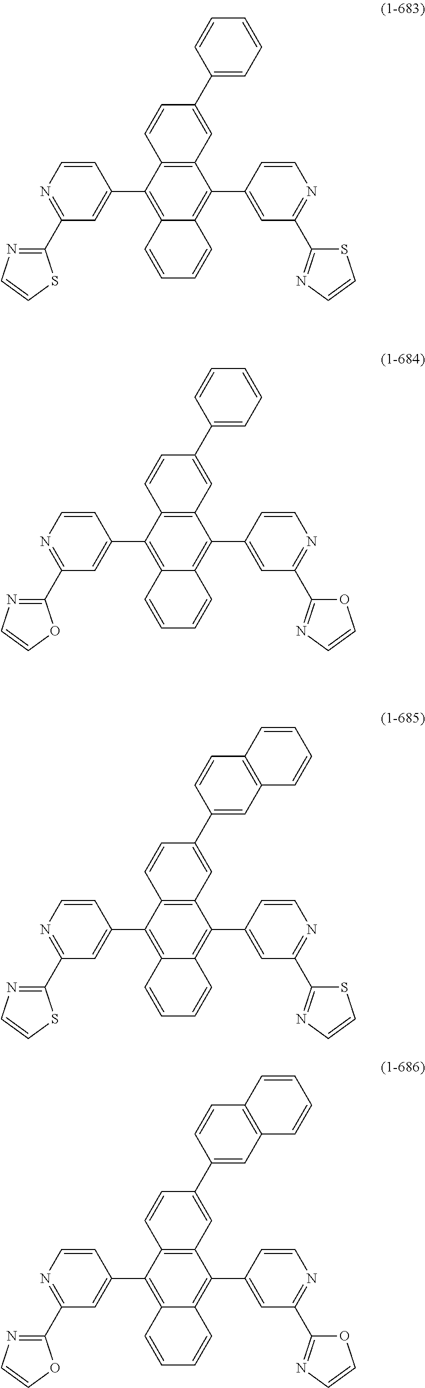 [SMILES: c1ccc(-c2ccc3c(-c4ccnc(-c5nccs5)c4)c4ccccc4c(-c4ccnc(-c5nccs5)c4)c3c2)cc1 ; c1ccc(-c2ccc3c(-c4ccnc(-c5ncco5)c4)c4ccccc4c(-c4ccnc(-c5ncco5)c4)c3c2)cc1 ; c1ccc2cc(-c3ccc4c(-c5ccnc(-c6nccs6)c5)c5ccccc5c(-c5ccnc(-c6nccs6)c5)c4c3)ccc2c1 ; c1ccc2cc(-c3ccc4c(-c5ccnc(-c6ncco6)c5)c5ccccc5c(-c5ccnc(-c6ncco6)c5)c4c3)ccc2c1]

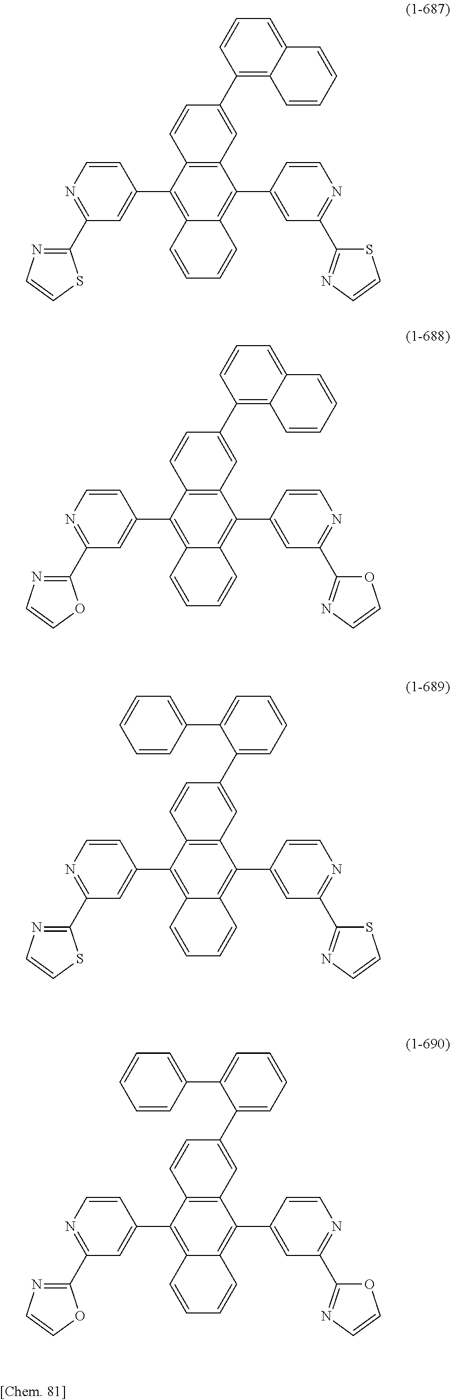 [SMILES: c1ccc2c(-c3ccc4c(-c5ccnc(-c6nccs6)c5)c5ccccc5c(-c5ccnc(-c6nccs6)c5)c4c3)cccc2c1 ; c1ccc2c(-c3ccc4c(-c5ccnc(-c6ncco6)c5)c5ccccc5c(-c5ccnc(-c6ncco6)c5)c4c3)cccc2c1 ; c1ccc(-c2ccccc2-c2ccc3c(-c4ccnc(-c5nccs5)c4)c4ccccc4c(-c4ccnc(-c5nccs5)c4)c3c2)cc1 ; c1ccc(-c2ccccc2-c2ccc3c(-c4ccnc(-c5ncco5)c4)c4ccccc4c(-c4ccnc(-c5ncco5)c4)c3c2)cc1]

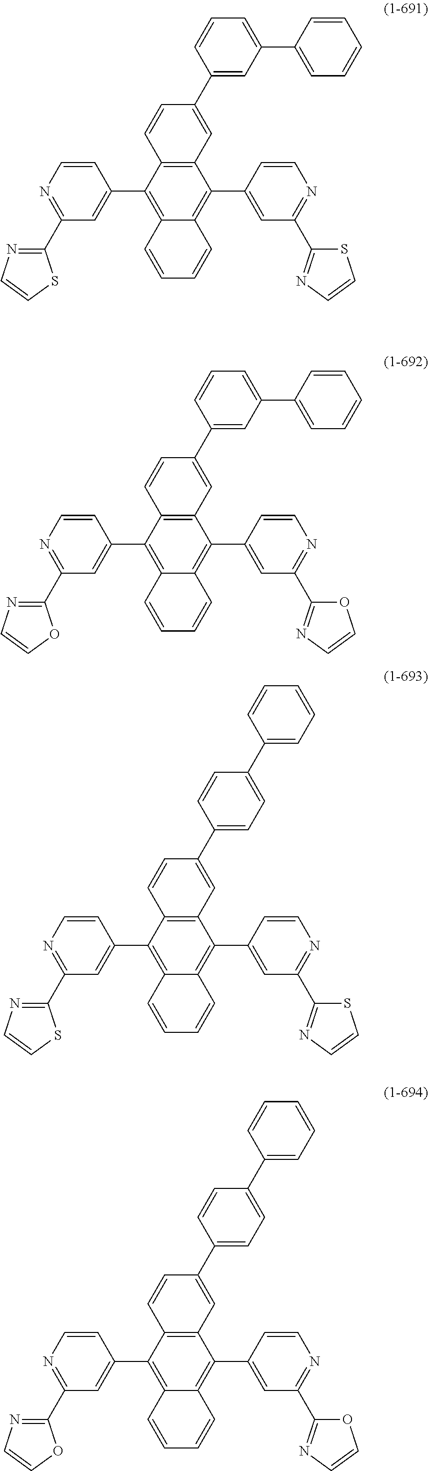

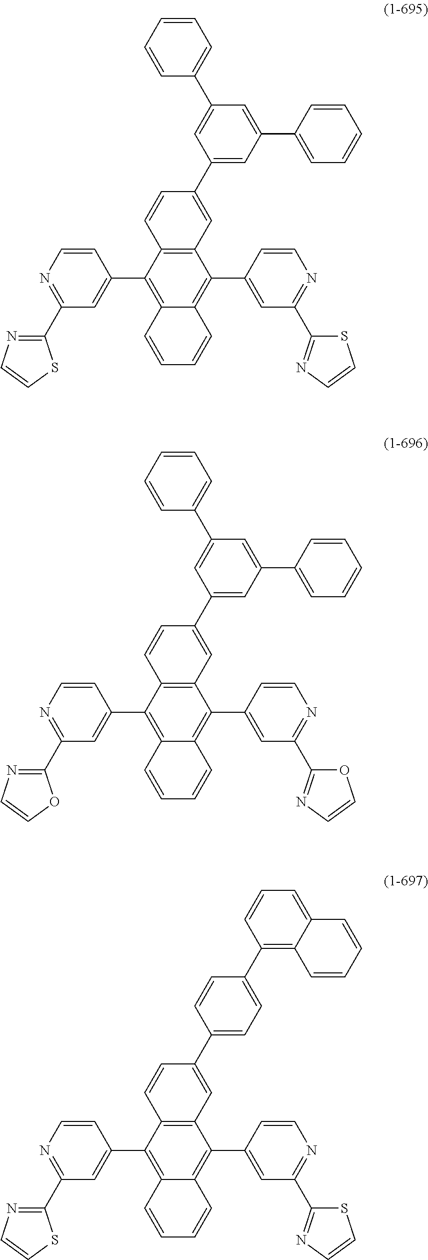 [SMILES: c1ccc(-c2cc(-c3ccccc3)cc(-c3ccc4c(-c5ccnc(-c6nccs6)c5)c5ccccc5c(-c5ccnc(-c6nccs6)c5)c4c3)c2)cc1 ; c1ccc(-c2cc(-c3ccccc3)cc(-c3ccc4c(-c5ccnc(-c6ncco6)c5)c5ccccc5c(-c5ccnc(-c6ncco6)c5)c4c3)c2)cc1 ; c1ccc2c(-c3ccc(-c4ccc5c(-c6ccnc(-c7nccs7)c6)c6ccccc6c(-c6ccnc(-c7nccs7)c6)c5c4)cc3)cccc2c1]

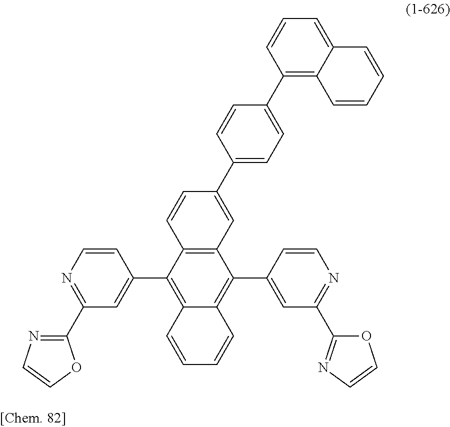 [SMILES: c1ccc2c(-c3ccc(-c4ccc5c(-c6ccnc(-c7ncco7)c6)c6ccccc6c(-c6ccnc(-c7ncco7)c6)c5c4)cc3)cccc2c1]

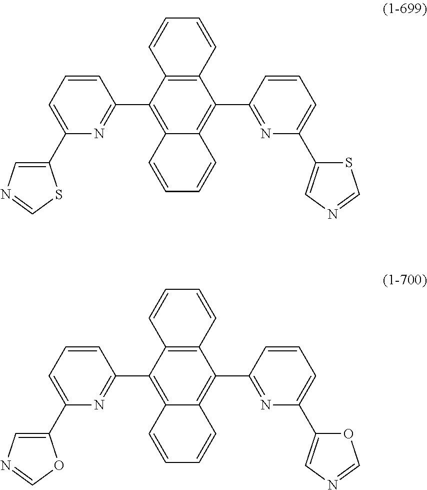 [SMILES: c1cc(-c2cncs2)nc(-c2c3ccccc3c(-c3cccc(-c4cncs4)n3)c3ccccc23)c1 ; c1cc(-c2cnco2)nc(-c2c3ccccc3c(-c3cccc(-c4cnco4)n3)c3ccccc23)c1]

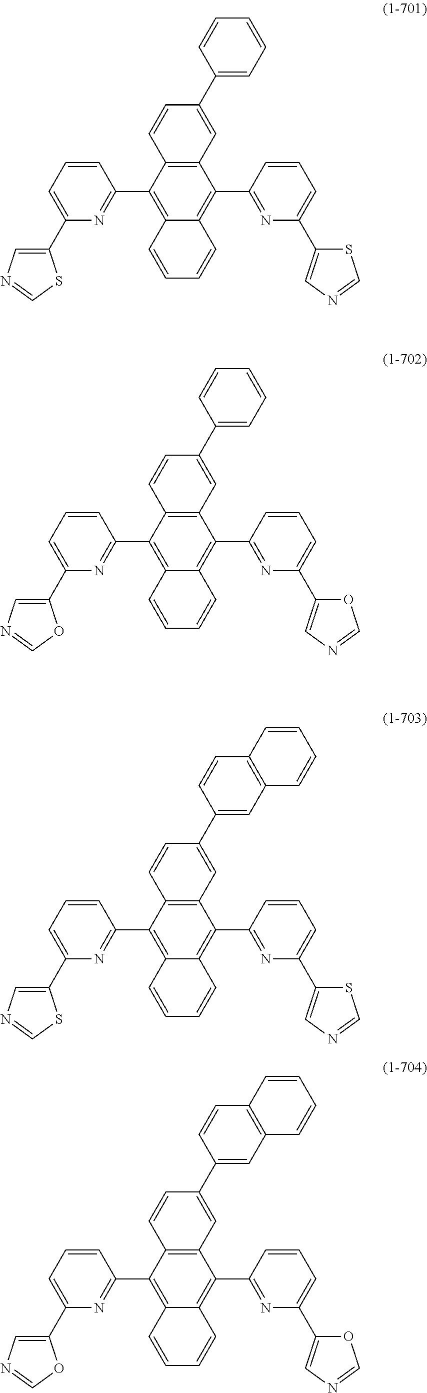 [SMILES: c1ccc(-c2ccc3c(-c4cccc(-c5cncs5)n4)c4ccccc4c(-c4cccc(-c5cncs5)n4)c3c2)cc1 ; c1ccc(-c2ccc3c(-c4cccc(-c5cnco5)n4)c4ccccc4c(-c4cccc(-c5cnco5)n4)c3c2)cc1 ; c1cc(-c2cncs2)nc(-c2c3ccccc3c(-c3cccc(-c4cncs4)n3)c3cc(-c4ccc5ccccc5c4)ccc23)c1 ; c1cc(-c2cnco2)nc(-c2c3ccccc3c(-c3cccc(-c4cnco4)n3)c3cc(-c4ccc5ccccc5c4)ccc23)c1]

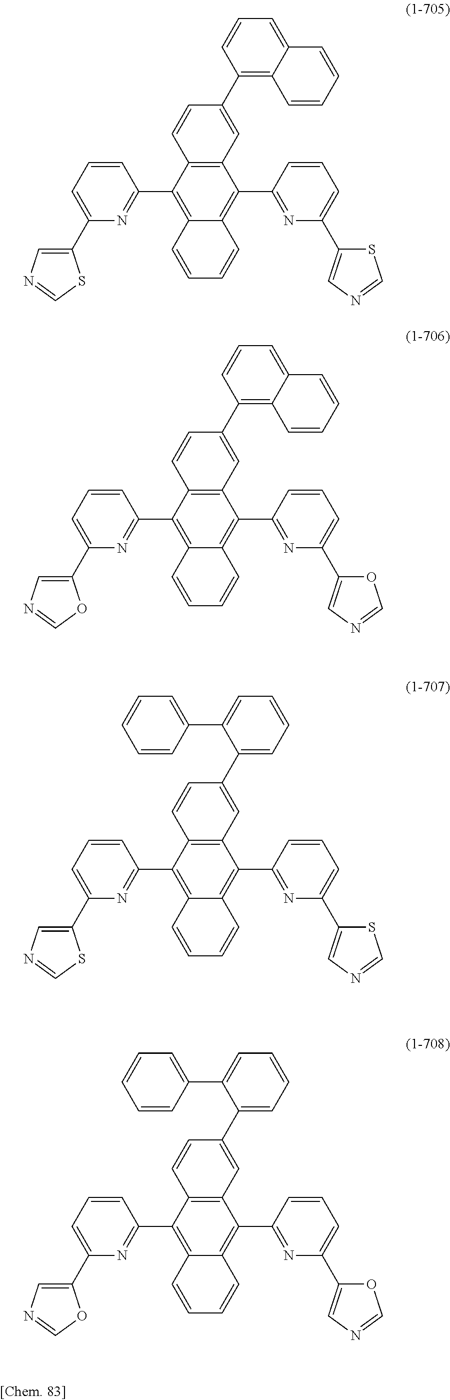 [SMILES: c1cc(-c2cncs2)nc(-c2c3ccccc3c(-c3cccc(-c4cncs4)n3)c3cc(-c4cccc5ccccc45)ccc23)c1 ; c1cc(-c2cnco2)nc(-c2c3ccccc3c(-c3cccc(-c4cnco4)n3)c3cc(-c4cccc5ccccc45)ccc23)c1 ; c1ccc(-c2ccccc2-c2ccc3c(-c4cccc(-c5cncs5)n4)c4ccccc4c(-c4cccc(-c5cncs5)n4)c3c2)cc1 ; c1ccc(-c2ccccc2-c2ccc3c(-c4cccc(-c5cnco5)n4)c4ccccc4c(-c4cccc(-c5cnco5)n4)c3c2)cc1]

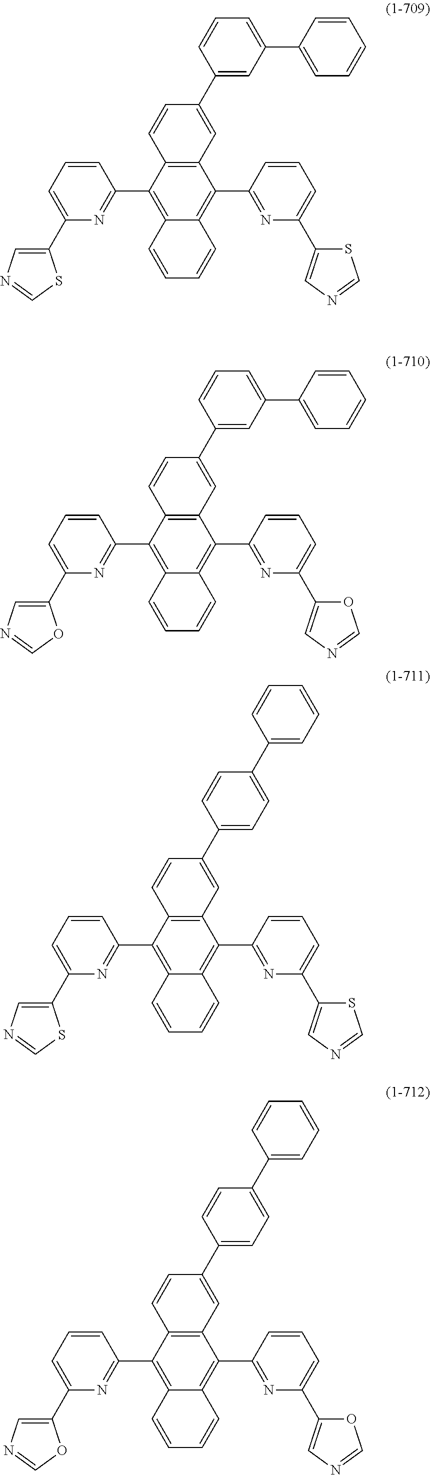

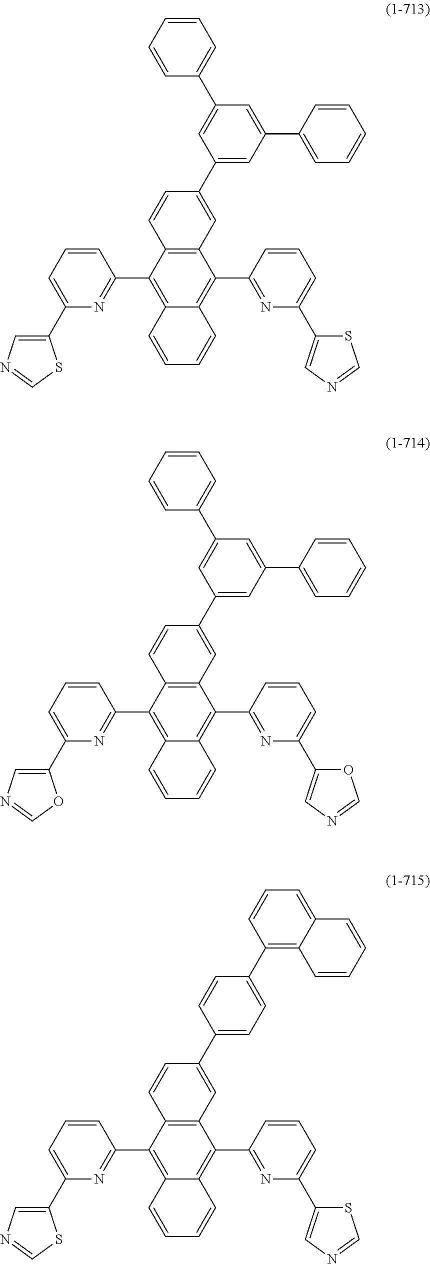 [SMILES: c1ccc(-c2cc(-c3ccccc3)cc(-c3ccc4c(-c5cccc(-c6cncs6)n5)c5ccccc5c(-c5cccc(-c6cncs6)n5)c4c3)c2)cc1 ; c1ccc(-c2cc(-c3ccccc3)cc(-c3ccc4c(-c5cccc(-c6cnco6)n5)c5ccccc5c(-c5cccc(-c6cnco6)n5)c4c3)c2)cc1 ; c1cc(-c2cncs2)nc(-c2c3ccccc3c(-c3cccc(-c4cncs4)n3)c3cc(-c4ccc(-c5cccc6ccccc56)cc4)ccc23)c1]

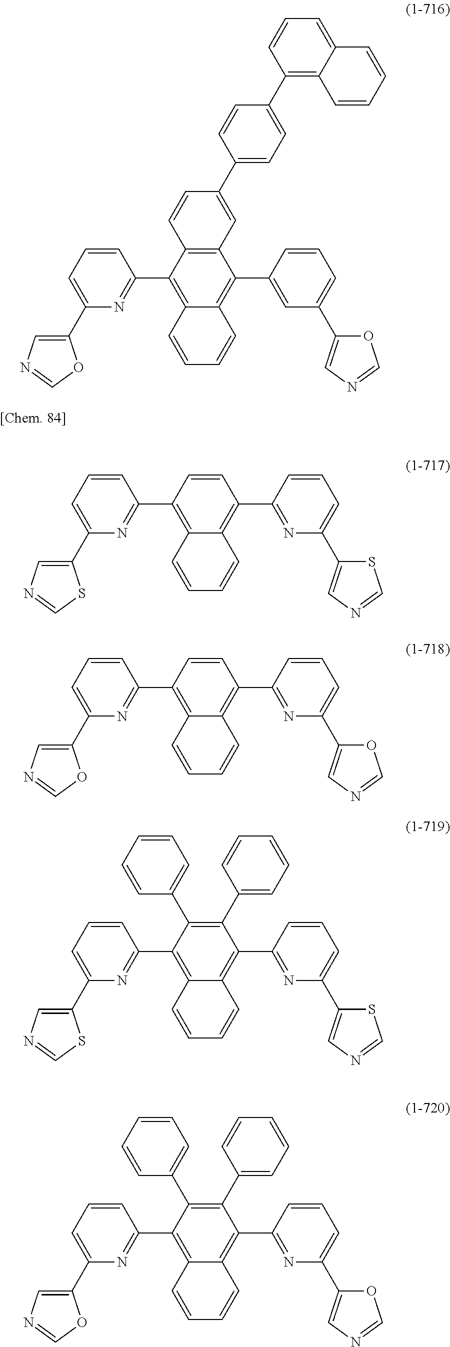 [SMILES: c1cc(-c2cnco2)cc(-c2c3ccccc3c(-c3cccc(-c4cnco4)n3)c3ccc(-c4ccc(-c5cccc6ccccc56)cc4)cc23)c1 ; c1cc(-c2cncs2)nc(-c2ccc(-c3cccc(-c4cncs4)n3)c3ccccc23)c1 ; c1cc(-c2cnco2)nc(-c2ccc(-c3cccc(-c4cnco4)n3)c3ccccc23)c1 ; c1ccc(-c2c(-c3ccccc3)c(-c3cccc(-c4cncs4)n3)c3ccccc3c2-c2cccc(-c3cncs3)n2)cc1 ; c1ccc(-c2c(-c3ccccc3)c(-c3cccc(-c4cnco4)n3)c3ccccc3c2-c2cccc(-c3cnco3)n2)cc1]

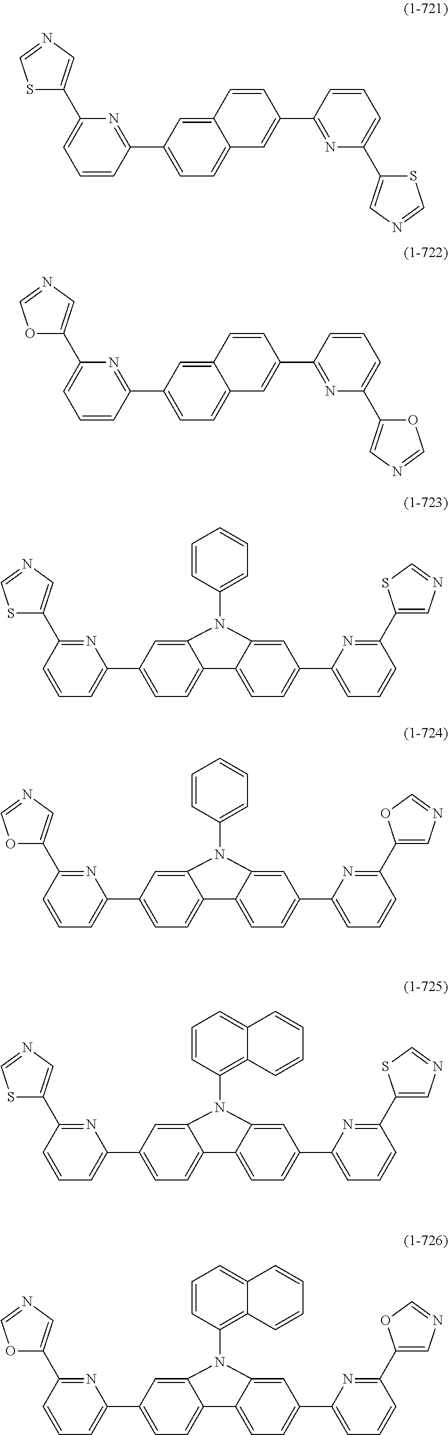 [SMILES: c1cc(-c2ccc3cc(-c4cccc(-c5cncs5)n4)ccc3c2)nc(-c2cncs2)c1 ; c1cc(-c2ccc3cc(-c4cccc(-c5cnco5)n4)ccc3c2)nc(-c2cnco2)c1 ; c1ccc(-n2c3cc(-c4cccc(-c5cncs5)n4)ccc3c3ccc(-c4cccc(-c5cncs5)n4)cc32)cc1 ; c1ccc(-n2c3cc(-c4cccc(-c5cnco5)n4)ccc3c3ccc(-c4cccc(-c5cnco5)n4)cc32)cc1 ; c1cc(-c2ccc3c4ccc(-c5cccc(-c6cncs6)n5)cc4n(-c4cccc5ccccc45)c3c2)nc(-c2cncs2)c1 ; c1cc(-c2ccc3c4ccc(-c5cccc(-c6cnco6)n5)cc4n(-c4cccc5ccccc45)c3c2)nc(-c2cnco2)c1]

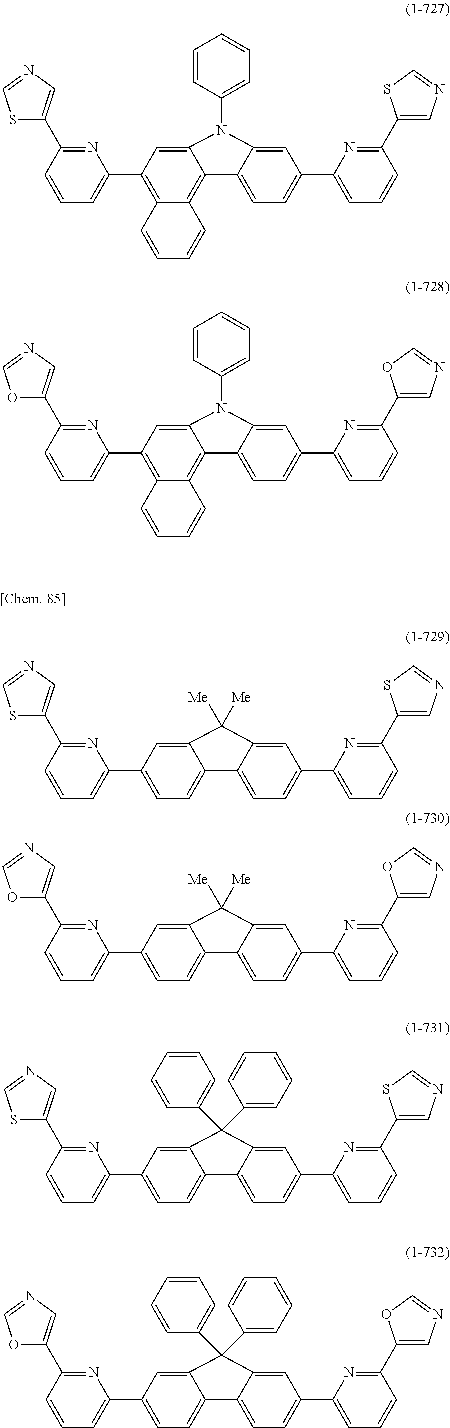 [SMILES: c1ccc(-n2c3cc(-c4cccc(-c5cncs5)n4)ccc3c3c4ccccc4c(-c4cccc(-c5cncs5)n4)cc32)cc1 ; c1ccc(-n2c3cc(-c4cccc(-c5cnco5)n4)ccc3c3c4ccccc4c(-c4cccc(-c5cnco5)n4)cc32)cc1 ; CC1(C)c2cc(-c3cccc(-c4cncs4)n3)ccc2-c2ccc(-c3cccc(-c4cncs4)n3)cc21 ; CC1(C)c2cc(-c3cccc(-c4cnco4)n3)ccc2-c2ccc(-c3cccc(-c4cnco4)n3)cc21 ; c1ccc(C2(c3ccccc3)c3cc(-c4cccc(-c5cncs5)n4)ccc3-c3ccc(-c4cccc(-c5cncs5)n4)cc32)cc1 ; c1ccc(C2(c3ccccc3)c3cc(-c4cccc(-c5cnco5)n4)ccc3-c3ccc(-c4cccc(-c5cnco5)n4)cc32)cc1]

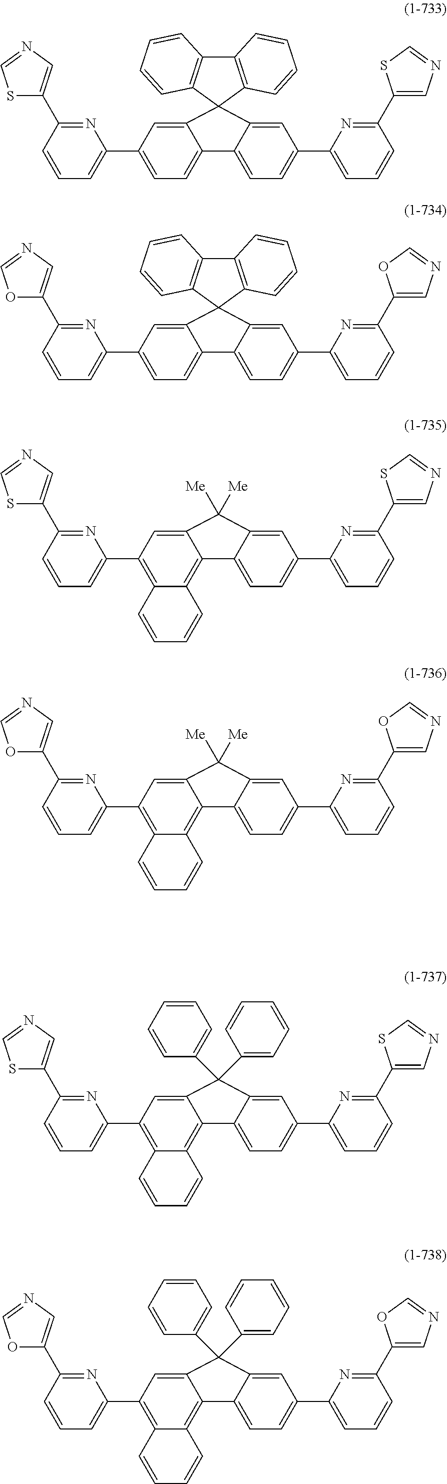

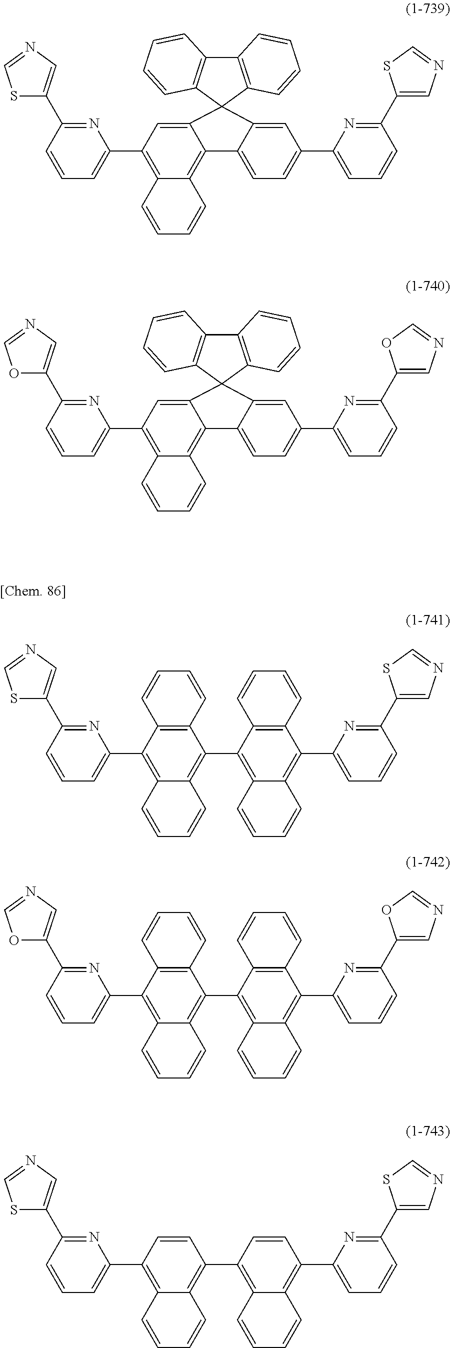 [SMILES: c1cc(-c2ccc3c(c2)C2(c4ccccc4-c4ccccc42)c2cc(-c4cccc(-c5cncs5)n4)c4ccccc4c2-3)nc(-c2cncs2)c1 ; c1cc(-c2ccc3c(c2)C2(c4ccccc4-c4ccccc42)c2cc(-c4cccc(-c5cnco5)n4)c4ccccc4c2-3)nc(-c2cnco2)c1 ; c1cc(-c2cncs2)nc(-c2c3ccccc3c(-c3c4ccccc4c(-c4cccc(-c5cncs5)n4)c4ccccc34)c3ccccc23)c1 ; c1cc(-c2cnco2)nc(-c2c3ccccc3c(-c3c4ccccc4c(-c4cccc(-c5cnco5)n4)c4ccccc34)c3ccccc23)c1 ; c1cc(-c2cncs2)nc(-c2ccc(-c3ccc(-c4cccc(-c5cncs5)n4)c4ccccc34)c3ccccc23)c1]

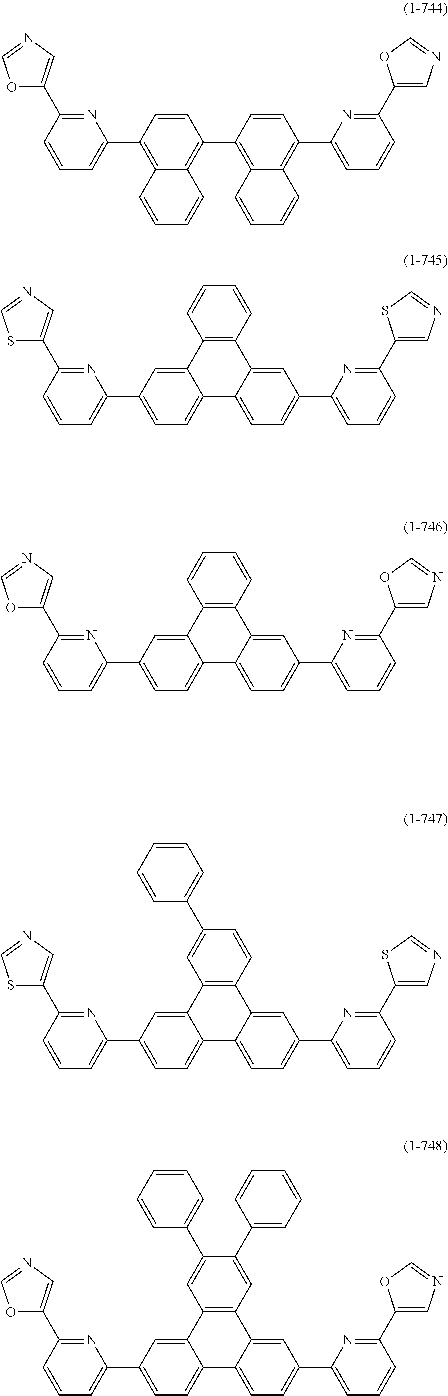 [SMILES: c1cc(-c2cnco2)nc(-c2ccc(-c3ccc(-c4cccc(-c5cnco5)n4)c4ccccc34)c3ccccc23)c1 ; c1cc(-c2ccc3c4ccc(-c5cccc(-c6cncs6)n5)cc4c4ccccc4c3c2)nc(-c2cncs2)c1 ; c1cc(-c2ccc3c4ccc(-c5cccc(-c6cnco6)n5)cc4c4ccccc4c3c2)nc(-c2cnco2)c1 ; c1ccc(-c2ccc3c(c2)c2cc(-c4cccc(-c5cncs5)n4)ccc2c2ccc(-c4cccc(-c5cncs5)n4)cc23)cc1 ; c1ccc(-c2cc3c4cc(-c5cccc(-c6cnco6)n5)ccc4c4ccc(-c5cccc(-c6cnco6)n5)cc4c3cc2-c2ccccc2)cc1]

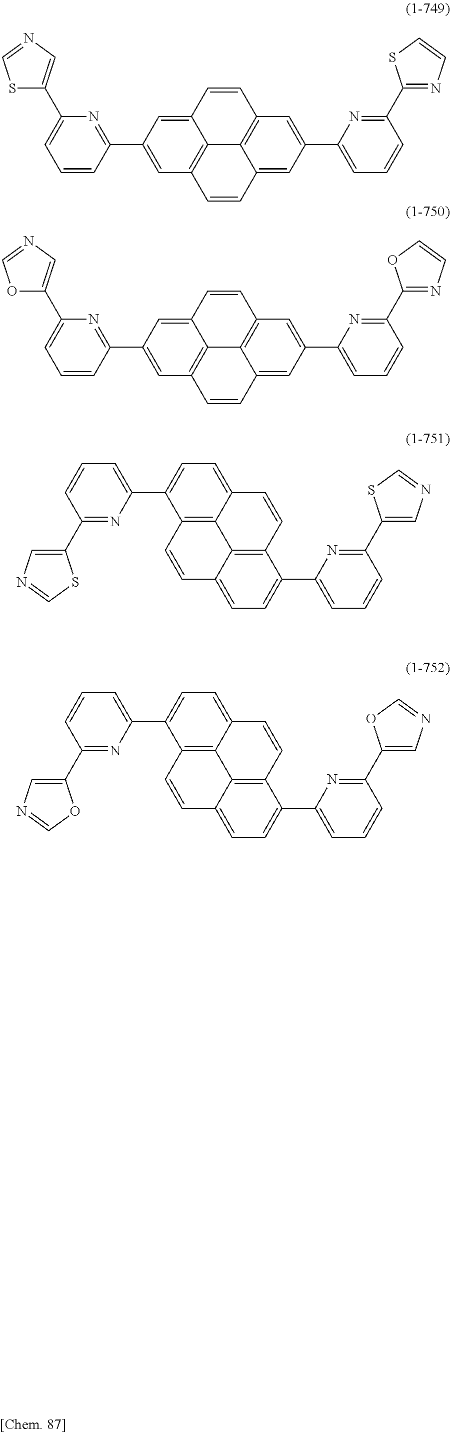 [SMILES: c1cc(-c2cc3ccc4cc(-c5cccc(-c6nccs6)n5)cc5ccc(c2)c3c45)nc(-c2cncs2)c1 ; c1cc(-c2cc3ccc4cc(-c5cccc(-c6ncco6)n5)cc5ccc(c2)c3c45)nc(-c2cnco2)c1 ; c1cc(-c2cncs2)nc(-c2ccc3ccc4c(-c5cccc(-c6cncs6)n5)ccc5ccc2c3c54)c1 ; c1cc(-c2cnco2)nc(-c2ccc3ccc4c(-c5cccc(-c6cnco6)n5)ccc5ccc2c3c54)c1]

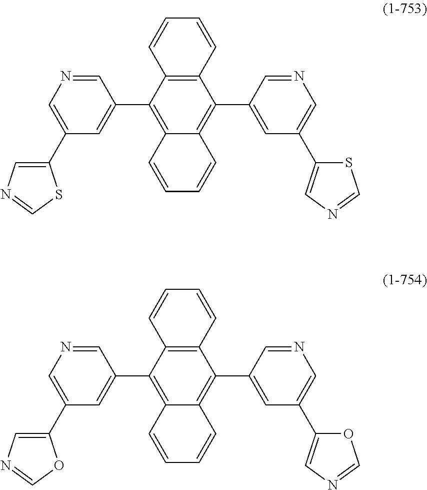 [SMILES: c1ccc2c(-c3cncc(-c4cncs4)c3)c3ccccc3c(-c3cncc(-c4cncs4)c3)c2c1 ; c1ccc2c(-c3cncc(-c4cnco4)c3)c3ccccc3c(-c3cncc(-c4cnco4)c3)c2c1]

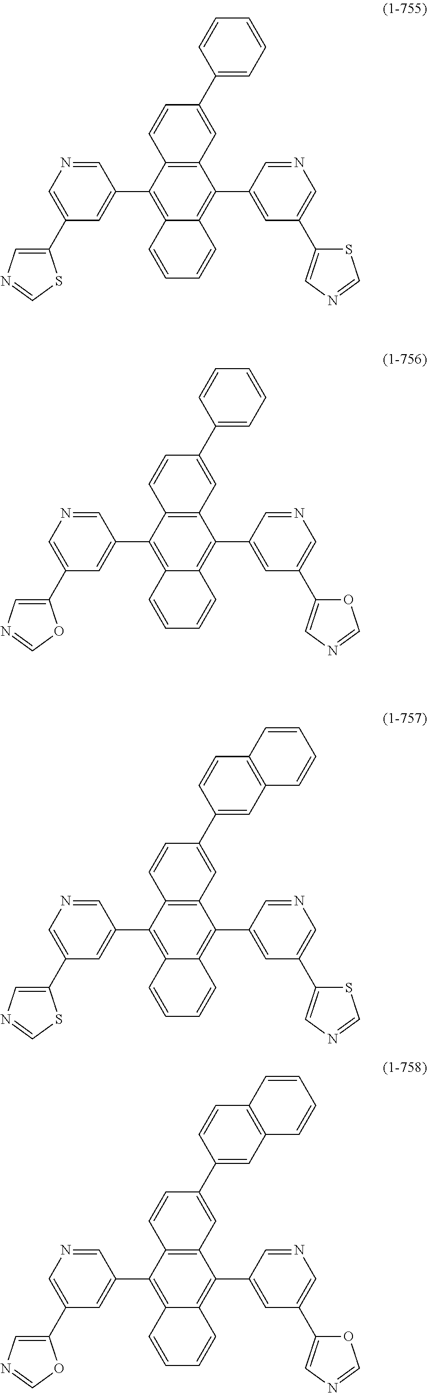 [SMILES: c1ccc(-c2ccc3c(-c4cncc(-c5cncs5)c4)c4ccccc4c(-c4cncc(-c5cncs5)c4)c3c2)cc1 ; c1ccc(-c2ccc3c(-c4cncc(-c5cnco5)c4)c4ccccc4c(-c4cncc(-c5cnco5)c4)c3c2)cc1 ; c1ccc2cc(-c3ccc4c(-c5cncc(-c6cncs6)c5)c5ccccc5c(-c5cncc(-c6cncs6)c5)c4c3)ccc2c1 ; c1ccc2cc(-c3ccc4c(-c5cncc(-c6cnco6)c5)c5ccccc5c(-c5cncc(-c6cnco6)c5)c4c3)ccc2c1]

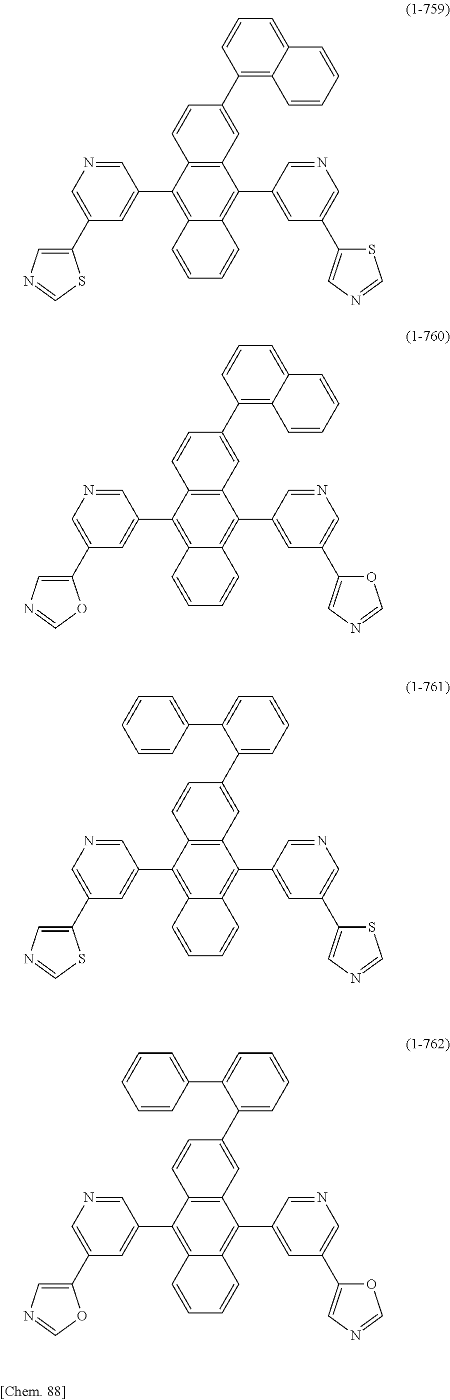 [SMILES: c1ccc2c(-c3ccc4c(-c5cncc(-c6cncs6)c5)c5ccccc5c(-c5cncc(-c6cncs6)c5)c4c3)cccc2c1 ; c1ccc2c(-c3ccc4c(-c5cncc(-c6cnco6)c5)c5ccccc5c(-c5cncc(-c6cnco6)c5)c4c3)cccc2c1 ; c1ccc(-c2ccccc2-c2ccc3c(-c4cncc(-c5cncs5)c4)c4ccccc4c(-c4cncc(-c5cncs5)c4)c3c2)cc1 ; c1ccc(-c2ccccc2-c2ccc3c(-c4cncc(-c5cnco5)c4)c4ccccc4c(-c4cncc(-c5cnco5)c4)c3c2)cc1]

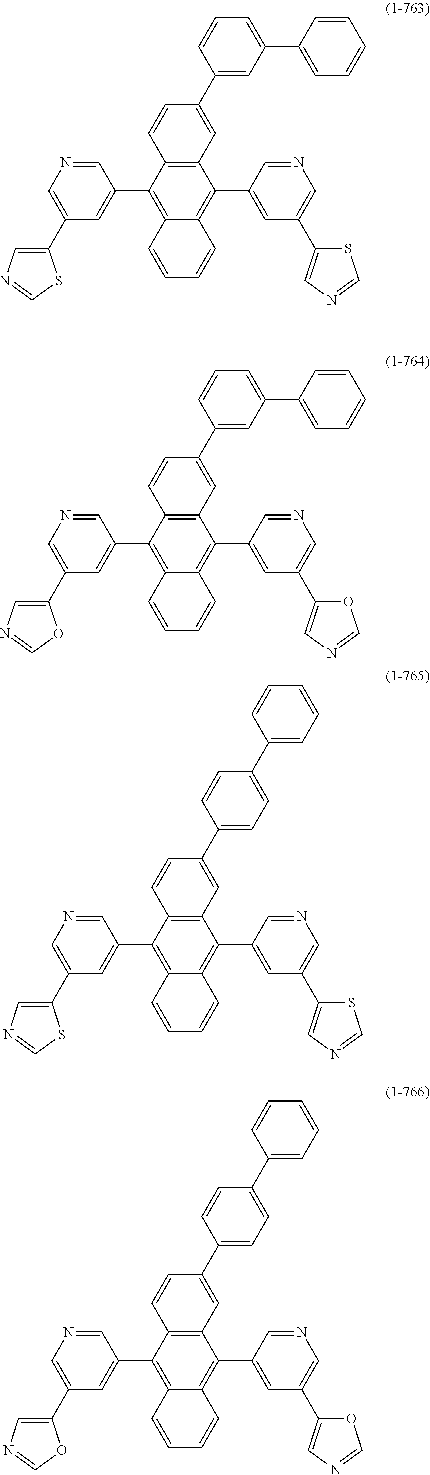

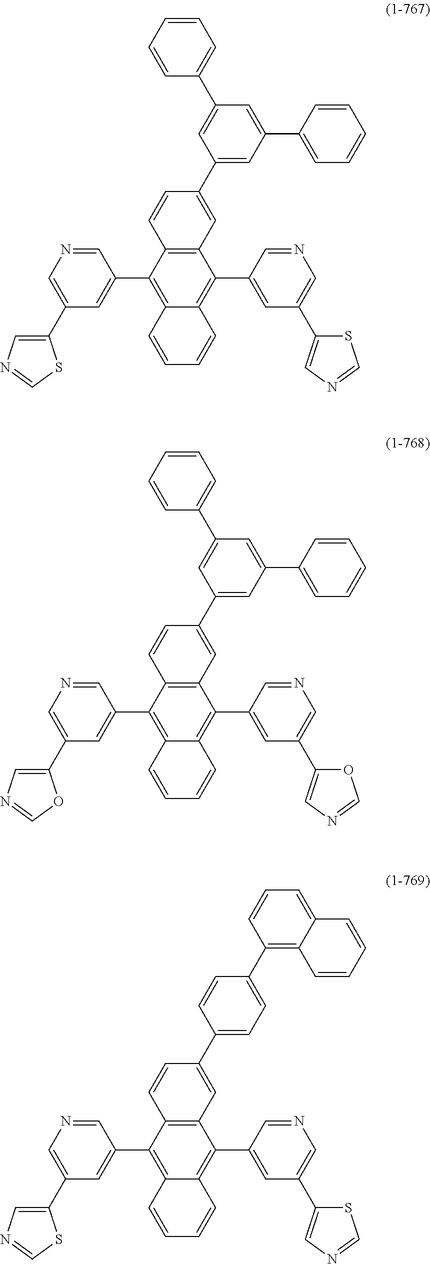 [SMILES: c1ccc(-c2cc(-c3ccccc3)cc(-c3ccc4c(-c5cncc(-c6cncs6)c5)c5ccccc5c(-c5cncc(-c6cncs6)c5)c4c3)c2)cc1 ; c1ccc(-c2cc(-c3ccccc3)cc(-c3ccc4c(-c5cncc(-c6cnco6)c5)c5ccccc5c(-c5cncc(-c6cnco6)c5)c4c3)c2)cc1 ; c1ccc2c(-c3ccc(-c4ccc5c(-c6cncc(-c7cncs7)c6)c6ccccc6c(-c6cncc(-c7cncs7)c6)c5c4)cc3)cccc2c1]

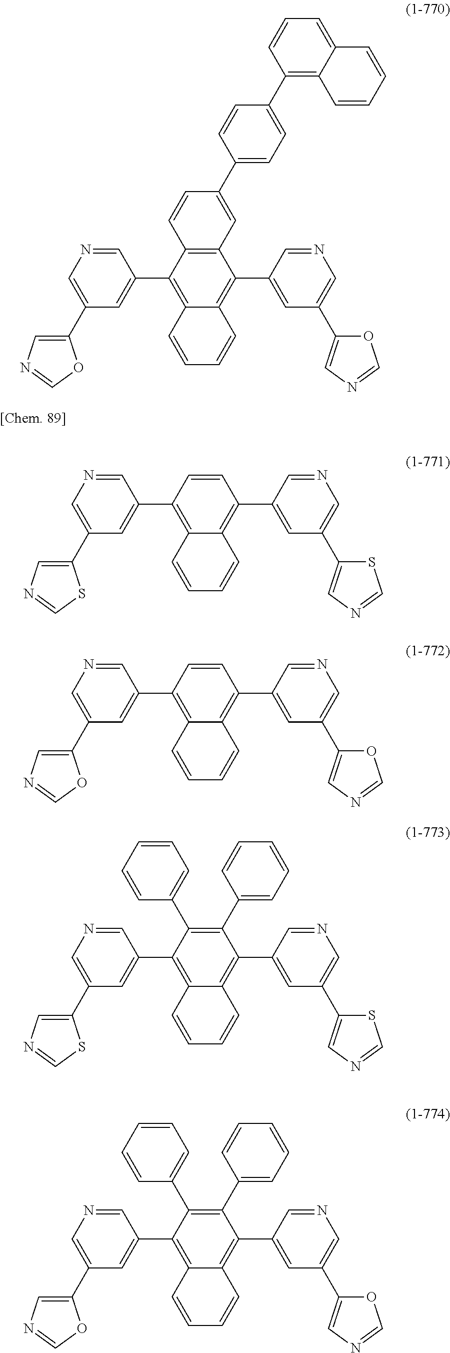 [SMILES: c1ccc2c(-c3ccc(-c4ccc5c(-c6cncc(-c7cnco7)c6)c6ccccc6c(-c6cncc(-c7cnco7)c6)c5c4)cc3)cccc2c1 ; c1ccc2c(-c3cncc(-c4cncs4)c3)ccc(-c3cncc(-c4cncs4)c3)c2c1 ; c1ccc2c(-c3cncc(-c4cnco4)c3)ccc(-c3cncc(-c4cnco4)c3)c2c1 ; c1ccc(-c2c(-c3ccccc3)c(-c3cncc(-c4cncs4)c3)c3ccccc3c2-c2cncc(-c3cncs3)c2)cc1 ; c1ccc(-c2c(-c3ccccc3)c(-c3cncc(-c4cnco4)c3)c3ccccc3c2-c2cncc(-c3cnco3)c2)cc1]

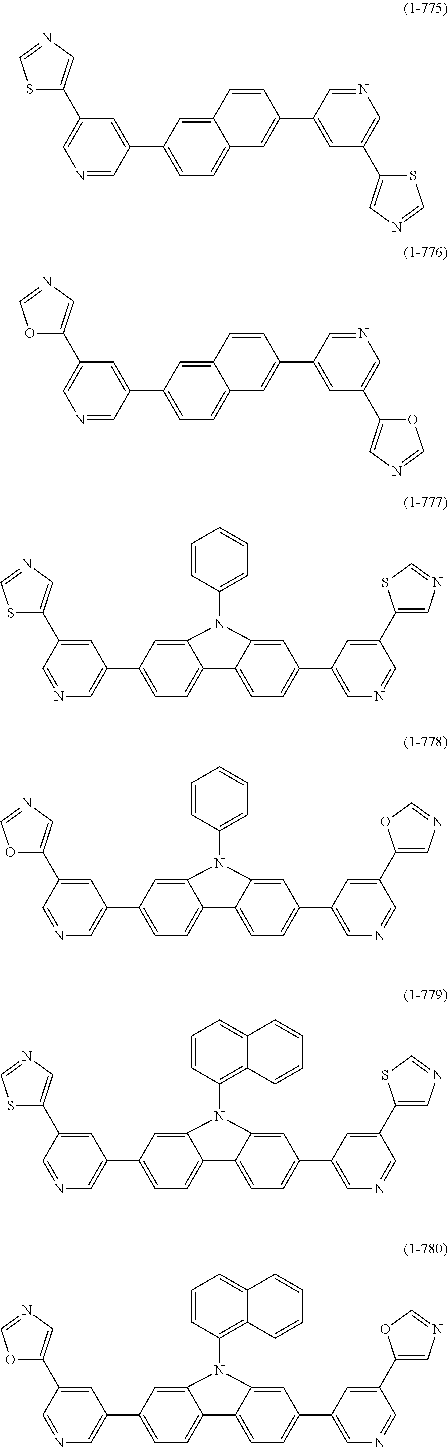 [SMILES: c1ncc(-c2cncc(-c3ccc4cc(-c5cncc(-c6cncs6)c5)ccc4c3)c2)s1 ; c1ncc(-c2cncc(-c3ccc4cc(-c5cncc(-c6cnco6)c5)ccc4c3)c2)o1 ; c1ccc(-n2c3cc(-c4cncc(-c5cncs5)c4)ccc3c3ccc(-c4cncc(-c5cncs5)c4)cc32)cc1 ; c1ccc(-n2c3cc(-c4cncc(-c5cnco5)c4)ccc3c3ccc(-c4cncc(-c5cnco5)c4)cc32)cc1 ; c1ccc2c(-n3c4cc(-c5cncc(-c6cncs6)c5)ccc4c4ccc(-c5cncc(-c6cncs6)c5)cc43)cccc2c1 ; c1ccc2c(-n3c4cc(-c5cncc(-c6cnco6)c5)ccc4c4ccc(-c5cncc(-c6cnco6)c5)cc43)cccc2c1]

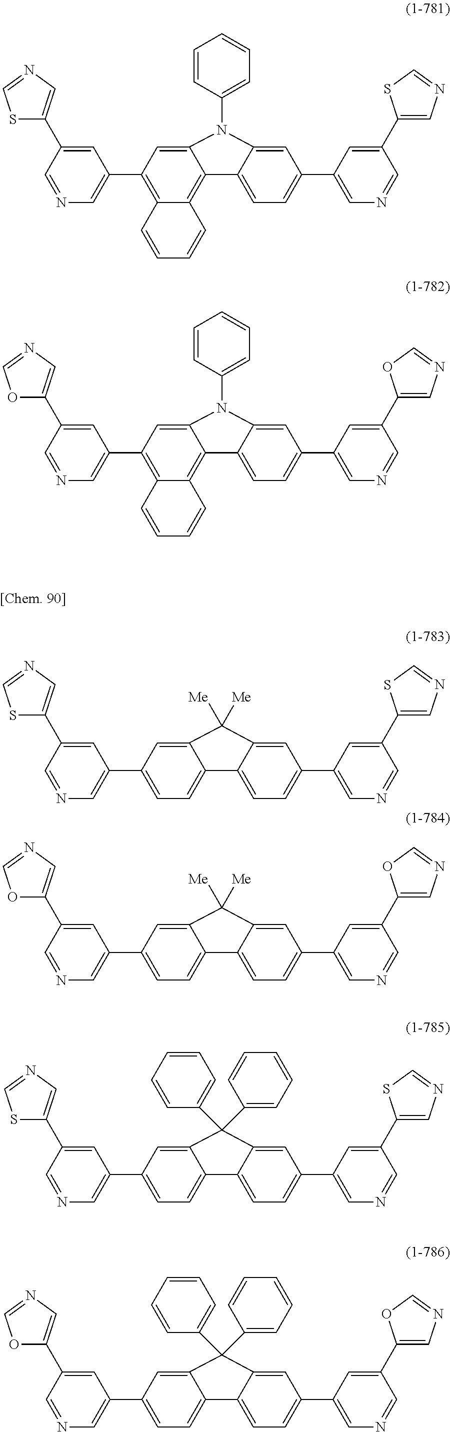 [SMILES: c1ccc(-n2c3cc(-c4cncc(-c5cncs5)c4)ccc3c3c4ccccc4c(-c4cncc(-c5cncs5)c4)cc32)cc1 ; c1ccc(-n2c3cc(-c4cncc(-c5cnco5)c4)ccc3c3c4ccccc4c(-c4cncc(-c5cnco5)c4)cc32)cc1 ; CC1(C)c2cc(-c3cncc(-c4cncs4)c3)ccc2-c2ccc(-c3cncc(-c4cncs4)c3)cc21 ; CC1(C)c2cc(-c3cncc(-c4cnco4)c3)ccc2-c2ccc(-c3cncc(-c4cnco4)c3)cc21 ; c1ccc(C2(c3ccccc3)c3cc(-c4cncc(-c5cncs5)c4)ccc3-c3ccc(-c4cncc(-c5cncs5)c4)cc32)cc1 ; c1ccc(C2(c3ccccc3)c3cc(-c4cncc(-c5cnco5)c4)ccc3-c3ccc(-c4cncc(-c5cnco5)c4)cc32)cc1]

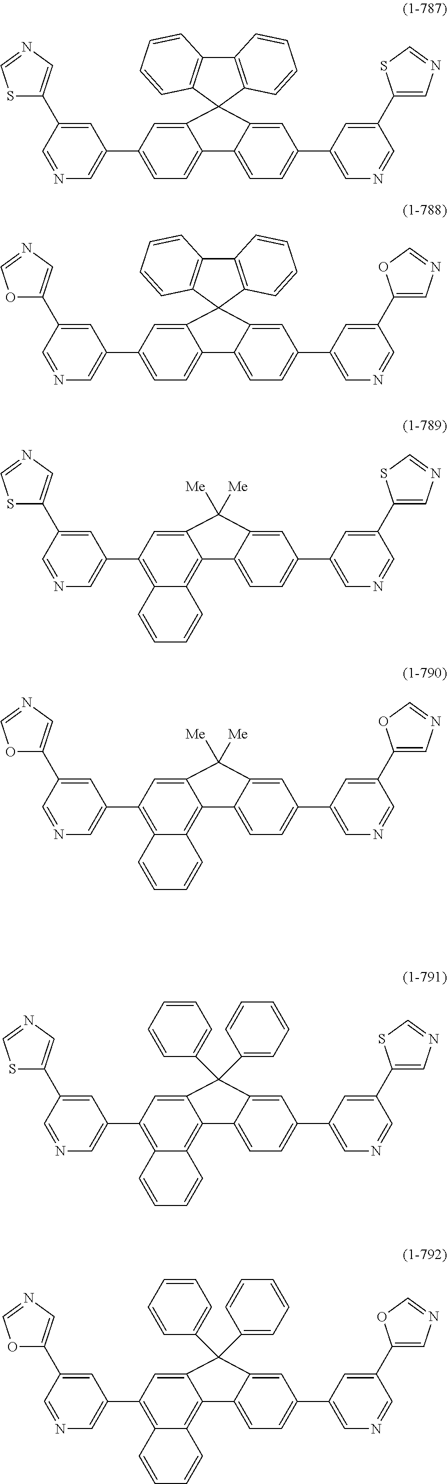

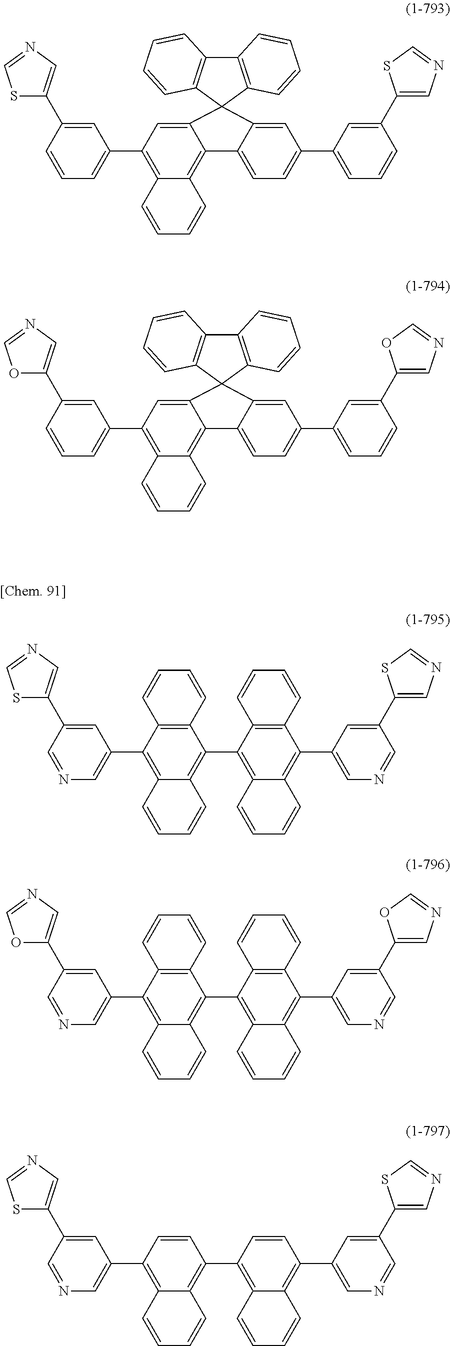 [SMILES: c1cc(-c2ccc3c(c2)C2(c4ccccc4-c4ccccc42)c2cc(-c4cccc(-c5cncs5)c4)c4ccccc4c2-3)cc(-c2cncs2)c1 ; c1cc(-c2ccc3c(c2)C2(c4ccccc4-c4ccccc42)c2cc(-c4cccc(-c5cnco5)c4)c4ccccc4c2-3)cc(-c2cnco2)c1 ; c1ccc2c(-c3c4ccccc4c(-c4cncc(-c5cncs5)c4)c4ccccc34)c3ccccc3c(-c3cncc(-c4cncs4)c3)c2c1 ; c1ccc2c(-c3c4ccccc4c(-c4cncc(-c5cnco5)c4)c4ccccc34)c3ccccc3c(-c3cncc(-c4cnco4)c3)c2c1 ; c1ccc2c(-c3ccc(-c4cncc(-c5cncs5)c4)c4ccccc34)ccc(-c3cncc(-c4cncs4)c3)c2c1]

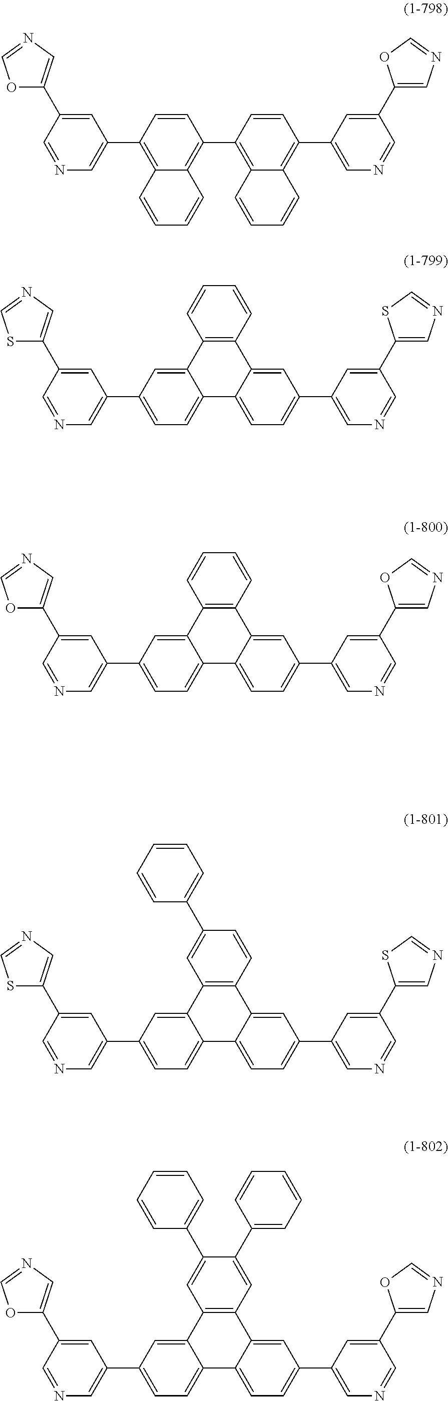 [SMILES: c1ccc2c(-c3ccc(-c4cncc(-c5cnco5)c4)c4ccccc34)ccc(-c3cncc(-c4cnco4)c3)c2c1 ; c1ccc2c(c1)c1cc(-c3cncc(-c4cncs4)c3)ccc1c1ccc(-c3cncc(-c4cncs4)c3)cc21 ; c1ccc2c(c1)c1cc(-c3cncc(-c4cnco4)c3)ccc1c1ccc(-c3cncc(-c4cnco4)c3)cc21 ; c1ccc(-c2ccc3c4cc(-c5cncc(-c6cncs6)c5)ccc4c4ccc(-c5cncc(-c6cncs6)c5)cc4c3c2)cc1 ; c1ccc(-c2cc3c4cc(-c5cncc(-c6cnco6)c5)ccc4c4ccc(-c5cncc(-c6cnco6)c5)cc4c3cc2-c2ccccc2)cc1]

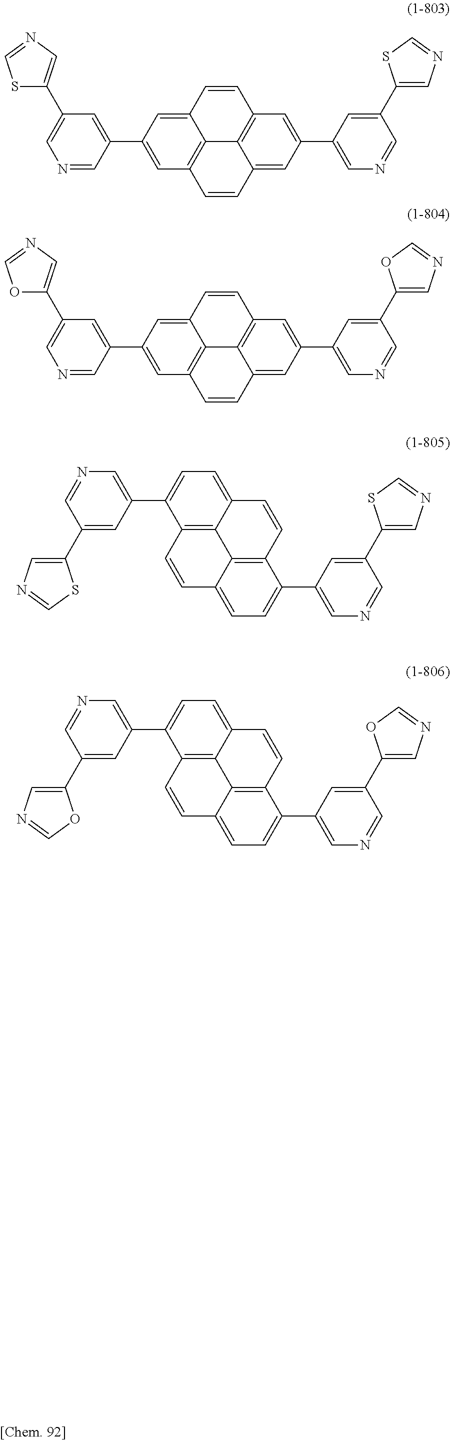 [SMILES: c1ncc(-c2cncc(-c3cc4ccc5cc(-c6cncc(-c7cncs7)c6)cc6ccc(c3)c4c56)c2)s1 ; c1ncc(-c2cncc(-c3cc4ccc5cc(-c6cncc(-c7cnco7)c6)cc6ccc(c3)c4c56)c2)o1 ; c1ncc(-c2cncc(-c3ccc4ccc5c(-c6cncc(-c7cncs7)c6)ccc6ccc3c4c65)c2)s1 ; c1ncc(-c2cncc(-c3ccc4ccc5c(-c6cncc(-c7cnco7)c6)ccc6ccc3c4c65)c2)o1]

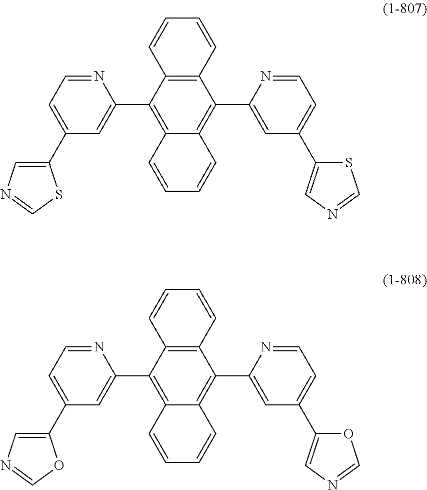 [SMILES: c1ccc2c(-c3cc(-c4cncs4)ccn3)c3ccccc3c(-c3cc(-c4cncs4)ccn3)c2c1 ; c1ccc2c(-c3cc(-c4cnco4)ccn3)c3ccccc3c(-c3cc(-c4cnco4)ccn3)c2c1]

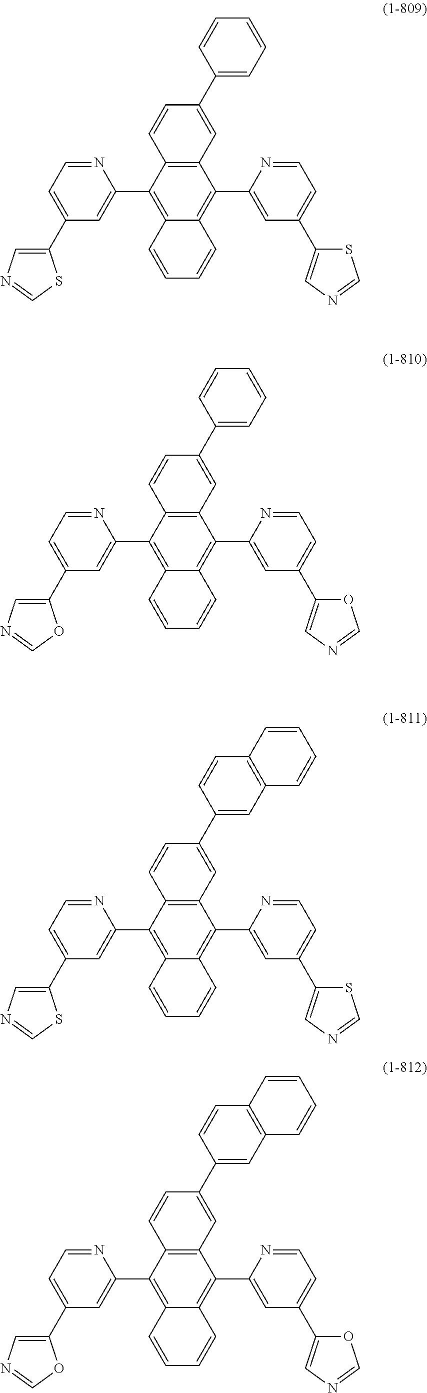 [SMILES: c1ccc(-c2ccc3c(-c4cc(-c5cncs5)ccn4)c4ccccc4c(-c4cc(-c5cncs5)ccn4)c3c2)cc1 ; c1ccc(-c2ccc3c(-c4cc(-c5cnco5)ccn4)c4ccccc4c(-c4cc(-c5cnco5)ccn4)c3c2)cc1 ; c1ccc2cc(-c3ccc4c(-c5cc(-c6cncs6)ccn5)c5ccccc5c(-c5cc(-c6cncs6)ccn5)c4c3)ccc2c1 ; c1ccc2cc(-c3ccc4c(-c5cc(-c6cnco6)ccn5)c5ccccc5c(-c5cc(-c6cnco6)ccn5)c4c3)ccc2c1]

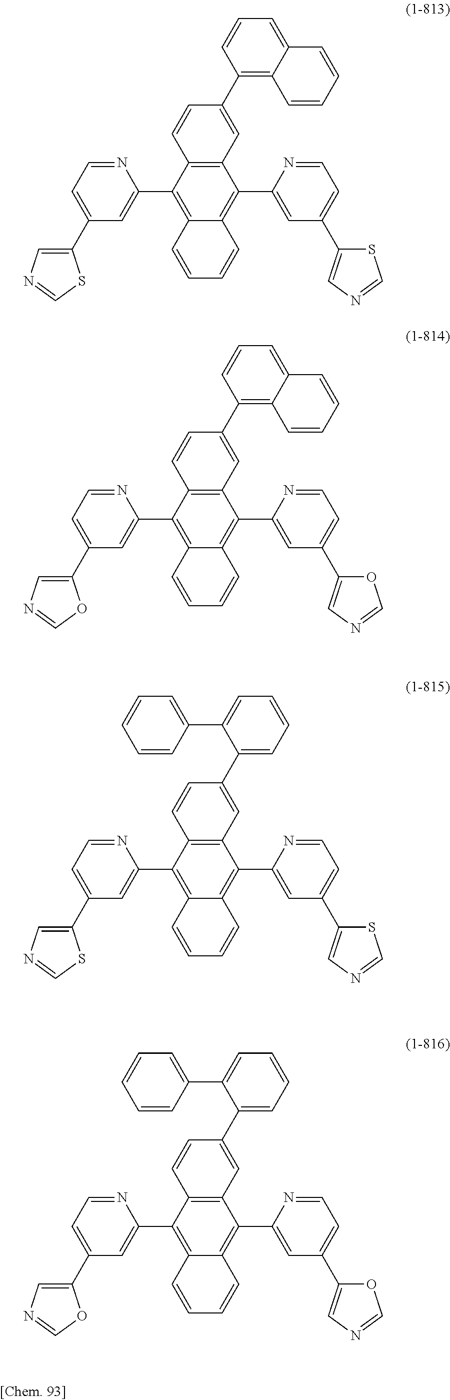 [SMILES: c1ccc2c(-c3ccc4c(-c5cc(-c6cncs6)ccn5)c5ccccc5c(-c5cc(-c6cncs6)ccn5)c4c3)cccc2c1 ; c1ccc2c(-c3ccc4c(-c5cc(-c6cnco6)ccn5)c5ccccc5c(-c5cc(-c6cnco6)ccn5)c4c3)cccc2c1 ; c1ccc(-c2ccccc2-c2ccc3c(-c4cc(-c5cncs5)ccn4)c4ccccc4c(-c4cc(-c5cncs5)ccn4)c3c2)cc1 ; c1ccc(-c2ccccc2-c2ccc3c(-c4cc(-c5cnco5)ccn4)c4ccccc4c(-c4cc(-c5cnco5)ccn4)c3c2)cc1]

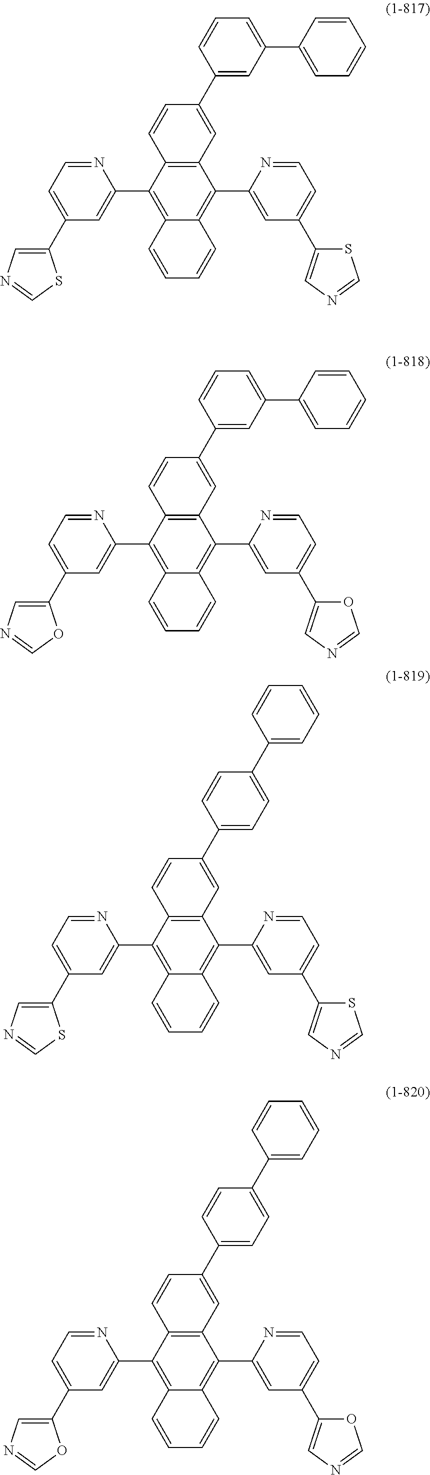

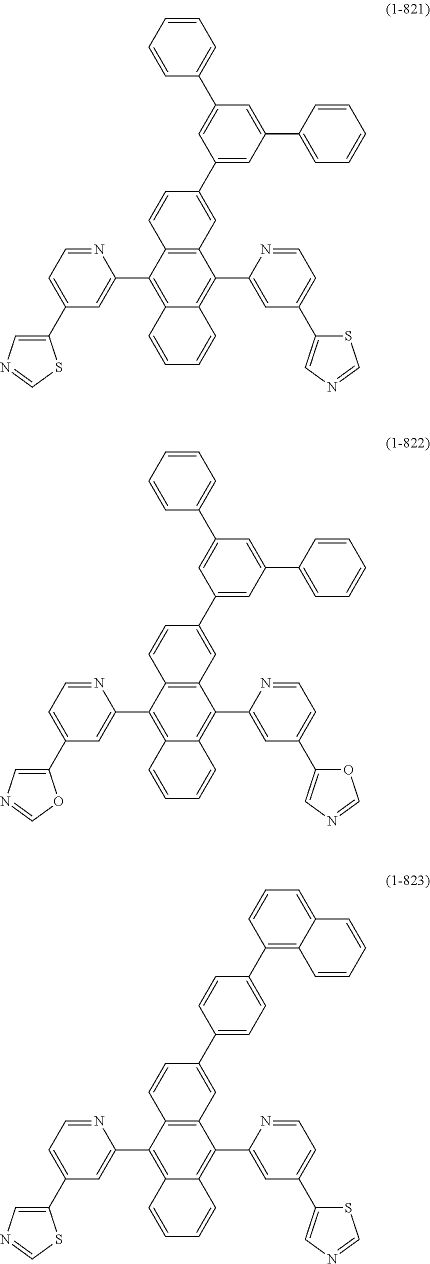 [SMILES: c1ccc(-c2cc(-c3ccccc3)cc(-c3ccc4c(-c5cc(-c6cncs6)ccn5)c5ccccc5c(-c5cc(-c6cncs6)ccn5)c4c3)c2)cc1 ; c1ccc(-c2cc(-c3ccccc3)cc(-c3ccc4c(-c5cc(-c6cnco6)ccn5)c5ccccc5c(-c5cc(-c6cnco6)ccn5)c4c3)c2)cc1 ; c1ccc2c(-c3ccc(-c4ccc5c(-c6cc(-c7cncs7)ccn6)c6ccccc6c(-c6cc(-c7cncs7)ccn6)c5c4)cc3)cccc2c1]

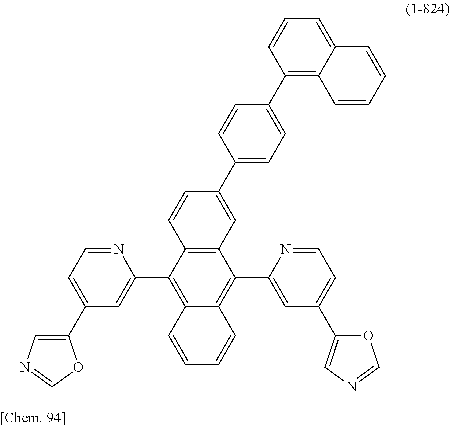 [SMILES: c1ccc2c(-c3ccc(-c4ccc5c(-c6cc(-c7cnco7)ccn6)c6ccccc6c(-c6cc(-c7cnco7)ccn6)c5c4)cc3)cccc2c1]

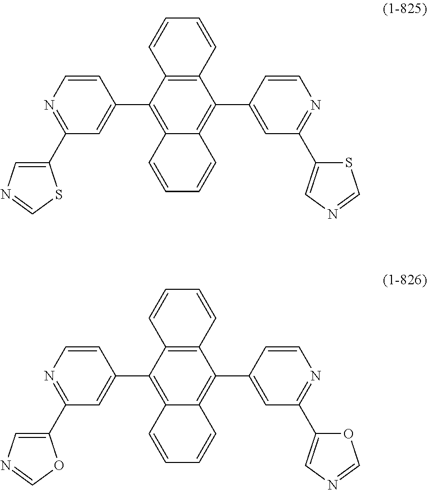 [SMILES: c1ccc2c(-c3ccnc(-c4cncs4)c3)c3ccccc3c(-c3ccnc(-c4cncs4)c3)c2c1 ; c1ccc2c(-c3ccnc(-c4cnco4)c3)c3ccccc3c(-c3ccnc(-c4cnco4)c3)c2c1]

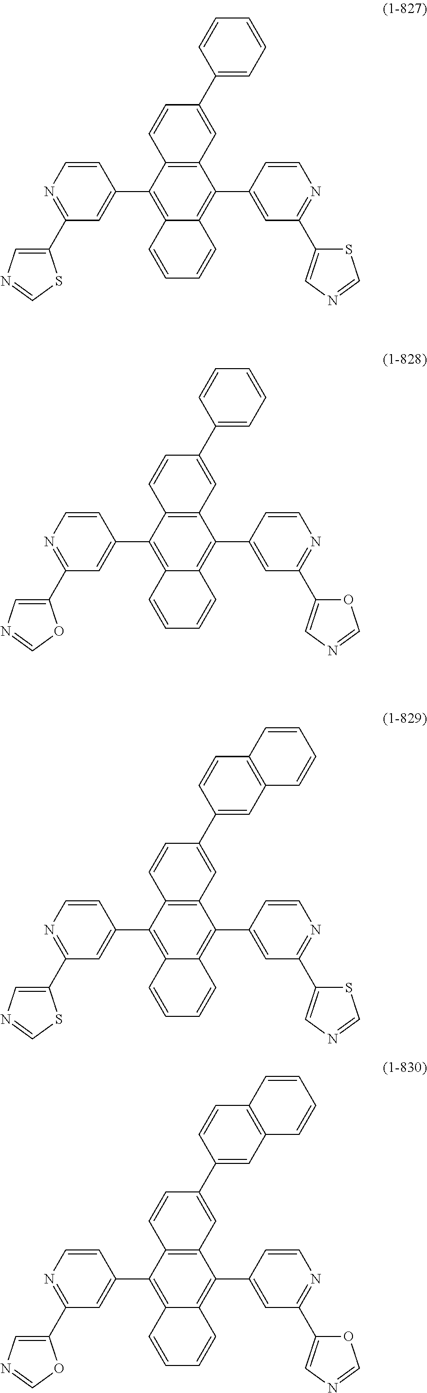 [SMILES: c1ccc(-c2ccc3c(-c4ccnc(-c5cncs5)c4)c4ccccc4c(-c4ccnc(-c5cncs5)c4)c3c2)cc1 ; c1ccc(-c2ccc3c(-c4ccnc(-c5cnco5)c4)c4ccccc4c(-c4ccnc(-c5cnco5)c4)c3c2)cc1 ; c1ccc2cc(-c3ccc4c(-c5ccnc(-c6cncs6)c5)c5ccccc5c(-c5ccnc(-c6cncs6)c5)c4c3)ccc2c1 ; c1ccc2cc(-c3ccc4c(-c5ccnc(-c6cnco6)c5)c5ccccc5c(-c5ccnc(-c6cnco6)c5)c4c3)ccc2c1]

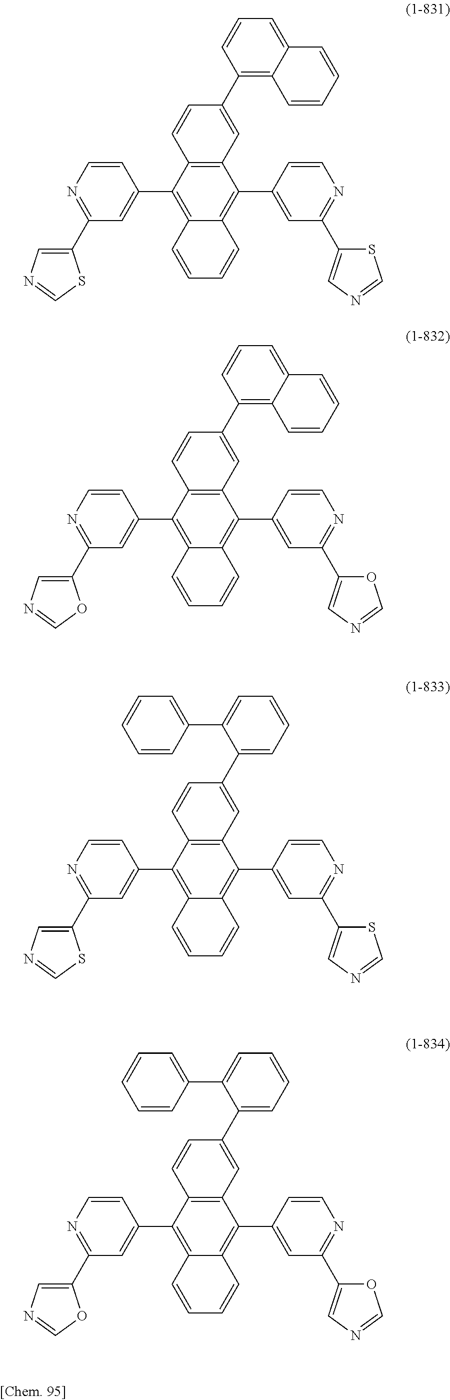 [SMILES: c1ccc2c(-c3ccc4c(-c5ccnc(-c6cncs6)c5)c5ccccc5c(-c5ccnc(-c6cncs6)c5)c4c3)cccc2c1 ; c1ccc2c(-c3ccc4c(-c5ccnc(-c6cnco6)c5)c5ccccc5c(-c5ccnc(-c6cnco6)c5)c4c3)cccc2c1 ; c1ccc(-c2ccccc2-c2ccc3c(-c4ccnc(-c5cncs5)c4)c4ccccc4c(-c4ccnc(-c5cncs5)c4)c3c2)cc1 ; c1ccc(-c2ccccc2-c2ccc3c(-c4ccnc(-c5cnco5)c4)c4ccccc4c(-c4ccnc(-c5cnco5)c4)c3c2)cc1]

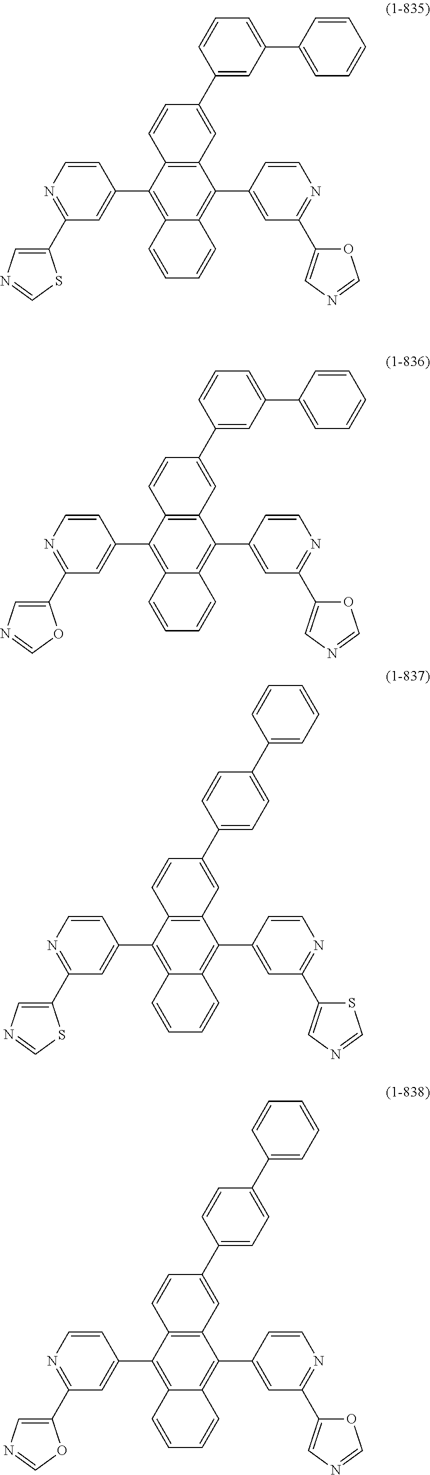

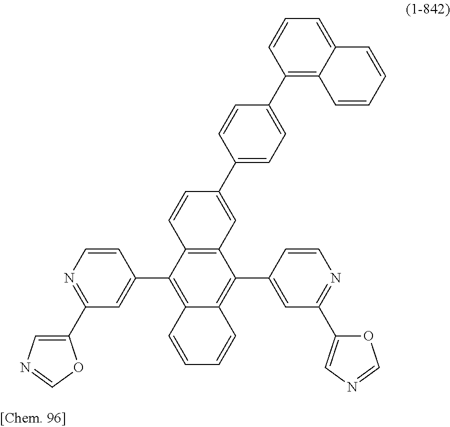 [SMILES: c1ccc2c(-c3ccc(-c4ccc5c(-c6ccnc(-c7cnco7)c6)c6ccccc6c(-c6ccnc(-c7cnco7)c6)c5c4)cc3)cccc2c1]

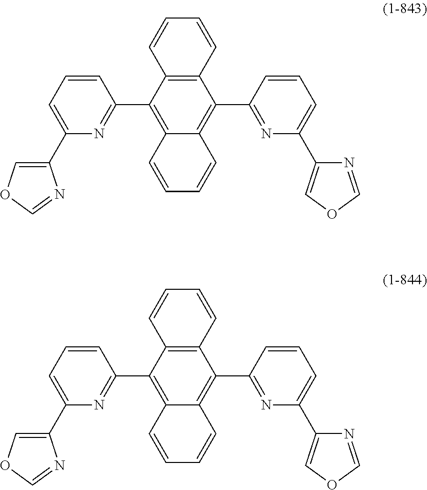 [SMILES: c1cc(-c2cocn2)nc(-c2c3ccccc3c(-c3cccc(-c4cocn4)n3)c3ccccc23)c1 ; c1cc(-c2cocn2)nc(-c2c3ccccc3c(-c3cccc(-c4cocn4)n3)c3ccccc23)c1]

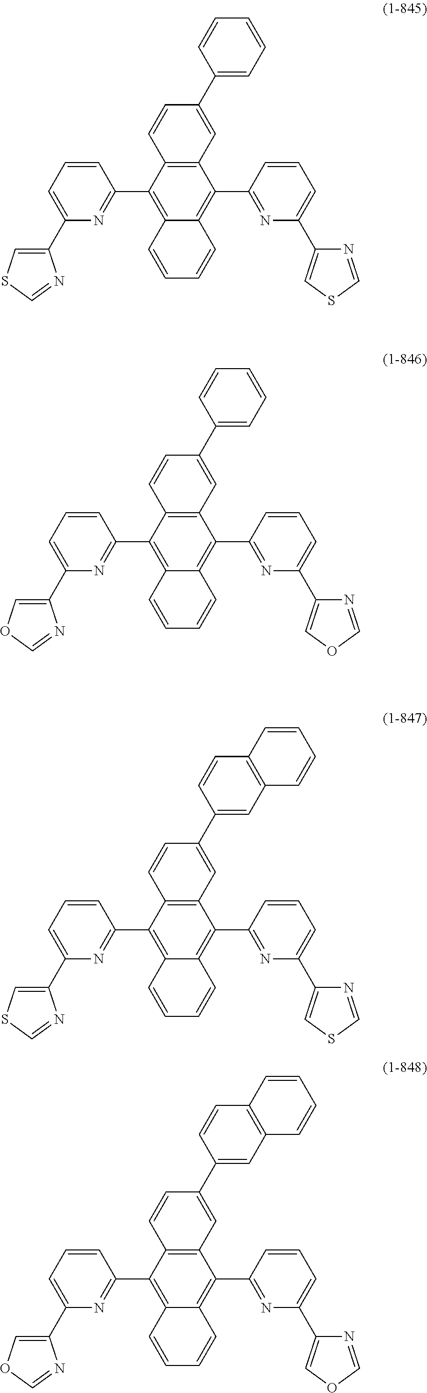 [SMILES: c1ccc(-c2ccc3c(-c4cccc(-c5cscn5)n4)c4ccccc4c(-c4cccc(-c5cscn5)n4)c3c2)cc1 ; c1ccc(-c2ccc3c(-c4cccc(-c5cocn5)n4)c4ccccc4c(-c4cccc(-c5cocn5)n4)c3c2)cc1 ; c1cc(-c2cscn2)nc(-c2c3ccccc3c(-c3cccc(-c4cscn4)n3)c3cc(-c4ccc5ccccc5c4)ccc23)c1 ; c1cc(-c2cocn2)nc(-c2c3ccccc3c(-c3cccc(-c4cocn4)n3)c3cc(-c4ccc5ccccc5c4)ccc23)c1]

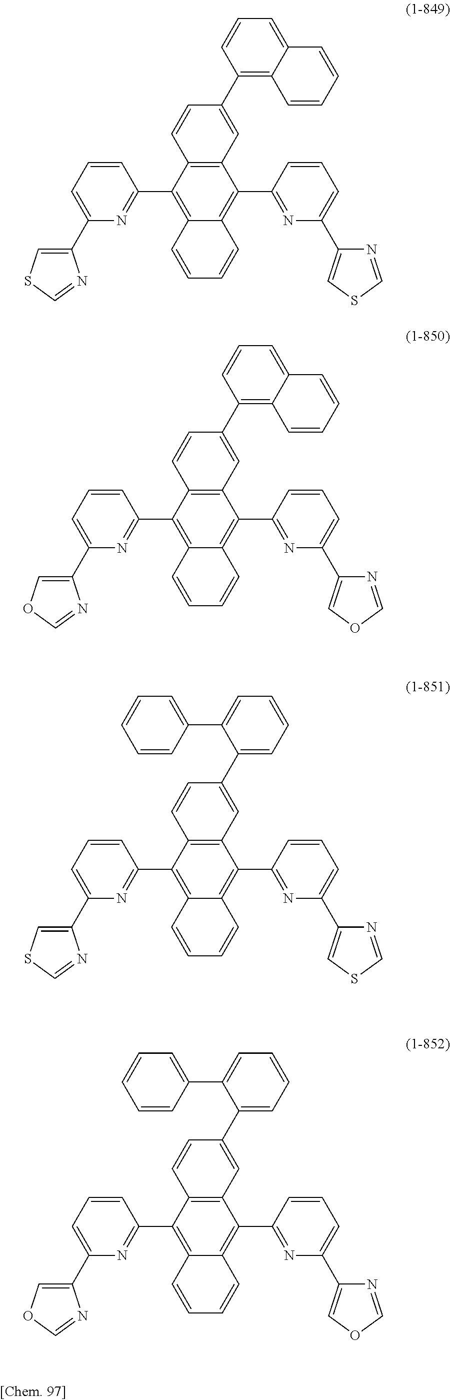 [SMILES: c1cc(-c2cscn2)nc(-c2c3ccccc3c(-c3cccc(-c4cscn4)n3)c3cc(-c4cccc5ccccc45)ccc23)c1 ; c1cc(-c2cocn2)nc(-c2c3ccccc3c(-c3cccc(-c4cocn4)n3)c3cc(-c4cccc5ccccc45)ccc23)c1 ; c1ccc(-c2ccccc2-c2ccc3c(-c4cccc(-c5cscn5)n4)c4ccccc4c(-c4cccc(-c5cscn5)n4)c3c2)cc1 ; c1ccc(-c2ccccc2-c2ccc3c(-c4cccc(-c5cocn5)n4)c4ccccc4c(-c4cccc(-c5cocn5)n4)c3c2)cc1]

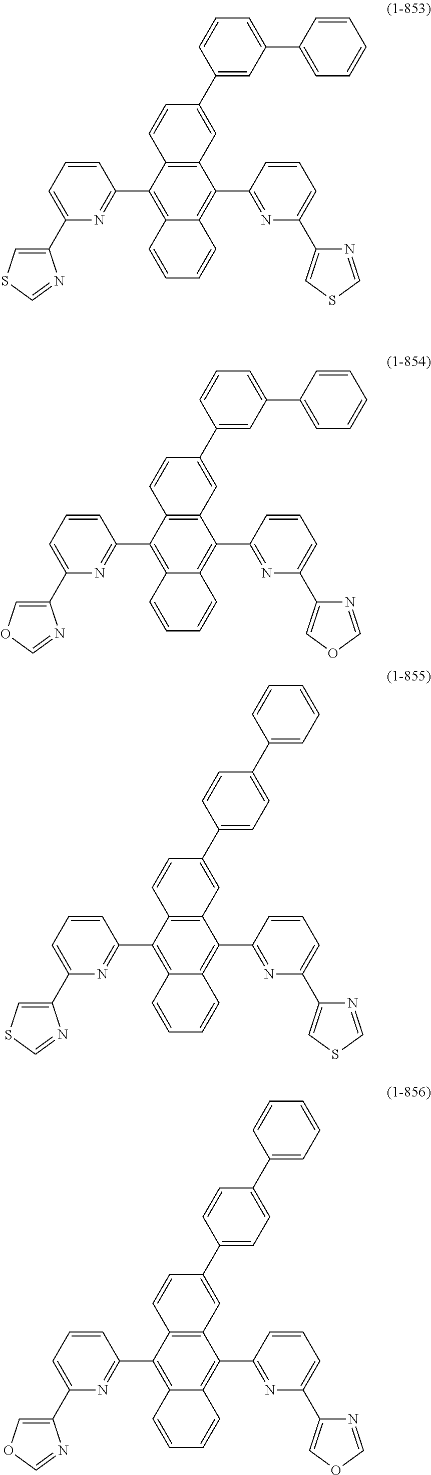

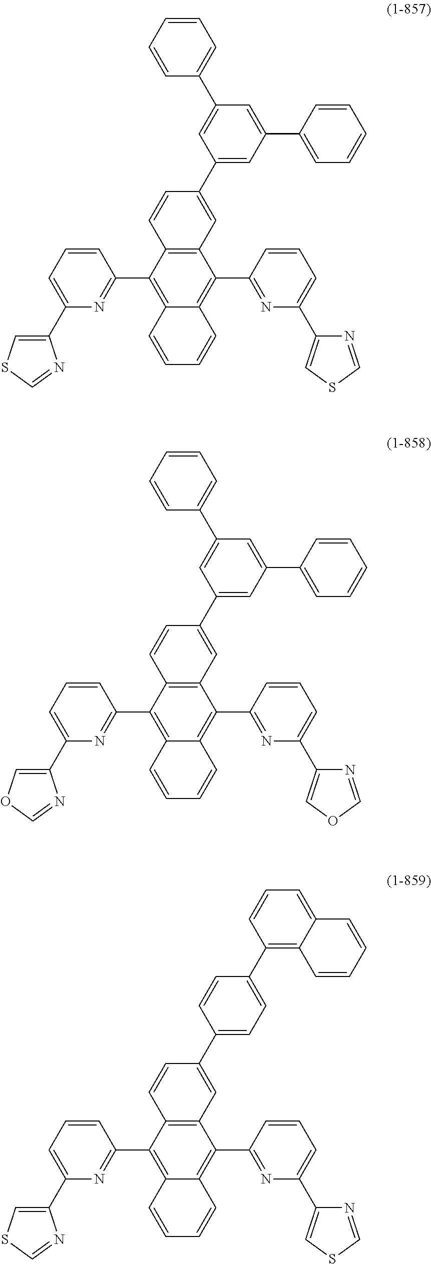 [SMILES: c1ccc(-c2cc(-c3ccccc3)cc(-c3ccc4c(-c5cccc(-c6cscn6)n5)c5ccccc5c(-c5cccc(-c6cscn6)n5)c4c3)c2)cc1 ; c1ccc(-c2cc(-c3ccccc3)cc(-c3ccc4c(-c5cccc(-c6cocn6)n5)c5ccccc5c(-c5cccc(-c6cocn6)n5)c4c3)c2)cc1 ; c1cc(-c2cscn2)nc(-c2c3ccccc3c(-c3cccc(-c4cscn4)n3)c3cc(-c4ccc(-c5cccc6ccccc56)cc4)ccc23)c1]

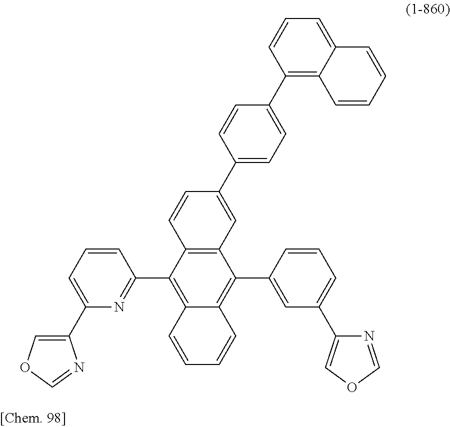 [SMILES: c1cc(-c2cocn2)cc(-c2c3ccccc3c(-c3cccc(-c4cocn4)n3)c3ccc(-c4ccc(-c5cccc6ccccc56)cc4)cc23)c1]

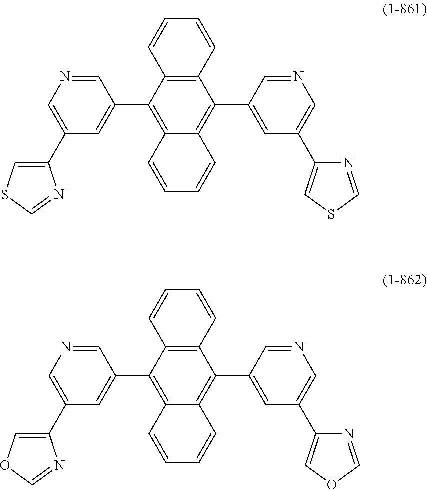 [SMILES: c1ccc2c(-c3cncc(-c4cscn4)c3)c3ccccc3c(-c3cncc(-c4cscn4)c3)c2c1 ; c1ccc2c(-c3cncc(-c4cocn4)c3)c3ccccc3c(-c3cncc(-c4cocn4)c3)c2c1]

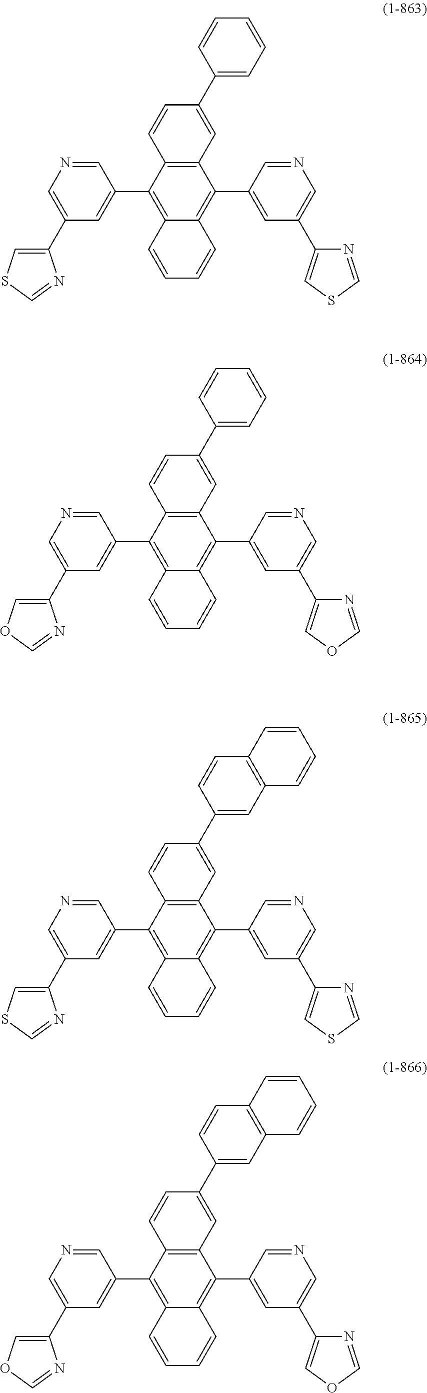 [SMILES: c1ccc(-c2ccc3c(-c4cncc(-c5cscn5)c4)c4ccccc4c(-c4cncc(-c5cscn5)c4)c3c2)cc1 ; c1ccc(-c2ccc3c(-c4cncc(-c5cocn5)c4)c4ccccc4c(-c4cncc(-c5cocn5)c4)c3c2)cc1 ; c1ccc2cc(-c3ccc4c(-c5cncc(-c6cscn6)c5)c5ccccc5c(-c5cncc(-c6cscn6)c5)c4c3)ccc2c1 ; c1ccc2cc(-c3ccc4c(-c5cncc(-c6cocn6)c5)c5ccccc5c(-c5cncc(-c6cocn6)c5)c4c3)ccc2c1]

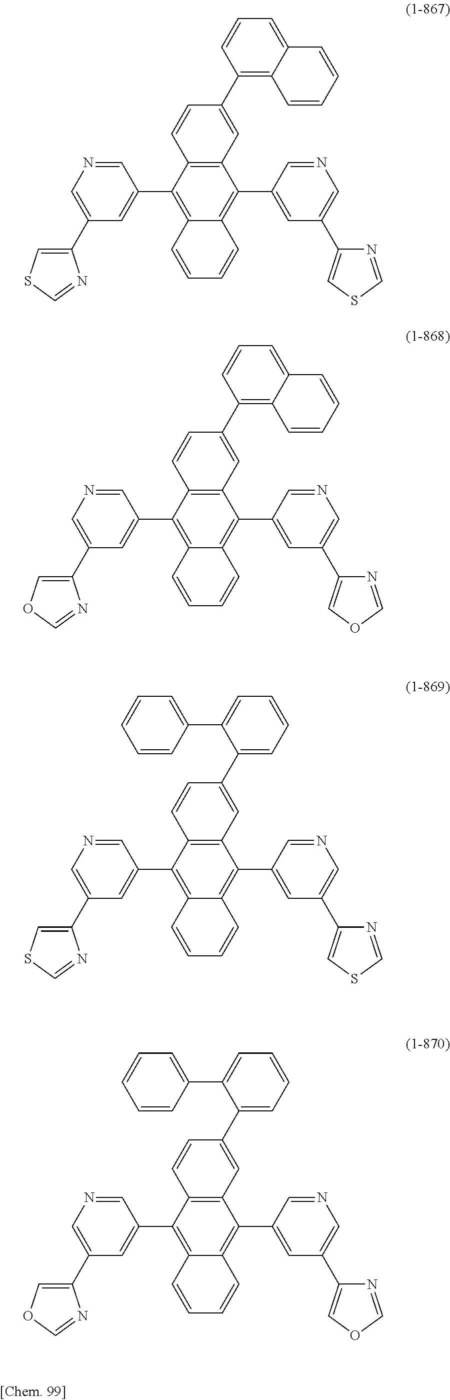 [SMILES: c1ccc2c(-c3ccc4c(-c5cncc(-c6cscn6)c5)c5ccccc5c(-c5cncc(-c6cscn6)c5)c4c3)cccc2c1 ; c1ccc2c(-c3ccc4c(-c5cncc(-c6cocn6)c5)c5ccccc5c(-c5cncc(-c6cocn6)c5)c4c3)cccc2c1 ; c1ccc(-c2ccccc2-c2ccc3c(-c4cncc(-c5cscn5)c4)c4ccccc4c(-c4cncc(-c5cscn5)c4)c3c2)cc1 ; c1ccc(-c2ccccc2-c2ccc3c(-c4cncc(-c5cocn5)c4)c4ccccc4c(-c4cncc(-c5cocn5)c4)c3c2)cc1]

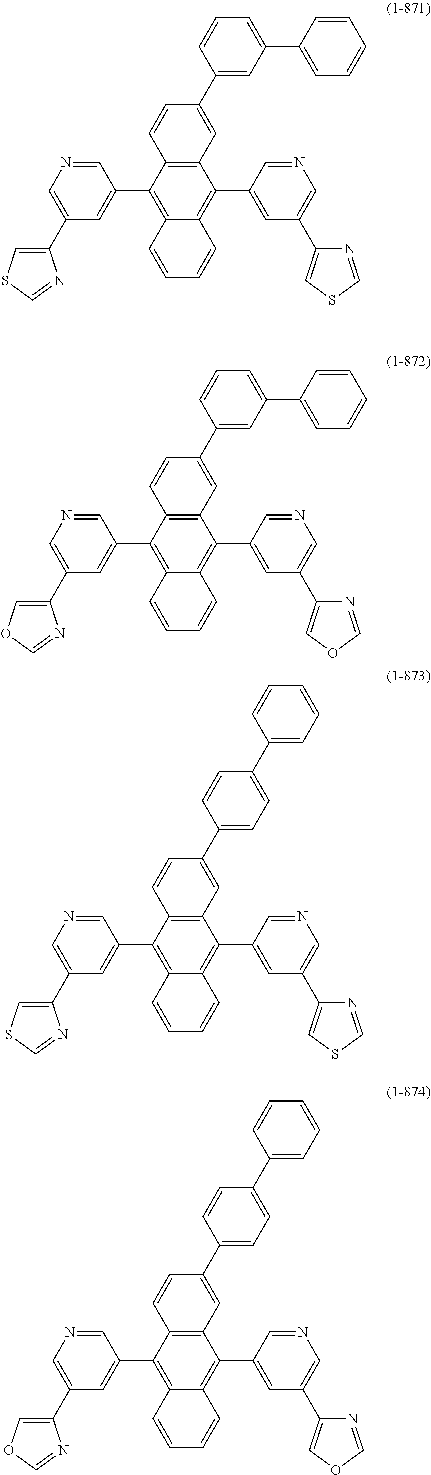

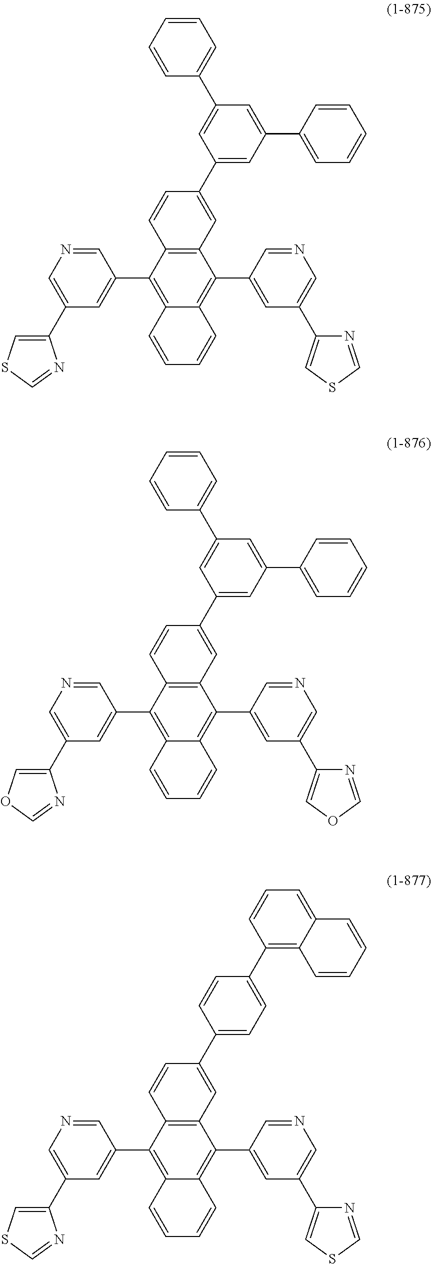 [SMILES: c1ccc(-c2cc(-c3ccccc3)cc(-c3ccc4c(-c5cncc(-c6cscn6)c5)c5ccccc5c(-c5cncc(-c6cscn6)c5)c4c3)c2)cc1 ; c1ccc(-c2cc(-c3ccccc3)cc(-c3ccc4c(-c5cncc(-c6cocn6)c5)c5ccccc5c(-c5cncc(-c6cocn6)c5)c4c3)c2)cc1 ; c1ccc2c(-c3ccc(-c4ccc5c(-c6cncc(-c7cscn7)c6)c6ccccc6c(-c6cncc(-c7cscn7)c6)c5c4)cc3)cccc2c1]

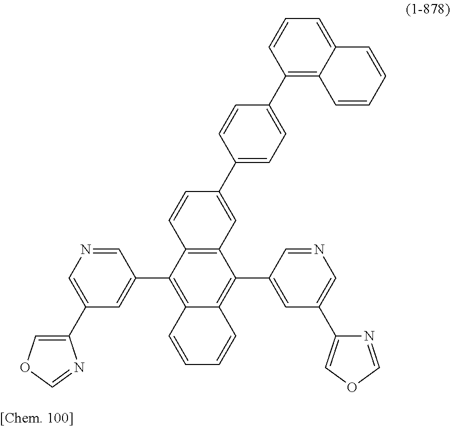 [SMILES: c1ccc2c(-c3ccc(-c4ccc5c(-c6cncc(-c7cocn7)c6)c6ccccc6c(-c6cncc(-c7cocn7)c6)c5c4)cc3)cccc2c1]

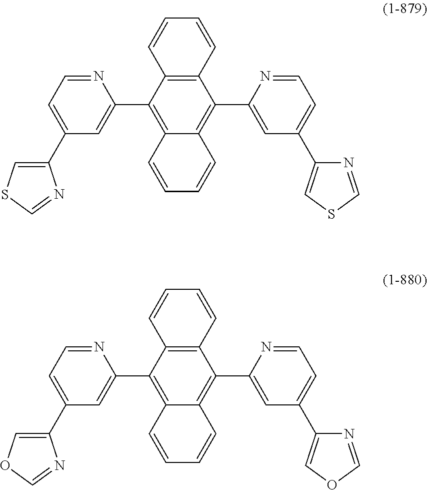 [SMILES: c1ccc2c(-c3cc(-c4cscn4)ccn3)c3ccccc3c(-c3cc(-c4cscn4)ccn3)c2c1 ; c1ccc2c(-c3cc(-c4cocn4)ccn3)c3ccccc3c(-c3cc(-c4cocn4)ccn3)c2c1]

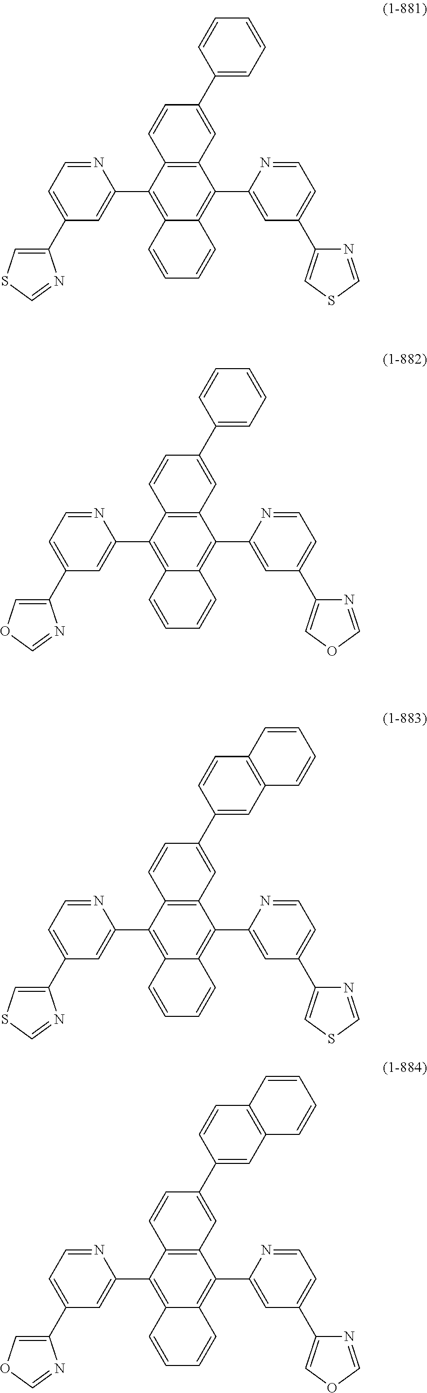 [SMILES: c1ccc(-c2ccc3c(-c4cc(-c5cscn5)ccn4)c4ccccc4c(-c4cc(-c5cscn5)ccn4)c3c2)cc1 ; c1ccc(-c2ccc3c(-c4cc(-c5cocn5)ccn4)c4ccccc4c(-c4cc(-c5cocn5)ccn4)c3c2)cc1 ; c1ccc2cc(-c3ccc4c(-c5cc(-c6cscn6)ccn5)c5ccccc5c(-c5cc(-c6cscn6)ccn5)c4c3)ccc2c1 ; c1ccc2cc(-c3ccc4c(-c5cc(-c6cocn6)ccn5)c5ccccc5c(-c5cc(-c6cocn6)ccn5)c4c3)ccc2c1]

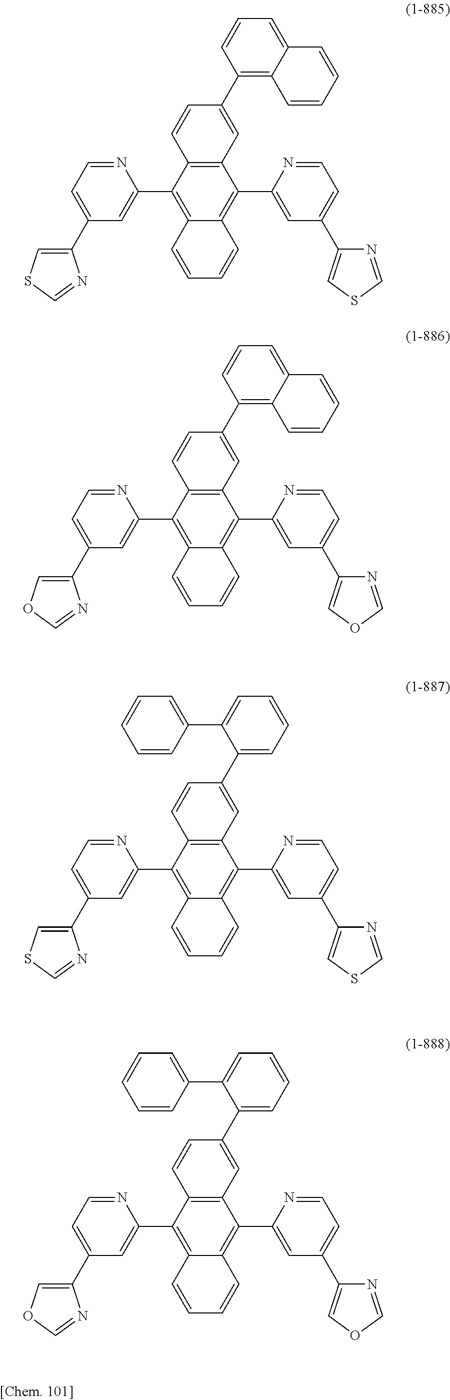 [SMILES: c1ccc2c(-c3ccc4c(-c5cc(-c6cscn6)ccn5)c5ccccc5c(-c5cc(-c6cscn6)ccn5)c4c3)cccc2c1 ; c1ccc2c(-c3ccc4c(-c5cc(-c6cocn6)ccn5)c5ccccc5c(-c5cc(-c6cocn6)ccn5)c4c3)cccc2c1 ; c1ccc(-c2ccccc2-c2ccc3c(-c4cc(-c5cscn5)ccn4)c4ccccc4c(-c4cc(-c5cscn5)ccn4)c3c2)cc1 ; c1ccc(-c2ccccc2-c2ccc3c(-c4cc(-c5cocn5)ccn4)c4ccccc4c(-c4cc(-c5cocn5)ccn4)c3c2)cc1]

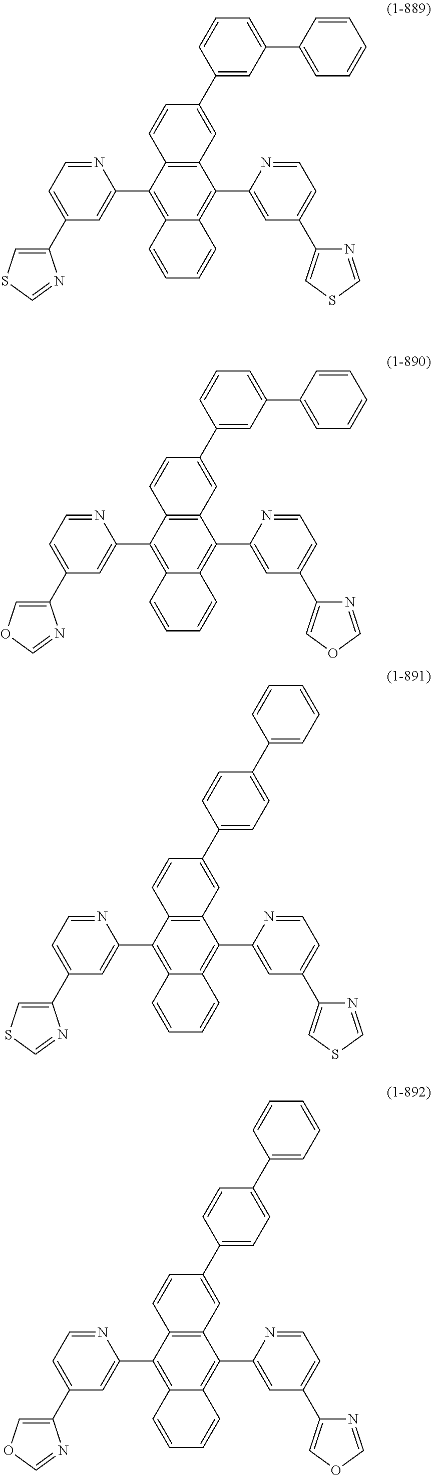

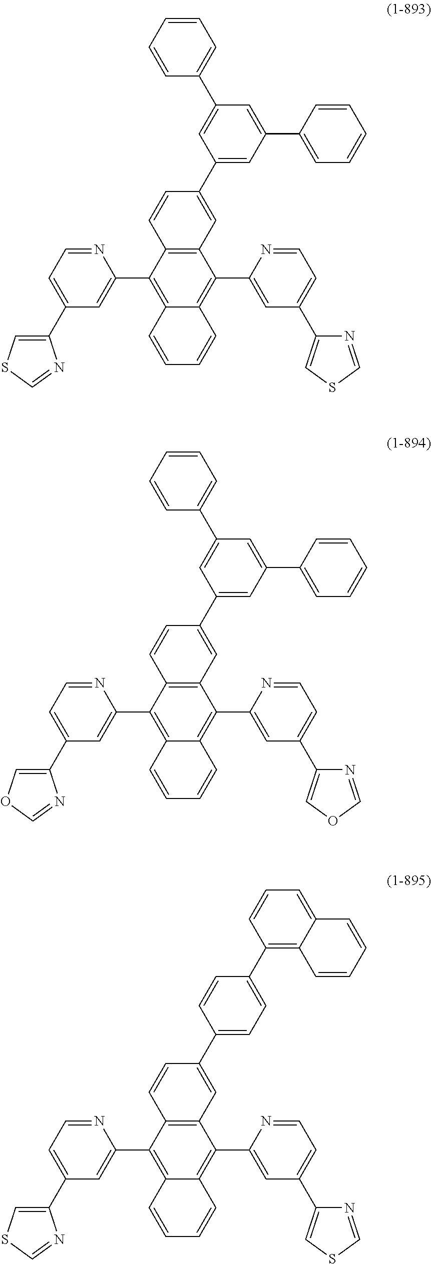 [SMILES: c1ccc(-c2cc(-c3ccccc3)cc(-c3ccc4c(-c5cc(-c6cscn6)ccn5)c5ccccc5c(-c5cc(-c6cscn6)ccn5)c4c3)c2)cc1 ; c1ccc(-c2cc(-c3ccccc3)cc(-c3ccc4c(-c5cc(-c6cocn6)ccn5)c5ccccc5c(-c5cc(-c6cocn6)ccn5)c4c3)c2)cc1 ; c1ccc2c(-c3ccc(-c4ccc5c(-c6cc(-c7cscn7)ccn6)c6ccccc6c(-c6cc(-c7cscn7)ccn6)c5c4)cc3)cccc2c1]

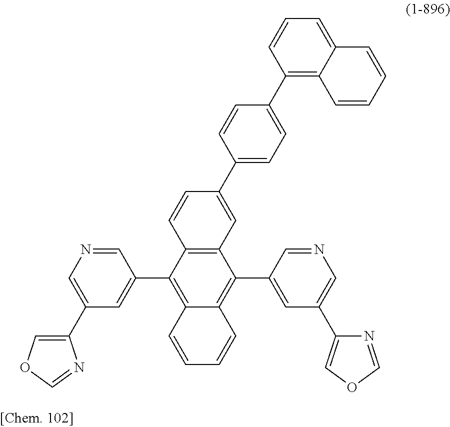 [SMILES: c1ccc2c(-c3ccc(-c4ccc5c(-c6cncc(-c7cocn7)c6)c6ccccc6c(-c6cncc(-c7cocn7)c6)c5c4)cc3)cccc2c1]

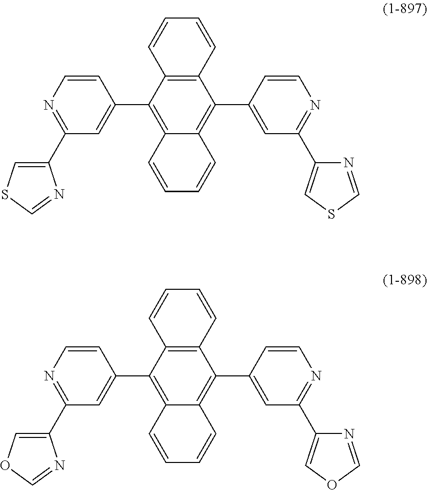 [SMILES: c1ccc2c(-c3ccnc(-c4cscn4)c3)c3ccccc3c(-c3ccnc(-c4cscn4)c3)c2c1 ; c1ccc2c(-c3ccnc(-c4cocn4)c3)c3ccccc3c(-c3ccnc(-c4cocn4)c3)c2c1]

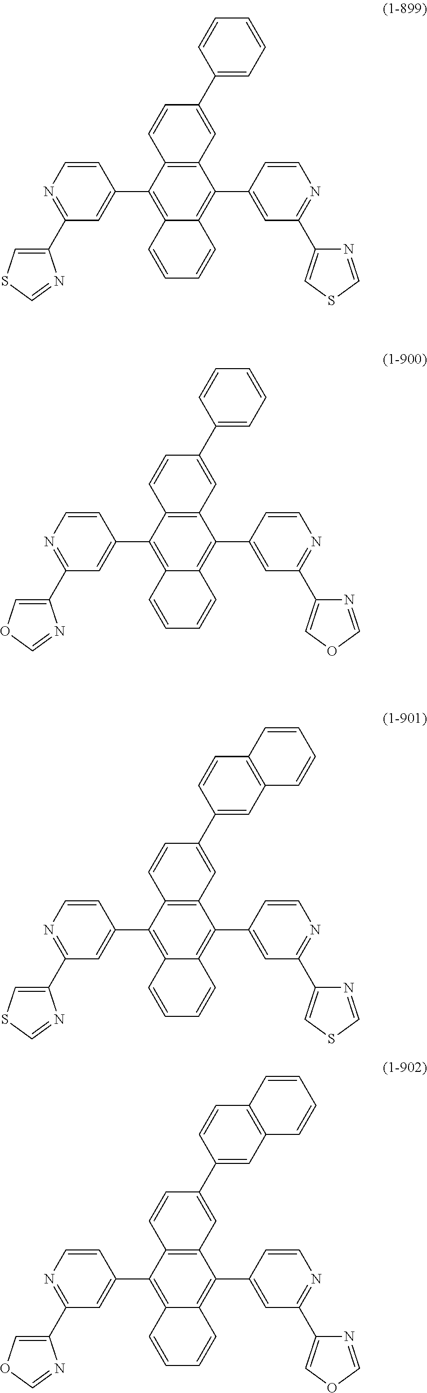 [SMILES: c1ccc(-c2ccc3c(-c4ccnc(-c5cscn5)c4)c4ccccc4c(-c4ccnc(-c5cscn5)c4)c3c2)cc1 ; c1ccc(-c2ccc3c(-c4ccnc(-c5cocn5)c4)c4ccccc4c(-c4ccnc(-c5cocn5)c4)c3c2)cc1 ; c1ccc2cc(-c3ccc4c(-c5ccnc(-c6cscn6)c5)c5ccccc5c(-c5ccnc(-c6cscn6)c5)c4c3)ccc2c1 ; c1ccc2cc(-c3ccc4c(-c5ccnc(-c6cocn6)c5)c5ccccc5c(-c5ccnc(-c6cocn6)c5)c4c3)ccc2c1]

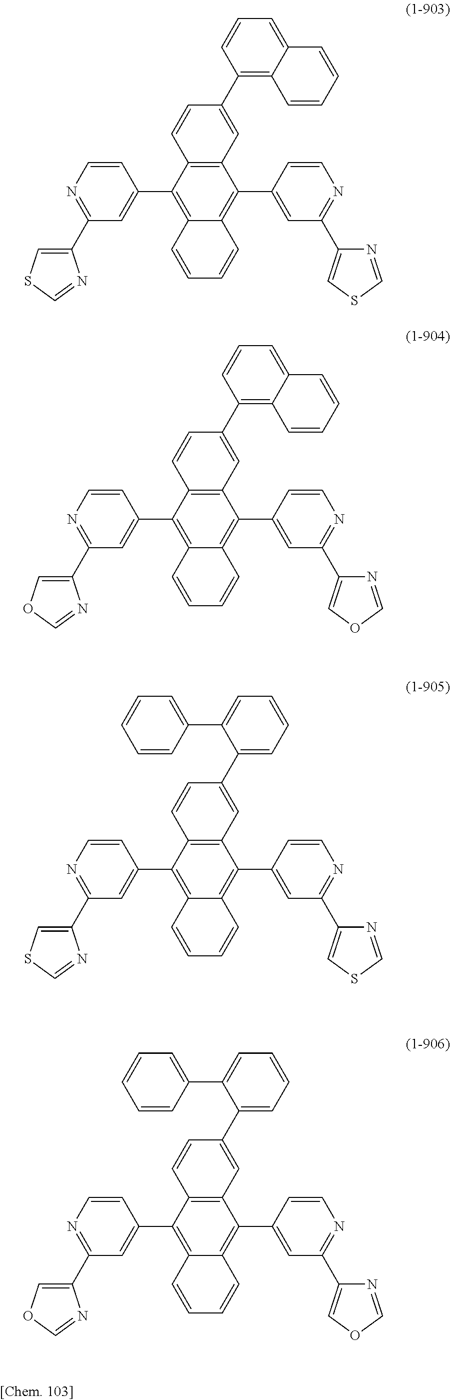 [SMILES: c1ccc2c(-c3ccc4c(-c5ccnc(-c6cscn6)c5)c5ccccc5c(-c5ccnc(-c6cscn6)c5)c4c3)cccc2c1 ; c1ccc2c(-c3ccc4c(-c5ccnc(-c6cocn6)c5)c5ccccc5c(-c5ccnc(-c6cocn6)c5)c4c3)cccc2c1 ; c1ccc(-c2ccccc2-c2ccc3c(-c4ccnc(-c5cscn5)c4)c4ccccc4c(-c4ccnc(-c5cscn5)c4)c3c2)cc1 ; c1ccc(-c2ccccc2-c2ccc3c(-c4ccnc(-c5cocn5)c4)c4ccccc4c(-c4ccnc(-c5cocn5)c4)c3c2)cc1]

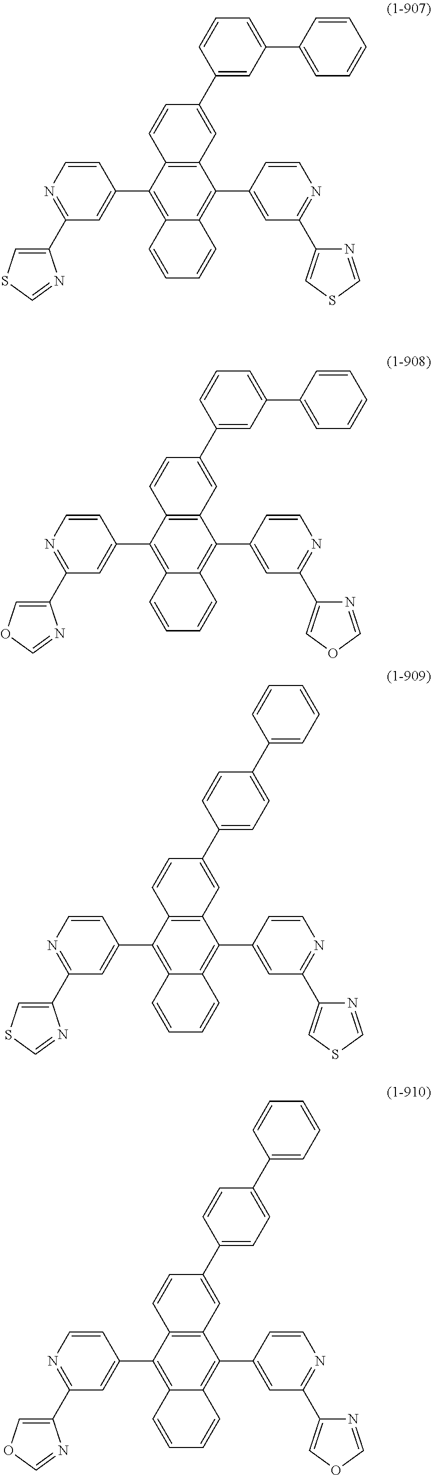

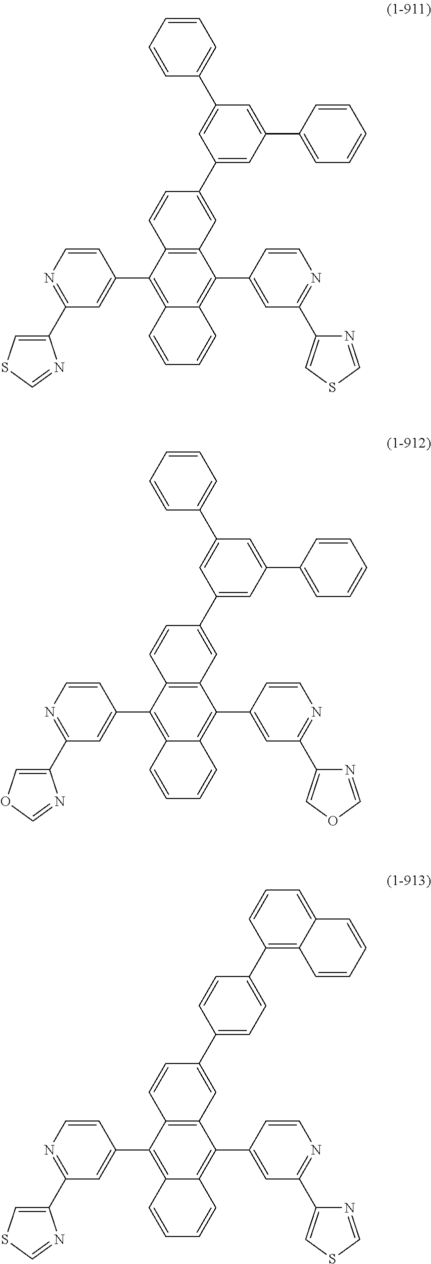 [SMILES: c1ccc(-c2cc(-c3ccccc3)cc(-c3ccc4c(-c5ccnc(-c6cscn6)c5)c5ccccc5c(-c5ccnc(-c6cscn6)c5)c4c3)c2)cc1 ; c1ccc(-c2cc(-c3ccccc3)cc(-c3ccc4c(-c5ccnc(-c6cocn6)c5)c5ccccc5c(-c5ccnc(-c6cocn6)c5)c4c3)c2)cc1 ; c1ccc2c(-c3ccc(-c4ccc5c(-c6ccnc(-c7cscn7)c6)c6ccccc6c(-c6ccnc(-c7cscn7)c6)c5c4)cc3)cccc2c1]

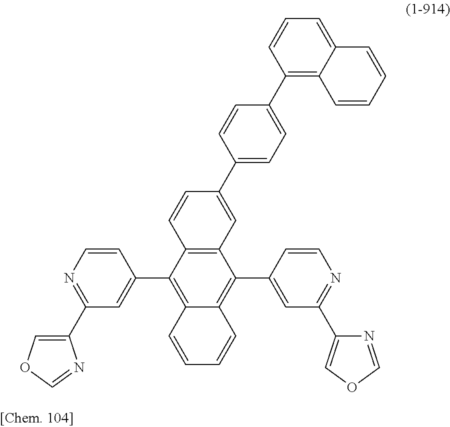 [SMILES: c1ccc2c(-c3ccc(-c4ccc5c(-c6ccnc(-c7cocn7)c6)c6ccccc6c(-c6ccnc(-c7cocn7)c6)c5c4)cc3)cccc2c1]

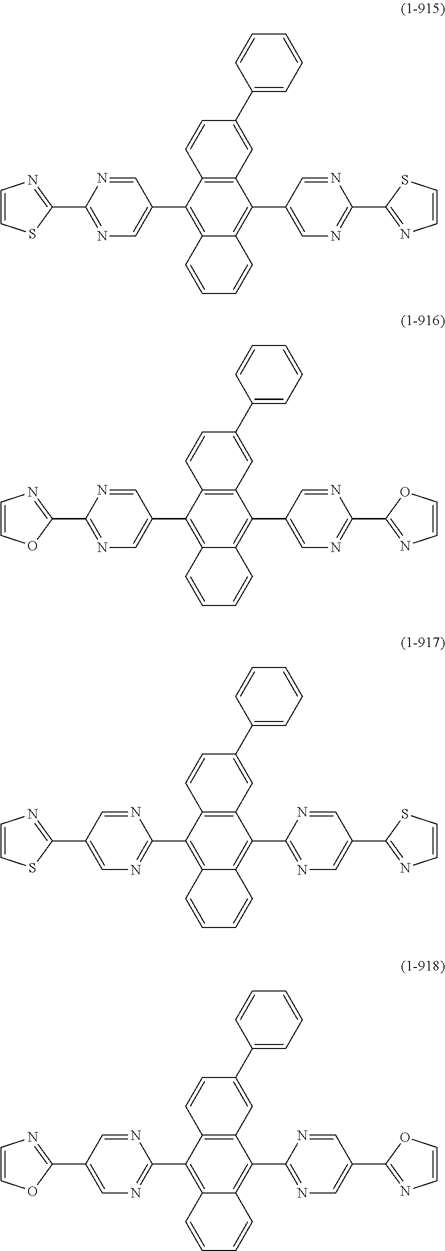 [SMILES: c1ccc(-c2ccc3c(-c4cnc(-c5nccs5)nc4)c4ccccc4c(-c4cnc(-c5nccs5)nc4)c3c2)cc1 ; c1ccc(-c2ccc3c(-c4cnc(-c5ncco5)nc4)c4ccccc4c(-c4cnc(-c5ncco5)nc4)c3c2)cc1 ; c1ccc(-c2ccc3c(-c4ncc(-c5nccs5)cn4)c4ccccc4c(-c4ncc(-c5nccs5)cn4)c3c2)cc1 ; c1ccc(-c2ccc3c(-c4ncc(-c5ncco5)cn4)c4ccccc4c(-c4ncc(-c5ncco5)cn4)c3c2)cc1]

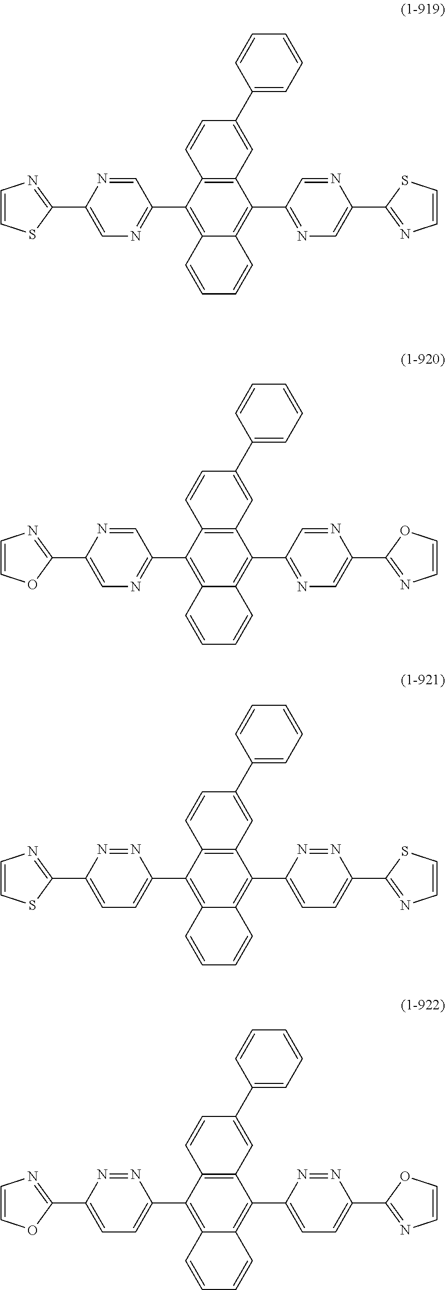 [SMILES: c1ccc(-c2ccc3c(-c4cnc(-c5nccs5)cn4)c4ccccc4c(-c4cnc(-c5nccs5)cn4)c3c2)cc1 ; c1ccc(-c2ccc3c(-c4cnc(-c5ncco5)cn4)c4ccccc4c(-c4cnc(-c5ncco5)cn4)c3c2)cc1 ; c1ccc(-c2ccc3c(-c4ccc(-c5nccs5)nn4)c4ccccc4c(-c4ccc(-c5nccs5)nn4)c3c2)cc1 ; c1ccc(-c2ccc3c(-c4ccc(-c5ncco5)nn4)c4ccccc4c(-c4ccc(-c5ncco5)nn4)c3c2)cc1]

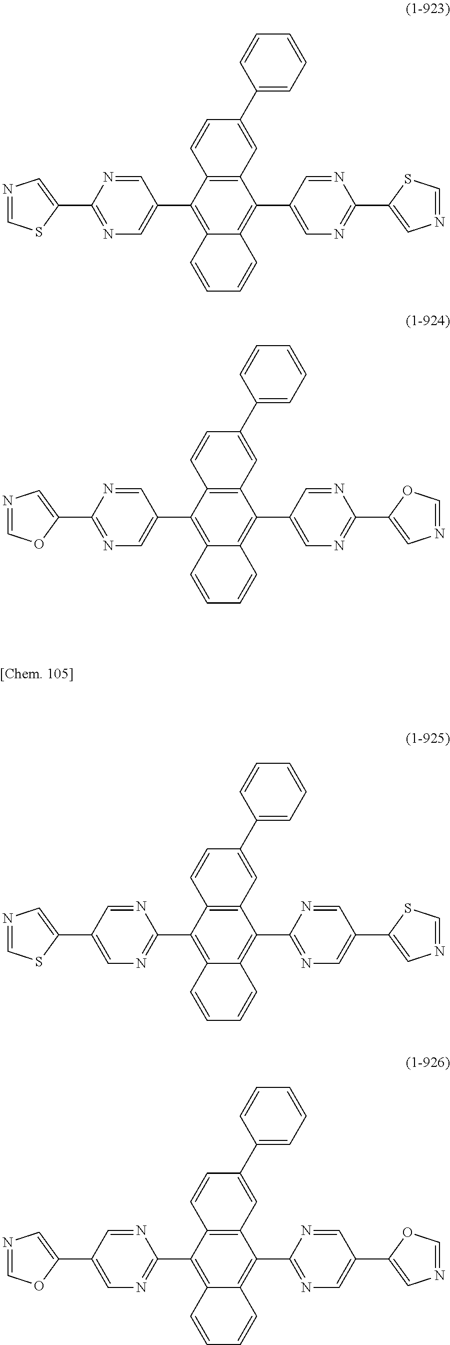 [SMILES: c1ccc(-c2ccc3c(-c4cnc(-c5cncs5)nc4)c4ccccc4c(-c4cnc(-c5cncs5)nc4)c3c2)cc1 ; c1ccc(-c2ccc3c(-c4cnc(-c5cnco5)nc4)c4ccccc4c(-c4cnc(-c5cnco5)nc4)c3c2)cc1 ; c1ccc(-c2ccc3c(-c4ncc(-c5cncs5)cn4)c4ccccc4c(-c4ncc(-c5cncs5)cn4)c3c2)cc1 ; c1ccc(-c2ccc3c(-c4ncc(-c5cnco5)cn4)c4ccccc4c(-c4ncc(-c5cnco5)cn4)c3c2)cc1]

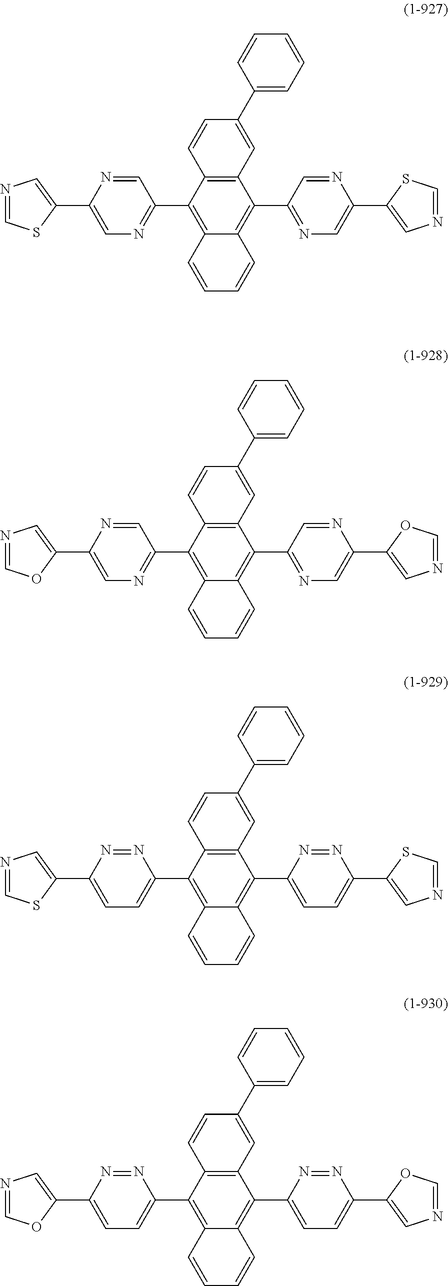 [SMILES: c1ccc(-c2ccc3c(-c4cnc(-c5cncs5)cn4)c4ccccc4c(-c4cnc(-c5cncs5)cn4)c3c2)cc1 ; c1ccc(-c2ccc3c(-c4cnc(-c5cnco5)cn4)c4ccccc4c(-c4cnc(-c5cnco5)cn4)c3c2)cc1 ; c1ccc(-c2ccc3c(-c4ccc(-c5cncs5)nn4)c4ccccc4c(-c4ccc(-c5cncs5)nn4)c3c2)cc1 ; c1ccc(-c2ccc3c(-c4ccc(-c5cnco5)nn4)c4ccccc4c(-c4ccc(-c5cnco5)nn4)c3c2)cc1]

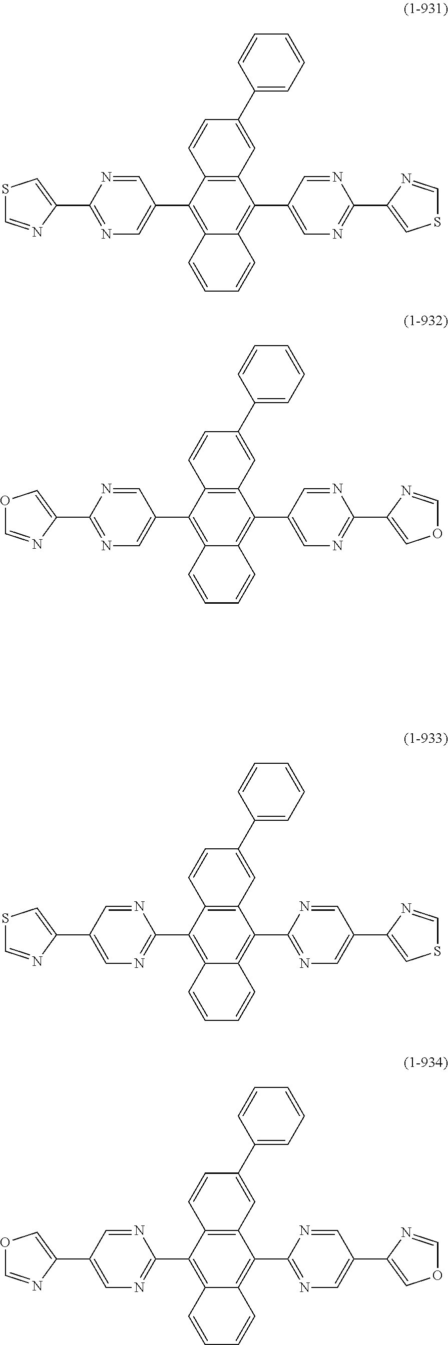 [SMILES: c1ccc(-c2ccc3c(-c4cnc(-c5cscn5)nc4)c4ccccc4c(-c4cnc(-c5cscn5)nc4)c3c2)cc1 ; c1ccc(-c2ccc3c(-c4cnc(-c5cocn5)nc4)c4ccccc4c(-c4cnc(-c5cocn5)nc4)c3c2)cc1 ; c1ccc(-c2ccc3c(-c4ncc(-c5cscn5)cn4)c4ccccc4c(-c4ncc(-c5cscn5)cn4)c3c2)cc1 ; c1ccc(-c2ccc3c(-c4ncc(-c5cocn5)cn4)c4ccccc4c(-c4ncc(-c5cocn5)cn4)c3c2)cc1]

### Synthetic Method of Compound

Next, a method of manufacturing the compound according to the invention will be described. The compound of the invention can be basically prepared by using a known compound according to a known method, for example a Suzuki coupling reaction or Negishi coupling reaction (described in “Metal-Catalyzed Cross-Coupling Reactions—/sec, Completely Revised and Enlarged Edition,” for example). Moreover, the compound can be prepared by combining both of the reactions. An example of a scheme for synthesizing the compound represented by formula (1) by the Suzuki coupling reaction or Negishi coupling reaction is shown below.

When the compound of the invention is manufactured, specific examples include (1) a method of preparing a group formed by binding thiazolyl or oxazolyl with a benzene ring, a pyridine ring, a pyrimidine ring, a pyrazine ring, a pyridazine ring or a triazole ring (hereinafter, the group is generically expressed as “site formed of thiazole or oxazole derivative” in several cases) to bind the formed groups with various kinds of aromatic hydrocarbon or aromatic heterocycles, and (2) a method of binding a benzene ring, a pyridine ring, a pyrimidine ring, a pyrazine ring, a pyridazine ring or a triazole ring with aromatic hydrocarbon or an aromatic heterocycle, and then binding thiazolyl or oxazolyl therewith. Moreover, for each bond in the above methods, a coupling reaction between a halogen functional group or a trifluoromethanesulfonate functional group and a zinc chloride complex or boronic acid or boronic acid ester can also basically used. Furthermore, when arylene has an anthracene skeleton, for example, a reaction can be applied in which anthraquinone is allowed to react with a lithium or magnesium reagent having a benzene ring, a pyridine ring, a pyrimidine ring, a pyrazine ring, a pyridazine ring or a triazole ring to be converted into a diol form, and then the diol form is aromatized.

(1) Method of Binding “Site Formed of Thiazole or Oxazole Derivative” with Aromatic Hydrocarbon or Aromatic Heterocycle Synthesis of Phenylthiazole or Phenyloxazole Having Reactive Substituent

Then, 2-(4-bromophenyl)thiazole can be prepared by, first, preparing a zinc chloride complex of thiazole according to scheme (1) below, and then allowing the zinc chloride complex of thiazole to react with p-dibromobenzene according to scheme (2) below. In addition, “ZnCl₂.TMEDA” in scheme (1) represents a tetramethylethylenediamine complex of zinc chloride. In “R′Li” or “R′MgX” in scheme (1), R′ represents a straight-chain or branched-chain alkyl group, and such alkyl is preferably a straight-chain alkyl group having 1 to 4 carbons or a branched-chain alkyl group having 3 to 4 carbons, and X is halogen.

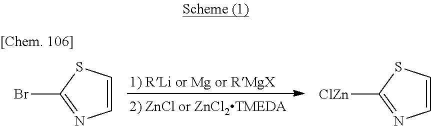

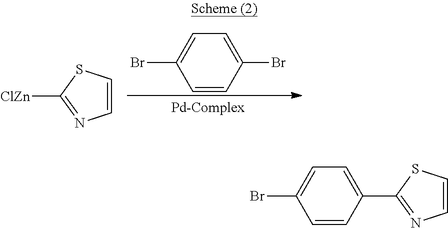

An example of a synthetic method using 2-bromothiazole as a raw material of a thiazolyl group is shown herein. However, an object corresponding to each can be prepared by using 4-bromothiazole, 5-bromothiazole or 2-, 4-, 5-oxazole is used as a raw material.

Moreover, an example of a synthesis method using p-dibromobenzene is shown herein. However, a corresponding object can be obtained by using as a raw material m-dibromobenzene, 2,6-dibromopyridine, 2,5-dibromopyridine, 3,5-dibromopyridine, 2,4-dibromopyrimidine, 2,5-dibromopyrimidine, 4,6-dibromopyrimidine, 2,6-dibromopyrazine, 2,5-dibromopyrazine, 3,6-dibromopyridazine or the like, or further using, in place of a diboromo form, a dichloro form such as 2,4-dichlorotriazine or a diiodo form or bis(trifluoromethanesulfonate) or a mixture thereof (for example: 1-bromo-4-iodobenzene). Moreover, an object can also be obtained by allowing a benzene or pyridine derivative having as a substituent a halogen atom and alkoxy group as in bromoanisole to react with a zinc chloride complex of thiazole or oxazole, and passing through demethylation using boron tribromide or pyridine hydrochloride and subsequently esterification of trifluoromethanesulfonic acid.

Further, an example of a synthesis method in the case of non-substitution is shown herein. However, an object having a substituent can be obtained by using a raw material having the substituent in a desired position.

Moreover, the object can also be obtained by a coupling reaction for allowing p-dibromobenzene to react with boronic acid of thiazole or oxazole, or boronic acid ester of thiazole or oxazole, in place of the zinc chloride complex of thiazole or oxazole.

Method of Converting Reactive Substituent into Boronic Acid or Boronic Acid Ester

According to scheme (3) below, 4-(2-thiazolyl)phenylboronic acid ester can be prepared by lithiating 2-(4-bromophenyl)thiazole using an organolithium reagent, or converting 2-(4-bromophenyl)thiazole into a Grignard reagent using magnesium or an organic magnesium reagent and allowing the resulting material with trimethyl borate, triethyl borate or triisopropyl borate. Further, according to scheme (4) below, 4-(2-thiazolyl)phenylboronic acid can be prepared by hydrolyzing 4-(2-thiazolyl)phenylboronic acid ester. In “R′Li” or “R′MgX” in scheme (3), R′ represents a straight-chain or branched-chain alkyl group, and such alkyl is preferably a straight-chain alkyl group having 1 to 4 carbons or a branched-chain alkyl group having 3 to 4 carbons, and X is halogen.

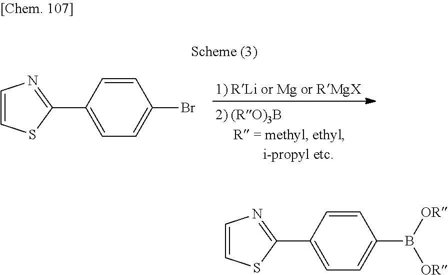

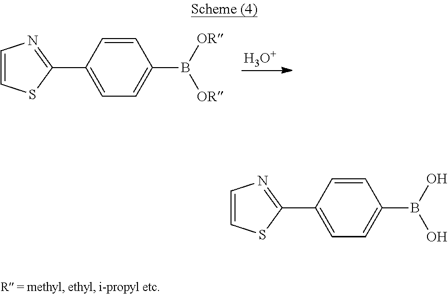

Moreover, according to scheme (5) below, similar 4-(2-thiazolyl)phenylboronic acid ester can be prepared by allowing a coupling reaction between 2-(4-bromophenyl)thiazole and bis(pinacolate)diboron or 4,4,5,5-tetramethyl-1,3,2-dioxaborolane in the presence of a palladium catalyst and a base. In “R′Li” and “R′MgX” in scheme (5), R′ represents a straight-chain or branched-chain alkyl group, and such alkyl is preferably a straight-chain alkyl group having 1 to 4 carbons or a branched-chain having 3 to 4 carbons, and X is halogen.

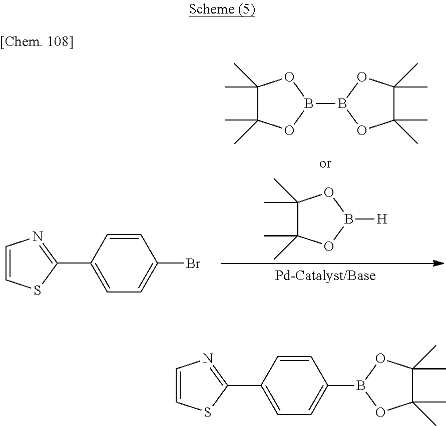

In addition, in scheme (3) or (5) above, corresponding boronic acid or boronic acid ester can also be prepared by using any other position isomer in place of 2-(4-bromophenyl)thiazole, or an oxazole ring in place of a thiazole ring. Furthermore, even if a bromopyridyl group, a bromopyrimidinyl group, a bromopyrazinyl group, a bromopyridazinyl group or a bromotriazinyl group is substituted for a bromophenyl group, the boronic acid or boronic acid ester can also be prepared in a similar manner. Moreover, even if chloride or iodide is used in place of bromide in scheme (3), or chloride, iodide or trifluoromethanesulfonate is used in place of bromide in scheme (6), the boronic acid or boronic acid ester can also be prepared in a similar manner.

### Synthesis of Anthracene Having Reactive Substituent 9,10-Dibromoanthracene

As shown in scheme (6) below, 9,10-dibromoanthracene is obtained by brominating anthracene with a suitable brominating agent. Specific examples of the suitable brominating agent include bromine or N-bromosuccinimide (NBS).

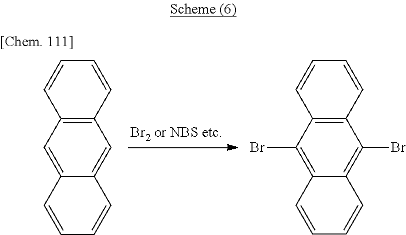

Then, when an anthracene derivative having a substituent (alkyl, cycloalkyl, aryl or the like) in 2-position is desired, the anthracene derivative having the substituent in 2-position can be prepared according to Suzuki coupling between anthracene 2-position of which is replaced by halogen or triflate, and boronic acid corresponding to the substituent. Moreover, specific examples of another method include a synthetic method according to Negishi coupling between anthracene 2-position of which is replaced by halogen or triflate, and a zinc complex corresponding to the substituent. Further, specific examples also include a synthetic method according to Suzuki coupling between 2-anthracene boronic acid (or boronic acid ester), and a group corresponding to the substituent in which replacement by halogen or triflate is made, and also a synthetic method according to Negishi coupling between a 2-anthracene zinc complex, and a group corresponding to the substituent in which replacement by halogen or triflate is made. In addition, an anthracene derivative having a substituent in a position other than 2-position can also be prepared in a similar manner by using a raw material prepared by placing in a desired position a position of halogen, triflate, boronic acid (or boronic acid ester) or a zinc complex in which anthracene is replaced thereby.

### 9,10-Dianthracene-Zinc Complex

As shown in scheme (7) below, a 9,10-dianthracene-zinc complex can be prepared by lithiating 9,10-dibromoanthracene using an organolithium reagent, or converting 9,10-dibromoanthracene into a Grignard reagent using magnesium or an organic magnesium reagent, and allowing the resulting material to react with zinc chloride or a zinc chloride-tetramethylethylenediamine complex (ZnCl₂.TMEDA). In scheme (7), R′ represents a straight-chain or branched-chain alkyl group, and such alkyl is preferably a straight-chain alkyl group having 1 to 4 carbons or a branched-chain alkyl group having 3 to 4 carbons. In addition, the 9,10-dianthracene-zinc complex can also be prepared in a similar manner by using chloride or iodide in place of bromide such as 9,10-dibromoanthracene.

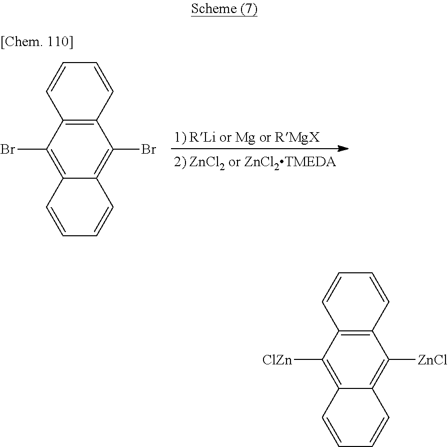

### 9,10-Anthracene Diboronic Acid (or Boronic Acid Ester)

As shown in scheme (8) below, 9,10-anthracene diboronic acid ester can be prepared by lithiating 9,10-dibromoanthracene using an organolithium reagent, or converting 9,10-dibromoanthracene into Grignard reagent using magnesium or an organic magnesium reagent, and allowing the resulting material to react with trimethyl borate, triethyl borate or triisopropyl borate. Further, in scheme (9) below, 9,10-anthracene diboronic acid can be prepared by hydrolyzing 9,10-anthracene diboronic acid ester. In scheme (8), R′ represents a straight-chain or branched-chain alkyl group, and such alkyl is preferably a straight-chain alkyl group having 1 to 4 carbons or a branched-chain alkyl group having 3 to 4 carbons.

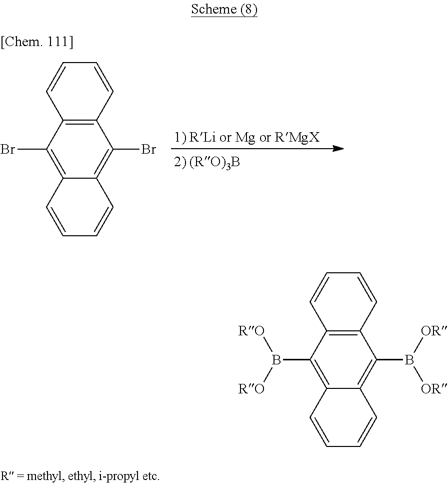

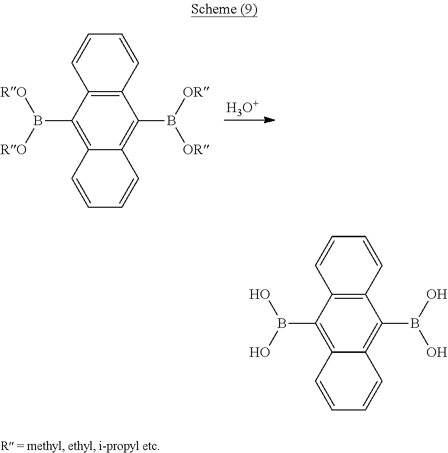

Moreover, as shown in scheme (10) below, similar 9,10-anthracene diboronic acid ester can be prepared by allowing a coupling reaction between 9,10-dibromoanthracene and bis(pinacolate)diboron or 4,4,5,5-tetramethyl-1,3,2-dioxaborolane by using a palladium catalyst and a base.

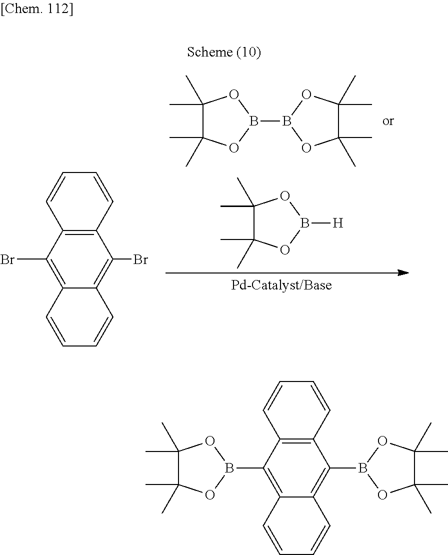

In addition, 9,10-anthracene diboronic acid ester can be prepared in a similar manner also by using chloride or iodide in place of bromide such as 9,10-dibromoanthracene in scheme (8) or (10) above, by using chloride, iodide or triflate in place of bromide in scheme (10) above.

Specific examples of the aromatic hydrocarbon or aromatic heterocycle to be bonded with “site formed of thiazole or oxazole derivative” herein include the anthracene derivative having the reactive substituent. However, aromatic hydrocarbon or an aromatic heterocycle having various kinds of reactive substituents can be obtained by using as a raw material aromatic hydrocarbon or an aromatic heterocycle having halogen or triflate in 2 to 4 places. Moreover, a substituent can be appropriately introduced into the aromatic hydrocarbon or the aromatic heterocycle having various kinds of reactive substituents by using a raw material having a substituent in a desired position.

Method of Binding Anthracene Having Reactive Substituent with “Site Formed of Thiazole or Oxazole Derivative”

As described above, with regard to “site formed of thiazole or oxazole derivative,” the bromo form (schemes (1) to (2)), boronic acid and boronic acid ester (schemes (3) to (5)) can be prepared, and with regard to anthracene having the reactive substituent, the bromo form (scheme (6)), the zinc chloride complex (scheme (7)), boronic acid and boronic acid ester (schemes (8) to (10)) can be prepared. Therefore, referring to the coupling reaction applied in the description so far, the thiazole derivative or oxazole derivative according to the invention can be prepared by binding “site formed of thiazole or oxazole derivative” with anthracene.

In the final coupling reaction, in order to form two or more of “site formed of thiazole or oxazole derivative” in the compound represented by formula (1) into different structure, first, anthracene having a reactive substituent is allowed to react with a compound having “site formed of thiazole or oxazole derivative” in an amount corresponding to one-fold mole, and then the resulting intermediate is allowed to react with a compound having “site formed of thiazole or oxazole derivative” different from the compound previously used (namely, the reaction is allowed in two or more steps).

(2) Method of Binding Benzene Ring, Pyridine Ring, Pyrimidine Ring, Pyrazine Ring, Pyridazine Ring or Triazole Ring with Arylene or Heteroarylene, and then Binding Thiazolyl or Oxazolyl Group with the Resulting Material

Also with regard to the above method, referring to various coupling reactions described above, a benzene ring, a pyridine ring, a pyrimidine ring, a pyrazine ring, a pyridazine ring or a triazole ring may be bonded with arylene or heteroarylene in two or more places, and then thiazolyl or oxazolyl may be bonded therewith. On the above occasion, in order to form two of “site formed of thiazole or oxazole derivative” in the compound represented by formula (1) into different structure, a desired derivative can be prepared by binding different kinds in a two-stage reaction in a stage of binding a benzene ring, a pyridine ring, a pyrimidine ring, a pyrazine ring, a pyridazine ring or a triazole ring with arylene or heteroarylene, or bonding different thiazolyl or oxazolyl in a two-stage reaction in a stage of bonding thiazolyl or oxazolyl.

### Reagent Used in Reaction

Specific examples of the palladium catalyst used in the coupling reaction include tetrakis(triphenylphosphine)Palladium(0): Pd(PPh₃)₄, bis(triphenylphosphine)palladium(II) dichloride: PdCl₂(PPh₃)₂, palladium(II) acetate: Pd(OAc)₂, tris(dibenzylideneacetone)dipalladium(0): Pd₂ (dba) a tris(dibenzylideneacetone)dipalladium(0) chloroform complex: Pd₂(dba)₃.CHCl₃, bis(dibenzylideneacetone)palladium(0): Pd(dba)₂, bis(tri-t-butylphosphino)palladium(0): Pd(t-Bu₃P)₂, [1,1′-bis(diphenylphosphino)ferrocene]palladium(II) dichloride: Pd(dppf)Cl₂, [1,1′-bis(diphenylphosphino)ferrocene]palladium(II) dichloride dichloromethane complex (1:1): Pd(dppf)Cl₂.CH₂Cl₂ or PdCl₂[P(t-Bu)₂-(p-NMe₂-Ph)]₂: (A-^{ta}Phos)₂PdCl₂(Pd-132: trademark; Johnson Matthey Catalysis and Chiral Technologies).

Moreover, in order to promote the reaction, a phosphine compound may be added to the palladium compounds in several cases. Specific examples of the phosphine compound include tri(t-butyl)phosphine, tricyclohexylphosphine, 1-(N,N-dimethylaminomethyl)-2-(di-t-butylphosphino)ferrocene, 1-(N,N-dibutylaminomethyl)-2-(di-t-butylphosphino)ferrocene, 1-(methoxymethyl)-2-(di-t-butylphosphino)ferrocene, 1,1′-bis(di-t-butylphosphino)ferrocene, 2,2′-bis(di-t-butylphosphino)-1,1′-binaphtyl, 2-methoxy-2′-(di-t-butylphosphino)-1,1′-binaphtyl or 2-dicyclohexylphosphino-2′,6′-dimethoxybiphenyl.

Specific examples of the base used in the reaction include sodium carbonate, potassium carbonate, cesium carbonate, sodium hydrogencarbonate, sodium hydroxide, potassium hydroxide, barium hydroxide, sodium ethoxide, sodium t-butoxide, sodium acetate, potassium acetate, tripotassium phosphate or potassium fluoride.

Moreover, specific examples of a solvent used in the reaction include benzene, toluene, xylene, 1,2,4-trimethylbenzene, N,N-dimethylformamide, tetrahydrofuran, diethyl ether, t-butylmethyl ether, 1,4-dioxane, methanol, ethanol, cyclopentyl methyl ether or isopropyl alcohol. The solvent can be appropriately selected, and may be used alone or as a mixed solvent. Moreover, at least one solvent and water can also be mixed and used.

When the compound of the invention is used in an electron injection layer or an electron transport layer in an organic EL device, the organic EL device is stable during applying an electric field. Such performance represents that the compound of the invention is excellent an electron injection material or an electron transport material of an electroluminescent device. The electron injection layer herein means a layer for receiving an electron from a cathode to an organic layer, and the electron transport layer means a layer for transporting an injected electron to an emission layer. Moreover, the electron transport layer can simultaneously serve as the electron injection layer. A material used in each layer is referred to as the electron injection material and the electron transport material, respectively.

### Description of Organic EL Device

A second aspect of the invention refers to an organic EL device including the compound represented by formula (1) according to the invention in an electron injection layer or electron transport layer. In the organic EL device of the invention, driving voltage is low, and durability during driving is high.

A structure of the organic EL device according to the invention includes various aspects, and is basically a multilayer structure in which at least a hole transport layer, an emission layer and the electron transport layer are interposed between an anode and a cathode. Specific examples of structure of the device include (1) anode/hole transport layer/emission layer/electron transport layer/cathode, (2) anode/hole injection layer/hole transport layer/emission layer/electron transport layer/cathode, and (3) anode/hole injection layer/hole transport layer/emission layer/electron transport layer/electron injection layer/cathode.

The compound of the invention has high electron injection properties and high electron transport properties. Therefore, the compound can be used in the electron injection layer or electron transport layer in the form of a simple substance or in combination with other materials. According to the organic EL device of the invention, blue, green, red or white emission can be obtained by combining the hole injection layer, the hole transport layer, the emission layer, and so forth in which other materials are used in the electron transport material according to the invention.

An emission material or emissive dopant that can be used in the organic EL device according to the invention is an emission material such as a daylight fluorescent material, a fluorescent whitening agent, a laser coloring matter, an organic scintillator, and various kinds of fluorescence analysis reagents as described in “Optical Functional Materials,” Polymer Functional Material Series, edited by the Society of Polymer Science, Japan, Kyoritsu Shuppan Co., Ltd., 1991, p. 236, a dopant material as described in “Organic EL Material and Display”, under editorship of Junji Kido, CMC Co., Ltd., 2001, pp. 155-156, an emission material of a triplet light emitting material as described in ibid., pp. 170-172, and so forth.

A compound that can be used as the emission material or the emissive dopant is a polycyclic aromatic compound, a heteroaromatic compound, an organometallic complex, a coloring matter, a polymer emission material, a styryl derivative, an aromatic amine derivative, a coumarin derivative, a borane derivative, an oxazine derivative, a compound having a spiro ring, an oxadiazole derivative, a fluorene derivative and so forth. Specific examples of the polycyclic aromatic compound includes an anthracene derivative, a phenanthrene derivative, a naphthacene derivative, a pyrene derivative, a chrysene derivative, a perylene derivative, a coronene derivative and a rubrene derivative. Specific examples of the heteroaromatic compound include an oxadiazole derivative having a dialkylamino group or diarylamino group, a pyrazoloquinoline derivative, a pyridine derivative, a pyran derivative, a phenanthroline derivative, a silole derivative, a thiophene derivative having a triphenylamino group and a quinacridone derivative. Specific examples of the organometallic complex include a complex between zinc, aluminum, beryllium, europium, terbium, dysprosium, iridium, platinum, osmium, gold or the like and a quinolinol derivative, a benzoxazole derivative, a benzothiazole derivative, an oxadiazole derivative, a thiadiazole derivative, a benzimidazole derivative, a pyrrole derivative, a pyridine derivative or a phenanthroline derivative. Specific examples of the coloring matter include a xanthene derivative, a polymethine derivative, a porphyrin derivative, a coumarin derivative, a dicyanomethylene pyran derivative, a dicyanomethylene thiopyran derivative, an oxobenzanthracene derivative, a carbostyryl derivative, a perylene derivative, a benzooxazole derivative, a benzothiazole derivative and a benzimidazole derivative. Specific examples of the polymer emission material include a polyparaphenylvinylene derivative, a polythiophene derivative, a polyvinylcarbazole derivative, a polysilane derivative, a polyfluorene derivative and a polyparaphenylene derivative. Specific examples of the styryl derivative include an amine-containing styryl derivative and styrylarylene derivative.

Other electron transport materials used in the organic EL device according to the invention can be arbitrarily selected from compounds that can be used in an electron transfer compound in a photoconductive material, or compounds that can be used in the electron transport layer and the electron injection layer of the organic EL device.

Specific examples of such an electron transport material include a quinolinol metal complex, a metal complex of a 2,2′-bipyridyl derivative, a phenanthroline derivative, a diphenylquinone derivative, a perylene derivative, an oxadiazole derivative, a thiophene derivative, a triazole derivative, a thiadiazole derivative or an oxine derivative, a polymer of quinoxaline derivative or a quinoxaline derivative, a benzazoles compound, a gallium complex, a pyrazol derivative, a perfluorated phenylene derivative, a triazine derivative, a pyrazine derivative, a benzoquinoline derivative, an imidazopyridine derivative and a borane derivative.

With regard to a hole injection material and a hole transport material used in the organic EL device according to the invention, an arbitrary material can be selected and used from compounds that have been used so far as a hole charge transport material in the photoconductive material, or publicly-known compounds used in the hole injection layer and the hole transport layer of the organic EL device. Specific examples thereof include a carbazole derivative, a triarylamine derivative and a phthalocyanine derivative.

Each layer composing the organic EL device according to the invention can be formed by processing the material composing each layer into a thin film by a method such as a vacuum evaporation method, a spin coating method or a casting method. Thickness of the thus formed each layer is not particularly limited, and can be set up appropriately according to properties of the material. The thickness is ordinarily in a range of 2 nanometers to 5,000 nanometers. In addition, as a method for processing the emission material into the thin film, a vacuum evaporation method is preferably applied from viewpoints of easiness in obtaining a uniform film, difficulty in generating pin holes, and so forth. When the material is processed into the thin film by applying the vacuum evaporation method, vacuum evaporation conditions thereof are different depending on kinds of emission materials according to the invention. In general, the vacuum evaporation conditions are preferably set up appropriately in the range of 50 to 400° C. in a boat heating temperature, 10⁻⁶ to 10⁻³ Pa in a degree of vacuum, 0.01 to 50 nm/sec in a vacuum evaporation rate, −150 to +300° C. in a substrate temperature, and 5 nanometers to 5 micrometers in thickness.

The organic EL device of the invention is preferably supported on a substrate even in any structure described above. Any substrate may be used if the substrate has mechanical strength, thermal stability and transparency, and glass, a transparent plastic film or the like can be used. As an anode material, metal, alloy, an electric conductive compound having a work function larger than 4 eV or a mixture thereof can be used. Specific examples thereof include metal such as gold, CuI, indium tin oxide (hereafter, abbreviated as ITO), SnO₂ and ZnO.

As a cathode material, metal, alloy or an electric conductive compound having a work function smaller than 4 eV, and a mixture thereof can be used. Specific examples thereof include aluminum, calcium, magnesium, lithium, magnesium alloy, and aluminum alloy. Specific examples of the alloy include aluminum/lithium fluoride, aluminum/lithium, magnesium/silver and magnesium/indium. In order to efficiently extract emission of the organic EL device, at least one of electrodes is desirably set to have a transmittance of 10% or more. Sheet resistance as the electrode is preferably set to be several hundred Ω/square or less. In addition, thickness is set up in the range of ordinarily 10 nanometers to 1 micrometer, and preferably 10 nanometers to 400 nanometers, although a level depends on properties of an electrode material. Such an electrode can be prepared by forming a thin film using the electrode material according to a method such as vacuum evaporation and sputtering.

Next, as one example of a method of preparing the organic EL device by using the emission material according to the invention, a method of preparing the organic EL device composed of the anode/hole injection layer/hole transport layer/emission layer/electron transport material of the invention/cathode as described above is described. The thin film of the anode material is formed on a suitable substrate by the vacuum evaporation method to prepare the anode, and then the thin films of the hole injection layer and the hole transport layer are formed on the anode. A thin film of the emission layer is formed thereon. The electron transport material of the invention is vacuum-evaporated on the emission layer to form the thin film, and the thin film is served as the electron transport layer. Further, an objective organic EL device is obtained by forming the thin film composed of a material for the cathode by the vacuum evaporation method to be served as the cathode. In addition, in preparation of the organic EL device, the organic EL device can also be prepared in the order of the cathode, the electron transport layer, the emission layer, the hole transport layer, the hole injection layer and the anode by reversing the order of preparation.

In the case where direct current voltage is applied to the thus obtained organic EL device, the voltage may be applied by setting polarity of the anode as plus and polarity of the cathode as minus. If a degree of 2 to 40 V is applied thereto, emission can be observed from a transparent or translucent electrode side (anode or cathode, or both of the anode and the cathode). Moreover, the organic EL device emits light also when alternating current voltage is applied thereto. In addition, a waveform of alternating current to be applied may be arbitrary.

### EXAMPLES

The invention will be described in more detail based on Examples below. First, Synthesis Example of a compound used in Examples will be described below.

### Synthesis Example 1 Synthesis of Compound (1-3)

### Synthesis of 2-(4-bromophenyl)thiazole

A flask in which 2-bromothiazole (16.4 g) and 50 mL of THF solution were put was cooled in an iced bath, and then 55 mL of a THF solution of 2 M isopropyl magnesium chloride was added dropwise thereto under a nitrogen atmosphere while stirring the mixture. After completion of dropwise addition, the resulting mixture was stirred for 1 hour, and then a zinc chloride-tetramethylethylenediamine complex (30.3 g) was added thereto while stirring the mixture. Then, the resulting mixture was stirred for 1 hour at room temperature, and then 1-bromo-4-iodobenzene (28.3 g) and Pd(PPh₃)₄ (3.5 g) were added thereto, and heated and stirred at reflux temperature for 1.5 hours. After the reaction mixture was cooled to room temperature, in order to remove metal ion of a catalyst, a solution (hereinafter, abbreviated as EDTA.4Na aqueous solution) prepared by dissolving into a proper amount of water ethylenediaminetetraacetic acid.tetrasodium salt dihydrate corresponding to about 2-fold moles based on an objective compound was added thereto, and the resulting mixture was stirred. Subsequently, toluene was further added to the solution to separate a liquid, and a solvent was distilled off under reduced pressure, and then the resulting concentrate was purified by silica gel column chromatography (eluent: toluene/ethyl acetate=50/1 (in a volume ratio)) to obtain 2-(4-bromophenyl)thiazole (18.3 g).

### Synthesis of Compound (1-3)

A flask in which 2,2′-(2-phenylanthracene-9,10-diyl)bis(4,4,5,5-tetramethyl-1,3,2-dioxaborolane) (3.5 g) prepared with reference to a method described in JP 2008-247895 A, 2-(4-bromophenyl)thiazole (3.7 g), tripotassium phosphate (5.9 g), Pd(PPh₃)₄ (0.2 g), 1,2,4-trimethylbenzene (20 mL) and water (2 mL) were put was heated and the resulting mixture was stirred at reflux temperature for 7.5 hours. The reaction mixture was cooled to room temperature, and water and toluene were added thereto to separate a liquid. Then, a solvent was distilled off under reduced pressure, and the concentrate was purified by silica gel column chromatography (eluent: toluene/ethyl acetate=50/1 (in a volume ratio)). A solid obtained by distilling the solvent off under reduced pressure was recrystallized in chlorobenzene to obtain compound (1-3): 2,2′-((2-phenylanthracene-9,10-diyl)bis(4,1-phenylene))dithiazole (1.9 g).

¹H-NMR (CDCl₃): δ=8.23 (d, 4H), 7.95 (m, 3H), 7.83 (d, 1H), 7.74 (m, 2H), 7.58-7.67 (m, 5H), 7.56 (d, 2H), 7.34-7.43 (m, 6H) and 7.30 (t, 1H).

### Synthesis of Compound (1-4)

A flask in which 2,2′-(2-phenylanthracene-9,10-diyl)bis(4,4,5,5-tetramethyl-1,3,2-dioxaborolane) (4.0 g), 2-(4-bromophenyl)oxazole (4.2 g) prepared by a method described in J. Med. Chem. 2000, 43, pp. 3111-3117, potassium carbonate (4.4 g), tetrabutylammonium bromide (0.5 g), Pd-132 (trademark; made by Johnson Matthey Catalysis and Chiral Technologies) (0.2 g), 1,2,4-trimethylbenzene (20 mL) and water (2 mL) were put was heated and the resulting mixture was stirred at reflux temperature for 5.5 hours. The reaction mixture was cooled to room temperature, and water and toluene were added thereto to separate a liquid. Subsequently, the separated liquid was purified by silica gel column chromatography (eluent: toluene/ethyl acetate). On the above occasion, referring to a method described in 94 page of “YukiChem.gakujikken no Tebiki (Guideline to Organic Chemistry Experiment) (1)—Busshitsu ToriatsuChem.iho to Bunriseiseiho (Material Handling Method and Separation Purification Method),” published by Chem. gaku-Dojin Publishing Company, INC, a ratio of ethyl acetate in the eluent was slowly increased to elute an object. Further, the resulting material was recrystallized from chlorobenzene to obtain compound (1-4): 2,2′-(2-phenylanthracene-9,10-diyl)(bis(4,1-phenylene))dioxazole (1.0 g).

¹H-NMR (CDCl₃): δ=8.32 (d, 4H), 7.90 (m, 1H), 7.79 (m, 3H), 7.71 (m, 2H), 7.64 (m, 5H), 7.55 (d, 2H), 7.27-7.44 (m, 7H).

### Synthesis of Compound (1-21)

First, 2,2′-((2,3-diphenylnaphthalene-1,4-diyl)bis(4,1-phenylene))bis(4, 4,5,5-tetramethyl-1,3,2-dioxaborolane) serving as a raw material was prepared as described below.

A flask in which 1,4-bis(4-bromophenyl)-2,3-diphenylnaphthalene (6.0 g) prepared by a method described in WO 2007/105884 A, bis(pinacolato)diboron (6.2 g), Pd(dppf)Cl₂.CH₂Cl₂ (0.3 g), potassium acetate (4.0 g) and cyclopentyl methyl ether (30 mL) were put was heated, and the resulting mixture was stirred at reflux temperature for 3 hours under a nitrogen atmosphere. After completion of heating, the reaction mixture was cooled to room temperature, and toluene and water were added thereto to separate a liquid. Subsequently, the separated organic layer was purified by activated-carbon column chromatography (eluent: toluene) to obtain 2,2′-((2,3-diphenylnaphthalene-1,4-diyl)bis(4,1-phenylene))bis(4,4,5,5-tetramethyl-1,3,2-dioxaborolane (4.1 g). Compound (1-21) was prepared by using the thus obtained 2,2′-((2,3-diphenylnaphthalene-1,4-diyl)bis(4,1-phenylene))bis(4,4,5,5-tetramethyl-1,3,2-dioxaborolane as described below.

A flask in which 2,2′-((2,3-diphenylnaphthalene-1,4-diyl)bis(4,1-phenylene))bis(4,4,5,5-tetramethyl-1,3,2-dioxaborolane (2.5 g), 2-bromothiazole (1.4 g), tripotassium phosphate (3.1 g), tetrabutylammonium bromide (0.2 g), Pd-132 (trademark; made by Johnson Matthey Catalysis and Chiral Technologies) (0.1 g), 1,2,4-trimethylbenzene (20 mL) and water (2 mL) were put was heated, and the resulting mixture was stirred at reflux temperature for 3.5 hours. The reaction mixture was cooled to room temperature, and water and toluene were added thereto to separate a liquid. Subsequently, the separated liquid was purified by silica gel column chromatography (eluent: toluene/ethyl acetate). On the above occasion, a ratio of ethyl acetate in the eluent was slowly increased to elute an object. Further, the resulting object was purified by NH-modified silica gel (DM1020: made by Fuji Silysia Chemical Ltd.) chromatography (eluent: toluene), and then recrystallized from anisole to obtain compound (1-21): 2,2′-(2,3-diphenylnaphthalene-1,4-diyl)bis(4,1-phenylene)dithiazole (0.4 g).

¹H-NMR (CDCl₃): δ=7.87 (d, 4H), 7.84 (m, 2H), 7.66 (m, 2H), 7.42 (m, 2H), 7.30 (m, 6H), 6.80-6.92 (m, 10H).

### Synthesis of Compound (1-25)

First, 2-(4-(4,4,5,5-tetramethyl-1,3,2-dioxaborolane-2-yl)phenyl)thiazole serving as a raw material was prepared as described below.

A flask in which 2-(4-bromophenyl)thiazole (9.0 g), bispinacolatodiboron (11.4 g), Pd(dppf)Cl₂.CH₂Cl₂ (0.9 g), potassium acetate (7.4 g) and cyclopentyl methyl ether (50 mL) were put was heated, and the resulting mixture was stirred at reflux temperature for 3 hours under a nitrogen atmosphere. After completion of heating, the reaction mixture was cooled to room temperature, and an insoluble matter was removed by suction filtration. A solvent was distilled off under reduced pressure, and then the concentrate was purified by an activated-carbon short column and then an NH-modified silica gel short column to obtain 2-(4-(4,4,5,5-tetramethyl-1,3,2-dioxaborolane-2-yl)phenyl)thiazole (7.7 g). Then, compound (1-25) was prepared by using the thus obtained 2-(4-(4,4,5,5-tetramethyl-1,3,2-dioxaborolane-2-yl)phenyl)thiazole as described below.

A flask in which 2-(4-(4,4,5,5-tetramethyl-1,3,2-dioxaborolane-2-yl)phenyl)thiazole (4.3 g), 2,7-dibromo-9-phenyl-9H-carbazole (2.5 g), tripotassium phosphate (5.3 g), tetrabutylammonium bromide (0.1 g), Pd(PPh₃)₄ (0.2 g), 1,2,4-trimethylbenzene (20 mL) and water (2 mL) were put was heated, and the resulting mixture was stirred at reflux temperature for 4 hours. The reaction mixture was cooled to room temperature and then to dissolve inorganic salt by adding water thereto, and subjected to suction filtration. The resulting solid was purified by silica gel column chromatography (eluent: toluene/ethylacetate=9/1 (in a volume ratio)), and then recrystallized from orthodichlorobenzene to obtain compound (1-25): 2,2′-(9-phenyl-9H-carbazole-2,7-diyl)bis(4,1-phenylene)dithiazole (1.7 g).

¹H-NMR (CDCl₃): δ=8.23 (d, 2H), 8.04 (d, 4H), 7.89 (m, 2H), 7.74 (d, 4H), 7.59-7.70 (m, 8H), 7.54 (t, 1H), 7.34 (m, 2H).

### Synthesis of Compound (1-29)

A flask in which 2-(4-(4,4,5,5-tetramethyl-1,3,2-dioxaborolane-2-yl)phenyl)thiazole (5.4 g), 7-phenyl-7H-benzo[c]carbazole-5,9-diylbis(trifluoromethanesulfonate) (5.0 g), tripotassium phosphate (7.2 g), Pd(PPh₃)₄ (0.3 g), 1,2,4-trimethylbenzene (20 mL), t-butyl alcohol (5 mL) and water (1 mL) were put was heated, and the resulting mixture was stirred at reflux temperature for 3 hours. The reaction mixture was cooled to room temperature and then to dissolve inorganic salt by adding water thereto, and subjected to suction filtration. The resulting solid was purified by NH-modified silica gel column chromatography (eluent: toluene), and further recrystallized from chlorobenzene to obtain compound (1-29): 2,2′-(7-phenyl-7H-benzo[c]carbazole-5,9-diyl)bis(4,1-phenylene)dithiazole (0.5 g).

¹H-NMR (CDCl₃): δ=8.97 (d, 1H), 8.73 (d, 1H), 8.09 (d, 2H), 8.05 (m, 3H), 7.90 (m, 2H), 7.50-7.81 (m, 13H), 7.47 (t, 1H), 7.36 (m, 2H).

### Synthesis of Compound (1-37)

A flask in which 2-(4-(4,4,5,5-tetramethyl-1,3,2-dioxaborolane-2-yl)phenyl)thiazole (3.6 g), 5,9-dibromo-7,7-dimethyl-7H-benzo[c]fluorene (2.1 g), tripotassium phosphate (4.4 g), tetrabutylammonium bromide (0.1 g), Pd(PPh₃)₄ (0.2 g), 1,2,4-trimethylbenzene (20 mL) and water (10 mL) were put was heated, and the resulting mixture was stirred at reflux temperature for 3.5 hours. The reaction mixture was cooled to Loom temperature, and water and toluene were added thereto to separate a liquid. Subsequently, the separated liquid was purified by silica gel column chromatography (eluent: toluene/ethylacetate=19/1 (in a volume ratio)), and then a solvent was distilled off under reduced pressure. Then, heptane was added thereto to allow reprecipitation to obtain compound (1-37): 2,2′-(7,7-dimethyl-7H-benzo[c]fluorene-5,9-diyl)bis(4,1-phenylene)dithiazole (1.0 g).

¹H-NMR (CDCl₃): δ=8.85 (d, 1H), 8.43 (d, 1H), 8.13 (d, 2H), 8.09 (d, 2H), 8.04 (d, 1H), 7.91 (dd, 2H), 7.81 (m, 3H), 7.76 (d, 1H), 7.63-7.71 (m, 3H), 7.60 (s, 1H), 7.50 (t, 1H), 7.36 (dd, 2H), 1.67 (s, 6H).

### Synthesis of Compound (1-45)

A flask in which 2-(4-(4,4,5,5-tetramethyl-1,3,2-dioxaborolane-2-yl)phenyl)thiazole (7.5 g), [1,1′-binaphthalene]-4,4′-diylbis(trifluoromethanesulfonate) (6.5 g), tripotassium phosphate (10.0 g), Pd(PPh₃)₄ (0.4 g), 1,2,4-trimethylbenzene (20 mL), t-butyl alcohol (5 mL) and water (1 mL) were put was heated, and the resulting mixture was stirred at reflux temperature for 7.5 hours. The reaction mixture was cooled to room temperature, and water and toluene were added thereto to separate a liquid. Subsequently, the separated liquid was purified by an activated-carbon short column, and washed with ethyl acetate to obtain compound (1-45): 4,4′-bis(4-(thiazole-2-yl)phenyl-1,1′-binaphthalene (0.4 g).

¹H-NMR (CDCl₃): δ=8.18 (d, 4H), 8.01 (m, 4H), 7.87 (m, 2H), 7.77 (d, 4H), 7.67 (m, 4H), 7.56 (t, 2H), 7.43 (m, 4H).

### Synthesis of Compound (1-53)

A flask in which 2-(4-(4,4,5,5-tetramethyl-1,3,2-dioxaborolane-2-yl)phenyl)thiazole (2.3 g), triphenylene-2,7-diylbis(trifluoromethanesulfonate) (1.9 g), tripotassium phosphate (3.1 g), tetrabutylammonium bromide (0.2 g), Pd(dba)₂ (0.1 g), 4-(di-t-butylphosphino)-N,N-dimethylaniline (0.1 g), toluene (20 mL) and water (2 mL) were put was heated, and the resulting mixture was stirred at reflux temperature for 17 hours. The reaction mixture was cooled to room temperature and then to dissolve inorganic salt by adding water thereto, and subjected to suction filtration to obtain a precipitate. The resulting solid was washed with methanol and toluene, and then dissolved in orthodichlorobenzene, and purified by an NH-modified silica gel short column. The resulting material was further recrystallized from chlorobenzene to obtain compound (1-53): 2,7-bis(4-(thiazole-2-yl)phenyl)triphenylene (0.5 g).

¹H-NMR (CDCl₃): δ=8.93 (s, 2H), 8.79 (m, 4H), 8.15 (d, 4H), 7.97 (d, 2H), 7.92 (m, 6H), 7.74 (m, 2H), 7.39 (m, 2H).

### Synthesis of Compound (1-85)

First, 9,10-bis(3-bromophenyl)-2-phenyl-9,10-dihydroanthracene-9,10-diol serving as a raw material was prepared as described below.

A CPME solution (250 mL) of 1,3-dibromobenzene (47.7 g) was cooled to −78° C., and a hexane solution (81.7 mL) of 2.6 M butyllithium was added dropwise thereto. After stirring for 30 minutes, 2-phenyl-9,10-anthraquinone (23.0 g) was added thereto, and further the resulting mixture was stirred for 5 hours. The reaction mixture was cooled to room temperature, and water and toluene were added thereto to separate a liquid. Subsequently, the separated liquid was purified by a silica gel short column to obtain 9,10-bis(3-bromophenyl)-2-phenyl-9,10-dihydroanthracene-9,10-dio 1 (46.3 g). Then, 9,10-bis(3-bromophenyl)-2-phenylanthracene was prepared by using the 9,10-bis(3-bromophenyl)-2-phenyl-9,10-dihydroanthracene-9,10-dio 1 as described below.

A flask in which 9,10-bis(3-bromophenyl)-2-phenyl-9,10-dihydroanthracene-9,10-dio 1 (46.3 g), sodium hypophosphite monohydrate (79.4 g), potassium iodide (32.3 g) and acetic acid (200 mL) were put was heated, and the resulting mixture was stirred at reflux temperature for 2.5 hours. The reaction mixture was cooled to room temperature, and then subjected to suction filtration to obtain a precipitate, and the resulting solid was washed with water. Subsequently, the solid was purified by silica gel column chromatography (eluent: heptane/toluene 4/1 (in a volume ratio)) to obtain 9,10-bis(3-bromophenyl)-2-phenylanthracene (29.2 g). Then, 2,2′-((2-phenylanthracene-9,10-diyl)bis(3,1-phenylene))bis(4,4,5,5-tetramethyl-1,3,2-dioxaborolane) was prepared by using the thus obtained 9,10-bis(3-bromophenyl)-2-phenylanthracene as described below.

A flask in which 9,10-bis(3-bromophenyl)-2-phenylanthracene (29.0 g), bispinacolato diboron (31.3 g), Pd(dppf)Cl₂.CH₂Cl₂ (2.1 g), potassium acetate (20.2 g) and cyclopentyl methyl ether (200 mL) were put was heated, and the resulting mixture was stirred at reflux temperature for 6 hours under a nitrogen atmosphere. After completion of heating, the reaction mixture was cooled to room temperature to remove inorganic salt by suction filtration. Subsequently, the resulting solution was passed through an activated-carbon short column to perform discoloration, and a solvent was distilled off under reduced pressure, and the resulting solid was washed with toluene to obtain 2,2′-((2-phenylanthracene-9,10-diyl)bis(3,1-phenylene))bis(4,4,5,5-tetramethyl-1,3,2-dioxaborolane (11.0 g). Then, compound (1-85) was prepared by using the thus obtained 2,2′-((2-phenylanthracene-9,10-diyl)bis(3,1-phenylene))bis(4,4,5,5-tetramethyl-1,3,2-dioxaborolane as described below.

A flask in which 2,2′-((2-phenylanthracene-9,10-diyl)bis(3,1-phenylene))bis(4,4,5,5-tetramethyl-1,3,2-dioxaborolane (5.6 g), 2-bromothiazole (3.1 g), tripotassium phosphate (7.2 g), tetrabutylammonium bromide (0.1 g), Pd-132 (trademark; made by Johnson Matthey Catalysis and Chiral Technologies) (0.1 g), 1,2,4-trimethylbenzene (50 mL) and water (10 mL) were put was heated, and the resulting mixture was stirred at reflux temperature for 8 hours. The reaction mixture was cooled to room temperature to separate a liquid by adding water and toluene thereto. Subsequently, the separated liquid was purified by an activated-carbon short column, and further recrystallized from toluene to obtain compound (1-85): 2,2′-((2-phenylanthracene-9,10-diyl)bis(3,1-phenylene))dithiazole (0.4 g).

¹H-NMR (CDCl₃): δ=8.23 (d, 1H), 8.12 (s, 2H), 7.93 (s, 1H), 7.90 (m, 2H), 7.83 (d, 1H), 7.74 (m, 5H), 7.65 (d, 1H), 7.60 (m, 1H), 7.55 (d, 2H), 7.36 (m, 7H), 7.30 (m, 1H).

### Synthesis of Compound (1-166)

First, 2,2′-((2-phenylanthracene-9,10-diyl)bis(4,1-phenylene))bis(4,4,5,5-tetramethyl-1,3,2-dioxaborolane) serving as a raw material was prepared by applying a method similar to the method of preparing 2,2′-((2-phenylanthracene-9,10-diyl)bis(3,1-phenylene))bis(4,4,5,5-tetramethyl-1,3,2-dioxaborolane) as described above and by changing 1,3-dibromobenzene serving as the raw material to 1,4-dibromobenzene. Compound (1-166) was prepared by using the thus obtained 2,2′-((2-phenylanthracene-9,10-diyl)bis(4,1-phenylene))bis(4,4,5,5-tetramethyl-1,3,2-dioxaborolane) as described below.

A flask in which 2,2′-((2-phenylanthracene-9,10-diyl)bis(4,1-phenylene))bis(4,4,5,5-tetramethyl-1,3,2-dioxaborolane) (3.3 g), 5-bromothiazole (2.0 g), potassium carbonate (2.8 g), Pd(PPh₃)₄ (0.2 g), 1,2,4-trimethylbenzene (20 mL) and water (2 mL) were put was heated, and the resulting mixture was stirred at reflux temperature for 7 hours. The reaction mixture was cooled to room temperature to separate a liquid by adding water and toluene thereto. Subsequently, the separated liquid was purified by silica gel chromatography (eluent: toluene/ethyl acetate=9/1 (in a volume ratio)), and then recrystallized from chlorobenzene to obtain compound (1-166): 5,5′-(2-phenylanthracene-9,10-diyl)bis(4,1-phenylene)dithiazole (1.6 g).

¹H-NMR (CDCl₃): δ=8.83 (m, 2H), 8.26 (s, 2H), 7.93 (m, 1H), 7.85 (m, 5H), 7.74 (m, 2H), 7.65 (dd, 1H), 7.57 (m, 6H), 7.35-7.44 (m, 4H), 7.32 (t, 1H).

### Synthesis of Compound (1-274)

A flask in which 2,2′-((2-phenylanthracene-9,10-diyl)bis(4,1-phenylene))bis(4,4,5,5-tetramethyl-1,3,2-dioxaborolane) (3.3 g), 4-bromothiazole (2.0 g), tetrabutylammonium bromide (0.2 g), potassium carbonate (2.8 g), Pd(PPh₃)₄ (0.2 g), 1,2,4-trimethylbenzene (20 mL) and water (2 mL) were put was heated, and the resulting mixture was stirred at reflux temperature for 6 hours. The reaction mixture was cooled to room temperature to separate a liquid by adding water and toluene thereto. Subsequently, the separated liquid was purified by silica gel chromatography (eluent: toluene/ethyl acetate=9/1 (in a volume ratio)), and then recrystallized from chlorobenzene, and further recrystallized from anisole to obtain compound (1-274): 4,4′-(2-phenylanthracene-9,10-diyl)bis(4,1-phenylene)dithiazole (1.4 g).

¹H-NMR (CDCl₃): δ=8.98 (m, 2H), 8.20 (m, 4H), 7.99 (m, 1H), 7.86 (d, 1H), 7.78 (m, 2H), 7.71 (m, 2H), 7.59-7.65 (m, 5H), 7.57 (d, 2H), 7.33-7.41 (m, 4H), 7.30 (t, 1H).

### Synthesis of Compound (1-382)

First, 2-(5-bromopyridine-2-yl)thiazole serving as a raw material was prepared as described below.

A flask in which 2-bromothiazole (22.2 g) and THF (50 mL) were put was cooled to 0° C., and a THF solution (75.0 mL) of 2 M isopropylmagnesium chloride was added thereto. After dropwise addition, the resulting mixture was stirred for 1 hour, and a zinc chloride-tetramethylethylenediamine complex (37.6 g) was added thereto, and slowly heated to room temperature. Then, 2,5-dibromopyridine (35.2 g) and Pd(PPh₃)₄ (4.7 g) were added thereto, and the resulting mixture was heated and stirred at reflux temperature for 4 hours. The reaction mixture was cooled to room temperature to separate a liquid by adding an EDTA aqueous solution and toluene thereto. Subsequently, the separated liquid was purified by silica gel chromatography (eluent: toluene/ethyl acetate=4/1 (in a volume ratio)) to obtain 2-(5-bromopyridine-2-yl)thiazole (14.0 g). Compound (1-382) was prepared by using the thus obtained 2-(5-bromopyridine-2-yl)thiazole as described below.

A flask in which 2,2′-(2-phenylanthracene-9,10-diyl)bis(4,4,5,5-tetramethyl-1,3,2-dioxaborolane) (3.0 g), 2-(5-bromopyridine-2-yl)thiazole (3.1 g), tetrabutylammonium bromide (0.1 g), tripotassium phosphate (5.0 g), Pd(PPh₃)₄ (0.2 g), 1,2,4-trimethylbenzene (20 mL) and water (2 mL) were put was heated, and the resulting mixture was stirred at reflux temperature for 4.5 hours. The reaction mixture was cooled to room temperature to separate a liquid by adding water and toluene thereto. Subsequently, the separated liquid was purified by silica gel chromatography (eluent: toluene/ethyl acetate=7/3 (in a volume ratio)), and then recrystallized from a toluene/heptane mixed solvent to obtain compound (1-382): 2,2′-(5,5′-(2-phenylanthracene-9,10-diyl)bis(pyridine-5,2-phenylene)dithiazole (0.9 g).

¹H-NMR (CDCl₃): δ=8.80 (m, 2H), 8.51 (d, 2H), 7.98-8.03 (m, 4H), 7.88 (m, 1H), 7.81 (d, 1H), 7.68-7.78 (m, 3H), 7.51-7.60 (m, 4H), 7.38-7.46 (m, 4H), 7.35 (t, 1H).

### Synthesis of Compound (1-383)

First, 2-(5-bromopyridine-2-yl)oxazole serving as a raw material was prepared as described below.

A flask in which oxazole (4.5 g) and THF (150 mL) were put was cooled to −78° C., and a hexane solution (27.0 mL) of 2.6 M n-butyllithium was added thereto. After dropwise addition, the resulting mixture was stirred for 1 hour, and a zinc chloride-tetramethylethylenediamine complex (18.1 g) was added thereto, and slowly heated to room temperature. Then, 2,5-dibromopyridine (15.4 g) and Pd(PPh₃)₄ (3.8 g) were added thereto, and the resulting mixture was stirred at reflux temperature for 10 hours. The reaction mixture was cooled to room temperature to separate a liquid by adding an EDTA aqueous solution and toluene thereto. A solvent was distilled off under reduced pressure, and then the resulting solid was washed with heptane to obtain 2-(5-bromopyridine-2-yl)oxazole (10.2 g). Compound (1-383) was prepared by using the thus obtained 2-(5-bromopyridine-2-yl)oxazole as described below.

A flask in which 2,2′-(2-phenylanthracene-9,10-diyl)bis(4,4,5,5-tetramethyl-1,3,2-dioxaborolane) (3.0 g), 2-(5-bromopyridine-2-yl)oxazole (3.1 g), tetrabutylammonium bromide (0.1 g), potassium carbonate (1.6 g), Pd-132 (trademark; made by Johnson Matthey Catalysis and Chiral Technologies) (0.1 g), 1,2,4-trimethylbenzene (10 mL) and water (2 mL) were put was heated, and the resulting mixture was stirred at reflux temperature for 5 hours. The reaction mixture was cooled to room temperature, and subjected to suction filtration to obtain a precipitate, and the resulting solid was washed with water. Subsequently, the resulting solid was purified by NH-modified silica gel column chromatography (eluent: toluene/ethyl acetate=5/1 (in a volume ratio)) to obtain compound (1-383): 2,2′-(5,5′-(2-phenylanthracene-9,10-diyl)bis(pyridine-5,2-diyl)dioxazole (0.2 g).

¹H-NMR (CDCl₃): δ=8.90 (d, 2H), 8.46 (t, 2H), 8.03 (m, 2H), 7.92 (m, 2H), 7.85 (s, 1H), 7.78 (m, 1H), 7.65-7.74 (m, 3H), 7.55 (d, 2H), 7.39-7.48 (m, 6H), 7.34 (t, 1H).

### Synthesis of Compound (1-404)

First, 2-(5-(4,4,5,5-tetramethyl-1,3,2-dioxaborolane-2-yl)pyridine-2-yl)thiazole serving as a raw material was prepared as described below.

A flask in which 2-(5-bromopyridine-2-yl)thiazole (10.0 g), bis(pinacolato)diboron (12.6 g), Pd(dppf)Cl₂.CH₂Cl₂ (1.0 g), potassium acetate (8.1 g) and cyclopentyl methyl ether (50 mL) were put was heated, and the resulting mixture was stirred at reflux temperature for 3 hours. After completion of heating, the reaction mixture was cooled to room temperature to separate a liquid by adding water and toluene thereto. Subsequently, the separated liquid was purified by activated-carbon column chromatography to obtain 2-(5-(4,4,5,5-tetramethyl-1,3,2-dioxaborolane-2-yl)pyridine-2-yl)thiazole (10.7 g). Compound (1-404) was prepared by using the thus obtained 2-(5-(4,4,5,5-tetramethyl-1,3,2-dioxaborolane-2-yl)pyridine-2-yl)thiazole as described below.

A flask in which 2-(5-(4,4,5,5-tetramethyl-1,3,2-dioxaborolane-2-yl)pyridine-2-yl)thiazole (1.5 g), 2,7-dibromo-9-phenyl-9H-carbazole (1.3 g), potassium carbonate (0.7 g), tetrabutylammonium bromide (0.1 g), Pd-132 (trademark; made by Johnson Matthey Catalysis and Chiral Technologies) (0.2 g) and toluene (5 mL) were put was heated, and the resulting mixture was stirred at reflux temperature for 8 hours. The reaction mixture was cooled to room temperature to obtain a precipitate by suction filtration, and the resulting solid was washed with water. The solid was further recrystallized from chlorobenzene to obtain compound (1-404): 2,2′-(5,5′-(9-phenyl-9H-carbazole-2,7-diyl)bis(pyridine-5,2-diyl))dithiazole (0.1 g).

¹H-NMR (CDCl₃): δ=8.92 (m, 2H), 8.27 (m, 4H), 8.07 (dd, 2H), 7.94 (m, 2H), 7.53-7.72 (m, 9H) and 7.45 (m, 2H).

### Synthesis of Compound (1-408)

A flask in which 2-(5-(4,4,5,5-tetramethyl-1,3,2-dioxaborolane-2-yl)pyridine-2-yl)thiazole (2.7 g) 7-phenyl-7H-benzo[c]carbazole-5,9-diylbis(trifluoromethanesulfonate) (2.5 g), potassium carbonate (2.3 g), tetrabutylammonium bromide (0.3 g), Pd-132 (trademark; made by Johnson Matthey Catalysis and Chiral Technologies) (0.1 g), 1,2,4-trimethylbenzene (20 mL) and water (2 mL) were put was heated, and the resulting mixture was stirred at reflux temperature for 8 hours. The reaction mixture was cooled to room temperature to separate a liquid by adding water and toluene thereto. Subsequently, the separated liquid was purified by NH-modified silica gel column chromatography (eluent: toluene), and then a solvent was distilled off under reduced pressure, and heptane was added thereto to allow reprecipitation to obtain compound (1-408): 2,2′-(5,5′-(7-phenyl-7H-benzo[c]carbazole-5,9-diyl)bis(pyridine-5,2-diyl))dithiazole (1.9 g).

¹H-NMR (CDCl₃): δ=8.96 (m, 2H), 8.79 (m, 2H), 8.33 (d, 1H), 8.29 (d, 1H), 8.09 (dd, 1H), 7.92-8.03 (m, 4H), 7.81 (t, 1H), 7.74 (m, 2H), 7.60-7.71 (m, 4H), 7.40-7.59 (m, 5H).

### Synthesis of Compound (1-416)

A flask in which 2-(5-(4,4,5,5-tetramethyl-1,3,2-dioxaborolane-2-yl)pyridine-2-yl)thiazole (1.6 g), 5,9-dibromo-7,7-dimethyl-7H-benzo[c]fluorene (1.0 g), potassium carbonate (0.7 g), tetrabutylammonium bromide (0.1 g), Pd-132 (trademark; made by Johnson Matthey Catalysis and Chiral Technologies) (0.1 g), 1,2,4-trimethylbenzene (5 mL) and water were put was heated, and the resulting mixture was stirred at reflux temperature for 3 hours. The reaction mixture was cooled to room temperature to separate a liquid by adding water and toluene thereto. Subsequently, the separated liquid was purified by an activated-carbon short column, and then recrystallized from a toluene/heptane mixed solvent to obtain compound (1-416): 2,2′-(5,5′-(7,7-dimethyl-7H-benzo[c]fluorene-5,9-diyl)bis(5,2-phenylene-diyl)dithiazole (0.2 g).

¹H-NMR (CDCl₃): δ=9.00 (m, 1H), 8.88 (d, 1H), 8.83 (s, 1H), 8.50 (d, 1H), 8.38 (d, 1H), 8.32 (d, 1H), 8.15 (d, 1H), 7.95-8.05 (m, 4H), 7.81 (s, 1H), 7.77 (d, 1H), 7.73 (t, 1H), 7.61 (s, 1H), 7.55 (t, 1H), 7.49 (dd, 2H), 1.66 (s, 6H).

### Synthesis of Compound (1-424)

A flask in which 2-(5-(4,4,5,5-tetramethyl-1,3,2-dioxaborolane-2-yl)pyridine-2-yl)thiazole (5.5 g), [1,1′-binaphthalene]-4,4′-diylbis(trifluoromethanesulfonate (6.3 g), potassium carbonate (5.5 g), tetrabutylammonium bromide (0.1 g), Pd-132 (trademark; made by Johnson Matthey Catalysis and Chiral Technologies) (0.2 g), 1,2,4-trimethylbenzene (20 mL) and water (2 mL) were put was heated, and the resulting mixture was stirred at reflux temperature for 5 hours. The reaction mixture was cooled to room temperature to separate a liquid by adding water and toluene thereto. Subsequently, the separated liquid was purified by an activated-carbon short column, and then recrystallized from a toluene/heptane mixed solvent to obtain compound (1-424): 4,4′-bis(6-(thiazole-2-yl)pyridine-3-yl-1,1′-binaphthalene (1.7 g).

¹H-NMR (CDCl₃): δ=8.89 (m, 2H), 8.40 (d, 2H), 8.08 (d, 2H), 7.99 (m, 4H), 7.56-7.67 (m, 6H), 7.50 (m, 4H), 7.40 (t, 2H).

### Synthesis of Compound (1-557)

First, 6,6′-(2-phenylanthracene-9,10-diyl)bis(2-bromopyridine) serving as a raw material was prepared as described below.

A flask in which 2,6-dibromopyridine (142.6 g) and toluene (600 mL) were put was cooled to −78° C., and a hexane solution (24.6 mL) of 2.6 M n-butyllithium was added thereto. After dropwise addition, the resulting mixture was stirred for 1 hour, and 2-phenylanthracene-9,10-dione (59.8 g) was added thereto. The resulting mixture was further stirred for 4 hours, and then a saturated aqueous solution of ammonium chloride was added thereto, and analyzed. Subsequently, the resulting liquid was purified by silica gel column chromatography (eluent: toluene/ethyl acetate=1/1 (in a volume ratio)) to obtain 9,10-bis(6-bromopyridine-2-yl)-2-phenyl-9,10-dihydroanthracene-9,10-diol (108.2 g). Then, 6,6′-(2-phenylanthracene-9,10-diyl)bis(2-bromopyridine) was prepared by using the thus obtained 9,10-bis(6-bromopyridine-2-yl)-2-phenyl-9,10-dihydroanthracene-9,10-diol as described below.

A flask in which 9,10-bis(6-bromopyridine-2-yl)-2-phenyl-9,10-dihydroanthracene-9,10-diol (105.1 g), sodium hypophosphite monohydrate (225.7 g), potassium iodide (75.5 g) and acetic acid (600 mL) were put was heated, and the resulting mixture was stirred at reflux temperature for 3.5 hours. The reaction mixture was cooled to room temperature and then to dissolve inorganic salt by adding water thereto to obtain a precipitate by suction filtration. Subsequently, the resulting solid was purified by silica gel column chromatography (eluent: toluene/ethyl acetate=1/1 (in a volume ratio)), and a proper amount of a solvent was distilled off under reduced pressure, and methanol was added thereto to allow reprecipitation to obtain 6,6′-(2-phenylanthracene-9,10-diyl)bis(2-bromopyridine) (40.1 g). Compound (1-557) was prepared by using the thus obtained 6,6′-(2-phenylanthracene-9,10-diyl)bis(2-bromopyridine) as described below.

A flask in which 6,6′-(2-phenylanthracene-9,10-diyl)bis(2-bromopyridine (4.7 g), a THF solution (50 mL) of 0.5 M 2-thiazolylzinc bromide, and Pd(PPh₃)₄ (1.5 g) were put was heated, and the resulting mixture was stirred at reflux temperature for 11 hours. The reaction mixture was cooled to room temperature to separate a liquid by adding an EDTA aqueous solution and toluene thereto. Subsequently, the separated liquid was purified by silica gel column chromatography (eluent: toluene/ethyl acetate=9/1 (in a volume ratio)), and further recrystallized from chlorobenzene to obtain compound (1-557): 2,2′-(6,6′-(2-phenylanthracene-9,10-diyl)bis(pyridine-6,2-diyl)dithiazole (2.4 g).

¹H-NMR (CDCl₃): δ=8.40 (m, 2H), 8.07 (m, 2H), 7.99 (m, 2H), 7.92 (m, 1H), 7.81 (d, 1H), 7.73 (m, 2H), 7.67 (d, 1H), 7.61 (m, 2H), 7.55 (d, 2H) 7.34-7.44 (m, 6H), 7.30 (t, 1H).

### Synthesis of Compound (1-558)

A flask in which oxazole (1.5 g) and THF (20 mL) were put was cooled to −78° C., and a hexane solution (8.6 mL) of 2.6 M n-butyllithium was added thereto. After dropwise addition, the resulting mixture was stirred for 1 hour, and a zinc chloride-tetramethylethylenediamine complex (6.4 g) was added thereto, and then the resulting mixture was slowly heated to room temperature. Then, 6,6′-(2-phenylanthracene-9,10-diyl)bis(2-bromopyridine) (4.0 g) and Pd(PPh₃)₄ (1.2 g) were added thereto, and the resulting mixture was stirred at reflux temperature for 24 hours. The reaction mixture was cooled to room temperature to separate a liquid by adding an EDTA aqueous solution and toluene thereto. Subsequently, the separated liquid was purified by silica gel column chromatography (eluent: toluene/ethyl acetate=7/3 (in a volume ratio)), and further recrystallized from orthodichlorobenzene to obtain compound (1-558): 2,2′-(6,6′-(2-phenylanthracene-9,10-diyl)bis(pyridine-6,2-diyl)oxazole (0.4 g).

¹H-NMR (CDCl₃): δ=8.39 (m, 2H), 8.11 (m, 2H), 7.71-7.81 (m, 3H), 7.55-7.69 (m, 6H), 7.53 (d, 2H), 7.28-7.40 (m, 7H).

### Synthesis of Compound (1-611)

First, 2-(5-bromopyridine-3-yl)thiazole serving as a raw material was prepared as described below.

A flask in which 2-bromothiazole (15.0 g) and THF (30 mL) was put was cooled to 0° C., and a THF solution (51.0 mL) of 2 M isopropylmagnesium chloride was added thereto. After dropwise addition, the resulting mixture was stirred for 1 hour, a zinc chloride-tetramethylethylenediamine complex (25.4 g) was added thereto, and the resulting mixture was slowly heated to room temperature. Then, 3,5-dibromopyridine (23.8 g) and Pd(PPh₃)₄ (3.2 g) were added thereto, and the resulting mixture was heated and stirred at reflux temperature for 6 hours. The reaction mixture was cooled to room temperature to separate a liquid by adding an EDTA aqueous solution and toluene thereto. Subsequently, the separated liquid was purified by silica gel column chromatography (eluent: toluene/ethyl acetate 9/1 (in a volume ratio)) to obtain 2-(5-bromopyridine-3-yl)thiazole (1.9 g). Compound (1-611) was prepared by using the thus obtained 2-(5-bromopyridine-3-yl)thiazole as described below.

A flask in which 2,2′-(2-phenylanthracene-9,10-diyl)bis(4,4,5,5-tetramethyl-1,3,2-dioxaborolane (1.8 g), 2-(5-bromopyridine-3-yl)thiazole (1.9 g), tripotassium phosphate (3.0 g), tetrabutylammonium bromide (0.1 g), Pd-132 (trademark; made by Johnson Matthey Catalysis and Chiral Technologies) (0.1 g), 1,2,4-trimethylbenzene (20 mL) and water (2 mL) were put was heated, and the resulting mixture was stirred at reflux temperature for 5.5 hours. The reaction mixture was cooled to room temperature, and then water and toluene were added thereto, and the resulting mixture was analyzed. Subsequently, the separated liquid was purified by NH-modified silica gel column chromatography (eluent: toluene), and further recrystallized from chlorobenzene to obtain compound (1-611): 2,2′-(5,5′-(2-phenylanthracene-9,10-diyl)bis(pyridine-5,3-diyl)dithiazole (0.8 g).

¹H-NMR (CDCl₃): δ=9.45 (m, 2H), 8.83 (m, 2H), 7.44 (m, 2H), 7.97 (m, 2H), 7.85 (s, 1H), 7.79 (m, 1H), 7.70 (m, 3H), 7.55 (d, 2H), 7.35-7.50 (m, 6H), 7.33 (t, 1H).

Any other compound of the invention can be prepared by appropriately changing raw material compounds by a method according to Synthesis Exampled described above.

Examples of organic EL devices using the compounds of the invention are described below in order to describe the invention in more detail below, but the invention is not limited thereto.

Devices related to Example 1 and Comparative Example 1 were prepared, and measurement was carried out on drive start voltage (V) and time (hr) retaining luminance of 80% or more of an initial value in a constant current drive test, respectively. Examples and Comparative Examples will be described in detail below.

Material structure in each layer in each of prepared devices related to Example 1 and Comparative Examples 1 to 2 is shown in Table 1 below.

TABLE 1

| | Hole | Hole | Hole | | | Electron | |
|---|---|---|---|---|---|---|---|
| | injection | injection | transport | Emission layer | | transport | |
| | layer 1 | layer 2 | layer | (20 nm) | | layer* | Cathode |
| | (60 nm) | (10 nm) | (10 nm) | Host | Dopant | (30 nm) | (1 nm/100 nm) |
| Example 1 | HI | HAT-CN | NPB | A | B | (1-3) | Liq/Mg + Ag |
| Comparative | HI | HAT-CN | NPB | A | B | D | Liq/Mg + Ag |
| Example 1 | | | | | | | |
| Comparative | HI | HAT-CN | NPB | A | B | C | Liq/Mg + Ag |
| Example 2 | | | | | | | |

*The electron transport layer was formed using a mixture of a compound and Liq in the table.

In Table 1, “HI” represents N⁴,N^{4′}-diphenyl-N⁴,N^{4′}-bis(9-phenyl-9H-carbazole-3-yl)-[1, 1′-biphenyl]-4,4′-diamine, “HAT-CN” represents 1,4,5,8,9,12-hexaaza-triphenylene-hexacarbonitrile, and “NPB” represents N⁴,N^{4′}-dinaphthalene-1-yl-N⁴,N^{4′}-diphenyl-biphenyl-4,4′-diamine, and compound (A) is 9-phenyl-10-(4-phenylnaphthalene-1-yl)anthracene, compound (B) is 7,7-dimethyl-N⁵,N⁹-diphenyl-N⁵,N⁹-bis(4-(trimethylsilyl)phenyl)-7H-benzo[c]fluorene-5,9-diamine, compound (C) is 4,4′-(2-phenylanthracene-9,10-diyl)bis(4,1-phenylene)dipyridine, and compound (D) is 2,2′-(2-phenylanthracene-9,10-diyl)bis(4,1-phenylene)bis(benzo[d]thiazole. Chemical structure thereof is shown below together with chemical structure of “Liq” used for a cathode.

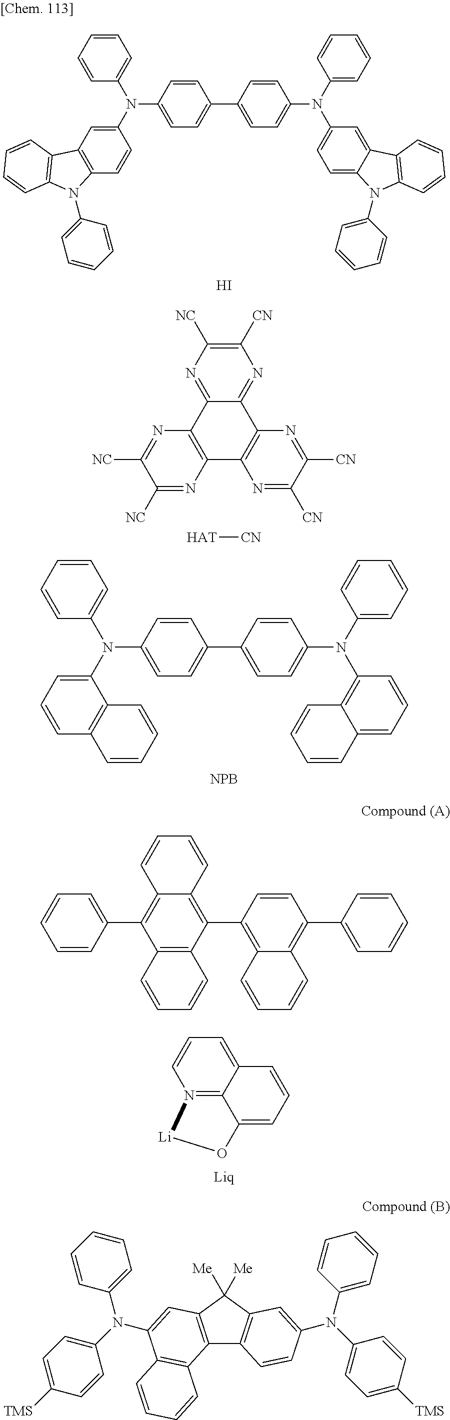

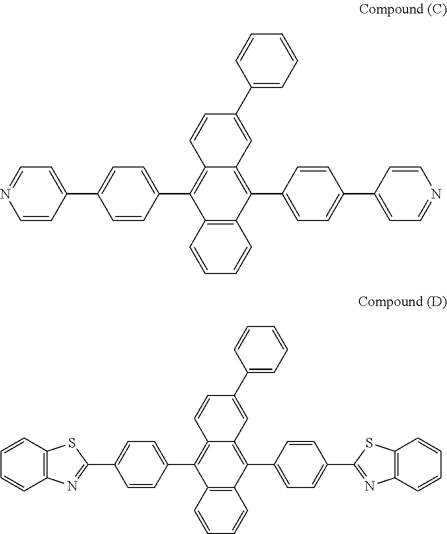 [SMILES: c1ccc(-c2ccc3c(-c4ccc(-c5ccncc5)cc4)c4ccccc4c(-c4ccc(-c5ccncc5)cc4)c3c2)cc1 ; c1ccc(-c2ccc3c(-c4ccc(-c5nc6ccccc6s5)cc4)c4ccccc4c(-c4ccc(-c5nc6ccccc6s5)cc4)c3c2)cc1]

### Example 1

### Device Using Compound (1-3) in Electron Transport Layer

A 26 mm×28 mm×0.7 mm glass substrate (made by Opto Science, Inc.) on which a film of ITO was formed at a thickness of 180 nm by sputtering and was polished to a thickness of 150 nm was applied as a transparent substrate. The transparent substrate was fixed to a substrate holder in commercially available evaporation equipment (made by Showa Shinku Co., Ltd.), and a molybdenum evaporation boat in which HI was put, a molybdenum evaporation boat in which HAT-CN was put, a molybdenum evaporation boat in which NPB was put, a molybdenum evaporation boat in which compound (A) was put, a molybdenum evaporation boat in which compound (B) was put, a molybdenum evaporation boat in which compound (1-3) of the invention was put, a molybdenum evaporation boat in which Liq was put, a molybdenum evaporation boat in which magnesium was put, and a tungsten evaporation boat in which silver was put were mounted.

Each layer described below was sequentially formed on an ITO film on the transparent substrate. A vacuum chamber was decompressed to 5×10⁻⁴ Pa, the evaporation boat in which HI was put was first heated to vacuum-evaporate HI to be 60 nm in thickness, and further the evaporation boat in which HAT-CN was put was heated to vacuum-evaporate HAT-CN to be 10 nm in thickness to forma two-layered hole injection layer, and then the evaporation boat in which NPB was put was heated to vacuum-evaporate NPB to be 10 nm in thickness to form a hole transport layer. Next, the evaporation boat in which compound (A) was put and the evaporation boat in which compound (B) was put were simultaneously heated to vacuum-evaporate compounds (A) and (B) to be 20 nm in thickness to form an emission layer. A vacuum evaporation rate was adjusted to be about 95 to 5 in a weight ratio of compound (A) to compound (B). Next, the evaporation boat in which compound (1-3) was put and the evaporation boat in which Liq was put were simultaneously heated to vacuum-evaporate compound (1-3) and Liq to be 30 nm in thickness to form an electron transport layer. On the above occasion, a vacuum evaporation rate was adjusted to be about 1 to 1 in a weight ratio of compound (1-3) to Liq. The vacuum evaporation rate in each layer was 0.01 to 1 nm/sec.

Then, the evaporation boat in which Liq was put was heated to vacuum-evaporate Liq to be 1 nm in thickness at a vacuum evaporation rate of 0.01 to 1 nm/sec. Subsequently, the evaporation boat in which magnesium was put and the evaporation boat in which silver was put were simultaneously heated to vacuum-evaporate magnesium and silver to be 100 nm in thickness to form cathode, and an organic EL device was obtained. On the above occasion, the vacuum evaporation rate was adjusted between 0.1 to 10 nm/sec to be about 10 to 1 in ratio of the number of atoms of magnesium to silver.

If direct current voltage was applied by using an ITO electrode as an anode and a magnesium/silver electrode as a cathode, and characteristics during emission of 1,000 cd/m² were measured, driving voltage was 3.45 V and external quantum efficiency was 4.75%. Moreover, when a constant current drive test was conducted at current density for obtaining an initial luminance of 2,000 cd/m², time retaining luminance of 90% (1,800 cd/m²) or more of an initial value was 76 hours.

### Comparative Example 1

An organic EL device was obtained in a manner similar to Example 1 except that compound (1-3) was changed to compound (D). If direct current voltage was applied by using an ITO electrode as an anode and a magnesium/silver electrode as a cathode, and characteristics during emission of 1,000 cd/m² were measured, driving voltage was 3.68 V and external quantum efficiency was 4.42%. Moreover, when a constant current drive test was conducted at current density for obtaining an initial luminance of 2,000 cd/m², time retaining luminance (1,800 cd/m²) or more of an initial value was 77 hours.

### Comparative Example 2

An organic EL device was obtained in a manner similar to Example 1 except that compound (1-3) was changed to compound (C). If direct current voltage was applied by using an ITO electrode as an anode and a magnesium/silver electrode as a cathode, and characteristics during emission of 1,000 cd/m² were measured, driving voltage was 3.62 V and external quantum efficiency was 4.28%. Moreover, when a constant current drive test was conducted at current density for obtaining an initial luminance of 2,000 cd/m², time retaining luminance of 90% (1,800 cd/m²) or more of an initial value was 80 hours.

Devices related to Examples 2 to 21 and Comparative Examples 3 and 4 were prepared, and measurement was carried out on drive start voltage (V) and external quantum efficiency (%) in a constant current drive test, respectively. Examples and Comparative Examples will be described in detail below.

Material structure in each layer in each of devices related to Examples 2 to 21 and Comparative Examples 3 and 4 will be shown in Table 2 below.

TABLE 2

| | Hole | Hole | Hole | Emission | | Electron | |
|---|---|---|---|---|---|---|---|
| | injection | injection | transport | layer | | transport | |
| | layer 1 | layer 2 | layer | (20 nm) | | layer* | Cathode |
| | (60 nm) | (10 nm) | (10 nm) | Host | Dopant | (30 nm) | (1 nm/100 nm) |
| Example 2 | HI | HAT-CN | HT | A | B | (1-3) | Liq/Mg + Ag |
| Example 3 | HI | HAT-CN | HT | A | B | (1-4) | Liq/Mg + Ag |
| Example 4 | HI | HAT-CN | HT | A | B | (1-21) | Liq/Mg + Ag |
| Example 5 | HI | HAT-CN | HT | A | B | (1-25) | Liq/Mg + Ag |
| Example 6 | HI | HAT-CN | HT | A | B | (1-29) | Liq/Mg + Ag |
| Example 7 | HI | HAT-CN | HT | A | B | (1-37) | Liq/Mg + Ag |
| Example 8 | HI | HAT-CN | HT | A | B | (1-45) | Liq/Mg + Ag |
| Example 9 | HI | HAT-CN | HT | A | B | (1-53) | Liq/Mg + Ag |
| Example 10 | HI | HAT-CN | HT | A | B | (1-85) | Liq/Mg + Ag |
| Example 11 | HI | HAT-CN | HT | A | B | (1-166) | Liq/Mg + Ag |
| Example 12 | HI | HAT-CN | HT | A | B | (1-274) | Liq/Mg + Ag |
| Example 13 | HI | HAT-CN | HT | A | B | (1-382) | Liq/Mg + Ag |
| Example 14 | HI | HAT-CN | HT | A | B | (1-383) | Liq/Mg + Ag |
| Example 15 | HI | HAT-CN | HT | A | B | (1-404) | Liq/Mg + Ag |
| Example 16 | HI | HAT-CN | HT | A | B | (1-408) | Liq/Mg + Ag |
| Example 17 | HI | HAT-CN | HT | A | B | (1-416) | Liq/Mg + Ag |
| Example 18 | HI | HAT-CN | HT | A | B | (1-424) | Liq/Mg + Ag |
| Example 19 | HI | HAT-CN | HT | A | B | (1-557) | Liq/Mg + Ag |
| Example 20 | HI | HAT-CN | HT | A | B | (1-558) | Liq/Mg + Ag |
| Example 21 | HI | HAT-CN | HT | A | B | (1-611) | Liq/Mg + Ag |
| Comparative | HI | HAT-CN | HT | A | B | D | Liq/Mg + Ag |
| Example 3 | | | | | | | |
| Comparative | HI | HAT-CN | HT | A | B | E | Liq/Mg + Ag |
| Example 4 | | | | | | | |

*The electron transport layer was formed using a mixture of a compound and Liq in the table.

In Table 2, “HT” represents N-([1,1′-biphenyl]-4-yl)-9,9-dimethyl-N-(4-(9-phenyl-9H-carbazole-3-yl)phenyl)-9H-fluorene-2-amine, and compound (E) is 4,4″-bis(benzo[d]thiazole-2-yl)-1,1′:3′,1″-terphenyl. Chemical structure is shown below.

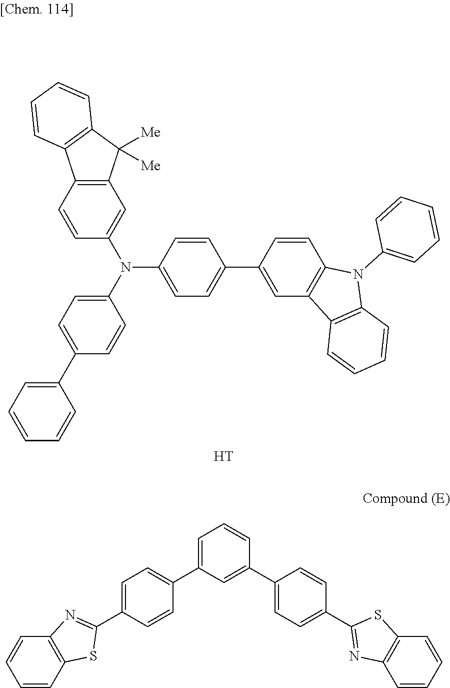

### Example 2

### Part 2 Device Using Compound (1-3) in Electron Transport Layer

A 26 mm×28 mm×0.7 mm glass substrate (made by Opto Science, Inc.) on which a film of ITO was formed at a thickness of 180 nm by sputtering and was polished to a thickness of 150 nm was applied as a transparent substrate. The transparent substrate was fixed to a substrate holder in commercially available evaporation equipment (made by Choshu Industry Co., Ltd.), and a tantalum evaporation boat in which HI was put, a molybdenum evaporation boat in which HAT-CN was put, a tantalum evaporation boat in which HT was put, a molybdenum evaporation boat in which compound (A) was put, a tantalum evaporation crucible in which compound (B) was put, a molybdenum evaporation boat in which compound (1-3) of the invention was put, a tantalum evaporation crucible in which Liq was put, a tantalum evaporation crucible in which magnesium was put, and a tantalum evaporation crucible in which silver was put were mounted.

Each layer described below was sequentially formed on an ITO film on the transparent substrate. A vacuum chamber was decompressed to 2.0×10⁻⁴ Pa, the evaporation boat in which HI was put was heated to vacuum-evaporate HI to be 60 nm in thickness, and the evaporation boat in which HAT-CN was put was further heated to vacuum-evaporate HAT-CN to be 10 nm in thickness to form a two-layered hole injection layer, and subsequently, the evaporation boat in which HT is put was heated to vacuum-evaporate HT to be 10 nm in thickness to form a hole transport layer. Next, the evaporation boat in which compound (A) was put and the evaporation crucible in which compound (B) was put were simultaneously heated to vacuum-evaporate compounds (A) and (B) to be 20 nm in thickness to form an emission layer. A vacuum evaporation rate was adjusted to be about 95 to 5 in a weight ratio of compound (A) to compound (B). Next, the evaporation boat in which compound (1-3) was put and the evaporation crucible in which Liq was put were simultaneously heated to vacuum-evaporate compound (1-3) and Liq to be 30 nm in thickness to form an electron transport layer. On the above occasion, a vacuum evaporation rate was adjusted to be about 1 to 1 in a weight ratio of compound (1-3) to Liq. The vacuum evaporation rate in each layer was 0.01 to 1 nm/sec.

Then, the evaporation crucible in which Liq was put was heated to vacuum-evaporate Liq to be 1 nm in thickness at a vacuum evaporation rate of 0.01 to 1 nm/sec. Subsequently, the evaporation crucible in which magnesium was put and the evaporation crucible in which silver was put were simultaneously heated to vacuum-evaporate magnesium and silver to be 100 nm in thickness to form a cathode, and an organic EL device was obtained. On the above occasion, the vacuum evaporation rate was adjusted between 0.1 to 10 nm/sec to be about 10 to 1 in a ration of the number of atoms of magnesium to silver.

If direct current voltage was applied by using an ITO electrode as an anode, and a magnesium/silver electrode as a cathode, and characteristics during emission of 1,000 cd/m² were measured, driving voltage was 3.41 V and external quantum efficiency was 6.23%.

### Example 3

An organic EL device was obtained in a manner similar to Example 2 except that compound (1-3) was changed to compound (1-4). If direct current voltage was applied by using an ITO electrode as an anode and a magnesium/silver electrode as a cathode, and characteristics during emission of 1,000 cd/m² were measured, driving voltage was 3.38 V and external quantum efficiency was 6.60%.

### Example 4

An organic EL device was obtained in a manner similar to Example 2 except that compound (1-3) was changed to compound (1-21). If direct current voltage was applied by using an ITO electrode as an anode and a magnesium/silver electrode as a cathode, and characteristics during emission of 1,000 cd/m² were measured, driving voltage was 5.24 V and external quantum efficiency was 6.35%.

### Example 5

An organic EL device was obtained in a manner similar to Example 2 except that compound (1-3) was changed to compound (1-25). If direct current voltage was applied by using an ITO electrode as an anode and a magnesium/silver electrode as a cathode, and characteristics during emission of 1,000 cd/m² were measured, driving voltage was 3.73 V and external quantum efficiency was 7.15%.

### Example 6

An organic EL device was obtained in a manner similar to Example 2 except that compound (1-3) was changed to compound (1-29). If direct current voltage was applied by using an ITO electrode as an anode and a magnesium/silver electrode as a cathode, and characteristics during emission of 1,000 cd/m² were measured, driving voltage was 3.79 V and external quantum efficiency was 6.53%.

### Example 7

An organic EL device was obtained in a manner similar to Example 2 except that compound (1-3) was changed to compound (1-37). If direct current voltage was applied by using an ITO electrode as an anode and a magnesium/silver electrode as a cathode, and characteristics during emission of 1,000 cd/m² were measured, driving voltage was 3.49 V and external quantum efficiency was 7.82%.

### Example 8

An organic EL device was obtained in a manner similar to Example 2 except that compound (1-3) was changed to compound (1-45). If direct current voltage was applied by using an ITO electrode as an anode and a magnesium/silver electrode as a cathode, and characteristics during emission of 1,000 cd/m² were measured, driving voltage was 3.83 V and external quantum efficiency was 7.73%.

### Example 9

An organic EL device was obtained in a manner similar to Example 2 except that compound (1-3) was changed to compound (1-53). If direct current voltage was applied by using an ITO electrode as an anode and a magnesium/silver electrode as a cathode, and characteristics during emission of 1,000 cd/m² were measured, driving voltage was 3.85 V and external quantum efficiency was 6.68%.

### Example 10

An organic EL device was obtained in a manner similar to Example 2 except that compound (1-3) was changed to compound (1-85). If direct current voltage was applied by using an ITO electrode as an anode and a magnesium/silver electrode as a cathode, and characteristics during emission of 1,000 cd/m² were measured, driving voltage was 3.34 V and external quantum efficiency was 6.21%.

### Example 11

An organic EL device was obtained in a manner similar to Example 2 except that compound (1-3) was changed to compound (1-166). If direct current voltage was applied by using an ITO electrode as an anode and a magnesium/silver electrode as a cathode, and characteristics during emission of 1,000 cd/m² were measured, driving voltage was 3.53 V and external quantum efficiency was 5.66%.

### Example 12

An organic EL device was obtained in a manner similar to Example 2 except that compound (1-3) was changed to compound (1-274). If direct current voltage was applied by using an ITO electrode as an anode and a magnesium/silver electrode as a cathode, and characteristics during emission of 1,000 cd/m² were measured, driving voltage was 3.48 V and external quantum efficiency was 6.53%.

### Example 13

An organic EL device was obtained in a manner similar to Example 2 except that compound (1-3) was changed to compound (1-382). If direct current voltage was applied by using an ITO electrode as an anode and a magnesium/silver electrode as a cathode, and characteristics during emission of 1,000 cd/m² were measured, driving voltage was 4.35 V and external quantum efficiency was 4.98%.

### Example 14

An organic EL device was obtained in a manner similar to Example 2 except that compound (1-3) was changed to compound (1-383). If direct current voltage was applied by using an ITO electrode as an anode and a magnesium/silver electrode as a cathode, and characteristics during emission of 1,000 cd/m² were measured, driving voltage was 4.65 V and external quantum efficiency was 4.41%.

### Example 15

An organic EL device was obtained in a manner similar to Example 2 except that compound (1-3) was changed to compound (1-404). If direct current voltage was applied by using an ITO electrode as an anode and a magnesium/silver electrode as a cathode, and characteristics during emission of 1,000 cd/m² were measured, driving voltage was 4.21 V and external quantum efficiency was 5.79%.

### Example 16

An organic EL device was obtained in a manner similar to Example 2 except that compound (1-3) was changed to compound (1-408). If direct current voltage was applied by using an ITO electrode as an anode and a magnesium/silver electrode as a cathode, and characteristics during emission of 1,000 cd/m² were measured, voltage was 4.33 V and external quantum efficiency was 5.70%.

### Example 17

An organic EL device was obtained in a manner similar to Example 2 except that compound (1-3) was changed to compound (1-416). If direct current voltage was applied by using an ITO electrode as an anode and a magnesium/silver electrode as a cathode, and characteristics during emission of 1,000 cd/m² were measured, driving voltage was 4.11 V and external quantum efficiency was 6.22%.

### Example 18

An organic EL device was obtained in a manner similar to Example 2 except that compound (1-3) was changed to compound (1-424). If direct current voltage was applied by using an ITO electrode as an anode and a magnesium/silver electrode as a cathode, and characteristics during emission of 1,000 cd/m² were measured, driving voltage was 4.79 V and external quantum efficiency was 5.88%.

### Example 19

An organic EL device was obtained in a manner similar to Example 2 except that compound (1-3) was changed to compound (1-557). If direct current voltage was applied by using an ITO electrode as an anode and a magnesium/silver electrode as a cathode, and characteristics during emission of 1,000 cd/m² were measured, driving voltage was 4.65 V and external quantum efficiency was 6.41%.

### Example 20

An organic EL device was obtained in a manner similar to Example 2 except that compound (1-3) was changed to compound (1-558). If direct current voltage was applied by using an ITO electrode as an anode and a magnesium/silver electrode as a cathode, and characteristics during emission of 1,000 cd/m² were measured, driving voltage was 3.91 V and external quantum efficiency was 5.91%.

### Example 21

An organic EL device was obtained in a manner similar to Example 2 except that compound (1-3) was changed to compound (1-611). If direct current voltage was applied by using an ITO electrode as an anode and a magnesium/silver electrode as a cathode, and characteristics during emission of 1,000 cd/m² were measured, driving voltage was 4.30 V and external quantum efficiency was 5.18%.

### Comparative Example 3

An organic EL device was obtained in a manner similar to Example 2 except that compound (1-3) was changed to compound (D). If direct current voltage was applied by using an ITO electrode as an anode and a magnesium/silver electrode as a cathode, and characteristics during emission of 1,000 cd/m² were measured, driving voltage was 3.64 V and external quantum efficiency was 5.40%.

### Comparative Example 4

An organic EL device was obtained in a manner similar to Example 2 except that compound (1-3) was changed to compound (E). If direct current voltage was applied by using an ITO electrode as an anode and a magnesium/silver electrode as a cathode, and characteristics during emission of 1,000 cd/m² were measured, driving voltage was 8.59 V and external quantum efficiency was 0.46%.

### INDUSTRIAL APPLICABILITY

According to a preferred aspect of the invention, an organic EL device can be provided in which characteristics required for the organic EL device, such as low driving voltage, high efficiency and long service life, above all, high efficiency, are attained, can provide a high performance display apparatus, such as a full-color display.

1. A compound represented by formula (1) below: 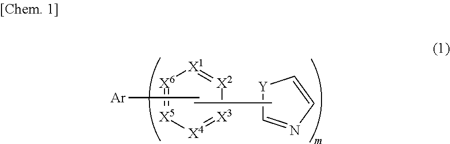
wherein, in formula (1),
Ar is an m-valent group derived from aromatic hydrocarbon having 6 to 40 carbons or an m-valent group derived from an aromatic heterocycle having 2 to 40 carbons, and at least one of hydrogen in the group may be replaced by alkyl having 1 to 12 carbons or cycloalkyl having 3 to 12 carbons;
X¹ to X⁶ are independently ═CR¹— or ═N—, at least two of X¹ to X⁶ is ═CR¹—, R¹ in two of ═CR¹— in X¹ to X⁶ is a bonding hand to be bonded with Ar or an azole ring, and R¹ in ═CR¹— other than the above is hydrogen or alkyl having 1 to 4 carbons;
Y is independently —O— or —S—;
at least one of hydrogen in an azole ring may be replaced by alkyl having 1 to 4 carbons, phenyl or naphthyl;
m is an integer from 2 to 4, and a group formed by an azole ring and a six-membered ring may be identical or different; and
at least one of hydrogen in each ring and alkyl in the formula may be replaced by deuterium.

2. The compound according to claim 1, wherein Ar is at least one selected from the group of groups represented by formulas (Ar-1) to (Ar-22) below: 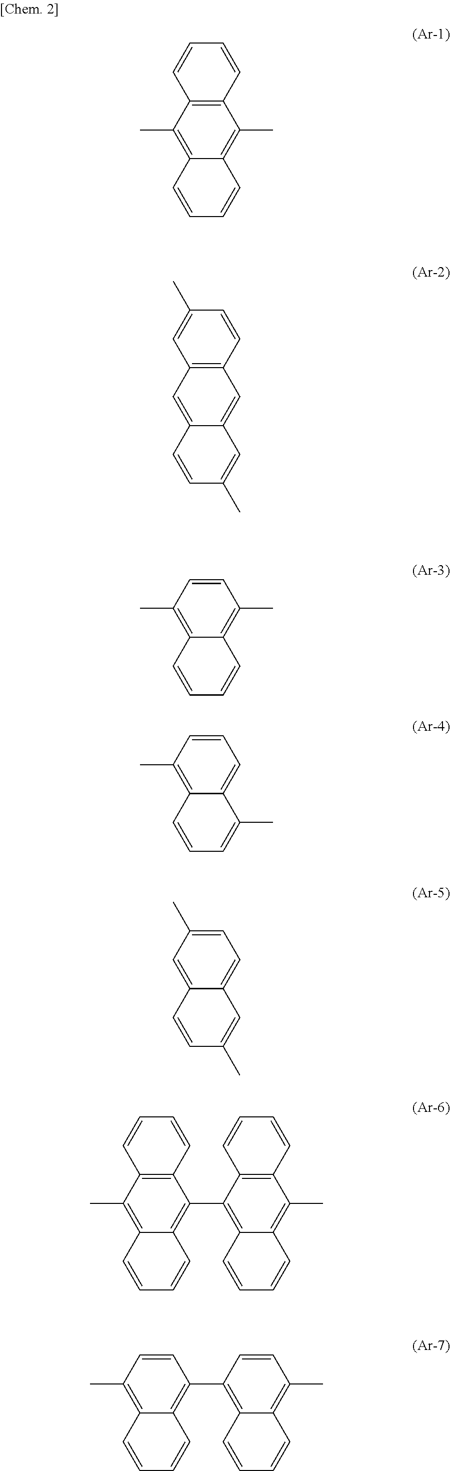 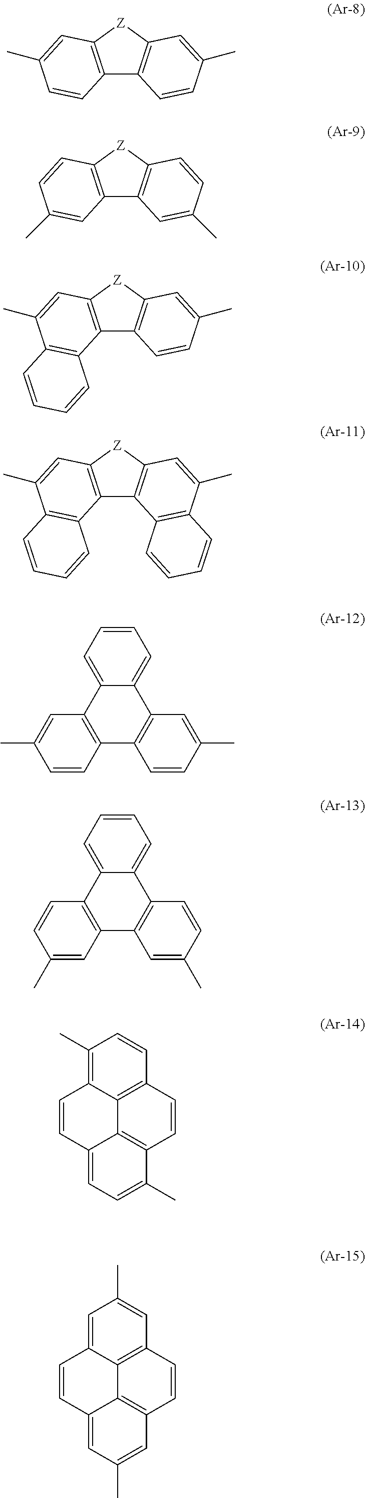 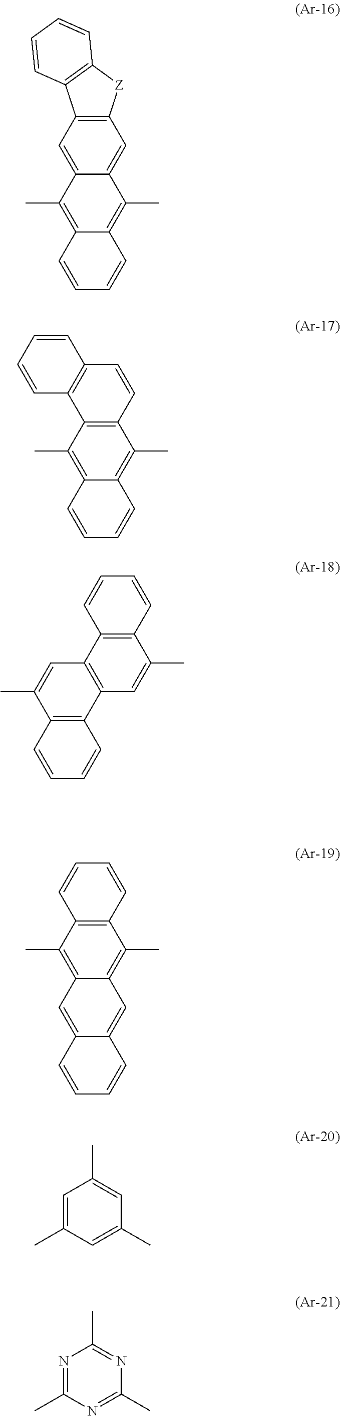 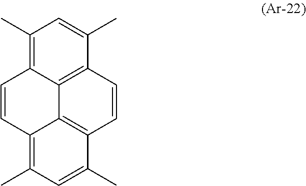
wherein, in formulas (Ar-1) to (Ar-22), Z is independently —O—, —S— or a divalent group represented by formula (2) or (3) below, and at least one of hydrogen in each group may be replaced by alkyl having 1 to 12 carbons, cycloalkyl having 3 to 12 carbons or aryl having 6 to 24 carbons; 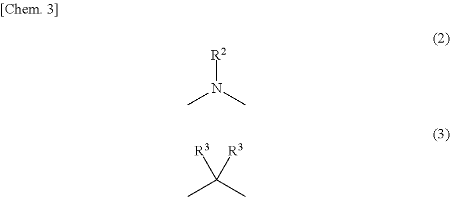
wherein, in formula (2), R² is phenyl, naphthyl, biphenylyl or terphenylyl, and in formula (3), R³ is independently methyl or phenyl, and two of R³ may be linked with each other to form a ring.

3. The compound according to claim 1, wherein Ar is one selected from the group of groups represented by formulas (Ar-1) to (Ar-13) 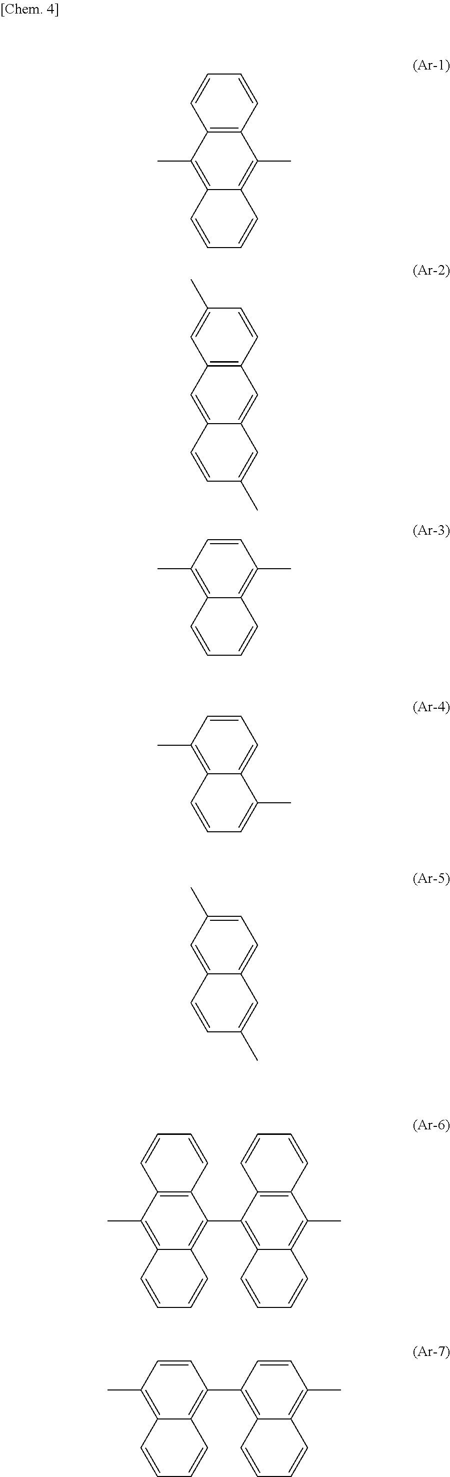 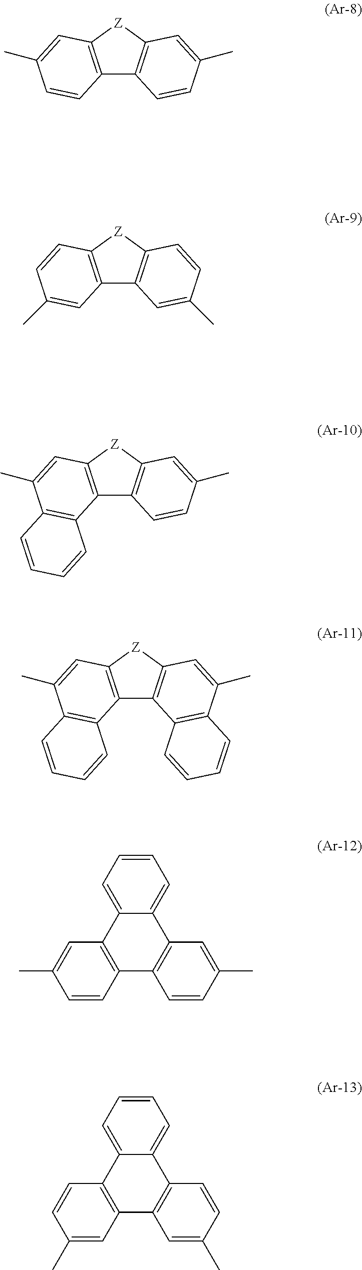
wherein, in formulas (Ar-1) to (Ar-13), Z is independently —O—, —S— or a divalent group represented by formula (2) or (3) below, and at least one of hydrogen in each group may be replaced by alkyl having 1 to 12 carbons, cycloalkyl having 3 to 12 carbons or aryl having 6 to 24 carbons; 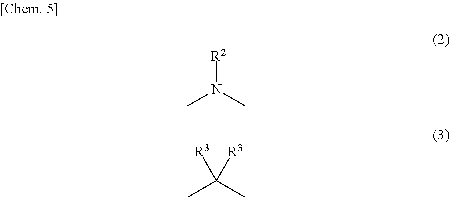
wherein, in formula (2), R² is phenyl, naphthyl, biphenylyl or terphenylyl, and in formula (3), R³ is independently methyl or phenyl, and two of R³ may be linked with each other to form a ring.

4. The compound according to claim 1, represented by formula (1-3) below:

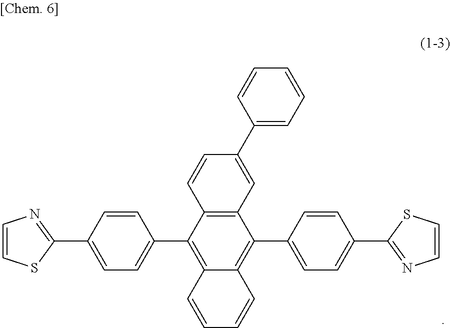 [SMILES: c1ccc(-c2ccc3c(-c4ccc(-c5nccs5)cc4)c4ccccc4c(-c4ccc(-c5nccs5)cc4)c3c2)cc1]

5. The compound according to claim 1, represented by one selected from formulas (1-4), (1-21), (1-25), (1-29), (1-37), (1-45), (1-53) and (1-85) below:

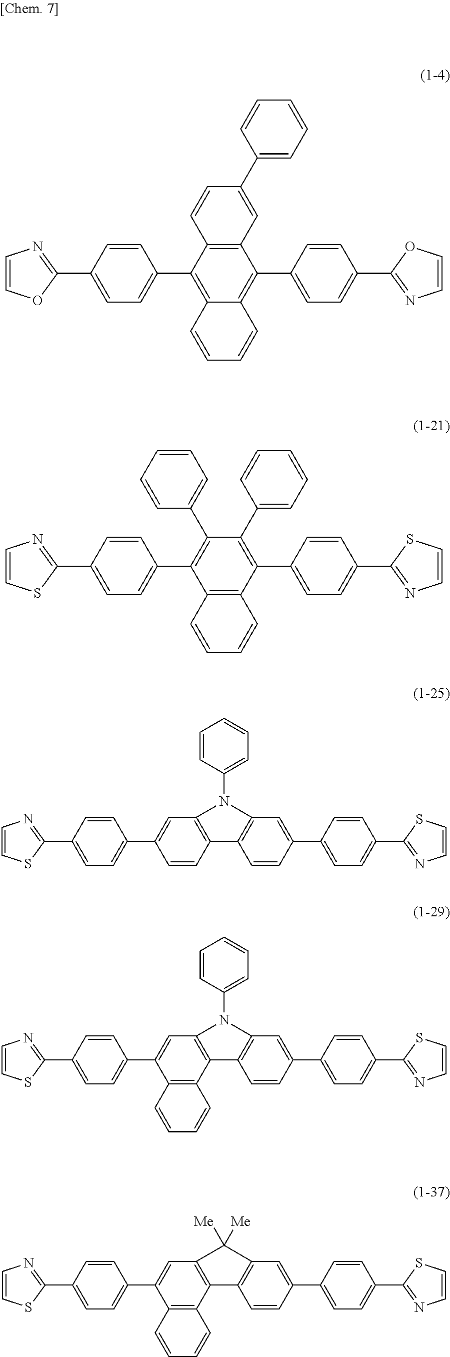 [SMILES: c1ccc(-c2ccc3c(-c4ccc(-c5ncco5)cc4)c4ccccc4c(-c4ccc(-c5ncco5)cc4)c3c2)cc1 ; c1ccc(-c2c(-c3ccccc3)c(-c3ccc(-c4nccs4)cc3)c3ccccc3c2-c2ccc(-c3nccs3)cc2)cc1 ; c1ccc(-n2c3cc(-c4ccc(-c5nccs5)cc4)ccc3c3ccc(-c4ccc(-c5nccs5)cc4)cc32)cc1 ; c1ccc(-n2c3cc(-c4ccc(-c5nccs5)cc4)ccc3c3c4ccccc4c(-c4ccc(-c5nccs5)cc4)cc32)cc1 ; CC1(C)c2cc(-c3ccc(-c4nccs4)cc3)ccc2-c2c1cc(-c1ccc(-c3nccs3)cc1)c1ccccc21]

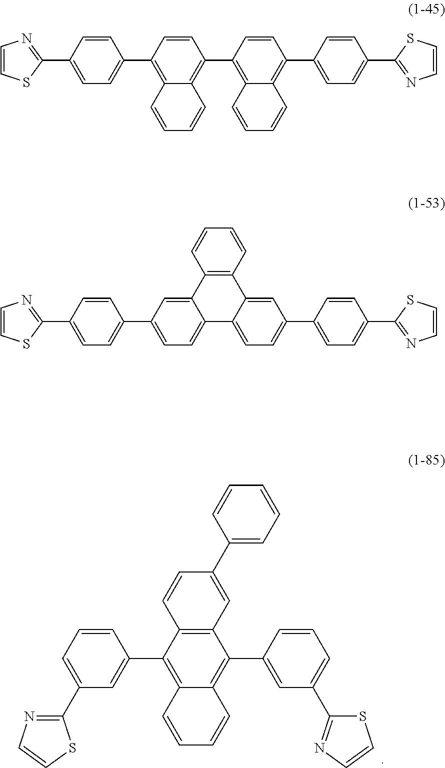 [SMILES: c1ccc2c(-c3ccc(-c4ccc(-c5nccs5)cc4)c4ccccc34)ccc(-c3ccc(-c4nccs4)cc3)c2c1 ; c1ccc2c(c1)c1cc(-c3ccc(-c4nccs4)cc3)ccc1c1ccc(-c3ccc(-c4nccs4)cc3)cc21 ; c1ccc(-c2ccc3c(-c4cccc(-c5nccs5)c4)c4ccccc4c(-c4cccc(-c5nccs5)c4)c3c2)cc1]

6. The compound according to claim 1, represented by formula (1-166) or (1-274) below:

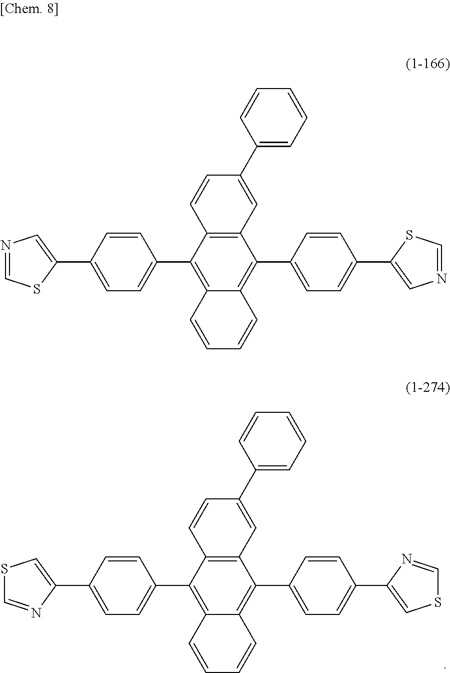 [SMILES: c1ccc(-c2ccc3c(-c4ccc(-c5cncs5)cc4)c4ccccc4c(-c4ccc(-c5cncs5)cc4)c3c2)cc1 ; c1ccc(-c2ccc3c(-c4ccc(-c5cscn5)cc4)c4ccccc4c(-c4ccc(-c5cscn5)cc4)c3c2)cc1]

7. The compound according to claim 1, represented by one selected from formulas (1-382), (1-383), (1-404), (1-408), (1-416), (1-424), (1-557), (1-558) and (1-611) below:

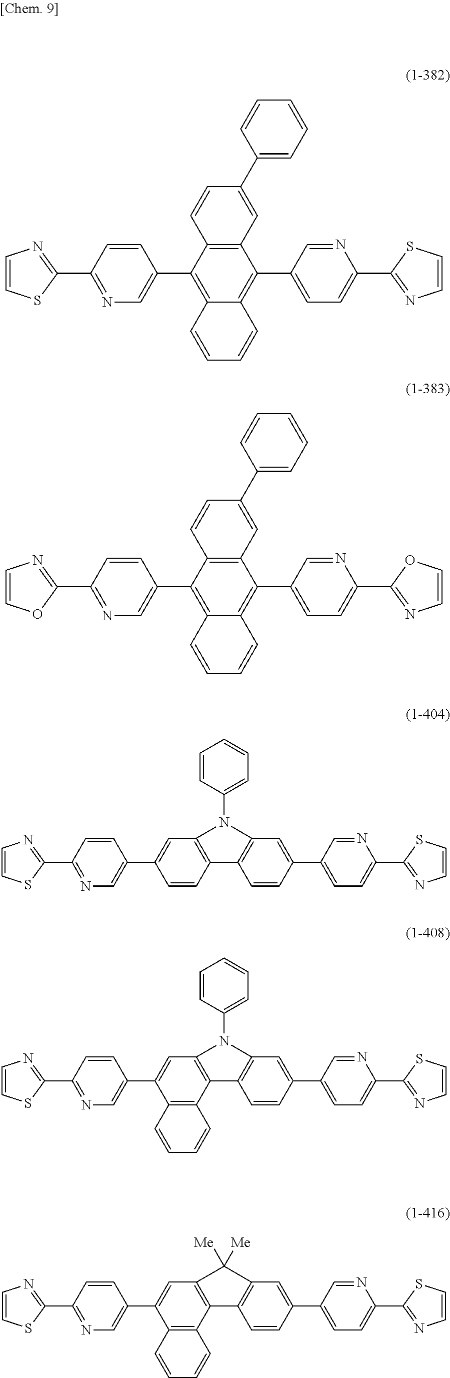 [SMILES: c1ccc(-c2ccc3c(-c4ccc(-c5nccs5)nc4)c4ccccc4c(-c4ccc(-c5nccs5)nc4)c3c2)cc1 ; c1ccc(-c2ccc3c(-c4ccc(-c5ncco5)nc4)c4ccccc4c(-c4ccc(-c5ncco5)nc4)c3c2)cc1 ; c1ccc(-n2c3cc(-c4ccc(-c5nccs5)nc4)ccc3c3ccc(-c4ccc(-c5nccs5)nc4)cc32)cc1 ; c1ccc(-n2c3cc(-c4ccc(-c5nccs5)nc4)ccc3c3c4ccccc4c(-c4ccc(-c5nccs5)nc4)cc32)cc1 ; CC1(C)c2cc(-c3ccc(-c4nccs4)nc3)ccc2-c2c1cc(-c1ccc(-c3nccs3)nc1)c1ccccc21]

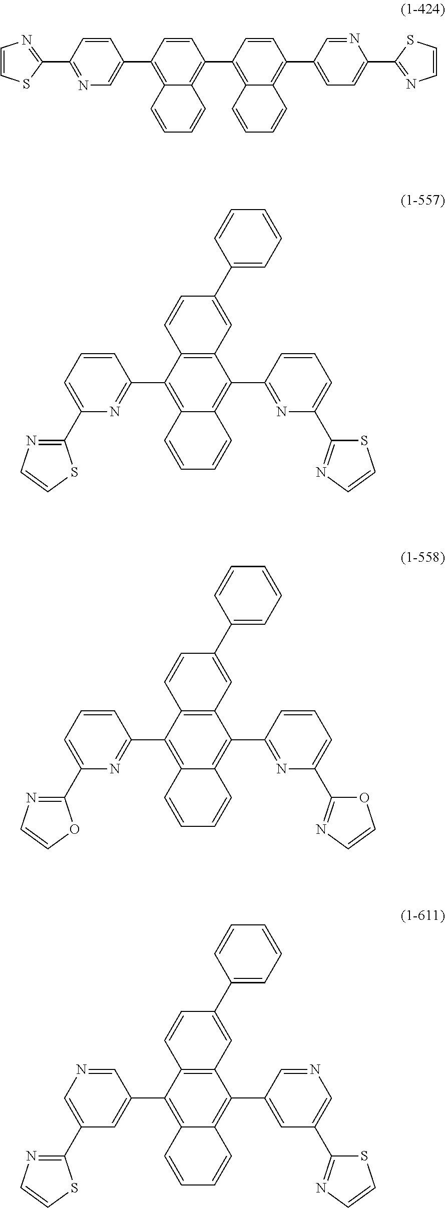 [SMILES: c1ccc2c(-c3ccc(-c4ccc(-c5nccs5)nc4)c4ccccc34)ccc(-c3ccc(-c4nccs4)nc3)c2c1 ; c1ccc(-c2ccc3c(-c4cccc(-c5nccs5)n4)c4ccccc4c(-c4cccc(-c5nccs5)n4)c3c2)cc1 ; c1ccc(-c2ccc3c(-c4cccc(-c5ncco5)n4)c4ccccc4c(-c4cccc(-c5ncco5)n4)c3c2)cc1 ; c1ccc(-c2ccc3c(-c4cncc(-c5nccs5)c4)c4ccccc4c(-c4cncc(-c5nccs5)c4)c3c2)cc1]

8. An electron transport material, containing the compound according to claim 1.

9. An organic electroluminescent device, having a pair of electrodes formed of an anode and a cathode, an emission layer arranged between the pair of electrodes, and an electron transport layer and/or an electron injection layer, containing the electron transport material according to claim 8, and arranged between the cathode and the emission layer.

10. The organic electroluminescent device according to claim 9, wherein at least one of the electron transport layer and the electron injection layer further contains at least one selected from the group of a quinolinol metal complex, a bipyridine derivative, a phenanthroline derivative and a borane derivative.

11. The organic electroluminescent device according to claim 9, wherein at least one of the electron transport layer and the electron injection layer further contains at least one selected from the group of alkali metal, alkaline earth metal, rare earth metal, alkali metal oxide, alkali metal halide, alkaline earth metal oxide, alkaline earth metal halide, rare earth metal oxide, rare earth metal halide, an organic complex of alkali metal, an organic complex of alkaline earth metal and an organic complex of rare earth metal.
